(19) United States
(12) Patent Application Publication
(10) Pub. No.: US 2024/0101627 A1
(43) Pub. Date: Mar. 28, 2024

(54) MHC CLASS II T-CELL MODULATORY POLYPEPTIDES FOR TREATING TYPE 1 DIABETES MELLITUS (T1D) AND METHODS OF USE THEREOF

(71) Applicant: Cue Biopharma, Inc., Boston, MA (US)

(72) Inventors: Ronald D. Seidel, III, Boston, MA (US); Rodolfo J. Chaparro, Cambridge, MA (US); Chee Meng Low, Boston, MA (US)

(21) Appl. No.: 18/375,848

(22) Filed: Oct. 2, 2023

Related U.S. Application Data
(63) Continuation of application No. PCT/US22/25547, filed on Apr. 20, 2022.
(60) Provisional application No. 63/177,641, filed on Apr. 21, 2021.

(51) Int. Cl. C07K 14/495; C07K 14/55; C07K 14/62; C07K 14/71; C07K 14/74; C07K 16/22; C12N 9/88

(52) U.S. Cl. CPC C07K 14/495; C07K 14/55; C07K 14/62; C07K 14/70539; C07K 14/71; C07K 16/22; C12N 9/88; C12Y 401/01015; A61K 38/00

(57) ABSTRACT

The present disclosure provides T-cell modulatory polypeptides (TMPs) comprising a type 1 diabetes (T1D)-associated peptide epitope, MHC class II polypeptides, one or more immunomodulatory polypeptides, a TGF-β polypeptide, and a masking polypeptide. A TMP of the present disclosure is useful for modulating activity of a T cell. Thus, the present disclosure provides compositions and methods for modulating the activity of T cells, as well as compositions and methods for treating persons who have T1D.

### CROSS-REFERENCE

This application claims the benefit of U.S. Provisional Patent Application No. 63/177,641, filed Apr. 21, 2021, which application is incorporated herein by reference in its entirety.

### INCORPORATION BY REFERENCE OF SEQUENCE LISTING PROVIDED AS A TEXT FILE

A Sequence Listing is provided herewith as a text file, “CUEB-142WO_SEQ_LIST_ST25.txt” created on Mar. 25, 2022 and having a size of 498 KB. The contents of the text file are incorporated by reference herein in their entirety.

### INTRODUCTION

Central to the proper functioning of the mammalian immune system are the coordinated activities and communications between two specialized cell types, antigen-presenting cells (“APCs”) and T cells. APCs serve to capture and break the proteins from foreign organisms, or abnormal proteins (e.g., from genetic mutation in cancer cells), into smaller fragments suitable as signals for scrutiny by the larger immune system, including T cells. In particular, APCs break down proteins into small peptide fragments, which are then paired with proteins of the major histocompatibility complex (“MHC”) and displayed on the cell surface. Cell surface display of an MHC together with a peptide fragment, also known as a T cell epitope, provides the underlying scaffold surveilled by T cells, allowing for specific recognition. The peptide fragments can be pathogen-derived, tumor-derived, or derived from natural host proteins (self-proteins). Moreover. APCs can recognize other foreign components, such as bacterial toxins, viral proteins, viral DNA, viral RNA, etc., whose presence denotes an escalated threat level. The APCs relay this information to T cells through additional costimulatory signals in order to generate a more effective response.

T cells recognize peptide-major histocompatibility complex (“pMHC”) complexes through a specialized cell surface receptor, the T cell receptor (“TCR”). The TCR is unique to each T cell; as a consequence, each T cell is highly specific for a particular pMHC target. In order to adequately address the universe of potential threats, a very large number (˜10,000,000) of distinct T cells with distinct TCRs exist in the human body. Further, any given T cell, specific for a particular T cell peptide, is initially a very small fraction of the total T cell population. Although normally dormant and in limited numbers. T cells bearing specific TCRs can be readily activated and amplified by APCs to generate highly potent T cell responses that involve many millions of T cells. Such activated T cell responses are capable of attacking and clearing viral infections, bacterial infections, and other cellular threats including tumors, as illustrated below. Conversely, the broad, non-specific activation of overly active T cell responses against self or shared antigens can give rise to T cells inappropriately attacking and destroying healthy tissues or cells.

### SUMMARY

The present disclosure provides T-cell modulatory polypeptides (TMPs) comprising a type I diabetes (T1D)-associated peptide epitope, MHC class II polypeptides, one or more immunomodulatory polypeptides, a TGF-β polypeptide, and a masking polypeptide. A TMP of the present disclosure is useful for modulating activity of a T cell. Thus, the present disclosure provides compositions and methods for modulating the activity of T cells, as well as compositions and methods for treating persons who have T1D.

### BRIEF DESCRIPTION OF THE DRAWINGS

FIG. 1A-1H present schematic depictions of TMPs of the present disclosure.

FIG. 2A-2D present schematic depictions of TMPs of the present disclosure, in which the immunomodulatory polypeptide is in Position 2.

FIG. 3A-3D present schematic depictions of TMPs of the present disclosure, in which the immunomodulatory polypeptide is in Position 3.

FIG. 4 provides an amino acid sequence of an HLA Class II DRA (sometimes referred to as DRA1) α chains (SEQ ID NO:1).

FIG. 5 provides amino acid sequences of HLA Class II DRB1β chains.

FIG. 6 provides amino acid sequences of HLA Class II DRB3β chains (from top to bottom SEQ ID NOs:37-40).

FIG. 7 provides an amino acid sequence of an HLA Class II DRB4 β chain (SEQ ID NOs:41-42).

FIG. 8 provides an amino acid sequence of an HLA Class II DRB5 β chain (SEQ ID NO:43).

FIG. 9 provides an amino acid sequence of an HLA Class II DMA α chain (SEQ ID NO:44).

FIG. 10 provides an amino acid sequence of an HLA Class II DMB β chain (SEQ ID NO:45).

FIG. 11 provides an amino acid sequence of an HLA Class II DOA α chain (SEQ ID NO:46).

FIG. 12 provides an amino acid sequence of an HLA Class II DOB β chain (SEQ ID NO:47).

FIG. 13 provides amino acid sequences of HLA Class II DPA1 α chains (SEQ ID NOs:48-49).

FIG. 14 provides amino acid sequences of HLA Class II DPB1 β chains (from top to bottom SEQ ID NOs:50-61).

FIG. 15 provides amino acid sequences of HLA Class II DQA1 α chains (from top to bottom SEQ ID NOs:62-72).

FIG. 16 provides an amino acid sequence of an HLA Class II DQA2 α chain (SEQ ID NO:73).

FIG. 17 provides amino acid sequences of HLA Class II DQB1 β chains (from top to bottom SEQ ID NOs:74-85).

FIG. 18A-18B provide amino acid sequences of HLA Class II DQB2 β chains (SEQ ID NO:86 and SEQ ID NO:87, respectively).

FIG. 19A-190 provide amino acid sequences of wild-type (FIG. 19A) and variant (FIG. 19B-9N) DRA*0101 α chains.

FIG. 20A-20J provide amino acid sequences of wild-type (FIG. 20A) and variant (FIG. 20B-20J) DRB1*0401 β chains.

FIG. 21A-21M provide amino acid sequences of immunoglobulin Fc polypeptides.

FIG. 22A-22F provide amino acid sequences of a wild-type IL-2 polypeptide (FIG. 22A), IL-2Rα (FIG. 22B), IL-2Rβ (FIG. 22C), IL-2Rγ (FIG. 22D), a PD-L1 ectodomain polypeptide (FIG. 22E), and a 4-1BBL polypeptide (FIG. 22F).

FIG. 23A-23G provides amino acid sequences of three different isoforms of TGF-β preproproteins (FIG. 23A, FIG. 23C, and FIG. 23E), TGF-β mature form proteins (FIG. 23B, FIG. 23D, and FIG. 23F), and mature form of TGF-β3 with a C77S substitution (FIG. 23G).

FIG. 24 provides an alignment of amino acid sequences of TGF-β isoforms 1-3 with the residues corresponding to the mature form of TGF-β2 bolded, except aa residues Lys 25, Cys 77. Ile 92, and Lys 94 of TGF-β2 and their corresponding residues in the other forms of TGF-β isoforms 1 and 3 that are underlined and italicized but not bolded.

FIG. 25A-25N provide amino acid sequences of a type 1 TGF-β receptor (TβRI) (FIG. 25A) and its ectodomain (FIG. 25B); a type 2 TGF-β receptor (TβRII) (FIG. 25C and FIG. 25E), its ectodomain (FIG. 25D and FIG. 25F), and fragments of the ectodomain (FIG. 25G-25J); and a type 3 TGF-β receptor (TORI) (FIG. 25K and FIG. 25M) and its ectodomain (FIG. 25L and FIG. 25N).

FIG. 26A-26B provide amino acid sequences of an exemplary TMP (construct 4415-4417).

FIG. 27A-27B provide amino acid sequences of an exemplary TMP (construct 4418-4420).

FIG. 28 provides a schematic depiction of a TMP comprising scaffold polypeptides with interspecific binding sequences.

FIG. 29A-29B provide amino acid sequences of a TMP lacking MODs (construct 4415-4416).

FIG. 30A-30B provide amino acid sequences of a TMP lacking MODs (construct 4418-4419).

FIG. 31A-31B provide amino acid sequences of a TMP lacking MODs (construct 3858-3859).

FIG. 32A-32B provide amino acid sequences of an exemplary TMP (construct 3858-3869).

FIG. 33A-33B provide amino acid sequences of an exemplary TMP (construct 3870-3871).

FIG. 34A-34B provide a schematic depiction of certain TMPs (FIG. 34A) and depict SDS-PAGE analysis of the TMPs.

FIG. 35 depicts the effect of certain TMP, on the proportion of cells, in a population of T cells, that are FoxP3 positive.

FIG. 36 depicts SDS-PAGE analysis of certain TMPs.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA. DNA-RNA hybrids, or a polymer comprising purine and pyrimidine bases or other natural, chemically or biochemically modified, non-natural, or derivatized nucleotide bases.

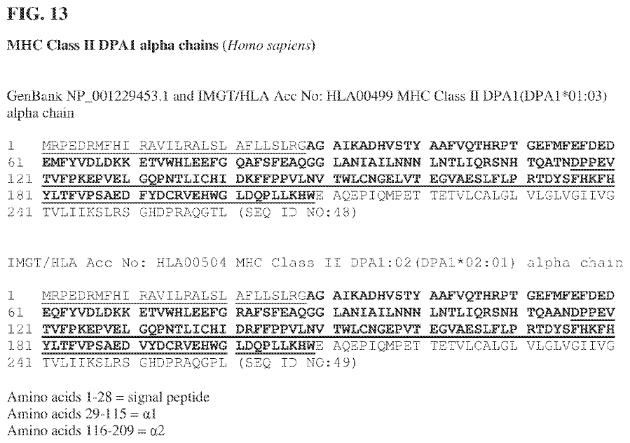

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References herein to a specific residue or residue number in a known polypeptide. e.g., position 72 or 75 of human DRA MHC class II polypeptide, are understood to refer to the amino acid at that position in the wild-type polypeptide (i.e. 172 or K75). To the extent that the sequence of the wild-type polypeptide is altered, either by addition or deletion of one or more amino acids, one of ordinary skill will understand that a reference to the specific residue or residue number will be correspondingly altered so as to refer to the same specific amino acid in the altered polypeptide, which would be understood to reside at an altered position number. For example, if a human DRA MHC class II polypeptide is altered by the addition of one amino acid at the N-terminus, then a reference to position 72 or 75, or to residue 172 or K75, will be understood to indicate the amino acids that are at positions 73 or 76, or to residues 173 and K76. Likewise, a reference herein to substitution of a specific amino acid as a specific position. e.g., 172C. is understood to refer to a substitution of a cysteine for the amino acid at position 72 in the wild-type polypeptide, i.e., isoleucine. If, e.g., the wild-type polypeptide is altered to change the amino acid at position 72 from isoleucine to an alternate amino acid, then the reference to 172C will be understood to refer to the substitution of a cysteine for the alternate amino acid. If in such case the polypeptide is also altered by the addition or deletion of one or more amino acids, then the reference to 172C will be understood to refer to the substitution of a cysteine for the alternate amino acid at the altered position number. A reference to a “non-naturally occurring Cys residue” in a polypeptide, e.g., a DRA MHC class II polypeptide, means that the polypeptide comprises a Cys residue in a location where there is no Cys in the corresponding wild-type polypeptide. This can be accomplished through routine protein engineering in which a cysteine is substituted for the amino acid that occurs in the wild-type sequence, e.g., at position 72 or 75 of the DRA*0101 polypeptide (see FIGS. 13G and H) instead of the isoleucine (I) or lysine (K) residues that are present in the wild-type DRA*0101 polypeptide (sec FIG. 13A).

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990) J. Mol. Biol. 215:403-10. Unless otherwise stated. “sequence identity” as referred to herein is determined by BLAST (Basic Local Alignment Search Tool), as described in Altschul et al. (1990) J. Mol. Biol. 215:403.

As used herein “masked” means that a molecule (e.g., masked polypeptide or masked protein) is bound or otherwise engaged by a masking molecule (e.g., polypeptide, protein or protein fragment) that limits the availability of the masked molecule to other proteins (e.g., cell surface receptors) that also have affinity for the molecule.

Interspecific binding sequences are dimerization sequences that permit an asymmetric paring of polypeptides (heterodimer formation). Interspecific binding sequences favor formation of heterodimers with their counterpart interspecific binding sequence(s) (as opposed to forming homodimers), which is their cognate binding partner. Key-in-hole (or key-into-hole) Fc polypeptide pairs are an example of an interspecific binding sequence and its counterpart interspecific binding sequence.

“Tandem,” as used herein to describe the placement of MOD polypeptides, means having two or more MODs arranged adjacent to each other on a polypeptide separated, at most, by a linker.

As used herein the term “in vivo” refers to any process or procedure occurring inside of the body. e.g., of a T1D patient.

As used herein, “in vitro” refers to any process or procedure occurring outside of the body.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

The term “binding,” as used herein (e.g. with reference to binding of a T-cell modulatory antigen-presenting polypeptide to a polypeptide (e.g., a T-cell receptor) on a T cell), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. “Covalent binding” or “covalent bond,” as used herein, refers to the formation of one or more covalent chemical binds between two different molecules.

The term “immunological synapse” or “immune synapse” as used herein generally refers to the natural interface between two interacting immune cells of an adaptive immune response including, e.g., the interface between an antigen-presenting cell (APC) or target cell and an effector cell, e.g., a lymphocyte, an effector T cell, a natural killer cell, and the like. An immunological synapse between an APC and a T cell is generally initiated by the interaction of a T cell antigen receptor and major histocompatibility complex molecules, e.g., as described in Bromley et al., Annu Rev Immunol. 2001; 19:375-96; the disclosure of which is incorporated herein by reference in its entirety.

“T cell” includes all types of immune cells expressing CD3, including T-helper cells (CD4⁺ cells), cytotoxic T-cells (CD8⁺ cells), T-regulatory cells (Treg), and NK-T cells.

The term “immunomodulatory polypeptide” (also referred to as a “MOD”), as used herein, includes a polypeptide on an antigen presenting cell (APC) (e.g., a dendritic cell, a B cell, and the like), or a portion of the polypeptide on an APC, that specifically binds a cognate co-immunomodulatory polypeptide on a T cell, thereby providing a signal which, in addition to the primary signal provided by, for instance, binding of a TCR/CD3 complex with a major histocompatibility complex (MHC polypeptide loaded with peptide, mediates a T cell response, including, but not limited to, proliferation, activation, differentiation, and the like.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “affinity” refers to the equilibrium constant for the reversible binding of two agents (e.g., an antibody and an antigen) and is expressed as a dissociation constant (K_{D}). As used herein, the term “avidity” refers to the resistance of a complex of two or more agents to dissociation after dilution.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom. i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient.” are used interchangeably herein and refer to any mammalian subject for whom diagnosis, treatment, or therapy is desired. Mammals include. e.g., humans, non-human primates, rodents (e.g., rats; mice), lagomorphs (e.g., rabbits), ungulates (e.g., cows, sheep, pigs, horses, goats, and the like), etc.

Unless indicated otherwise, the term “substantially” is intended to encompass both “wholly” and “largely but not wholly”. For example, an Ig Fc that “substantially does not induce cell lysis” means an Ig Fc that induces no cell lysis at all or that largely but not wholly induces no cell lysis.

As used herein, the term “about” used in connection with an amount indicates that the amount can vary by 10%. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The terms “purifying”, “isolating”, and the like, refer to the removal of a desired substance. e.g., a TMP, from a solution containing undesired substances, e.g., contaminates, or the removal of undesired substances from a solution containing a desired substance, leaving behind essentially only the desired substance. In some instances, a purified substance may be essentially free of other substances. e.g., contaminates.

It is to be understood that this disclosure is not limited to particular embodiments described, as such may, of course, vary. It is also to be understood that the terminology used herein is for the purpose of describing particular embodiments only, and is not intended to be limiting, since the scope of the disclosure described in the appended claims will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an.” and “the” include plural referents unless the context clearly dictates otherwise. Thus, for example, reference to “an immunomodulatory polypeptide” includes a plurality of such immunomodulatory polypeptides and reference to “the Treg” includes reference to one or more Tregs and equivalents thereof known to those skilled in the art, and so forth. It is further noted that the claims may be drafted to exclude any optional element or any element that is included in a list or other recitation of elements that share a common generic or specific feature, property, or activity. As such, this statement is intended to serve as antecedent basis for use of such exclusive terminology as “solely,” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed. The headings of this disclosure are for convenience of reference only and do not define, describe or limit the scope or this disclosure or the scope of the appended claims.

### DETAILED DESCRIPTION

The present disclosure provides T-cell modulatory polypeptides (TMPs) comprising a type 1 diabetes (TD)-associated peptide epitope, MHC class II polypeptides, one or more optional immunomodulatory polypeptides (“MODs”), a TGF-β polypeptide, and a masking polypeptide (the TGF-β polypeptide and masking polypeptide together comprising a “masked TGF-β MOD”). A TMP of the present disclosure is useful for modulating activity of a T cell. Thus, the present disclosure provides compositions and methods for modulating the activity of T cells, as well as compositions and methods for treating individuals who have T1D. In any of the embodiments described herein, in some cases, a TMP does not include a MOD; such a TMP is useful for delivering TGFβ.

### T-Cell Modulatory Polypeptides (“TMPs”)

The present disclosure provides TMPs comprising: i) a peptide that displays a Type 1 Diabetes-associated epitope capable of being bound by a T-cell receptor (a “T1D peptide”); ii) a TGF-β polypeptide; iii) a masking polypeptide; iv) MHC class II polypeptides (i.e., an MHC class II α chain polypeptide and an MHC class II β chain polypeptide); and v) optionally, one or more MODs; where the TMP optionally comprises one or more scaffold polypeptides (e.g., one or more immunoglobulin (Ig) Fc polypeptides), and where the TMP optionally comprises one or more independently selected linker polypeptides (e.g., where each of the one or more independently selected linker polypeptides is between any two of the aforementioned polypeptides. TMPs can be in the form of single-chain polypeptides, heterodimeric polypeptides, and dimers or multimers of such single chain and heterodimeric polypeptides, and exemplary configurations are discussed below.

Further, although the TMPs of this disclosure may comprise both one or more masked TGF-β MODs and one or more additional MODs such as a wt or variant IL-2, PD-L1 and/or a 4-1BBL MOD (as discussed above), if desired, the TMPs of this disclosure may comprise only one or more masked TGF-β MODs. That is, the one or more additional MODs such as the wt or variant IL-2. PD-L1 and/or a 4-1BBL MOD need not be included in a TMP of this disclosure. In the discussion below, therefore, it should be understood that the presence of one or more MODs in addition to the one or more masked TGF-β MODs is optional.

### Brief Overview of Exemplary TMP Structures

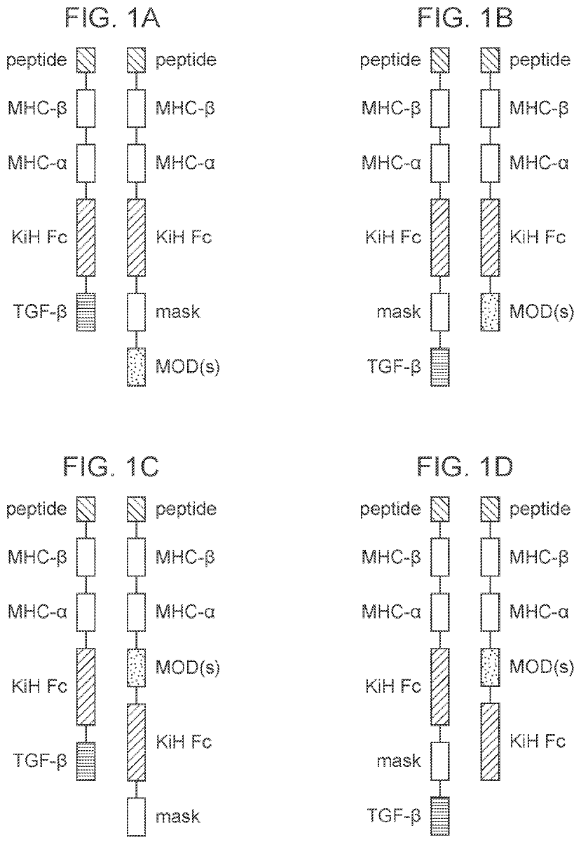

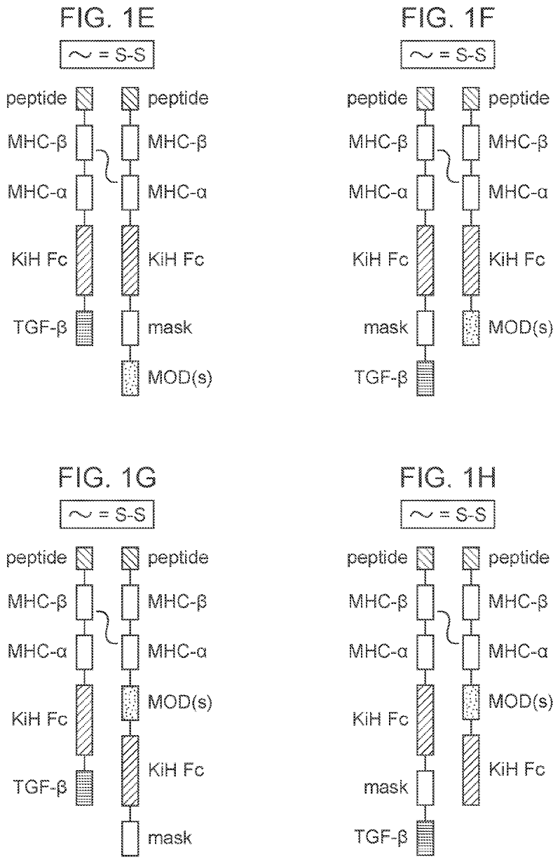

A) A TMP of the present disclosure in some cases comprises a heterodimer comprising scaffold polypeptides (e.g., Ig Fc) with interspecific binding sequences, where the heterodimer comprises: a) a first polypeptide comprising: i) a T1D peptide; ii) an MHC class II D polypeptide; iii) an MHC class II α polypeptide and iv) a first scaffold polypeptide comprising a first interspecific binding sequence; and b) a second polypeptide comprising: i) a T1D peptide; ii) an MHC class II D polypeptide; iii) an MHC class II α polypeptide and iv) a second scaffold polypeptide comprising a counterpart interspecific binding sequence to the interspecific binding sequence of the first polypeptide, wherein the interspecific binding sequence and the counterpart interspecific binding sequence interact with each other in the heterodimer, where the first or the second polypeptide comprises a TGF-β polypeptide, where the first or the second polypeptide comprises a masking polypeptide, and where the first and/or the second polypeptide optionally comprises one or more MODs; and where the TMP optionally comprises one or more independently selected linker polypeptides. Examples of such TMPs are depicted schematically in FIG. 1A-1D. In some cases, as depicted schematically in FIG. 1E-1H, the first and the second polypeptides are linked by a disulfide bond between. e.g., the MHC class II β polypeptide and the MHC class II α polypeptide.

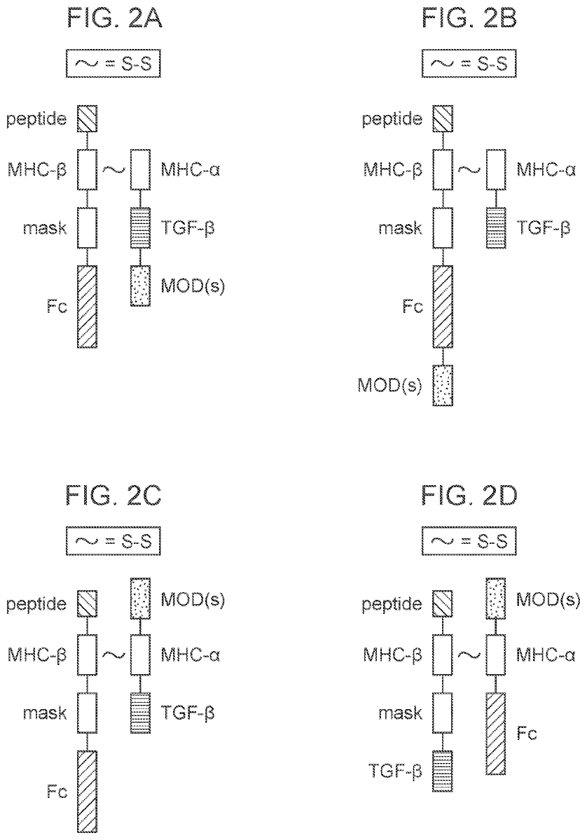

B) A TMP of the present disclosure in some cases comprises one or more heterodimers, where each heterodimer comprises: a) a first polypeptide chain comprising: i) a T1D peptide and a first MHC class II polypeptide; and b) a second polypeptide comprising a second MHC class II polypeptide, where the first and/or the second polypeptide optionally comprises one or more MODs, where the first or the second polypeptide comprises a TGF-β polypeptide, and where the first or the second polypeptide comprises a masking polypeptide; wherein the TMP optionally includes one or more independently selected linker polypeptides, and where the first or the second polypeptide chain comprises an Ig Fc polypeptide. A TIP of the present disclosure can be a homodimer comprising two such heterodimers, where the Ig Fc polypeptide of one of the heterodimers is disulfide linked to the Ig Fc polypeptide of the other heterodimer. Examples of such TMPs are depicted schematically in FIG. 2A-2D.

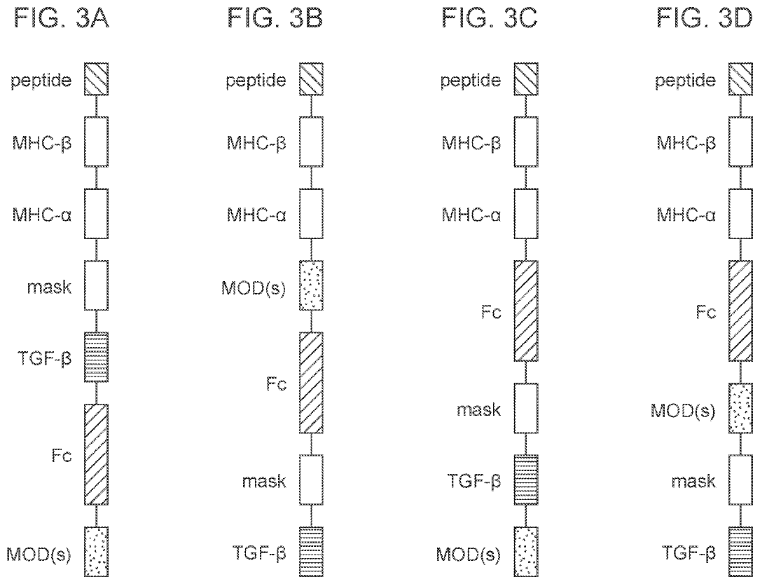

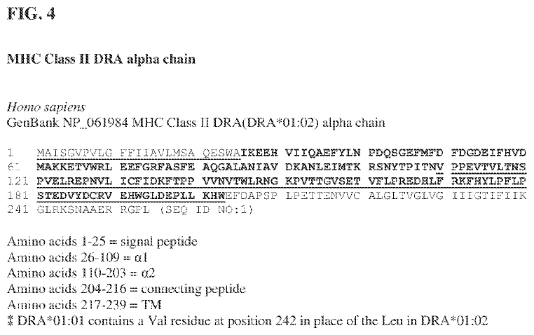

C) A TMP of the present disclosure in some cases is a single-chain polypeptide; i.e., a single polypeptide chain comprises: i) a T1D peptide; ii) MHC class II polypeptides (e.g., an MHC class II β polypeptide and an MHC class II α polypeptide); iii) optionally one or more MODS; iv) a TGF-β polypeptide; v) a masking polypeptide; and, optionally, one or more independently selected linker polypeptides, and, optionally, an Ig Fc polypeptide. Examples of such TMPs are depicted schematically in FIG. 3A-3D. A TMP of the present disclosure can be a homodimer comprising two copies of such single-chain polypeptides, where the single-chain polypeptide comprises an Ig Fc polypeptide, and where the Ig Fc polypeptide of one of the two single-chain polypeptides is disulfide linked to the Ig Fc polypeptide of the other of the two single-chain polypeptides. Alternatively, a TMP can comprise a heterodimer of two different single-chain TMPs that are joined by polypeptides comprising interspecific binding sequences discussed below.

The foregoing configurations are discussed in more detail below. References herein to MHC class II α polypeptides may include both the α1 and α2 domains of class II MHC α chains, and references herein to MHC class II β polypeptides may include both the β1 and β2 domains of class II MHC D chains, which four domains represent all or most of the extracellular class II protein required for presentation of an epitope peptide.

A) Heterodimeric TMPs Comprising Interspecific Binding Sequences

A TMP of the present disclosure in some cases comprises a heterodimer comprising scaffold polypeptides (e.g., Ig Fc polypeptides) with interspecific binding sequences, where the heterodimer comprises: a) a first polypeptide comprising: i) a T1D peptide; ii) an MHC class II polypeptide; iii) an MHC class II α polypeptide; and iv) a first scaffold polypeptide comprising a first interspecific binding sequence; and b) a second polypeptide comprising: i) a T1D peptide; ii) an MHC class II β polypeptide; iii) an MHC class II α polypeptide; and iv) a second scaffold polypeptide comprising a counterpart interspecific binding sequence to the interspecific binding sequence of the first polypeptide, wherein the interspecific binding sequence and the counterpart interspecific binding sequence interact with each other in the heterodimer, where the first or the second polypeptide comprises a TGF-β polypeptide, where the first or the second polypeptide comprises a masking polypeptide, and where the first and/or the second polypeptide optionally comprises one or more MODs. The TMP optionally comprises one or more independently selected linker polypeptides. Non-limiting examples of such TMPs are depicted schematically in FIGS. 1A-1D. Although FIGS. 1A-1D illustrate the scaffold polypeptides as Knob-in-Hole (“KiH”) Fc polypeptides, it should be understood that other scaffold polypeptides having interspecific binding sequences may be used.

Arrangement of Components

The components of such a TMP can be arranged in various ways. In some cases, a TMP comprises at least one heterodimer, where the heterodimer comprises: a) a first polypeptide comprising: i) a T1D peptide; ii) an MHC class II β polypeptide; iii) an MHC class II α polypeptide; iv) a first scaffold polypeptide comprising a first interspecific binding sequence; and v) a TGF-β polypeptide; and b) a second polypeptide comprising: i) a T1D peptide; ii) an MHC class II β polypeptide; iii) an MHC class II α polypeptide; iv) a second scaffold polypeptide comprising a counterpart interspecific binding sequence to the interspecific binding sequence of the first polypeptide, wherein the interspecific binding sequence and the counterpart interspecific binding sequence interact with each other in the heterodimer; and v) a masking polypeptide; where the first and/or the second polypeptide comprises one or more MODs. In some cases, the one or more MODs and the TGF-β polypeptide are on the same polypeptide. In some cases, the one or more MODs and the masking polypeptide are on the same polypeptide. In some cases, the interspecific binding sequence and the counterpart interspecific binding sequence comprise KiH sequences.

In some cases, a TMP comprises at least one heterodimer, where the heterodimer comprises: a) a first polypeptide comprising: i) a T1D peptide; ii) an MHC class II β polypeptide; iii) an MHC class II α polypeptide; iv) a first scaffold polypeptide comprising a first interspecific binding sequence; v) a masking polypeptide; and vi) a TGF-β polypeptide; and b) a second polypeptide comprising: i) a T1D peptide; ii) an MHC class II β polypeptide; iii) an MHC class II α polypeptide; and iv) a second scaffold polypeptide comprising a counterpart interspecific binding sequence to the interspecific binding sequence of the first polypeptide, wherein the interspecific binding sequence and the counterpart interspecific binding sequence interact with each other in the heterodimer; where the first and/or the second polypeptide comprises one or more MODs. In some cases, the one or more MODs are on the first polypeptide. In some cases, the one or more MODs are on the second polypeptide. In some cases, the interspecific binding sequence and the counterpart interspecific binding sequence comprise KiH sequences.

Further examples of various arrangements of the components of a TMP are depicted schematically in FIGS. 1A-1D. In FIGS. 1A-1D, the scaffold polypeptides are KiH Ig Fc polypeptides, and the masking polypeptide is referred to as “mask.” For example, as depicted schematically in FIG. 1A, in some cases, a TMP is a heterodimer comprising: a) a first polypeptide comprising, in order from N-terminus to C-terminus: i) a T1D peptide; ii) an MHC class II β chain polypeptide; iii) an MHC class II α chain polypeptide; iv) a first Ig Fc comprising an interspecific binding sequence (illustrated as a KiH sequence); and v) a TGF-β polypeptide; and h) a second polypeptide comprising, in order from N-terminus to C-terminus: i) a T1D peptide; ii) an MHC class II β chain polypeptide; iii) an MHC class II α chain polypeptide; iv) a second Ig Fc comprising an interspecific binding sequence (illustrated as a KiH sequence); v) a masking polypeptide; and vi) one or more MODs, where the first Ig Fc polypeptide dimerizes with the second Ig Fc polypeptide, and where the TMP comprises an optional peptide linker between any two of the components (any two adjacent components) of the TMP.

As another example, as depicted schematically in FIG. 1B, in some cases, a TMP is a heterodimer comprising: a) a first polypeptide comprising, in order from N-terminus to C-terminus: i) a T1D peptide; ii) an MHC class II chain polypeptide; iii) an MHC class II α chain polypeptide; iv) a first Ig Fc comprising an interspecific binding sequence (illustrated as a KiH sequence); v) a masking polypeptide; and vi) a TGF-β polypeptide; and b) a second polypeptide comprising, in order from N-terminus to C-terminus: i) a T1D peptide; ii) an MHC class II β chain polypeptide; iii) an MHC class II α chain polypeptide; iv) a second Ig Fc comprising an interspecific binding sequence (illustrated as a KiH sequence); and v) one or more MODs, where the first Ig Fc polypeptide dimerizes with the second Ig Fc polypeptide, and where the TMP comprises an optional peptide linker between any two of the components (any two adjacent components) of the TMP.

As another example, as depicted schematically in FIG. 1C, in some cases, a TMP is a heterodimer comprising: a) a first polypeptide comprising, in order from N-terminus to C-terminus: i) a T1D peptide; ii) an MHC class II β chain polypeptide; iii) an MHC class II α chain polypeptide; iv) a first Ig Fc comprising an interspecific binding sequence (illustrated as a KiH sequence); and v) a TGF-β polypeptide; and b) a second polypeptide comprising, in order from N-terminus to C-terminus: i) a T1D peptide; ii) an MHC class II β chain polypeptide; iii) an MHC class II α chain polypeptide; iv) one or more MODs; v) a second Ig Fc comprising an interspecific binding sequence (illustrated as a KiH sequence); and vi) a masking polypeptide, where the first Ig Fc polypeptide dimerizes with the second Ig Fc polypeptide, and where the TMP comprises an optional peptide linker between any two of the components (any two adjacent components) of the TMP.

As another example, as depicted schematically in FIG. 1D, in some cases, a TMP is a heterodimer comprising: a) a first polypeptide comprising, in order from N-terminus to C-terminus: i) a T1D peptide; ii) an MHC class II β chain polypeptide; iii) an MHC class II α chain polypeptide; iv) a first Ig Fc comprising an interspecific binding sequence (illustrated as a KiH sequence); v) a masking polypeptide; and vi) a TGF-β polypeptide, and b) a second polypeptide comprising, in order from N-terminus to C-terminus: i) a T1D peptide; ii) an MHC class II β chain polypeptide; iii) an MHC class II α chain polypeptide; iv) one or more MODs; and v) a second Ig Fc comprising an interspecific binding sequence (illustrated as a KiH sequence), where the first Ig Fc polypeptide dimerizes with the second Ig Fc polypeptide, and where the TMP comprises an optional peptide linker between any two of the components (any two adjacent components) of the TMP.

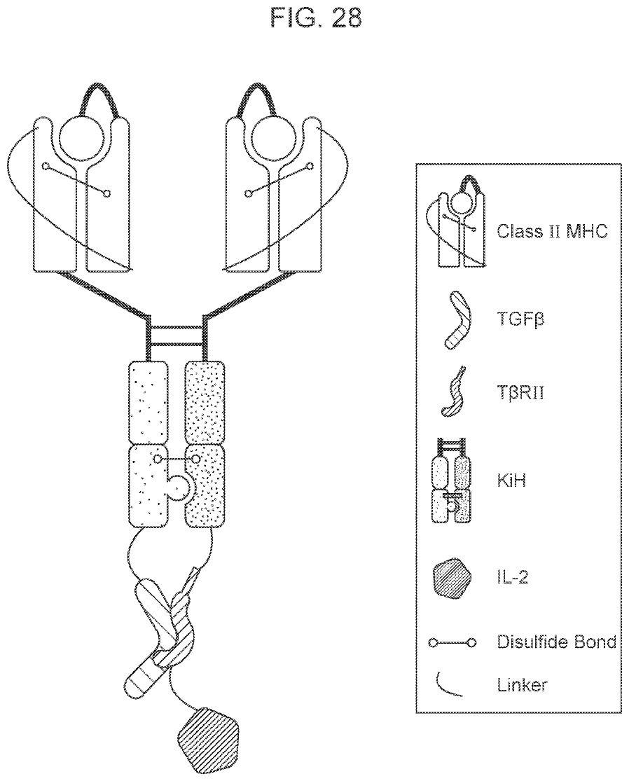

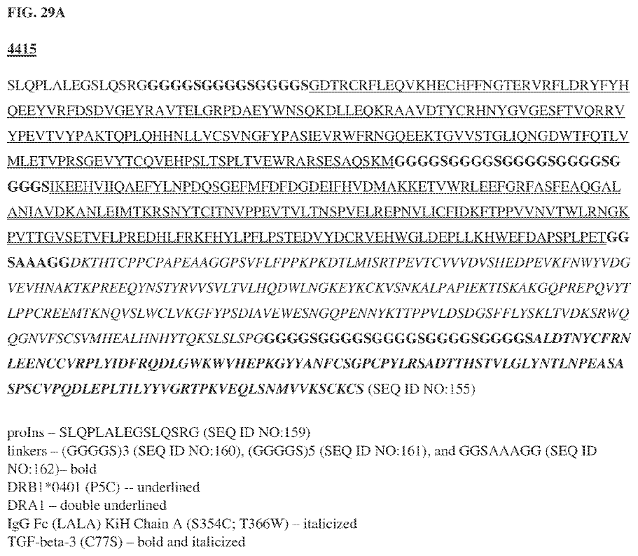

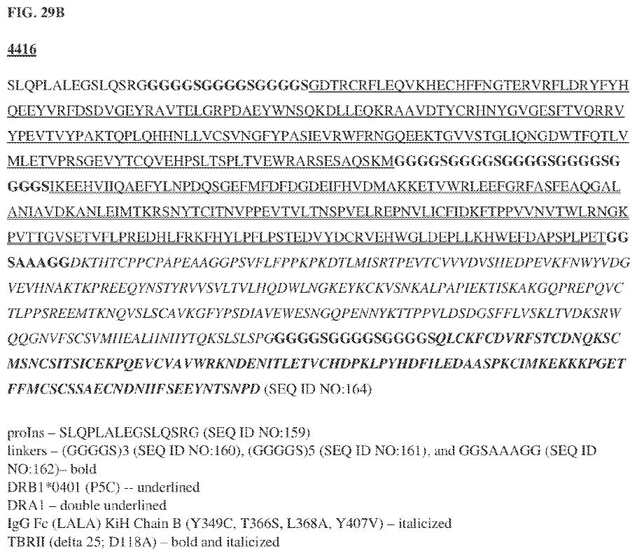

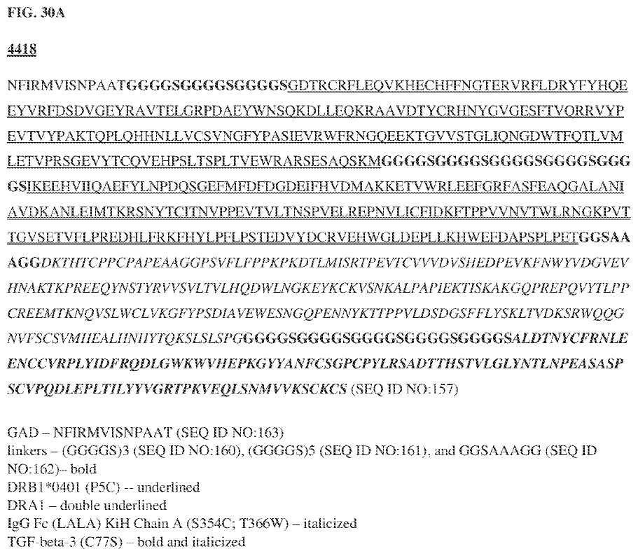

In some cases, as depicted schematically in FIG. 1E-1H, in some cases, the MHC class II α chain polypeptide and the MHC class II β chain polypeptide comprise Cys residues not present in naturally-occurring MHC class II α chain and MHC class II β chain polypeptides, where a disulfide bond is formed between the Cys residues, thereby disulfide linking the first polypeptide to the second polypeptide. Suitable MHC class II α chain and MHC class II β chain polypeptides comprising introduced Cys residues are described below. In addition, where the scaffold proteins are Ig Fc polypeptides, one or more disulfide bonds can form between an Ig Fc polypeptide in the first polypeptide and an Ig Fc polypeptide in the second polypeptide. Such disulfide bonds are depicted schematically in FIG. 28.

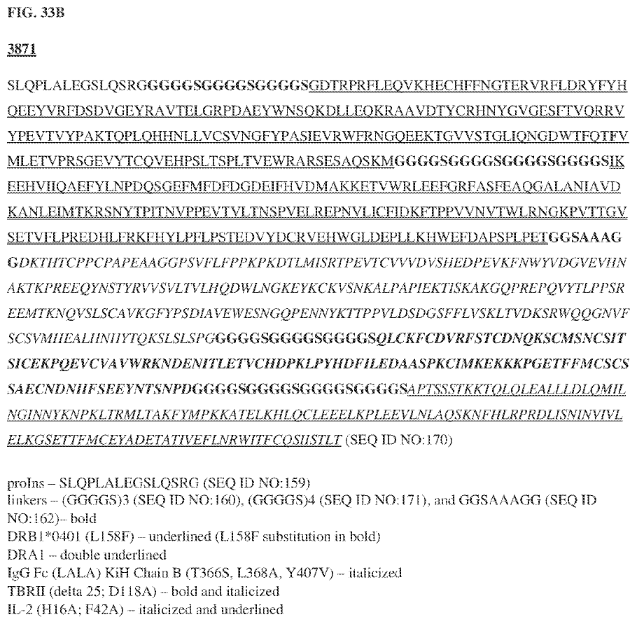

Amino acid sequences of exemplary TMPs that are heterodimers comprising Ig Fc polypeptides with interspecific binding sequences are depicted in 1) FIG. 26A and FIG. 26B, where the first polypeptide is “construct 4415” (FIG. 26A) and the second polypeptide is “construct 4417” (FIG. 26B); 2) FIG. 27A and FIG. 27B, where the first polypeptide is “construct 4418” (FIG. 27A) and the second polypeptide is “construct 4420” (FIG. 27B); 3) FIG. 32A-32B, where the first polypeptide is “construct 3858” (FIG. 32A) and the second polypeptide is “construct 3869” (FIG. 32B); and 4) FIG. 33A-33B, where the first polypeptide is “construct 3870” (FIG. 33A) and the second polypeptide is “construct 3871” FIG. 33B).

TMPs without Additional MODs

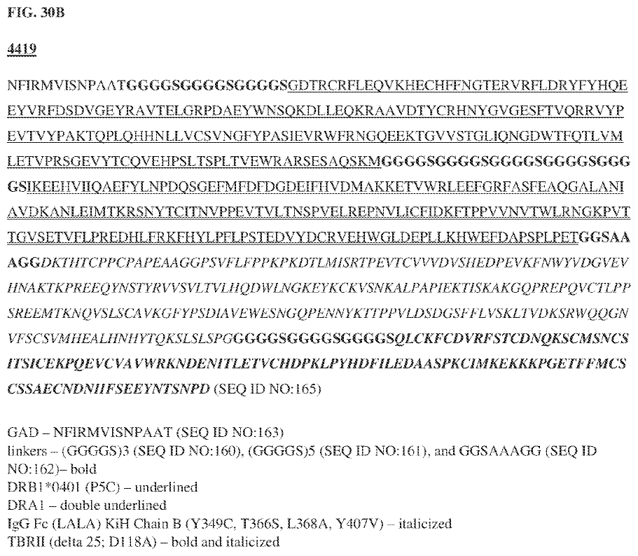

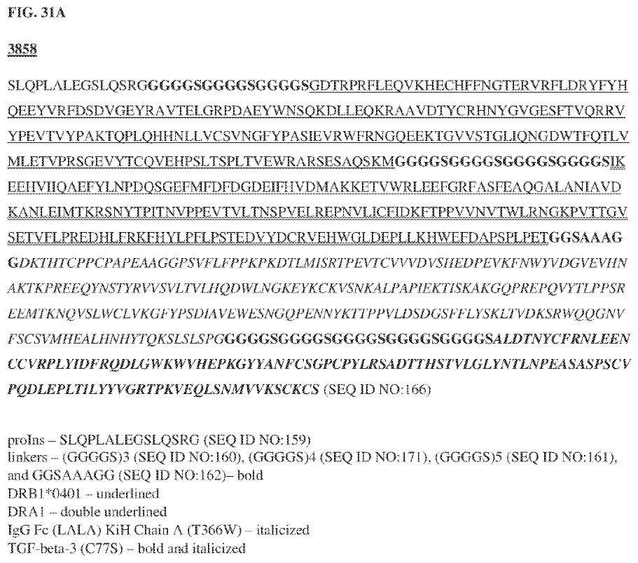

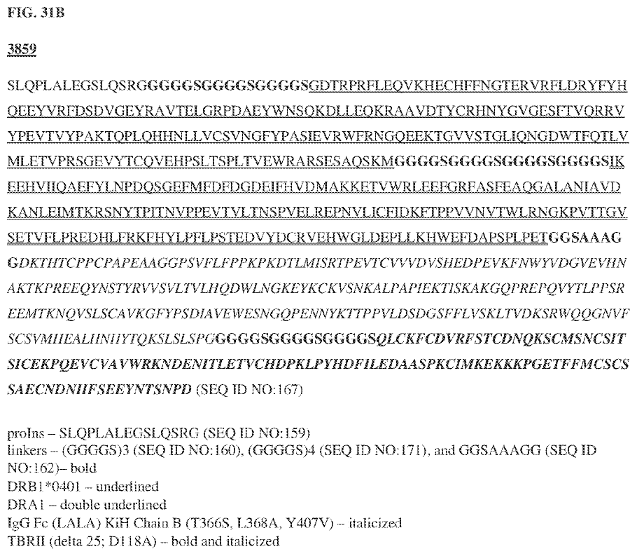

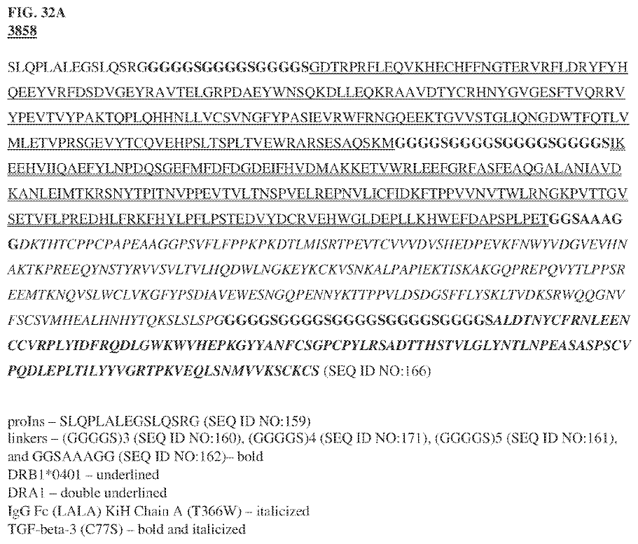

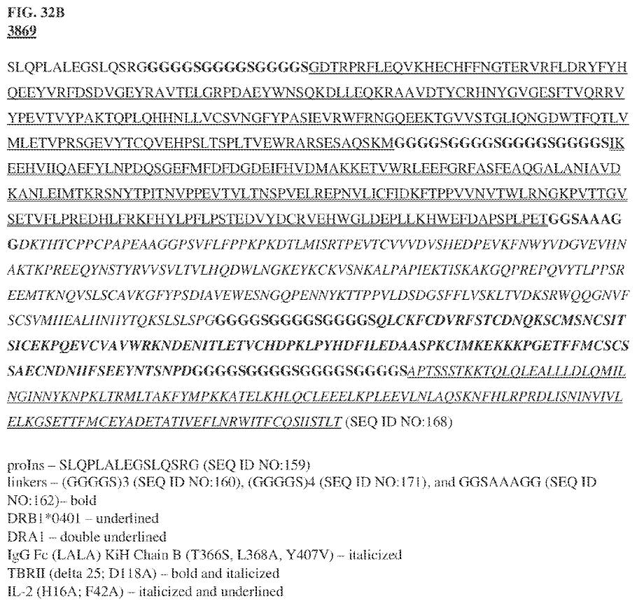

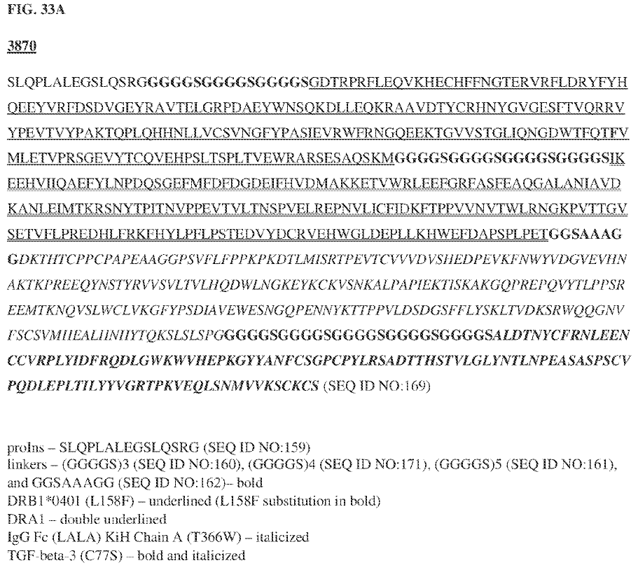

As noted above, any of the above-described embodiments can be provided without a MOD other than a masked TGF-β MOD. Non-limiting examples of such TMPs are depicted in: 1) FIG. 29A29B, where the first polypeptide is “construct 4415” (FIG. 29A) and the second polypeptide is “construct 4416” (FIG. 29B); 2) FIG. 30A-30B, where the first polypeptide is “construct 4418” (FIG. 30A) and the second polypeptide is “construct 4419” FIG. 30B); and 3) FIG. 31A-31B, where the first polypeptide is “construct 3858” (FIG. 31A) and the second polypeptide is “construct 3859” (FIG. 31B). Such TMPs are useful for, e.g., delivering TGFβ selectively to T cells expressing on their surface a TCR specific for the peptide present in the TMP, when complexed with MHC class II polypeptides present in the TMP.

Scaffold Polypeptides with Interspecific Binding Sequences

Scaffold polypeptides serve, among other things, as structural elements providing a framework upon which other components of a TMP are organized. Where the masking polypeptide and the TGF-β polypeptide are located in trans (on different polypeptides of the TMP), scaffolds polypeptides that form interspecific and non-interspecific duplexes (or higher order structures) can keep the masking polypeptide associated with the TGF-β polypeptide. Depending on the nature of the scaffold, it can also act as an organizational element providing higher order structure in terms of protein folding and dimerization or multimerization (e.g., homodimerization or heterodimerization). The scaffold can also contribute to serum stability, particularly where it is an Ig heavy chain constant region (e.g., an Ig Fc). Suitable scaffold polypeptides will, in some cases, be half-life extending polypeptides. In some cases, a suitable scaffold polypeptide increases the in vivo half-life (e.g., the serum half-life) of a TMP, compared to a control TMP having a scaffold polypeptide with a different, non-Ig sequence, by at least about 10%, at least about 15%, at least about 25%, at least about 50%, at least about 2-fold, at least about 5-fold, at least about 10-fold, at least about 25-fold, at least about 50-fold, at least about 100-fold, or more than 100-fold. As an example, in some cases an Ig Fc polypeptide sequence (e.g., including interspecific Ig sequence such as a KiH sequence pairs) increases the stability and/or in vivo half-life (e.g., the serum half-life) of a TMP, compared to a control TMP having the Ig Fc polypeptide sequence replaced by a linker (e.g., a GGGS as repeat of equal sequence length; SEQ ID NO:234). The increase in in vivo half-life can be by at least about 10%, at least about 15%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. Where an Ig Fc polypeptide is employed in a TMP, the Ig Fc can contain mutations that will prevent the spontaneous formation of dimers of the TMP (See. e.g., Tianlei Ying et al., J. Biol. Chem., 287 (23), pp 19399.19408 (Jun. 1, 2012)), and additionally may include mutations (e.g., the LALA mutations discussed below) that reduce or substantially eliminate the ability of the Ig polypeptide to induce cell lysis, e.g., though complement-dependent cytotoxicity (CDC) and/or antibody-dependent cellular cytotoxicity (ADCC).

Where scaffold polypeptides of a TMP comprise one or more amino acid sequences that permit a scaffold polypeptide to interact (specifically bind) with another scaffold polypeptide, the TMP can form a homodimer. A TMP may also comprise one or more as sequences that permit scaffold polypeptides to interact (specifically bind) with other scaffold polypeptides, forming higher order structures. Sequences that form higher order multimer structures permit the formation of TMPs of higher order (e.g., trimers, tetramers, pentamers etc.). By way of example, scaffold polypeptides comprising IgM Fc regions permit formation of pentameric (particularly when J-chain sequences are also expressed) or hexameric TMPs. Petrus̆ić et al., Med Hypotheses. 77(6):959-61 (2011).

A variety of polypeptides that specifically bind to each other or themselves with sufficient affinity may be utilized as dimerization sequences in a TMP (see, e.g., U.S. Patent Publication No. 2003/0138440). The polypeptides may be of relatively compact size (e.g., such as less than about 300, 250, 225, 200, 175, 150, 125, 100, 75, or 50 aa). Dimerization/multimerization polypeptides include, but are not limited to: immunoglobulin heavy chain constant region (Ig Fc) polypeptides (polypeptides comprising CH2-CH3 regions of Igs; see, e.g.); F KiH polypeptides; polypeptides of the collectin family (e.g., ACRP30 or ACRP30-like proteins) that contain collagen domains consisting of collagen repeats Gly-Xaa-Yaa; coiled-coil domains; leucine-zipper domains; Fos/Jun binding pairs; Ig heavy chain region 1 (CH1) and light chain constant region CL sequences (CH1/CL pairs such as a CH1 sequence paired with a κ or λ Ig light chain constant region sequence).

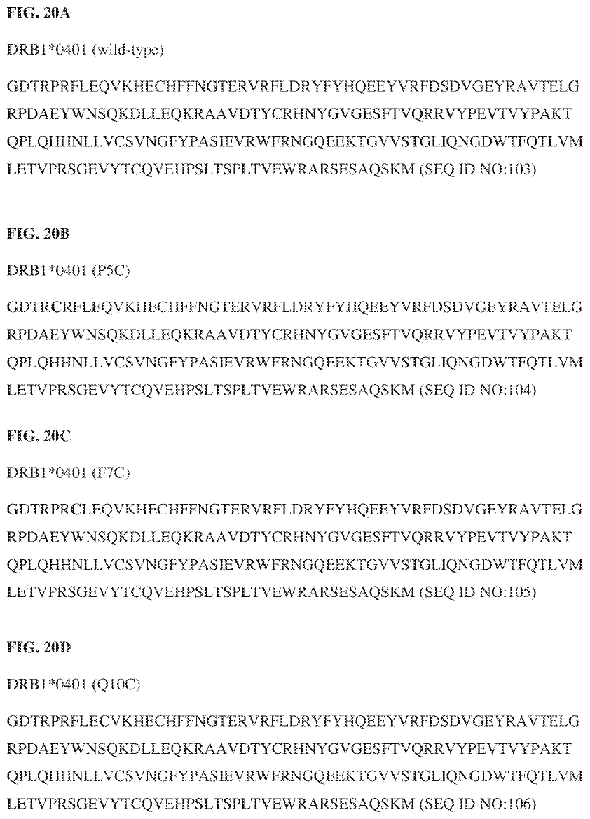

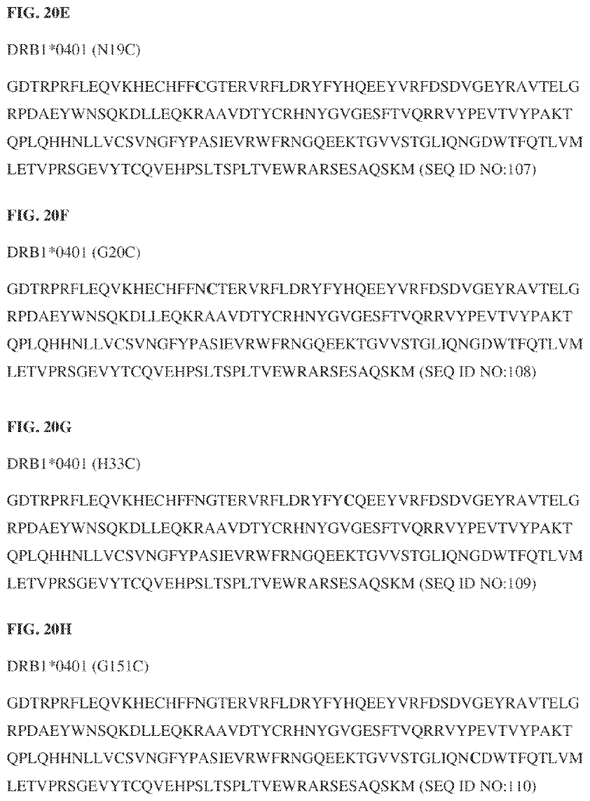

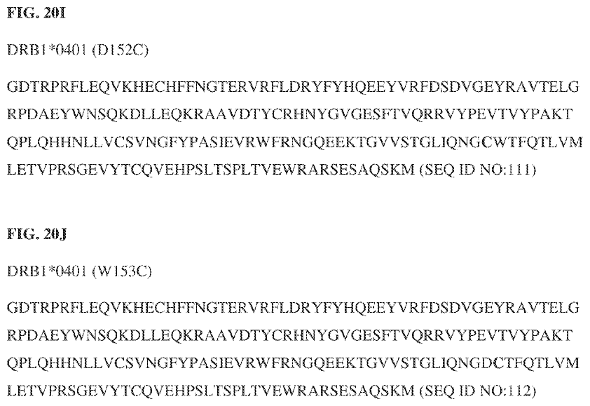

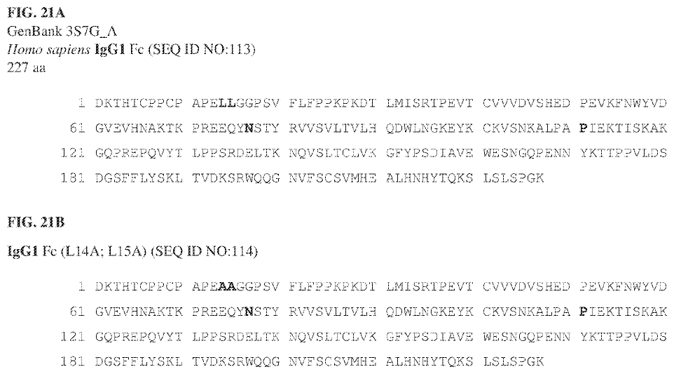

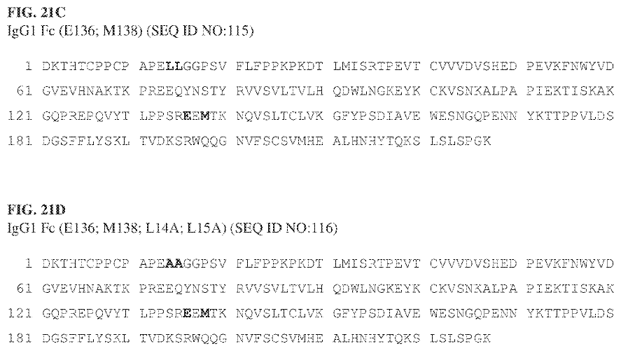

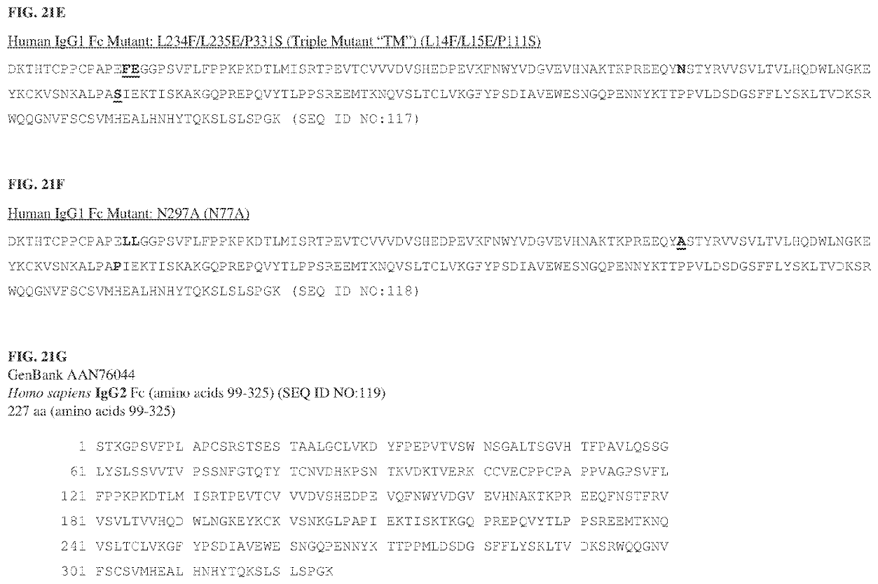

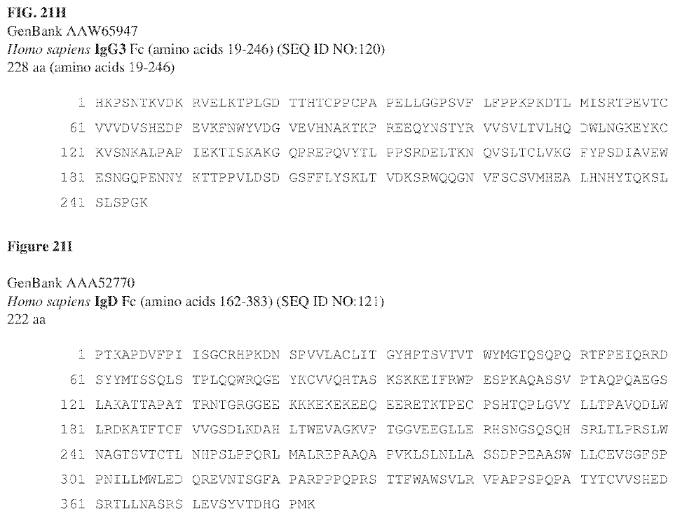

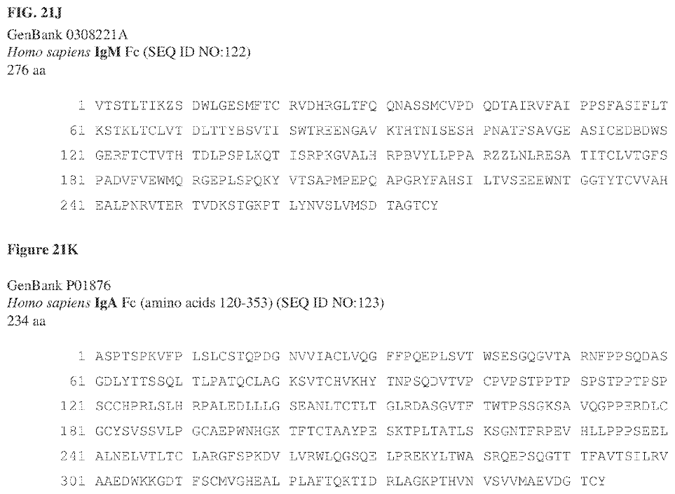

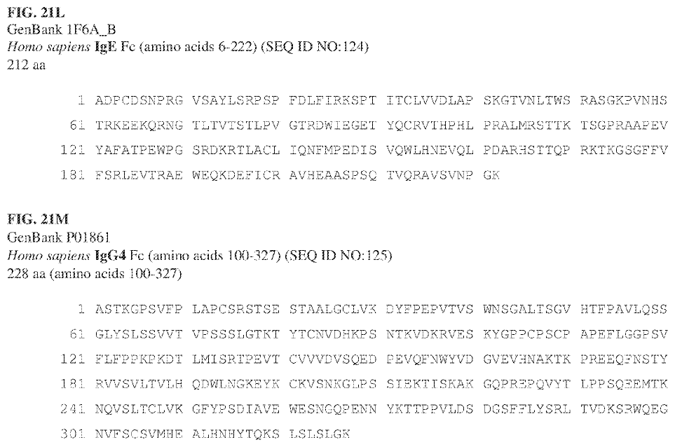

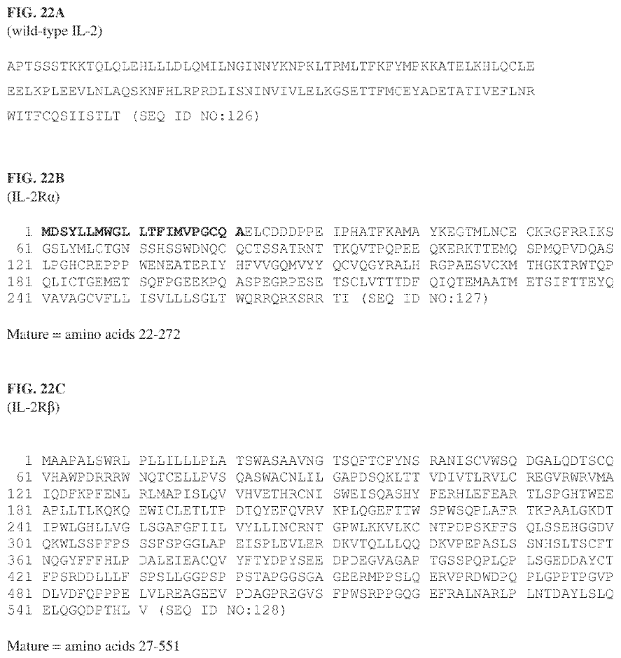

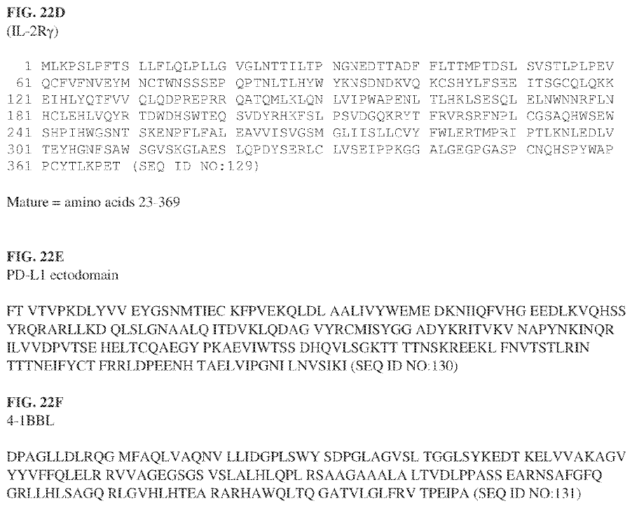

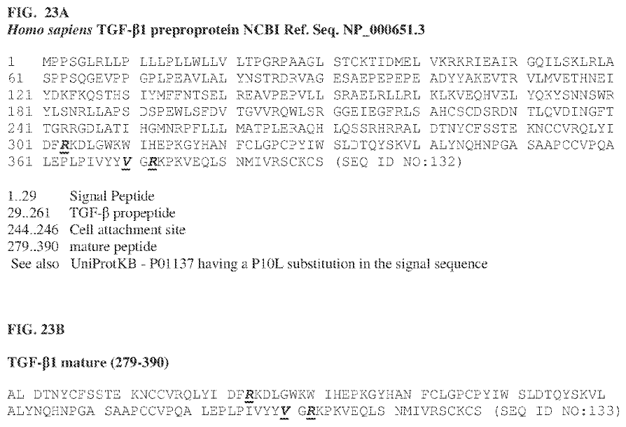

In some cases, the scaffold polypeptide comprises an Ig heavy chain constant region (CH2-C13) polypeptide sequence that functions as a dimerization or multimerization sequence (see, e.g., FIG. 21A-21M). In some cases, the Ig polypeptide will have a reduced ability to induce cell lysis, e.g., through activation of complement-dependent cytotoxicity (CDC) and/or antibody-dependent cellular cytotoxicity (ADCC), and thus may include mutations that reduce or substantially eliminate the ability of the Ig polypeptide to induce cell lysis. In some cases, the Ig Fc polypeptide comprises an amino acid sequence having at least about 70%, at least about 80%, at least about 90%, at least about 95%, at least about 98%, at least about 99%, or 100% aa sequence identity to an Ig Fc polypeptide depicted in any one of FIG. 21A-21M. Such Ig Fc polypeptides can covalently link the polypeptides of a TMP together, e.g., by forming one or two interchain disulfide bonds. As discussed below, an additional disulfide bond can be introduced to stabilize dimers, particularly where a pair of interspecific Ig sequence such Kill polypeptide pairs are employed.

In an embodiment, the scaffold polypeptide of a TMP comprises an amino acid sequence that has at least about 70%, at least about 80%, at least about 90%, at least about 95%, at least about 98%, at least about 99%, or 100% aa sequence identity to at least 150 contiguous as (at least 175, at least 200, at least 225, at least 250, at least 275, at least 300, at least 325, or at least 350 contiguous aas), or all aas, of the IgA Fe sequence depicted in FIG. 2K. In an embodiment, the scaffold polypeptide comprises an amino acid sequence that has at least about 70%, at least about 80%, at least about 90%, at least about 95%, at least about 98%, at least about 99%, or 100% aa sequence identity to at least 150 contiguous aas (at least 175, at least 200, at least 225, at least 250, at least 275, at least 300, at least 325, or at least 350 contiguous aas), or all aas, of the IgD Fc sequence depicted in FIG. 2I In an embodiment, the scaffold polypeptide comprises an amino acid sequence that has at least about 70%, at least about 80%, at least about 90%, at least about 95%, at least about 98%, at least about 99%, or 100% as sequence identity to least 125 contiguous aas (at least 150, at least 175, or at least 200 contiguous aas), or all aas, of the IgE Fe sequence depicted in FIG. 21L. In an embodiment, the scaffold polypeptide comprises an amino acid sequence that has at least about 70%, at least about 80%, at least about 90%, at least about 95%, at least about 98%, at least about 99%, or 100% aa sequence identity to least 125 contiguous aas (at least 150, at least 175, or at least 200 contiguous aas), or all aas, of a wt IgG Fe polypeptide, such as the IgG1 Fc amino acid sequence depicted in any one of FIG. 21A-21F. In an embodiment, the scaffold polypeptide comprises an amino acid sequence that has at least about 70%, at least about 80%, at least about 90%, at least about 95%, at least about 98%, at least about 99%, or 100% aa sequence identity to at least 125 contiguous aas (at least 150, at least 175, at least 200, at least 225, at least 250, at least 275, or at least 300), or all aas, of the IgG2 Fc polypeptide amino acid sequence depicted in FIG. 21G. In an embodiment, the scaffold polypeptide comprises a sequence that has at least about 70%, at least about 80%, at least about 90%, at least about 95%, at least about 98%, at least about 99%, or 100% aa sequence identity to at least 125 contiguous aas (at least 150, at least 175, at least 200, or at least 225), or all aas, of the IgG3 Fc amino acid sequence depicted in FIG. 21H. In an embodiment, the scaffold polypeptide comprises an amino acid sequence that has at least about 70%, at least about 80%, at least about 90%, at least about 95%, at least about 98%, at least about 99%, or 100% aa sequence identity to at least 125 contiguous aas (at least 150, at least 175, at least 200, at least 225, or at least 250), or all aas, of the IgG4 Fc amino acid sequence depicted in FIG. 21M. In an embodiment, the scaffold polypeptide comprises an amino acid sequence that has at least about 70%, at least about 80%, at least about 90%, at least about 95%, at least about 98%, at least about 99%, or 100% aa sequence identity to at least 125 contiguous aas (at least 150, at least 175, at least 200, at least 225, or at least 250), or all aas, of the IgM Fc polypeptide sequence depicted in FIG. 2U. The above-recited scaffold polypeptides can be covalently linked together by formation of one or two interchain disulfide bonds between cysteines adjacent to their hinge regions.

In some cases, the dimerization sequence of a scaffold polypeptide present in a TMP has at least about 70% (e.g., at least about 80%, 90%, 95%, 98%, 99% or 100%) aa sequence identity to the human IgG1 Fc polypeptide depicted in FIG. 21F, and comprises a substitution of N297 with an alanine (N297A substitution, or N77 as numbered in FIG. 21F) substitution. In some cases, the dimerization sequence of a scaffold polypeptide present in a TMP comprises an aa sequence depicted in FIG. 21A (human IgG1 Fe), except for a substitution of N297 (N77 of the aa sequence depicted in FIG. 21A) with an aa other than asparagine. Substitutions at N297 lead to the removal of carbohydrate modifications and result antibody sequences with reduced complement component 1q (“C1q”) binding compared to the wt. protein, and accordingly a reduction in complement-dependent cytotoxicity.

In some cases, the dimerization sequence of a scaffold polypeptide present in a TMP comprises an aa sequence depicted in FIG. 21A (human IgG1 Fc), except for a substitution of L234 (L14 of the as sequence depicted in FIG. 21A) with an aa other than leucine. L234 and other aas in the lower hinge region (e.g., aas 234-LLGGPS-239 (SEQ ID NO:172), which correspond to aas 14-19 of FIG. 21A) of IgG are involved in binding to the Fc lambda receptor (FcλR), and accordingly, mutations at that location reduce binding to the receptor (relative to the wt. protein). In some cases, the dimerization sequence of a scaffold polypeptide present in a TMP comprises an aa sequence depicted in FIG. 21A (human IgG1 Fc), except for a substitution of L235 (L15 of the an sequence depicted in FIG. 21A) with an aa other than leucine. In some cases, the dimerization sequence of a scaffold polypeptide present in a TMP comprises an aa sequence depicted in FIG. 21A (e.g., the wt. human IgG1 sequence) with L234A and L235A (“LALA”) substitutions (the positions corresponding to positions 14 and 15 of the wt. aa sequence depicted in FIG. 21A; see. e.g., SEQ ID NO: 113). See FIG. 21B. These two mutations will reduce or substantially eliminate the ability of the IgG1 Fc to induce cell lysis, e.g., through activation of complement-dependent cytotoxicity (CDC) and/or antibody-dependent cellular cytotoxicity (ADCC).

In some cases, the dimerization sequence of a scaffold polypeptide present in TMP comprises an aa sequence depicted in FIG. 21A (human IgG1 Fc), having a substitution of P331 (P111 of the aa sequence depicted in FIG. 21A) with an aa other than proline; in some cases, the substitution is a P331S substitution. Substitutions at P331, like those at N297, lead to reduced binding to C1q relative to the wt. protein, and thus a reduction in complement-dependent cytotoxicity. Substitutions of D270. K322, and/or P329 (corresponding to D50, K122, and P119 of FIG. 21A), for example with alanine, may be utilized alone or in place of a P331 substitution to reduce binding to C1q. As noted above, in some cases, the dimerization sequence of a scaffold polypeptide present in a TMP is an IgG1 Fc polypeptide that comprises L234A and/or L235A substitutions (substitutions of leucines at L14 and/or L15 of the aa sequences depicted in FIG. 21A with Ala). See FIG. 21B. In some cases, the dimerization sequence of a scaffold polypeptide present in a TMP comprises the aa sequence depicted in FIG. 21A (wt. human IgG1 Fc), except for substitutions at L234 and/or L235 (L14 and/or L15 of the aa sequence depicted in FIG. 21A) with aas other than leucine, and a substitution of P331 (P111 of the aa sequence depicted in FIG. 2D) with an aa other than proline. In some cases, the dimerization sequence of a scaffold polypeptide present in a TMP comprises the “Triple Mutant” aa sequence depicted in FIG. 21E (human IgG1 Fc) comprising L234F. L235E, and P331S substitutions (corresponding to aa positions 14, 15, and 111 of the aa sequence depicted in FIG. 21E).

As noted above, the Ig Fc polypeptides of each polypeptide chain of a heterodimeric TMP may comprise interspecific dimerization sequences. e.g., KiH sequences that permit the two chains to selectively dimerize. Interspecific binding sequences favor formation of heterodimers with their cognate polypeptide sequence (i.e., the interspecific sequence and its counterpart interspecific sequence), particularly those based on Ig Fc sequence variants. Such interspecific polypeptide sequences include KiH, and KiH sequences that facilitate the formation of one or more disulfide bonds. For example, one interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fc sequenec. See Xu et al. mAbs 7:1, 231-242 (2015). Another interspecific binding pair has a first Fc polypeptide with Y349C, T366S, L368A, and Y407V substitutions and a second Ig Fc polypeptide with S354C, and T366W substitutions (disulfide bonds can form between the Y349C and the S354C). See. e.g., Brinkmann and Konthermann, mAbs 9:2, 182-212 (2015). Ig Fc polypeptide sequences, either with or without KiH modifications, can be stabilized by the formation of disulfide bonds between the Ig Fc polypeptides (e.g., the hinge region disulfide bonds).

Several interspecific binding sequences based upon immunoglobulin sequences are summarized in Table 1, below, with cross reference to the numbering of the aa positions as they appear in the wt IgG1 sequence set forth in FIG. 21A shown in brackets “{ }”. In FIG. 21A: i) hinge: amino acids 1-10; ii) CH2: amino acids 11-120; iii) CH3: amino acids 121-227; iv) Fc amino acids 11-227.

TABLE 1
Interspecific immunoglobulin sequences and their cognate counterpart interspecific sequences

| | Substitutions in the first | Substitutions in the second | |
|---|---|---|---|
| Interspecific | interspecific polypeptide | (counterpart) interspecific | |
| Pair Name | sequence | polypeptide sequence | Comments |
| KiH | T366W | T366S/L368A/Y407V | Hydrophobic/steric |
| | {T146W} | {T146S/L148A/Y187V} | complementarity |
| KiHs-s | T366W/S354C* | T366S/L368A/Y407V/Y349C | KiH + inter-CH3 |
| | {T146W/S134C*} | {T146S/L148A/Y187V/Y129C} | domain S-S bond |
| HA-TF | S364H/F405A | Y349T/T394F | Hydrophobic/steric |
| | {S144H/F185A] | {Y129T/T174F} | complementarity |
| ZW1 | T350V/L351Y/F405A/Y407V | T350V/T366L/K392L/T394W | Hydrophobic/steric |
| | {T130V/L131Y/F185A/Y187V} | {T130V/T146L/K172L/T174W} | complementarity |
| 7.8.60 | K360D/D399M/Y407A | E345R/Q347R/T366V/K409V | Hydrophobic/steric |
| | {K140D/D179M/Y187A} | {E125R/Q127R/T146V/K189V} | complementarity + |
| | | | electrostatic |
| | | | complementarity |
| DD-KK | K409D/K392D | D399K/E356K | Electrostatic |
| | {K189D/K172D} | {D179K/E136K} | complementarity |
| EW-RVT | K360E/K409W | Q347R/D399V/F405T | Hydrophobic/steric |
| | {K140E/K189W} | {Q127R/D179V/F185T} | complementarity & |
| | | | long-range electro- |
| | | | static interaction |
| EW-RVTs-s | K360E/K409W/Y349C* | Q347R/D399V/F405T/S354C | EW-RVT + inter- |
| | {K140E/K189W/Y129C*} | {Q127R/D179V/F185T/S134C} | CH3 domain S-S |
| | | | bond |
| A107 | K370E/K409W | E357N/D399V/F405T | Hydrophobic/steric |
| | {K150E/K189W] | {E137N/D179V/F185T} | complementarity + |
| | | | hydrogen bonding |
| | | | complementarity |

Table 1 modified from Ha et al., Frontiers in Immunol. 7: 1-16 (2016).
*aa forms a stabilizing disulfide bond.

In addition to the interspecific pairs of sequences in Table 1, Ig Fc polypeptides may include interspecific “SEED” sequences having 45 residues derived from IgA in an IgG1 CH3 domain of the interspecific sequence, and 57 residues derived from IgG1 in the IgA CH3 in its counterpart interspecific sequence. See Ha et al., Frontiers in Immunol. 7:1-16 (2016).

An Ig Fc polypeptide present in a TMP may comprise an interspecific binding sequence or its counterpart interspecific binding sequence selected from the group consisting of: knob-in-hole (KiH); knob-in-hole with a stabilizing disulfide (KiHs-s); HA-TF; ZW-1; 7.8.60; DD-KK; EW-RVT; EW-RVTs-s; A107; or SEED sequences.

A TMP may comprise an Ig Fc polypeptide comprising a T146W KiH sequence substitution, and its counterpart interspecific binding partner polypeptide comprises an Ig Fc polypeptide having T146W, L148A, and Y187V Kill sequence substitutions, where the Ig Fc polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt IgG1 of FIG. 21A. One or both of the Ig Fc polypeptides optionally comprising substitutions at one of more of: L234 and L235 (e.g., L234A/L235A “LALA” or L234F/L235E); N297 (e.g., N297A); P331 (e.g., P331S); L351 (e.g., L351K); T366 (e.g., T366S); P395 (e.g., P395V); F405 (e.g., F405R); Y407 (e.g., Y407A); and K409 (e.g., K409Y). Those substitutions appear at: 1.14 and L15 (e.g., L14A/15A “LALA” or L14F/L15E); N77 (e.g., N77A); P111 (e.g., P111 S) L131 (e.g., L131K); T146 (e.g., T146S); P175 (e.g., P175V); F185 (e.g., F185R); Y187 (e.g., Y187A); and K189 (e.g., K189Y) in the wt IgG1 sequence of FIG. 21A.

A TMP in some cases comprises an Ig Fc polypeptide comprising a T146W KiH sequence substitution, and its counterpart interspecific binding partner Ig Fc polypeptide comprises T146S, L148A, and Y187V KiH sequence substitutions, where the Ig Fc polypeptides comprise an amino acid sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 21A; where one or both of the Ig Fc polypeptides may comprise additional substitutions such as L14 and/or L15 substitutions (e.g., “LALA” substitutions L234A and L235A), and/or N77 (N297 e.g., N297A or N297G).

A TMP in some cases comprises an Ig Fc polypeptide comprising T146W and S134C KiHs-s substitutions, and its counterpart interspecific binding partner Ig Fc polypeptide comprises T146S, L148A. Y187V and Y129C KiHs-s substitutions, where the Ig Fc polypeptides comprise an amino acid sequence having at least 80%, at least 90%, at least 95%, or at least 97% sequence identity to at least 100 (e.g., at least 125, 150, 170, 180, 190, 200, 210, 220, or all 227) contiguous aas of the wt IgG1 of FIG. 21A; where one or both of the Ig Fc polypeptides may comprise additional substitutions such as L14 and/or L15 substitutions (e.g., “LALA” substitutions L234A and L235A), and/or N77 (N297 e.g., N297A or N297G).

A TMP in some cases comprises an Ig Fc polypeptide comprising S144H and F185A HA-TF substitutions, and its counterpart interspecific binding partner Ig Fc polypeptide comprises Y129T and T174F HA-TF substitutions, where the Ig Fc polypeptides comprise an amino acid sequence having at least 80%, at least 90%, at least 95%, or at least 97% sequence identity to at least 100 (e.g., at least 125, 150, 170, 180, 190, 200, 210, 220, or all 227) contiguous aas of the wt IgG1 of FIG. 21A; where one or both of the Ig Fc polypeptides may comprise additional substitutions such as L14 and/or L15 substitutions (e.g., “LALA” substitutions L234A and L235A), and/or N77 (N297 e.g., N297A or N297G).

A TMP in some cases comprises an Ig Fc polypeptide comprising T130V. L131Y. F185A, and Y187V ZW1 substitutions, and its counterpart interspecific binding partner Ig Fc polypeptide comprises T130V, T146L, K172L, and T174W ZW1 substitutions, where the Ig Fc polypeptides comprise an amino acid sequence having at least 80%, at least 90%, at least 95%, or at least 97% sequence identity to at least 100 (e.g., at least 125, 150, 170, 180, 190, 200, 210, 220, or all 227) contiguous aas of the wt IgG1 of FIG. 21A; where one or both of the Ig Fc polypeptide sequences may comprise additional substitutions such as L14 and/or L15 substitutions (e.g., “LALA” substitutions L234A and L235A), and/or N77 (N297 e.g., N297A or N297G).

A TMP in some cases comprises an Ig Fc polypeptide comprising K140D. D179M, and Y187A 7.8.60 substitutions, and its counterpart interspecific binding partner Ig Fc polypeptide comprises T130V E125R. Q127R, T146V, and K189V 7.8.60 substitutions, where the Ig Fc polypeptides comprise an amino acid sequence having at least 80%, at least 90%, at least 95%, or at least 97% sequence identity to at least 100 (e.g., at least 125, 150, 170, 180, 190, 200, 210, 220, or all 227) contiguous aas of the wt IgG1 of FIG. 21A; where one or both of the Ig Fc polypeptide sequences may comprise additional substitutions such as L14 and/or L15 substitutions (e.g., “LALA” substitutions L234A and L235A), and/or N77 (N297 e.g., N297A or N297G).

A TMP in some cases comprises an Ig Fc polypeptide comprising K189D and K172D DD-KK substitutions, and its counterpart interspecific binding partner Ig Fc polypeptide comprises an IgG1 sequence having T130V D179K and E136K DD-KK substitutions, where the Ig Fc polypeptides comprise an amino acid sequence having at least 80%, at least 90%, at least 95%, or at least 97% sequence identity to at least 100 (e.g., at least 125, 150, 170, 180, 190, 200, 210, 220, or all 227) contiguous aas of the wt IgG1 of FIG. 21A; where one or both of the Ig Fc polypeptides may comprise additional substitutions such as L14 and/or L15 substitutions (e.g., “LALA” substitutions L234A and L235A), and/or N77 (N297 e.g., N297A or N297C).

A TMP in some cases comprises an Ig Fc polypeptide comprising K140E and K189W EW-RVT substitutions, its counterpart interspecific binding partner Ig Fc polypeptide comprises T130V Q127R, D179V, and F185T EW-RVT substitutions, where the Ig Fc polypeptides comprise an amino acid sequence having at least 80%, at least 90%, at least 95%, or at least 97% sequence identity to at least 100 (e.g., at least 125, 150, 170, 180, 190, 200, 210, 220, or all 227) contiguous aas of the wt IgG1 of FIG. 21A; where one or both of the Ig Fc polypeptides may comprise additional substitutions such as L14 and/or L15 substitutions (e.g., “LALA” substitutions L234A and L235A; corresponding to L14 and L15 of the amino acid sequence depicted in FIG. 21A), and/or N77 (N297 e.g., N297A or N297G).

A TMP in some cases comprises an Ig Fc polypeptide comprising K140E, K189W, and Y129C EW-RVTs-s substitutions, its counterpart interspecific binding partner Ig Fc polypeptide comprises T130V Q127R, D179V. F185T, and S134C EW-RVTs-s substitutions, where the Ig Fc polypeptides comprise an amino acid sequence having at least 80%, at least 90%, at least 95%, or at least 97% sequence identity to at least 100 (e.g., at least 125, 150, 170, 180, 190, 200, 210, 220, or all 227) contiguous aas of the wt IgG1 of FIG. 21A. One or both of the Ig Fc polypeptides may comprise additional substitutions such as L14 and/or L15 substitutions (e.g., “LALA” substitutions L234A and L235A; corresponding to L14 and L15 of the amino acid sequence depicted in FIG. 21A), and/or N77 (N297 e.g., N297A or N297G).

A TMP in some cases comprises an Ig Fc polypeptide comprising K150E and K189W A107 substitutions, its counterpart interspecific binding partner Ig Fc polypeptide comprises T130V E137N, D179V, and F185T A107 substitutions, where the Ig Fc polypeptides comprise an amino acid sequence having at least 80%, at least 90%, at least 95%, or at least 97% sequence identity to at least 100 (e.g., at least 125, 150, 170, 180, 190, 200, 210, 220, or all 227) contiguous aas of the wt IgG1 of FIG. 21A; where one or both of the Ig Fc polypeptides may comprise additional substitutions such as L14 and/or L15 substitutions (e.g., “LALA” substitutions L234A and L235A), and/or N77 (N297 e.g., N297A or N297G).

As one non-limiting example, in some cases: a) the first polypeptide of a TMP comprises an Ig Fc polypeptide (referred to in FIG. 26A as “IgG Fc (LALA) KiH Chain A (S354C; T366W)” comprising an amino acid sequence having at least 90%, at least 95%, or at least 98%, to the following amino acid sequence:

DKTHTCPPCPAPEAAGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQPREPQ VYTLPPCREEMTKNQVSLWCLVKGFYPSDIAVEWESNGQPENNYKTTPPVLDSDGSFFLYSKLT VDKSRWQQGNVFSCSVMHEALHNHYTQKSLSLSPG (SEQ ID NO:173), where amino acid 14 is Leu, amino acid 15 is Len, amino acid 134 is Cys, and amino acid 146 is Trp.

and b) the second polypeptide of the TMP comprises an Ig Fc polypeptide (referred to in FIG. 26B as “IgG Fc (LALA) KiH Chain B (Y349C, T366S, L368A, Y407V)”) comprising an amino acid sequence having at least 90%, at least 95%, or at least 98%, to the following amino acid sequence: DKTHTCPPCPAPEAAGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQPREPQ VCTLPPSREEMTKNQVSLSCAVKGFYPSDIAVEWESNGQPENNYKTITPPVLDSDGSFFLVSKLT VDKSRWQQGNVFSCSVMHEALHNHYTQKSLSLSPG (SEQ ID NO:174), where amino acid 14 is Leu, amino acid 15 is Leu, amino acid 129 is Cys, amino acid 146 is Ser, amino acid 148 is Ala, and amino acid 187 is Val.

In some cases, a) the first polypeptide of a TMP comprises an Ig Fc polypeptide (referred to in FIG. 26A as “IgG Fc (LALA) KiH Chain A (S354C; T366W)” having the following amino acid sequence: DKTHTCPPCPAPEAAGGPSVFLFPPKPKDTLMISRTPEVTCVVVDVSHEDPEVKFNWYVDVEV HNAKTKPREEQYNSTYRVVSVLTVLHQDWLNGKEYKCKVSNKALPAPIEKTISKAKGQPREPQ VYTLPPCREEMTKNQVSLWCLVKGFYPSDIAVEWESNCGQPENNYKTTPPVLDSDGSFFLYSKLT VDKSRWQQGNVFSCSVMHEALHNHYTQKSLSLSPG (SEQ ID NO:173); and b) the second the second polypeptide of the TMP comprises an Ig Fc polypeptide (referred to in FIG. 26B as “IgG Fc (LALA) KiH Chain B (Y349C, T366S, L368A, Y407V)”) having the following amino acid sequence:

(SEQ ID NO: 173)
DKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPCREEMTKNQVSLWC
LVKGFYPSDIAVEWESNGQPENNYKTTPPVLDSDGSFFLYSKLTVDKSR
WQQGNVFSCSVMHEALHNHYTQKSLSLSPG.

B) Heterodimeric TMPs Comprising Disulfide Linkages

A TMP of the present disclosure in some cases comprises one or more heterodimers, where each heterodimer comprises: a) a first polypeptide chain comprising: i) a T1D peptide; and a first MHC class II polypeptide; and b) a second polypeptide comprising a second MHC class II polypeptide, where the first and/or the second polypeptide comprises one or more MODs, where the first or the second polypeptide comprises a TGF-β polypeptide, and where the first or the second polypeptide comprises a masking polypeptide; where the TMP optionally includes one or more independently selected linker polypeptides, and where the first or the second polypeptide chain comprises an Ig Fc polypeptide. In some cases, the first polypeptide comprises the TGF-β polypeptide and the second polypeptide comprises the masking polypeptide. In some cases, the first polypeptide comprises the masking polypeptide and the second polypeptide comprises the TGF-β polypeptide. In some cases, the first polypeptide comprises the TGF-β polypeptide and the masking polypeptide, and the second polypeptide does not comprise a TGF-β polypeptide or a masking polypeptide. In some cases, the first polypeptide does not comprise a TGF-β polypeptide or a masking polypeptide, and the second polypeptide comprises the TGF-β polypeptide and the masking polypeptide. A TMP of the present disclosure can be a homodimer comprising two such heterodimers, where the Ig Fc polypeptide of one of the heterodimers is disulfide linked to the Ig Fc polypeptide of the other heterodimer. Non-limiting examples of such TMPs are depicted schematically in FIG. 2A-2D. A TMP can comprise two disulfide-linked heterodimers. When both heterodimers include Ig Fc polypeptides, disulfide bonds will spontaneously form between the respective Ig Fc polypeptides to covalently link the two heterodimers to one another. Alternatively, a TMP of the present disclosure can be a heterodimer comprising two such heterodimers, where the Ig Fc polypeptide of one of the heterodimers is disulfide linked to the Ig Fc polypeptide of the other heterodimer using interspecific binding sequences as discussed above.

Arrangement of Components

In some cases, a TMP comprise at least one heterodimer, wherein each heterodimer comprises: a) a first polypeptide comprising: i) a T1 D peptide; and ii) a first MHC class II polypeptide; and iii) optionally a linker that links the T1D peptide to the first MHC class II polypeptide; and b) a second polypeptide comprising the second MHC class II polypeptide, wherein the first and/or the second polypeptide comprises the one or more MODs, wherein the first or the second polypeptide comprises the TGF-β polypeptide, and wherein the first or the second polypeptide comprises the masking polypeptide, and optionally where the first polypeptide and the second polypeptide of the heterodimer are covalently linked to one another via at least one disulfide bond.

In some cases, a TMP comprise at least one heterodimer, wherein each heterodimer comprises: a) a first polypeptide comprising: i) a T1D peptide; ii) an MHC class II α chain polypeptide or an MHC class II β chain polypeptide; ii) optionally a linker that links the T1D peptide to the first MHC class II polypeptide; and b) a second polypeptide comprising i) an MHC class II α chain polypeptide if the first polypeptide comprises an MHC class II β chain polypeptide, or ii) an MHC class II β chain polypeptide if the first polypeptide comprises an MHC class II α chain polypeptide, wherein: i) the first and/or the second polypeptide comprises the one or more MODs; ii) the first or the second polypeptide comprises the TGF-β polypeptide; and iii) the first or the second polypeptide comprises the masking polypeptide; optionally wherein the first polypeptide and the second polypeptide of the heterodimer are covalently linked to one another via at least one disulfide bond; and optionally where the TMP optionally comprises one or more independently selected linker polypeptides (e.g., where each of the one or more independently selected linker polypeptides is between any two of the aforementioned polypeptides).

As a first non-limiting example, in some cases a TMP comprises: at) a first polypeptide comprising: i) a T1D peptide; ii) an MHC class II β polypeptide; and b1) a second polypeptide comprising: i) an MHC class II α polypeptide; ii) a TGF-β polypeptide; iii) a masking polypeptide; iv) one or more MODs; and v) an Ig Fc polypeptide. In some cases, the components of the first polypeptide are joined by one or more independently selected linkers, and in some cases the components of the second polypeptide are joined by one or more independently selected linkers.

As a second non-limiting example, in some cases a TMP comprises: a2) a first polypeptide comprising: i) aT1D peptide; ii) an MHC class II β § polypeptide; and iii) a TGF-β polypeptide or a masking polypeptide, and b2) a second polypeptide comprising: i) an MHC class II α polypeptide; ii) a masking polypeptide if the first polypeptide comprises the TGF-β polypeptide, or a TGF-β polypeptide if the first polypeptide comprises the masking polypeptide; iii) one or more MODs; and iv) an Ig Fc polypeptide. In some cases, the components of the first polypeptide are joined by one or more independently selected linkers, and in some cases the components of the second polypeptide are joined by one or more independently selected linkers.

As a third non-limiting example, in some cases a TMP comprises: a3) a first polypeptide comprising: i) a T1 D peptide; ii) an MHC class II β polypeptide; iii) a TGF-β polypeptide or a masking polypeptide; and iv) an Ig Fc polypeptide, and b3) a second polypeptide comprising: i) an MHC class II α polypeptide; ii) a masking polypeptide if the first polypeptide comprises the TGF-β polypeptide, or a TGF-β polypeptide if the first polypeptide comprises the masking polypeptide; iii) one or more MODs. In some cases, the components of the first polypeptide are joined by one or more independently selected linkers, and in some cases the components of the second polypeptide are joined by one or more independently selected linkers.

As a fourth non-limiting example, in some cases a TMP comprises: a4) a first polypeptide comprising: i) aT1D peptide; ii) an MHC class II β polypeptide; iii) a TGF-β polypeptide or a masking polypeptide; and iv) one or more MODs, and b4) a second polypeptide comprising: i) an MHC class II α polypeptide; ii) a masking polypeptide if the first polypeptide comprises the TGF-β polypeptide, or a TGF-β polypeptide if the first polypeptide comprises the masking polypeptide; iii) an Ig Fc polypeptide. In some cases, the components of the first polypeptide are joined by one or more independently selected linkers, and in some cases the components of the second polypeptide are joined by one or more independently selected linkers.

As a fifth non-limiting example, in some cases a TMP comprises: a5) a first polypeptide comprising: i) a T1D peptide; ii) an MHC class II β polypeptide; iii) a TGF-β polypeptide or a masking polypeptide; and iv) one or more MODs; and v) an Ig Fc polypeptide; and b5) a second polypeptide comprising: i) an MHC class II α polypeptide; and ii) a masking polypeptide if the first polypeptide comprises the TGF-β polypeptide, or a TGF-β polypeptide if the first polypeptide comprises the masking polypeptide. In some cases, the components of the first polypeptide are joined by one or more independently selected linkers, and in some cases the components of the second polypeptide are joined by one or more independently selected linkers.

As a sixth non-limiting example, in some cases a TMP comprises: a6) a first polypeptide comprising: i) a T1D peptide; ii) an MHC class II β polypeptide; iii) a TGF-β polypeptide; iv) a masking polypeptide; v) one or more MODs; and vi) an Ig Fc polypeptide; and b6) a second polypeptide comprising an MHC class II α polypeptide. In some cases, the components of the first polypeptide are joined by one or more independently selected linkers.

In some cases, as depicted schematically in FIG. 2A, a TMP comprises: a) a first polypeptide comprising, in order from N-terminus to C-terminus: i) a T1D peptide; ii) an MHC class II β chain polypeptide; iii) a masking polypeptide; and iv) an Ig Fc polypeptide; and b) a second polypeptide comprising, in order from N-terminus to C-terminus: i) an MHC class II α chain polypeptide; ii) a TGF-β polypeptide; and iii) one or more MODs, where the TMP optionally includes one or more independently selected linker polypeptides between any two of the components. The first and the second polypeptides are covalently linked to one another by disulfide bonds, e.g., disulfide bonds formed between Cys residues in the MHC class II α and β chains, as described below.

In some cases, a TMP comprises: a) a first polypeptide comprising, in order from N-terminus to C-terminus: i) a T1D peptide; ii) an MHC class II β chain polypeptide; iii) a TGF-β polypeptide; and iv) an Ig Fc polypeptide; and b) a second polypeptide comprising, in order from N-terminus to C-terminus: i) an MHC class II α chain polypeptide; ii) a masking polypeptide; and iii) one or more MODs, where the TMP optionally includes one or more independently selected linker polypeptides between any two of the components. The first and the second polypeptides are covalently linked to one another by disulfide bonds, e.g., disulfide bonds formed between Cys residues in the MHC class II α and β chains, as described below.

In some cases, as depicted schematically in FIG. 2B, a TMP comprises: a) a first polypeptide comprising, in order from N-terminus to C-terminus: i) a T1D peptide; ii) an MHC class II β chain polypeptide; iii) a masking polypeptide; iv) an Ig Fc polypeptide; and v) one or more MODs; and b) a second polypeptide comprising, in order from N-terminus to C-terminus: i) an MHC class II α chain polypeptide; ii) a TGF-β polypeptide, where the TMP optionally includes one or more independently selected linker polypeptides between any two of the components. The first and the second polypeptides are covalently linked to one another by disulfide bonds. e.g., disulfide bonds formed between Cys residues in the MHC class II α and β chains, as described below.

In some cases, a TMP comprises: a) a first polypeptide comprising, in order from N-terminus to C-terminus: i) a T1D peptide; ii) an MHC class II β chain polypeptide; iii) a TGF-β polypeptide; iv) an Ig Fc polypeptide; and v) one or more MODs; and b) a second polypeptide comprising, in order from N-terminus to C-terminus: i) an MHC class II α chain polypeptide; ii) a masking polypeptide, where the TMP optionally includes one or more independently selected linker polypeptides between any two of the components. The first and the second polypeptides are covalently linked to one another by disulfide bonds, e.g., disulfide bonds formed between Cys residues in the MHC class II α and β chains, as described below.

In some cases, as depicted schematically in FIG. 2C, a TMP comprises: a) a first polypeptide comprising, in order from N-terminus to C-terminus: i) a T1D peptide; ii) an MHC class II β chain polypeptide; iii) a masking polypeptide; and iv) an Ig Fc polypeptide; and b) a second polypeptide comprising, in order from N-terminus to C-terminus: i) one or more MODs; ii) an MHC class II α chain polypeptide; and iii) a TGF-β polypeptide, where the TMP optionally includes one or more independently selected linker polypeptides between any two of the components. The first and the second polypeptides are covalently linked to one another by disulfide bonds. e.g., disulfide bonds formed between Cys residues in the MHC class II α and β chains, as described below.

In some cases, a TMP comprises: a) a first polypeptide comprising, in order from N-terminus to C-terminus: i) a T1D peptide; ii) an MHC class II β chain polypeptide; iii) a TGF-β polypeptide; and iv) an Ig Fc polypeptide; and b) a second polypeptide comprising, in order from N-terminus to C-terminus: i) one or more MODs; ii) an MHC class II α chain polypeptide; and iii) a masking polypeptide, where the TMP optionally includes one or more independently selected linker polypeptides between any two of the components. The first and the second polypeptides are covalently linked to one another by disulfide bonds, e.g., disulfide bonds formed between Cys residues in the MHC class II α and β chains, as described below.

In some cases, as depicted schematically in FIG. 2D, a TMP comprises: a) a first polypeptide comprising, in order from N-terminus to C-terminus: i) a T1D peptide; ii) an MHC class II β chain polypeptide; iii) a masking polypeptide; and iv) a TGF-β polypeptide; and b) a second polypeptide comprising, in order from N-terminus to C-terminus: i) one or more MODs; ii) an MHC class II α chain polypeptide; and iii) an Ig Fc polypeptide, where the TMP optionally includes one or more independently selected linker polypeptides between any two of the components. The first and the second polypeptides are covalently linked to one another by disulfide bonds. e.g., disulfide bonds formed between Cys residues in the MHC class II α and β chains, as described below.

In some cases, a TMP comprises: a) a first polypeptide comprising, in order from N-terminus to C-terminus: i) a T1D peptide; ii) an MHC class II β chain polypeptide; iii) a TGF-β polypeptide; and iv) a masking polypeptide; and b) a second polypeptide comprising, in order from N-terminus to C-terminus: i) one or more MODs; ii) an MHC class II α chain polypeptide; and iii) an Ig Fc polypeptide, where the TMP optionally includes one or more independently selected linker polypeptides between any two of the components. The first and the second polypeptides are covalently linked to one another by disulfide bonds, e.g., disulfide bonds formed between Cys residues in the MHC class II α and β chains, as described below.

### Disulfide Linkages

As noted above, the first polypeptide and the second polypeptide of a heterodimer of a TMP are covalently linked to one another via at least one disulfide bond. For example, the at least one disulfide bond is present between: i) a Cys present in the first MHC class II polypeptide and a Cys present in the second MHC class II polypeptide; or ii) a Cys present in a peptide linker in the first polypeptide and a Cys present in an MHC class II polypeptide present in the second polypeptide; or iii) a Cys present in a peptide linker in the second polypeptide and a Cys present in an MHC class II polypeptide present in the first polypeptide; or iv) a Cys present in a linker in the first polypeptide and a Cys present in a linker in the second polypeptide.

1) Disulfide Bond Between the Two MHC Class II Polypeptides

As noted above, in some cases, the first polypeptide of a heterodimer of a TMP comprises a first MHC class II polypeptide comprising an amino acid substitution that results in a Cys (a “first Cys”); and the second polypeptide comprises a second MHC class II polypeptide comprising an amino acid substitution that results in a Cys (a “second Cys”); where the heterodimer comprises a disulfide bond formed between the first Cys and the second Cys. For example, in some cases, the first polypeptide comprises an MHC class II β polypeptide comprising an amino acid substitution that results in a Cys (a “first Cys”); and the second polypeptide comprises an MHC class II α polypeptide comprising an amino acid substitution that results in a Cys (a “second Cys”); where the heterodimer comprises a disulfide bond formed between the first Cys and the second Cys.

Arrangement of Components

In some cases, for example, a TMP comprises: a) a first polypeptide comprising, in order from N-terminus to C-terminus: i) a T1D peptide; and ii) a first MHC class II polypeptide comprising a substitution of an amino acid (other than a Cys) with a Cys (a “first Cys”), the components of the first polypeptide optionally being joined by a linker that does not comprise a Cys; and b) a second polypeptide comprising, in order from N-terminus to C-terminus: i) one or more MODs; ii) a second MHC class II polypeptide comprising a substitution of an amino acid (other than a Cys) with a Cys (a “second Cys”); and iii) optionally an Ig Fc polypeptide, the components of the second polypeptide optionally being joined by one or more linkers that do not comprise a Cys; where the first polypeptide and the second polypeptide are linked via a disulfide bond between the first Cys and the second Cys; where the first or the second polypeptide comprises a TGF-β polypeptide, and where the first or the second polypeptide comprises a masking polypeptide. In any of the above embodiments, in some cases the one or more MODs is a PD-L1 polypeptide or variant thereof, in some cases the one or more MODs is a 4-1BBL polypeptide or variant thereof, in some cases the one or more MODs is an IL-2 polypeptide or variant thereof, and in some cases the one or more MODs is a Fas, polypeptide or variant thereof. In some cases, the T1D peptide is a proinsulin peptide. In some cases, the T1D peptide is a GAD peptide. In any of the above embodiments, in some cases, the first MHC class II polypeptide is an MHC class II (i chain polypeptide and the second MHC class IT polypeptide is an MHC class II α chain polypeptide.

As another example, in some cases, a TMP comprises: a) a first polypeptide comprising, in order from N-terminus to C-terminus: i) a T1D peptide; and ii) a first MHC class II polypeptide comprising a substitution of an amino acid (other than a Cys) with a Cys (a “first Cys”), the components of the first polypeptide optionally being joined by a linker that does not comprise a Cys; and b) a second polypeptide comprising, in order from N-terminus to C-terminus: i) a second MHC class II polypeptide comprising a substitution of an amino acid (other than a Cys) with a Cys (a “second Cys”); ii) one or more MODs; and iii) optionally an Ig Fc polypeptide, the components of the second polypeptide optionally being joined by one or more linkers that do not comprise a Cys; where the first polypeptide and the second polypeptide are linked via a disulfide bond between the fast Cys and the second Cys; where the first or the second polypeptide comprises a TGF-β polypeptide, and where the first or the second polypeptide comprises a masking polypeptide. In any of the above embodiments, in some cases the one or more MODs is a PD-L1 polypeptide or variant thereof, in some cases the one or more MODs is a 4-1BBL polypeptide or variant thereof, in some cases the one or more MODs is an IL-2 polypeptide or variant thereof, and in some cases the one or more MODs is a FasL polypeptide or variant thereof. In some cases, the T1D peptide is a proinsulin peptide. In some cases, the T1D peptide is a GAD peptide. In any of the above embodiments, in some cases, the first MIC class II polypeptide is an MHC class II § chain polypeptide and the second MHC class II polypeptide is an MHC class II α chain polypeptide.

As another example, in some cases, a TMP comprises: a) a first polypeptide comprising, in order from N-terminus to C-terminus: i) a T1D peptide; and ii) a first MHC class II polypeptide comprising a substitution of an amino acid (other than a Cys) with a Cys (a “first Cys”), the components of the first polypeptide optionally being joined by a linker that does not comprise a Cys; and b) a second polypeptide comprising, in order from N-terminus to C-terminus: i) a second MHC class II polypeptide comprising a substitution of an amino acid (other than a Cys) with a Cys (a “second Cys”); ii) an Ig Fc polypeptide; and iii) one or more MODs, the components of the second polypeptide optionally being joined by a linker that does not comprise a Cys; where the first polypeptide and the second polypeptide are linked via a disulfide bond between the first Cys and the second Cys; where the first or the second polypeptide comprises a TGF-β polypeptide, and where the first or the second polypeptide comprises a masking polypeptide. In any of the above embodiments, in some cases the one or more MODs is a PD-L1 polypeptide or variant thereof, in some cases the one or more MODs is a 4-1BBL polypeptide or variant thereof, in some cases the one or more MODs is an IL-2 polypeptide or variant thereof, and in some cases the one or more MODs is a FasL polypeptide or variant thereof. In some cases, the T1D peptide is a proinsulin peptide. In some cases, the T1D peptide is a GAD peptide. In any of the above embodiments, in some cases, the first MHC class II polypeptide is an MHC class II β chain polypeptide and the second MHC class II polypeptide is an MHC class II α chain polypeptide.

As another example, in some cases, a TMP comprises: a) a first polypeptide comprising, in order from N-terminus to C-terminus: i) one or more MODs; ii) a T1D peptide; and iii) a first MHC class II polypeptide comprising a substitution of an amino acid (other than a Cys) with a Cys (a “first Cys”), the components of the first polypeptide optionally being joined by one or more linkers that do not comprise a Cys; and b) a second polypeptide comprising, in order from N-terminus to C-terminus: i) a second MHC class II polypeptide comprising a substitution of an amino acid (other than a Cys) with a Cys (a “second Cys”); and ii) optionally an Ig Fc polypeptide, where when the optional Ig Fc is present, the components of the second polypeptide are optionally joined by a linker that does not comprise a Cys; where the first polypeptide and the second polypeptide are linked via a disulfide bond between the first Cys and the second Cys; where the first or the second polypeptide comprises a TGF-β polypeptide, and where the first or the second polypeptide comprises a masking polypeptide. In any of the above embodiments, in some cases the one or more MODs is a PD-L1 polypeptide or variant thereof, in some cases the one or more MODs is a 4-1BBL polypeptide or variant thereof, in some cases the one or more MODs is an IL-2 polypeptide or variant thereof, and in some cases the one or more MODs is a FasL polypeptide or variant thereof. In some cases, the T1D peptide is a proinsulin peptide. In some cases, the T1D peptide is a GAD peptide. In any of the above embodiments, in some cases, the first MHC class II polypeptide is an MHC class II β chain polypeptide and the second MHC class II polypeptide is an MHC class II α chain polypeptide.

As another example, in some cases, a TMP comprises: a) a first polypeptide comprising, in order from N-terminus to C-terminus: i) a T1D peptide; ii) a first MHC class II polypeptide comprising a substitution of an amino acid (other than a Cys) with a Cys (a “first Cys”); and iii) one or more MODs, the components of the first polypeptide optionally being joined by one or more linkers that do not comprise a Cys; and b) a second polypeptide comprising, in order from N-terminus to C-terminus: i) a second MHC class II polypeptide comprising a substitution of an amino acid (other than a Cys) with a Cys (a “second Cys”); and ii) optionally an Ig Fc polypeptide, where when the optional Ig Fc is present, the components of the second polypeptide are optionally joined by a linker that does not comprise a Cys; where the first polypeptide and the second polypeptide are linked via a disulfide bond between the first Cys and the second Cys; where the first or the second polypeptide comprises a TGF-β polypeptide, and where the first or the second polypeptide comprises a masking polypeptide. In any of the above embodiments, in some cases the one or more MODs is a PD-L1 polypeptide or variant thereof, in some cases the one or more MODs is a 4-1BBL polypeptide or variant thereof, in some cases the one or more MODs is an IL-2 polypeptide or variant thereof, and in some cases the one or more MODs is a FasL polypeptide or variant thereof. In some cases, the T1D peptide is a proinsulin peptide. In some cases, the T1D peptide is a GAD peptide. In any of the above embodiments, in some cases, the first MHC class II polypeptide is an MHC class II β chain polypeptide and the second MHC class II polypeptide is an MHC class II α chain polypeptide.

Location of Cys Residues

As noted above, in some cases, a TMP in some cases comprises a heterodimer comprising a first polypeptide chain comprising: i) a T1D peptide; and ii) a first MHC class II polypeptide; and h) a second polypeptide comprising a second MHC class II polypeptide, where the first and/or the second polypeptides comprise one or more MODs, where the first or the second polypeptide comprises a masking polypeptide, and where the first or the second polypeptide comprises a TGF-β polypeptide, optionally wherein the first and/or the second polypeptide comprises an Ig Fc polypeptide; where the first MHC class II polypeptide comprises a substitution of an amino acid (other than a Cys) with a Cys (a “first Cys”), and the second MHC class II polypeptide comprises a substitution of an amino acid (other than a Cys) with a Cys (a “second” Cys), where the first polypeptide and the second polypeptide are joined by a disulfide bond formed between the first Cys and the second Cys.

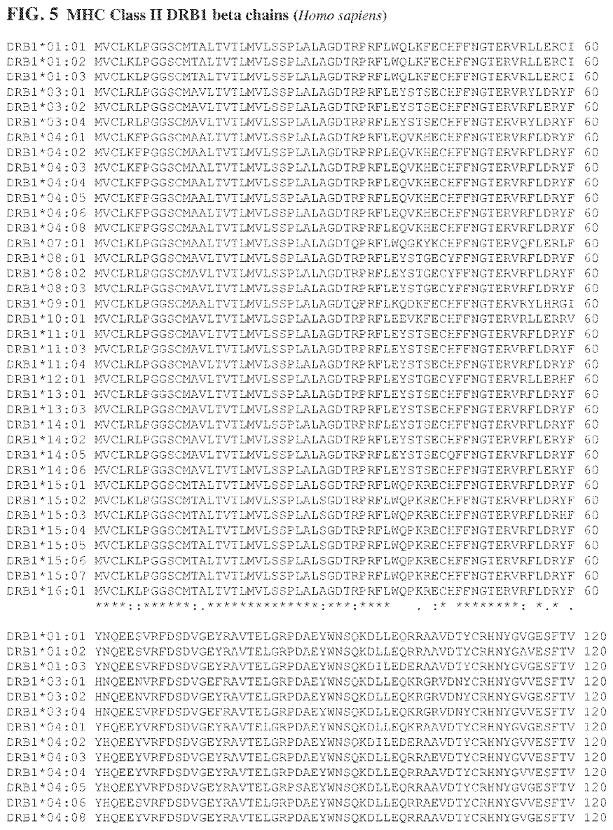

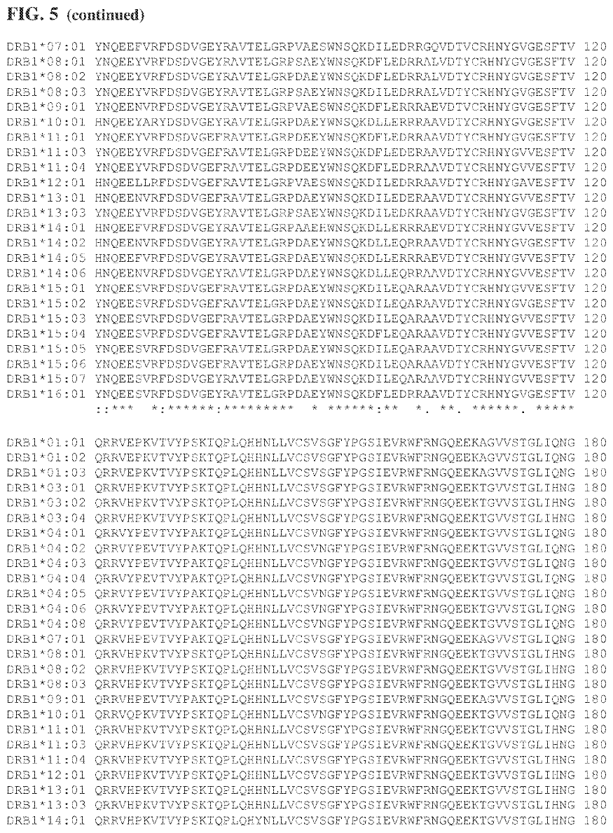

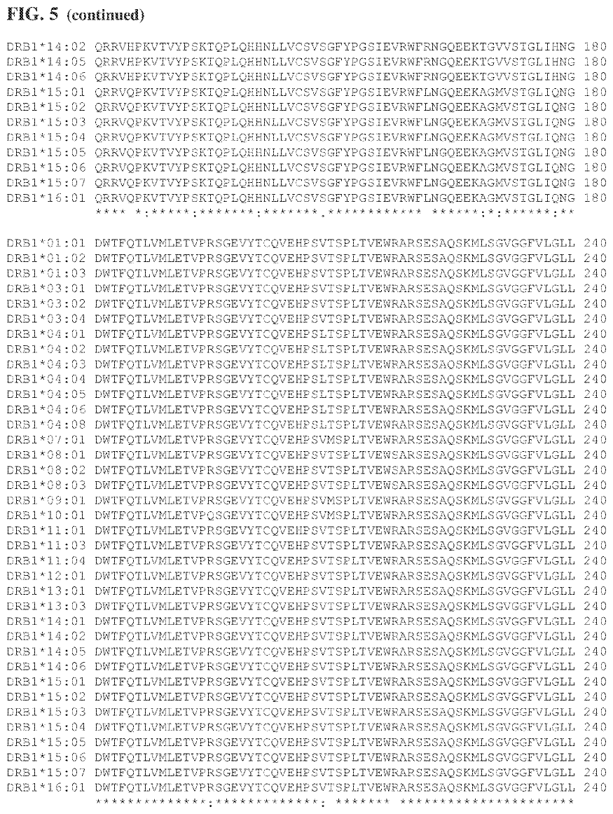

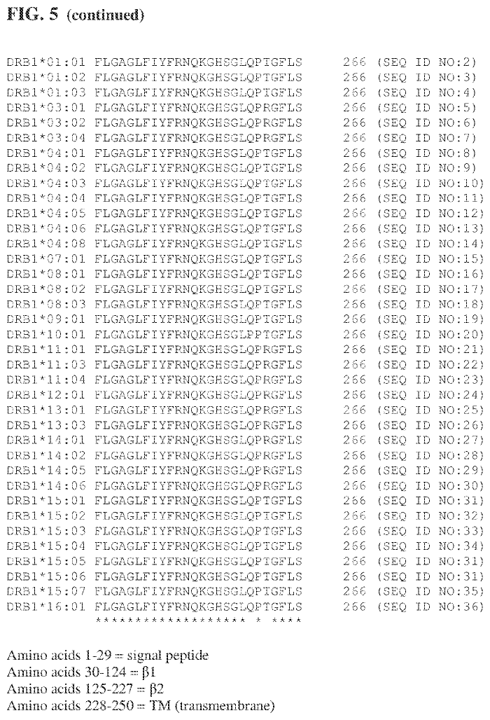

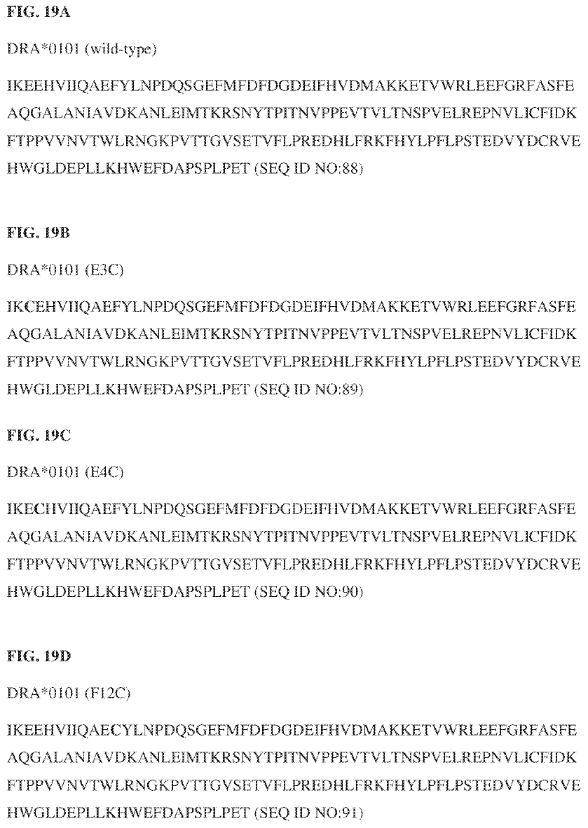

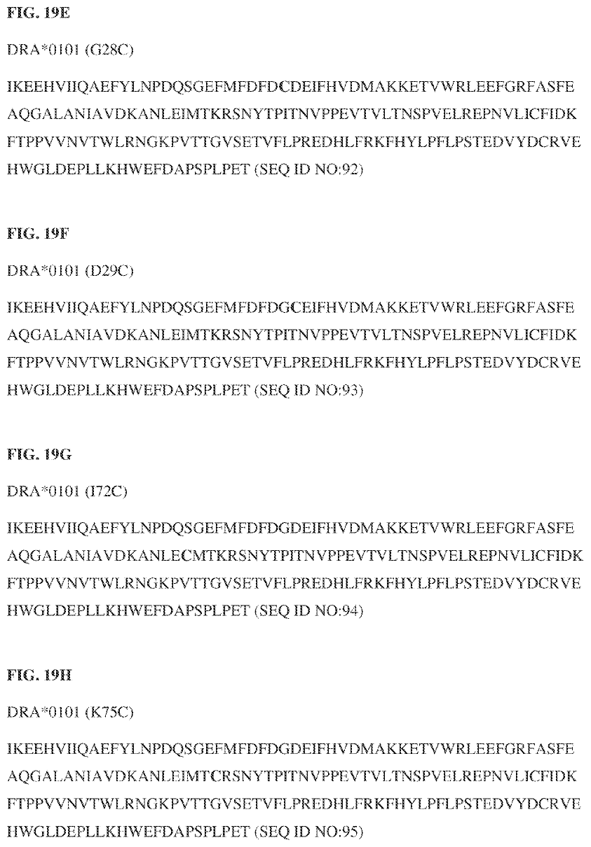

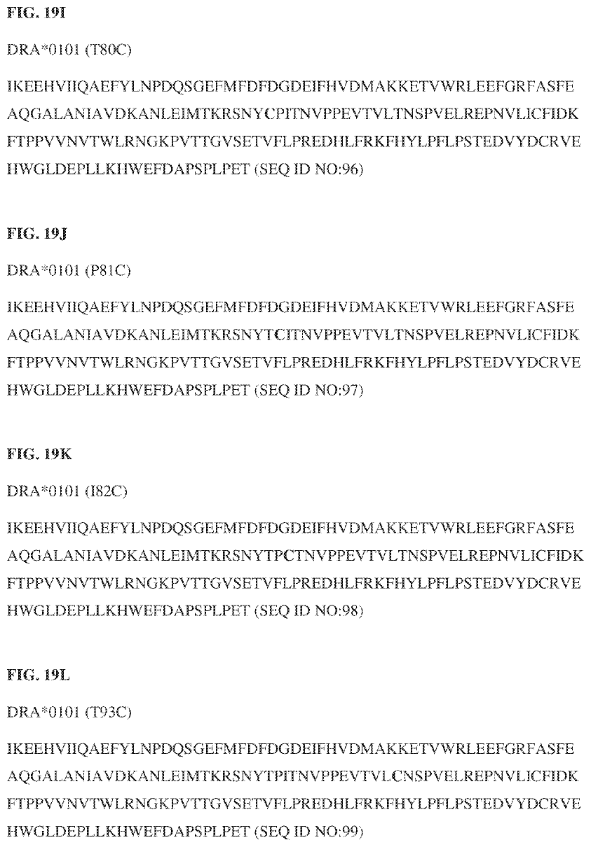

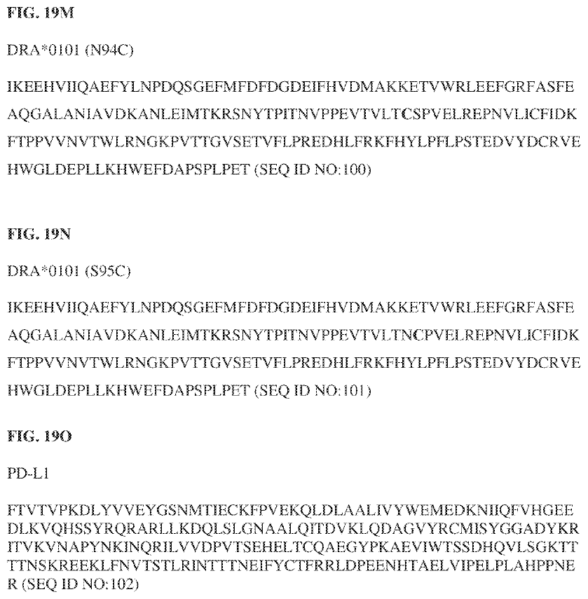

The first MHC class II polypeptide can be a DRB MHC class II polypeptide comprising an amino acid sequence having at least 80%, at least 85%, at least 90%, at least 95%, at least 98%, or at least 99% amino acid sequence identity to the amino acid sequence depicted in FIG. 5 or FIG. 20A and having an amino acid substitution selected from P5C, F7C, Q10C, N19C, G20C, 1133C, G151C, D152C, and W153C. The second MHC class II polypeptide can be a DRA MHC class II polypeptide comprising an amino acid sequence having at least 80%, at least 85%, at least 90%, at least 95%, at least 98%, or at least 99%, amino acid sequence identity to the amino acid sequence depicted in FIG. 4 or FIG. 19A and having an amino acid substitution selected from E3C, E4C, F12C, G28C, D29C, 172C, K75C, T80C, P81C, 182C, T93C, N94C, and S95C.

As one example, in some cases, the first MHC class II polypeptide is a DRB MHC class II polypeptide comprising a substitution at a residue selected from the group consisting of P5C, H33C, G151C, and W153, and the second MHC class II polypeptide is a DRA MHC class II polypeptide comprising a substitution selected from the group consisting of P81C, I82C, and D29C. For example, the MHC class II β chain polypeptide can comprise an amino acid sequence having at least 80%, at least 85%, at least 90%, at least 95%, at least 98%, or at least 99% amino acid sequence identity to the amino acid sequence depicted in FIG. 5 or FIG. 20A, where the polypeptide comprises a Cys at a residue position selected from the group consisting of 5, 33, 151, and 153; and the MHC class II α chain polypeptide can comprise an amino acid sequence having at least 80%, at least 85%, at least 90%, at least 95%, at least 98%, or at least 99% amino acid sequence identity to the amino acid sequence depicted in FIG. 4 or FIG. 19A, where the polypeptide comprises a Cys at a residue selected from the group consisting of 81, 82, and 29.

As another example, the disulfide can be formed between one of the specific pairs of Cys residues in Table 2, below:

TABLE 2

| DRB MHC Class II Polypeptide | DRA MHC Class II |
|---|---|
| Substitution | Polypeptide Substitution |
| N19C | E3C |
| G20C | E3C |
| N19C | E4C |
| G20C | E4C |
| F7C | F12C |
| Q10C | F12C |
| G151C | G28C |
| D152C | G28C |
| W153C | G28C |
| G151C | D29C |
| D152C | D29C |
| W153C | D29C |
| F7C | T80C |
| P5C | T80C |
| H33C | T80C |
| P5C | P81C |
| H33C | P81C |
| F7C | P81C |
| H33C | I82C |
| F7C | I82C |
| P5C | I82C |
| Q156C | T93C |
| W153C | T93C |
| Q156C | N94C |
| N120C | N94C |
| Q156C | S95C |
| N120C | S95C |

For example, the DRB MHC class II polypeptide can comprise an amino acid sequence having at least 80%, at least 85%, at least 90%, at least 95%, at least 98%, or at least 99% amino acid sequence identity to the amino acid sequence depicted in FIG. 5 or FIG. 20A, and the DRA MHC class II polypeptide can comprise an amino acid sequence having at least 80%, at least 85%, at least 90%, at least 95%, at least 98%, or at least 99% amino acid sequence identity to the amino acid sequence depicted in FIG. 4 or FIG. 19A, wherein the Q chain polypeptide and a chain polypeptides are joined by a disulfide formed between a pair of Cys residues selected from the group consisting of: β chain polypeptide residue 5 and α chain polypeptide residue 81; β chain polypeptide residue 33 and α chain polypeptide residue 81; β chain polypeptide residue 33 and α chain polypeptide residue 82; D chain polypeptide residue 151 and α chain polypeptide residue 29; and β chain polypeptide residue 153 and α chain polypeptide residue 29.

In some cases, a TMP comprises a DRB MHC class II polypeptide comprising a P5C substitution and a DRA MHC class II polypeptide comprising a P81C substitution; where the first polypeptide and the second polypeptide of the TMP are linked via a disulfide bond between the Cys at residue 5 in the DRB MHC class II polypeptide and the Cys at residue 81 in the DRA MHC class II polypeptide. In some cases, linkers that do not comprise a Cys optionally can be used to connect the components of the first or second polypeptide. In some cases, the one or more MODs present in the TMP is a PD-L1 polypeptide or variant thereof, an IL-2 polypeptide or variant thereof, or a FasL polypeptide or variant thereof. In some cases, the TMP comprises an Ig Fc polypeptide, and in some cases, the IgFc polypeptide is a human IgG1 Fc polypeptide, optionally comprising L234A and L235A substitutions (L14A and L15A of the amino acid sequence depicted in FIG. 21A). In some cases, the DRB MHC class II polypeptide comprises an amino acid sequence having at least 80%, at least 85%, at least 90%, at least 95%, at least 98%, or at least 99% amino acid sequence identity to the amino acid sequence depicted in FIG. 20B, and comprises a Cys at position 5. In some cases, the DRA MHC class II polypeptide comprises an amino acid sequence having at least 80%, at least 85%, at least 90%, at least 95%, at least 98%, or at least 99% amino acid sequence identity to the amino acid sequence depicted in FIG. 19J, and comprises a Cys at position 81. In some cases, the T1D peptide is a proinsulin peptide. In some cases, the T1D peptide is a proinsulin peptide selected from SLQPLALEGSLQKRG (SEQ ID NO:175; proIns 76-90). SLQPLALEGSLQSRG (SEQ ID NO:159; proIns 76-90; K88S), and GAGSLQPLALEGSLQKRG (SEQ ID NO:176; proIns 73-90). In some cases, the T1D peptide is a GAD peptide. In some cases, the T1D peptide is a GAD peptide selected from NFFRMVISNPAAT (SEQ ID NO:177; GAD65 555-567) and NFIRMVISNPAAT (SEQ ID NO:163; GAD65 555-567; F557I).

In some cases, a TMP comprises a DRB MHC class II polypeptide comprising an H33C substitution and a DRA MHC class II polypeptide comprising a P81C substitution, where the first polypeptide and the second polypeptide of the TMP are linked via a disulfide bond between the Cys at residue 33 in the DRB MHC class II polypeptide and the Cys at residue 81 in the DRA MHC class II polypeptide. In some cases, linkers that do not comprise a Cys optionally can be used to connect the components of the first or second polypeptide. In some cases, the one or more MODs present in the TMP is a PD-L1 polypeptide or variant thereof, an IL-2 polypeptide or variant thereof, or a Fast polypeptide or variant thereof. In some cases, the TMP comprises an Ig Fc polypeptide, and in some cases, the IgFc polypeptide is a human IgG1 Fe polypeptide, optionally comprising L234A and L235A substitutions (L14A and L15A of the amino acid sequence depicted in FIG. 21A). In some cases, the DRB MHC class II polypeptide comprises an amino acid sequence having at least 80%, at least 85%, at least 90%, at least 95%, at least 98%, or at least 99% amino acid sequence identity to the amino acid sequence depicted in FIG. 20G, and comprises a Cys at position 33. In some cases, the DRA MHC class II polypeptide comprises an amino acid sequence having at least 80%, at least 85%, at least 90%, at least 95%, at least 98%, or at least 99% amino acid sequence identity to the amino acid sequence depicted in FIG. 19J, and comprises a Cys at position 81. In some cases, the T1D peptide is a proinsulin peptide. In some cases, the T1D peptide is a proinsulin peptide selected from SLQPLALEGSLQKRG (SEQ ID NO:175; proIns 76-90), SLQPLALEGSLQSRG (SEQ ID NO:159; proIns 76-90; K88S), and GAGSLQPLALEGSLQKRG (SEQ ID NO:176; proIns 73-90). In some cases, the T1D peptide is a GAD peptide. In some cases, the T1D peptide is a GAD peptide selected from NFFRMVISNPAAT (SEQ ID NO:177; GAD65 555-567) and NFIRMVISNPAAT (SEQ ID NO:163; GAD65 555-567; F557I).

In some cases, a TMP comprises a DRB MHC class II polypeptide comprising an H33C substitution and a DRA MHC class II polypeptide comprising a 182C substitution, where the first polypeptide and the second polypeptide of the TMP are linked via a disulfide bond between the Cys at residue 33 in the DRB MHC class II polypeptide and the Cys at residue 81 in the DRA MHC class II polypeptide. In some cases, linkers that do not comprise a Cys optionally can be used to connect the components of the first or second polypeptide. In some cases, the one or more MODs present in the TMP is a PD-L1 polypeptide or variant thereof, an IL-2 polypeptide or variant thereof, or a FasL polypeptide or variant thereof. In some cases, the TMP comprises an Ig Fc polypeptide, and in some cases, the IgFc polypeptide is a human IgG1 Fc polypeptide, optionally comprising L234A and L235A substitutions (L14A and L15A of the amino acid sequence depicted in FIG. 21A). In some cases, the DRB MHC class II polypeptide comprises an amino acid sequence having at least 80%, at least 85%, at least 90%, at least 95%, at least 98%, or at least 99% amino acid sequence identity to the amino acid sequence depicted in FIG. 20G, and comprises a Cys at position 33. In some cases, the DRA MHC class II polypeptide comprises an amino acid sequence having at least 80%, at least 85%, at least 90%, at least 95%, at least 98%, or at least 99% amino acid sequence identity to the amino acid sequence depicted in FIG. 19K, and comprises a Cys at position 82. In some cases, the T1D peptide is a proinsulin peptide. In some cases, the T1D peptide is a proinsulin peptide selected from SLQPLALEGSLQKRG (SEQ ID NO:175; proIns 76-90), SLQPLALEGSLQSRG (SEQ ID NO:159; proIns 76-90; K88S), and GAGSLQPLALEGSLQKRG (SEQ ID NO:176; proIns 73-90). In some cases, the T1D peptide is a GAD peptide. In some cases, the T1D peptide is a GAD peptide selected from NFFRMVISNPAAT (SEQ ID NO:177; GAD65 555-567) and NFIRMVISNPAAT (SEQ ID NO:163; GAD65 555-567; F557I).

In some cases, a TMP comprises a DRB MHC class II polypeptide comprising an G151C substitution and a DRA MHC class II polypeptide comprising a D29C substitution, where the first polypeptide and the second polypeptide of the TMP are linked via a disulfide bond between the Cys at residue 33 in the DRB MHC class II polypeptide and the Cys at residue 81 in the DRA MHC class II polypeptide. In some cases, linkers that do not comprise a Cys optionally can be used to connect the components of the first or second polypeptide. In some cases, the one or more MODs present in the TMP is a PD-L1 polypeptide or variant thereof, an IL-2 polypeptide or variant thereof, or a FasL polypeptide or variant thereof. In some cases, the TMP comprises an Ig Fc polypeptide, and in some cases, the IgFc polypeptide is a human IgG1 Fc polypeptide, optionally comprising L234A and L235A substitutions (L14A and L15A of the amino acid sequence depicted in FIG. 21A). In some cases, the DRB MHC class II polypeptide comprises an amino acid sequence having at least 80%, at least 85%, at least 90%, at least 95%, at least 98%, or at least 99% amino acid sequence identity to the amino acid sequence depicted in FIG. 20H, and comprises a Cys at position 151. In some cases, the DRA MHC class II polypeptide comprises an amino acid sequence having at least 80%, at least 85%, at least 90%, at least 95%, at least 98%, or at least 99% amino acid sequence identity to the amino acid sequence depicted in FIG. 19F, and comprises a Cys at position 29. In some cases, the T1D peptide is a proinsulin peptide. In some cases, the T1D peptide is a proinsulin peptide selected from SLQPLALEGSLQKRG (SEQ ID NO:175 proIns 76-90). SLQPLALEGSLQSRG (SEQ ID NO:159; proIns 76-90; K88S), and GAGSLQPLALEGSLQKRG (SEQ ID NO:176; proIns 73-90). In some cases, the T1D peptide is a GAD peptide. In some cases, the T1 D peptide is a GAD peptide selected from NFFRMVISNPAAT (SEQ ID NO:177; GAD65 555-567) and NFIRMVISNPAAIT (SEQ ID NO:163; GAD65 555-567; F557I).

In some cases, a TMP comprises a DRB MHC class II polypeptide comprising a W153C substitution and a DRA MHC class II polypeptide comprising a D29C substitution, where the first polypeptide and the second polypeptide of the TMP are linked via a disulfide bond between the Cys at residue 33 in the DRB MHC class II polypeptide and the Cys at residue 81 in the DRA MHC class II polypeptide. In some cases, linkers that do not comprise a Cys optionally can be used to connect the components of the first or second polypeptide. In some cases, the one or more MODs present in the TMP is a PD-L1 polypeptide or variant thereof, an IL-2 polypeptide or variant thereof, or a FasL polypeptide or variant thereof. In some cases, the TMP comprises an Ig Fc polypeptide, and in some cases, the IgFc polypeptide is a human IgG1 Fe polypeptide, optionally comprising L234A and L235A substitutions (L14A and L15A of the amino acid sequence depicted in FIG. 21A). In some cases, the DRB MHC class II polypeptide comprises an amino acid sequence having at least 80%, at least 85%, at least 90%, at least 95%, at least 98%, or at least 99% amino acid sequence identity to the amino acid sequence depicted in FIG. 20J, and comprises a Cys at position 153. In some cases, the DRA MHC class II polypeptide comprises an amino acid sequence having at least 80%, at least 85%, at least 90%, at least 95%, at least 98%, or at least 99% amino acid sequence identity to the amino acid sequence depicted in FIG. 19F and comprises a Cys at position 29. In some cases, the T1D peptide is a proinsulin peptide. In some cases, the T1D peptide is a proinsulin peptide selected from SLQPLALEGSLQKRG (SEQ ID NO:175; proIns 76-90), SLQPLALEGSLQSRG (SEQ ID NO:159; proIns 76-90; K88S), and GAGSLQPLALEGSLQKRG (SEQ ID NO:176; proIns 73-90). In some cases, the T1D peptide is a GAD peptide. In some cases, the T1D peptide is a GAD peptide selected from NFFRMVISNPAAT (SEQ ID NO:177; GAD65 555-567) and NFIRMVISNPAAT (SEQ ID NO:163; GAD65 555-567; F557I).

2) Disulfide Bond Between a Peptide Linker and an MHC Class II Polypeptide

In some cases, the TMP comprises a peptide linker between the T1D peptide and the MHC class II polypeptide. Where desired, the linker can comprise a Cys that can be used to form a disulfide bond between the two polypeptides in the heterodimer.

Arrangement of Components

In some cases, a TMP comprises: a) a first polypeptide comprising, in order from N-terminus to C-terminus: i) a T1D peptide; ii) a peptide linker comprising a Cys; iii) an MHC class III polypeptide; and b) a second polypeptide comprising, in order from N-terminus to C-terminus (optionally including one or more linkers that do not comprise a Cys); i) one or more MODs; ii) an MHC class II α chain; and (optionally) iii) an Ig Fc polypeptide; where the first or the second polypeptide comprises a TGF-β polypeptide, and where the first or the second polypeptide comprises a masking polypeptide. The peptide linker comprising a Cys can comprise an amino acid sequence selected from (CGGGS)(GGGGS)ₙ (SEQ ID NO:178), (GCGGS)(GGGGS)ₙ (SEQ ID NO:179), (GGCGS)(GGGGS)ₙ (SEQ ID NO:180), (GGGCS)(GGGGS)ₙ (SEQ ID NO:181), and (GGGGC)(GGGGS)ₙ (SEQ ID NO:182), where n is an integer from 1 to 10 (e.g., where n is 1, 2, 3, 4, 5, 6, 7, 8, 9, or 10). In any of the above embodiments, in some cases the MOD is a PD-L1 polypeptide or variant thereof, in some cases the MOD is an IL-2 polypeptide or variant thereof, and in some cases the MOD is a Fast polypeptide or variant thereof. In some cases, the T1D peptide is a proinsulin peptide. In some cases, the TD peptide is a GAD peptide. In any of the above embodiments, in some cases, the MHC class II α chain comprises an amino acid substitution of an amino acid (other than a Cys) with a Cys, such that the Cys in the MHC class II α chain forms a disulfide bond with the Cys in the first polypeptide linker.

In some cases, a TMP comprises: a) a first polypeptide comprising, in order from N-terminus to C-terminus: i) a T1D peptide; ii) a peptide linker comprising a Cys; and iii) an MHC class II β polypeptide; and b) a second polypeptide comprising, in order from N-terminus to C-terminus (optionally including one or more linkers that do not comprise a Cys); i) an MHC class II α chain; ii) one or more MODs; and iii) an Ig Fc polypeptide; where the first or the second polypeptide comprises a TGF-β polypeptide, and where the first or the second polypeptide comprises a masking polypeptide. The peptide linker comprising a Cys can comprise, e.g., an amino acid sequence selected from (CGGGS)(GGGGS)ₙ (SEQ ID NO:178). (GCGGS)(GGGS)ₙ (SEQ ID NO:179). (GGCGS)(GGGGS)ₙ (SEQ ID NO:180), (GGGCS)(GGGGS)ₙ (SEQ ID NO:181), and (GGGGC)(GGGGS)ₙ (SEQ ID NO:182), where n is an integer from 1 to 10 (e.g., where n is 1, 2, 3, 4, 5, 6, 7, 8, 9, or 10). Other peptide linkers comprising a Cys may be used. In any of the above embodiments, in some cases the one or more MODs is a PD-L1 polypeptide or variant thereof, in some cases the one or more MODs is a 4-1BBL polypeptide or variant thereof, in some cases the one or more MODs is an IL-2 polypeptide or variant thereof, and in some cases the one or more MODs is a FasL polypeptide or variant thereof. In some cases, the T1D peptide is a proinsulin peptide. In some cases, the T1D peptide is a GAD peptide. In any of the above embodiments, in some cases, the MHC class II α chain comprises an amino acid substitution of an amino acid (other than a Cys) with a Cys, such that the Cys in the MHC class II α chain forms a disulfide bond with the Cys in the linker.

In some cases, a TMP comprises: a) a first polypeptide comprising, in order from N-terminus to C-terminus: i) a T1D peptide; ii) a peptide linker comprising a Cys; and iii) an MHC class II § polypeptide; and b) a second polypeptide comprising, in order from N-terminus to C-terminus (optionally including one or more linkers that do not comprise a Cys); i) an MHC class II α chain; and ii) one or more MODs; where the first or the second polypeptide comprises an Ig Fc polypeptide, where the first or the second polypeptide comprises a TGF-β polypeptide, and where the first or the second polypeptide comprises a masking polypeptide. The peptide linker comprising a Cys can comprise. e.g., an amino acid sequence selected from (CGGGS)(GGGGS)ₙ (SEQ ID NO:178), (GCGGS)(GGGGS)ₙ (SEQ ID NO:179). (GGCGS)(GGGGS)ₙ (SEQ ID NO: 180), (GGGCS)(GGGGS)ₙ (SEQ ID NO:181), and (GGGGC)(GGGGS)ₙ (SEQ ID NO:182), where n is an integer from 1 to 10 (e.g., where n is 1, 2, 3, 4, 5, 6, 7, 8, 9, or 10). Other peptide linkers comprising a Cys may be used. In any of the above embodiments, in some cases the one or more MODs is a PD-L1 polypeptide or variant thereof, in some cases the one or more MODs is a 4-1BBL polypeptide or variant thereof, in some cases the one or more MODs is an IL-2 polypeptide or variant thereof, and in some cases the one or more MODs is a Fast, polypeptide or variant thereof. In some cases, the T1D peptide is a proinsulin peptide. In some cases, the T1D peptide is a GAD peptide. In any of the above embodiments, in some cases, the MHC class II α chain comprises an amino acid substitution of an amino acid (other than a Cys) with a Cys, such that the Cys in the MHC class II α chain forms a disulfide bond with the Cys in the linker.

In some cases, a TMP comprises: a) a first polypeptide comprising, in order from N-terminus to C-terminus: i) one or more MODs; ii) a T1D peptide; ii) a peptide linker comprising a Cys; and iii) an MHC class II β polypeptide; and b) a second polypeptide comprising, in order from N-terminus to C-terminus: i) an MHC class II α chain; and (optionally) ii) an Ig Fc polypeptide, optionally joined by a linker that does not comprise a Cys; where the first or the second polypeptide comprises a TGF-β polypeptide, and where the first or the second polypeptide comprises a masking polypeptide. The peptide linker comprising a Cys can comprise, e.g., an amino acid sequence selected from (CGGGS)(GGGGS)ₙ (SEQ ID NO:178), (GCGGS)(GGGGS)ₙ (SEQ ID NO:179). (GGCGS)(GGGGS)ₙ (SEQ ID NO:180). (GGGCS)(GGGGS)ₙ (SEQ ID NO:181), and (GGGGC)(GGGGS)ₙ (SEQ ID NO:182), where n is an integer from 1 to 10 (e.g., where n is 1, 2, 3, 4, 5, 6, 7, 8, 9, or 10). Other peptide linkers comprising a Cys may be used. In any of the above embodiments, in some cases the one or more MODs is a PD-L1 polypeptide or variant thereof, in some cases the one or more MODs is a 4-1BBL polypeptide or variant thereof, in some cases the one or more MODs is an IL-2 polypeptide or variant thereof, and in some cases the one or more MODs is a FasL polypeptide or variant thereof. In some cases, the T1D peptide is a proinsulin peptide. In some cases, the T1D peptide is a GAD peptide. In any of the above embodiments, in some cases, the MHC class II α chain comprises an amino acid substitution of an amino acid (other than a Cys) with a Cys, such that the Cys in the MIC class II α chain forms a disulfide bond with the Cys in the linker.

In some cases, a TMP comprises: a) a first polypeptide comprising, in order from N-terminus to C-terminus: i) a T1D peptide; ii) a peptide linker comprising a Cys; iii) an MHC class II β polypeptide; and iv) one or more MODs; and b) a second polypeptide comprising, in order from N-terminus to C-terminus: i) an MHC class II α chain; and (optionally) ii) an Ig Fc polypeptide, optionally joined by a linker that does not comprise a Cys; where the first or the second polypeptide comprises a TGF-β polypeptide, and where the first or the second polypeptide comprises a masking polypeptide. The peptide linker comprising a Cys can comprise, e.g., an amino acid sequence selected from (CGGGS)(GGGGS)ₙ (SEQ ID NO:178), (GCGGS)(GGGGS)ₙ (SEQ ID NO:179). (GGCGS)(GGGGS)ₙ (SEQ ID NO:180), (GGGCS)(GGGS)ₙ (SEQ ID NO:181), and (GGGGC)(GGGGS)ₙ (SEQ ID NO:182), where n is an integer from 1 to 10 (e.g., where n is 1, 2, 3, 4, 5, 6, 7, 8, 9, or 10). Other peptide linkers comprising a Cys may be used. In any of the above embodiments, in some cases the one or more MODs is a PD-L1 polypeptide or variant thereof, in some cases the one or more MODs is a 4-1BBL, polypeptide or variant thereof, in some cases the one or more MODs is an IL-2 polypeptide or variant thereof, and in some cases the one or more MODs is a FasL polypeptide or variant thereof. In some cases, the T1D peptide is a proinsulin peptide. In some cases, the T1D peptide is a GAD peptide. In any of the above embodiments, in some cases, the MHC class II α chain comprises an amino acid substitution of an amino acid (other than a Cys) with a Cys, such that the Cys in the MHC class II α chain forms a disulfide bond with the Cys in the linker.

Location of Cys Residues

As noted above, in some cases, a TMP comprises a heterodimer comprising a first polypeptide chain comprising: i) a T1D peptide; ii) a peptide linker comprising a Cys; and iii) a first MHC class II polypeptide; and b) a second polypeptide comprising a second MHC class II polypeptide, where the first and/or the second polypeptides comprise one or more MODs, where the first or the second polypeptide comprises a TGF-β polypeptide, where the first or the second polypeptide comprises a masking polypeptide, optionally wherein the first or the second polypeptide comprises an Ig Fc polypeptide, where the second MHC class II polypeptide comprises a substitution of an amino acid (other than a Cys) with a Cys; and where the first and the second polypeptides are linked via a disulfide bond between the Cys in the peptide linker and the Cys provided by the substitution in the second MHC class II polypeptide.

For example, in some cases, the peptide linker can comprise an amino acid sequence selected from: (CGGGS)(GGGGS)ₙ (SEQ ID NO:178), (GCGGS)(GGGGS)ₙ (SEQ ID NO:179), (GGCGS)(GGGGS)ₙ (SEQ ID NO:180). (GGGCS)(GGGGS)ₙ (SEQ ID NO:181), and (GGGGC)(GGGGS)ₙ (SEQ ID NO:182), where n is an integer from 1 to 10 (e.g., where n is 1, 2, 3, 4, 5, 6, 7, 8, 9, or 10, with n=2 or 3 generally being useful); the first MHC class II polypeptide is a DRB MHC class II polypeptide; and the second MHC class II polypeptide is a DRA MHC class II polypeptide that comprises a substitution of an amino acid (other than a Cys) with a Cys.

Specific examples of the Cys-containing linkers between the T1D peptide and a DRB MHC class II polypeptide and Cys residues in the DRA MHC class II polypeptide that can form a disulfide bond are provided in Table 3, below:

TABLE 3

| Cys-Containing Linker | |
|---|---|
| Between the T1D peptide | DRA MHC class II |
| and the DRB MHC Class II | polypeptide |
| Polypeptide | substitution |
| (CGGGS)(GGGGS)n (SEQ ID | I72C |
| NO: 178) | |
| (GCGGS)(GGGGS)n (SEQ ID | I72C |
| NO: 179) | |
| (GGCGS)(GGGGS)n (SEQ ID | I72C |
| NO: 180) | |
| (GGGCS)(GGGGS)n (SEQ ID | I72C |
| NO: 181) | |
| (GGGGC)(GGGGS)n (SEQ ID | I72C |
| NO: 182) | |
| (CGGGS)(GGGGS)n (SEQ ID | K75C |
| NO: 178) | |
| (GCGGS)(GGGGS)n (SEQ ID | K75C |
| NO: 179) | |
| (GGCGS)(GGGGS)n (SEQ ID | K75C |
| NO: 180) | |
| (GGGCS)(GGGGS)n (SEQ ID | K75C |
| NO: 181) | |
| (GGGGC)(GGGGS)n (SEQ ID | K75C |
| NO: 182) | |

In some cases, where the first and second MHC class II polypeptides are DRB and DRA polypeptides, respectively, the DRB MHC class II polypeptide can comprise an amino acid sequence having at least 80%, at least 85%, at least 90%, at least 95%, at least 98%, at least 99%, or 100%, amino acid sequence identity to the amino acid sequence depicted in FIG. 5 or FIG. 20A. The DRA MHC class II polypeptide can comprise an amino acid sequence having at least 80%, at least 85%, at least 90%, at least 95%, at least 98%, or at least 99%, amino acid sequence identity to the amino acid sequence depicted in FIG. 4 or FIG. 19A, and can comprise a Cys at, e.g., position 72 or position 75. In any of the above embodiments, n=0, 1, 2, 3, 4 or more, with 2 or 3 typically being used to create a total linker length of 15 or 20 amino acids, although longer lengths may be possible. In any of the above embodiments, linkers that do not comprise a Cys optionally can be used to connect the other components of the first or second polypeptide. In some cases, the Ig Fc polypeptide is present and is a human IgG1 Fc polypeptide, optionally comprising L234A and L235A substitutions (L14A and L15A of the amino acid sequence depicted in FIG. 21A). In some cases, the T1D peptide is a proinsulin peptide. In some cases, the T1D peptide is a proinsulin peptide selected from SLQPLALEGSLQKRG (SEQ ID NO:175; proIns 76-90). SLQPLALEGSLQSRG (SEQ ID NO:159; proIns 76-90; K88S), and GAGSLQPLALEGSLQKRG (SEQ ID NO:176; proIns 73-90). In some cases, the T1D peptide is a GAD peptide. In some cases, the T1D peptide is a GAD peptide selected from NFFRMVISNPAAT (SEQ ID NO:177; GAD65 555-567) and NFIRMVISNPAAT (SEQ ID NO:163; GAD65 555-567; F557I). In any of the above embodiments, in some cases, the one or more MODs is a PD-L1 polypeptide or variant thereof, a 4-1BBL polypeptide or variant thereof, an IL-2 polypeptide or variant thereof, or a FasL polypeptide or variant thereof.

3) Disulfide Bond Between Peptide Linkers in the First and Second Polypeptides

In some cases, the TMP comprises a first Cys-containing peptide linker between the T1D peptide and the MHC class IT polypeptide in the first polypeptide, and a second Cys-containing peptide linker between two of the components in the second polypeptide. In such cases, the Cys residues in the first and second Cys-containing linkers can be used to form a disulfide bond between the two polypeptides in the heterodimer.

In some cases, a TMP comprises: a) a first polypeptide comprising, in order from N-terminus to C-terminus: i) a T1D peptide; ii) a first peptide linker comprising a Cys; and iii) an MHC class II β polypeptide (e.g., a DRB MHC class II polypeptide comprising an amino acid sequence having at least 80%, at least 85%, at least 90%, at least 95%, at least 98%, at least 99%, or 100% amino acid sequence identity to the amino acid sequence depicted in FIG. 5 or FIG. 20A); and b) a second polypeptide comprising, in order from N-terminus to C-terminus: i) one or more MODs; ii) an MHC class II α chain (e.g., a DRA MHC class II polypeptide comprising an amino acid sequence having at least 80%, at least 85%, at least 90%, at least 95%, at least 98%, at least 99%, or 100% amino acid sequence identity to the amino acid sequence depicted in FIG. 4 or FIG. 19A); and (optionally) iii) an Ig Fc polypeptide; where the first or the second polypeptide comprises a TGF-β polypeptide, and where the first or the second polypeptide comprises a masking polypeptide. The second polypeptide comprises a second peptide linker that comprises a Cys, where the second peptide linker is positioned either between the MOD(s) and the MHC class II α chain or between the MHC class II α chain and the Ig Fc, if present. The second polypeptide optionally may include one or more linkers that do not comprise a Cys. The peptide linkers comprising a Cys can comprise an amino acid sequence selected from (CGGGS)(GGGGS)ₙ (SEQ ID NO:178), (GCGGS)(GGGGS)ₙ (SEQ ID NO:179). (GGCGS)(GGGGS)ₙ (SEQ ID NO:180). (GGGCS)(GGGGS)ₙ (SEQ ID NO:181), and (GGGGC)(GGGGS)ₙ (SEQ ID NO:182), where n is an integer from 1 to 10 (e.g., where n is 1, 2, 3, 4, 5, 6, 7, 8, 9, or 10). In any of the above embodiments, in some cases the one or more MODs is a PD-L1 polypeptide or variant thereof, in some cases the one or more MODs is a 4-1BBL polypeptide or variant thereof, in some cases the one or more MODs is an IL-2 polypeptide or variant thereof, and in some cases the one or more MODs is a FasL polypeptide or variant thereof. In some cases, the T1D peptide is a proinsulin peptide. In some cases, the T1D peptide is a GAD peptide.

In some cases, a TMP comprises: a) a first polypeptide comprising, in order from N-terminus to C-terminus: i) a T1D peptide; ii) a first peptide linker comprising a Cys; and iii) an MHC class II β polypeptide (e.g., a DRB MHC class II polypeptide comprising an amino acid sequence having at least 80%, at least 85%, at least 90%, at least 95%, at least 98%, at least 99%, or 100% amino acid sequence identity to the amino acid sequence depicted in FIG. 5 or FIG. 20A); and b) a second polypeptide comprising, in order from N-terminus to C-terminus: i) an MHC class II α chain (e.g., a DRA MHC class II polypeptide comprising an amino acid sequence having at least 80%, at least 85%, at least 90%, at least 95%, at least 98%, at least 99%, or 100% amino acid sequence identity to the amino acid sequence depicted in FIG. 4 or FIG. 19A); ii) an Ig Fc polypeptide; and iii) one or more MODs; where the first or the second polypeptide comprises a TGF-β polypeptide, and where the first or the second polypeptide comprises a masking polypeptide. The second polypeptide comprises a second peptide linker that comprises a Cys, where the second peptide linker is positioned either between the MHC class II α chain and the Ig Fc or between the Ig Fc and the one or more MODs. The second polypeptide optionally may include one or more linkers that do not comprise a Cys. The peptide linkers comprising a Cys can comprise an amino acid sequence selected from (CGGGS)(GGGGS)ₙ (SEQ ID NO:178). (GCGGS)(GGGGS)ₙ (SEQ ID NO:179). (GGCGS)(GGGGS)ₙ (SEQ ID NO:180), (GGGCS)(GGGGS)ₙ (SEQ ID NO:181), and (GGGGC)(GGGGS)ₙ (SEQ ID NO:182), where n is an integer from 1 to 10 (e.g., where n is 1, 2, 3, 4, 5, 6, 7, 8, 9, or 10). In any of the above embodiments, in some cases the one or more MODs is a PD-L1 polypeptide or variant thereof, in some cases the one or more MODs is a 4-1BBL polypeptide or variant thereof, in some cases the one or more MODs is an IL-2 polypeptide or variant thereof, and in some cases the one or more MODs is a FasL polypeptide or variant thereof. In some cases, the T1D peptide is a proinsulin peptide. In some cases, the T1D peptide is a GAD peptide.

C) Single-Chain TMPs

As noted above, in some cases, a TMP of the present disclosure is a single-chain (a single polypeptide chain) TMP. A single-chain TMP comprises: i) a T1D peptide; ii) an MHC class II β chain polypeptide; iii) an MHC class II α chain polypeptide; iv) one or more MODs; v) a TGF-β polypeptide; and vi) a masking polypeptide; and optionally one or more independently selected linker polypeptides. A single-chain TMP of the present disclosure can also include an Ig Fc polypeptide. A single-chain IMP can comprise two or more MODs, where the two or more MODs can have the same amino acid sequence or different amino acid sequences. Non-limiting examples single-chain TMPs are depicted schematically in FIG. 3A-3D.

Arrangement of Components

The arrangement of the components of a single-chain TMP can vary. As a first non-limiting example, in some cases, a single-chain TMP comprises, in order from N-terminus to C-terminus: i) a T1D peptide; ii) an MHC class II β polypeptide; iii) an MHC class II α polypeptide; iv) a TGF-β polypeptide or a masking polypeptide; v) a masking polypeptide or a TGF-β polypeptide; vi) an Ig Fc polypeptide; and vii) one or more MODs. The components of the single-chain TMP may be joined by one or more independently selected linkers.

As a second non-limiting example, in some cases, a single-chain TMP comprises, in order from N-terminus to C-terminus: i) a T1D peptide; ii) an MHC class II β polypeptide; iii) an MHC class II α polypeptide; iv) a TGF-β polypeptide or a masking polypeptide; v) a masking polypeptide or a TGF-β polypeptide; vi) one or more MODs; and vii) an Ig Fc polypeptide. The components of the first polypeptide may be joined by one or more independently selected linkers.

As a third non-limiting example, in some cases, a single-chain TMP comprises, in order from N-terminus to C-terminus: i) a T1D peptide; ii) an MHC class II β polypeptide; iii) an MHC class II α polypeptide; iv) one or more MODs; v) a TGF-β polypeptide or a masking polypeptide; vi) a masking polypeptide or a TGF-β polypeptide; and vii) an Ig Fc polypeptide. The components of the single-chain TMP may be joined by one or more independently selected linkers.

As a fourth non-limiting example, in some cases, a single-chain TMP comprises, in order from N-terminus to C-terminus: i) a T1D peptide; ii) an MHC class II β polypeptide; iii) an MHC class II α polypeptide; iv) one or more MODs; v) an Ig Fc polypeptide; vi) a TGF-β polypeptide or a masking polypeptide; and vii) a masking polypeptide or a TGF-β polypeptide. The components of the single-chain TMP may be joined by one or more independently selected linkers.

As a fifth non-limiting example, in some cases, a single-chain TMP comprises, in order from N-terminus to C-terminus: i) a T1D peptide; ii) an MHC class II β polypeptide; iii) an MHC class II α polypeptide; iv) an Ig Fc polypeptide; v) one or more MODs; vi) a TGF-β polypeptide or a masking polypeptide; and vii) a masking polypeptide or the TGF-β polypeptide. The components of the single-chain TMP may be joined by one or more independently selected linkers.

As a sixth non-limiting example, in some cases, a single-chain TMP comprises, in order from N-terminus to C-terminus: i) a T1D peptide; ii) an MHC class II β polypeptide; iii) an MHC class II α polypeptide; iv) one or more MODs; v) a TGF-β polypeptide or a masking polypeptide; vi) a masking polypeptide or a TGF-β polypeptide; and vii) an Ig Fc polypeptide. The components of the single-chain TMP may be joined by one or more independently selected linkers.

As a seventh non-limiting example, in some cases, a single-chain TMP comprises, in order from N-terminus to C-terminus: i) a T1D peptide; ii) an MHC class II β polypeptide; iii) an MHC class II α polypeptide; iv) an Ig Fc polypeptide; v) a TGF-β polypeptide or a masking polypeptide; vi) a masking polypeptide or the TGF-β polypeptide; and vii) one or more MODs. The components of the single-chain TMP may be joined by one or more independently selected linkers.

In some cases, as depicted schematically in FIG. 3A, in some cases, a single-chain TMP comprises, in order from N-terminus to C-terminus: i) a T1D peptide; ii) an MHC class II H chain polypeptide; iii) an MHC class II α chain polypeptide; iv) a masking polypeptide; v) a TGF-β polypeptide; vi) an Ig Fc polypeptide; and vii) one or more MODs; and optionally one or more independently selected linker polypeptides. In some cases, a single-chain TMP comprises, in order from N-terminus to C-terminus: i) a T1D peptide; ii) an MHC class II β chain polypeptide; iii) an MHC class II α chain polypeptide; iv) a TGF-β polypeptide; v) a masking polypeptide; vi) an Ig Fc polypeptide; and vii) one or more MODs; and optionally one or more independently selected linker polypeptides. In some cases, as depicted schematically in FIG. 3B, in some cases, a single-chain TMP comprises, in order from N-terminus to C-terminus: i) a T1D peptide; ii) an MHC class II D chain polypeptide; iii) an MHC class II α chain polypeptide; iv) one or more MODs; v) an Ig Fc polypeptide; vi) a masking polypeptide; and vii) a TGF-β polypeptide; and optionally one or more independently selected linker polypeptides. In some cases, a single-chain TMP comprises, in order from N-terminus to C-terminus: i) a T1 D peptide; ii) an MHC class II β chain polypeptide; iii) an MHC class II α chain polypeptide; iv) one or more MODs; v) an Ig Fc polypeptide; vi) a TGF-β polypeptide; and vii) a masking polypeptide; and optionally one or more independently selected linker polypeptides. In some cases, as depicted schematically in FIG. 3C, in some cases, a single-chain TMP comprises, in order from N-terminus to C-terminus: i) a T1D peptide; ii) an MHC class II β chain polypeptide; iii) an MHC class II α chain polypeptide; iv) an Ig Fc polypeptide; v) a masking polypeptide; vi) a TGF-β polypeptide; and vii) one or more MODs; and optionally one or more independently selected linker polypeptides. In some cases, a single-chain TMP comprises, in order from N-terminus to C-terminus: i) a T1D peptide; ii) an MHC class II β chain polypeptide; iii) an MHC class II α chain polypeptide; iv) an Ig Fc polypeptide; v) a TGF-β polypeptide; vi) a masking polypeptide; and vii) one or more MODs; and optionally one or more independently selected linker polypeptides. In some cases, as depicted schematically in FIG. 3D, in some cases, a single-chain TMP comprises, in order from N-terminus to C-terminus: i) a T1D peptide; ii) an MHC class II β chain polypeptide; iii) an MHC class II α chain polypeptide; iv) an Ig Fc polypeptide; v) one or more MODs; vi) a masking polypeptide; and vii) a TGF-β polypeptide; and optionally one or more independently selected linker polypeptides. In some cases, a single-chain TMP comprises, in order from N-terminus to C-terminus: i) a T1D peptide; ii) an MHC class II β chain polypeptide; iii) an MHC class II α chain polypeptide; iv) an Ig Fc polypeptide; v) one or more MODs; vi) a TGF-β polypeptide; and vii) a masking polypeptide; and optionally one or more independently selected linker polypeptides. In any one of the above embodiments, the TMP can include a single MOD. In any one of the above embodiments, the TMP can include 2 copies of a MOD; the 2 copies can be in tandem, or can be separated by a linker. In any one of the above embodiments, the TMP can include 3 copies of the MOD; the 3 copies can be in tandem, or can be separated by a linker.

In any one of the above embodiments, the TMP can comprise a peptide linker between one or more of: i) the T1D peptide and the MHC class II β chain polypeptide; ii) between the MHC class II β chain polypeptide and the MHC class II α chain polypeptide; iii) between the MHC class II α chain polypeptide and the masking polypeptide; iv) between the MHC class II α chain polypeptide and a MOD; v) between the MHC class II α chain polypeptide and an Ig Fc polypeptide; vi) between the masking polypeptide and the TGF-β polypeptide; vii) between the TGF-β polypeptide and the Ig Fc polypeptide; viii) between the Ig Fc polypeptide and a MOD; ix) between a MOD and an Ig Fc polypeptide; or between any other two components of the TMP. Exemplary suitable linkers include (GGGGS)ₙ (SEQ ID NO:183), where n is 1, 2, 3, 4, 5, 6, 7, or 8; AAAGG (SEQ ID NO:184), and GGSAAAGG (SEQ ID NO:162). In any of the above embodiments, in some cases, the Ig Fc is an IgG1 Fc polypeptide or a variant thereof. In any of the above embodiments, in some cases, the Ig Fc is an IgG4 Fe polypeptide or a variant thereof. In any of the above embodiments, in some cases the one or more MODs is a PD-L1 polypeptide or variant thereof, in some cases the one or more MODs is a 4-1BBL polypeptide or variant thereof, in some cases the one or more MODs is an IL-2 polypeptide or variant thereof, and in some cases the one or more MODs is a FasL polypeptide or variant thereof. In some cases, the T1D peptide is proinsulin peptide. In some cases, the T1D peptide is a GAD peptide. In any of the above embodiments, in some cases, the TMP includes one or more intrachain disulfide bonds.

In some cases, a single-chain TMP comprises one or more intrachain disulfide bonds, where an intrachain disulfide bond can form between: a) a Cys present in an MHC class II α chain polypeptide and a Cys present in an MHC class II β chain polypeptide; b) a Cys present in a peptide linker and an MHC class II polypeptide.

A TMP can comprise a homodimer of two identical single-chain TMPs. Where the single-chain TMP comprises an Ig Fc polypeptide, the homodimer can comprise one or more disulfide bonds formed between the Ig Fc polypeptides of each of the two single-chain TMPs present in the homodimer. Alternatively, a TMP can comprise a heterodimer of two different single-chain TMPs that comprise polypeptides having interspecific binding sequences as discussed above, e.g., Ig Fc polypeptides comprising KiH sequences.

### Homodimers and Heterodimers

The present disclosure provides a protein comprising two of the TMPs of the present disclosure. In some cases, the protein is a homodimer comprising two TMP heterodimers. In some cases, the protein is a homodimer comprising two single-chain TMPs. In some cases, the protein is a heterodimer comprising two TMP heterodimers. In some cases, the protein is a heterodimer comprising two single-chain TMPs.

The present disclosure provides a protein that is a homodimer comprising two TMP heterodimers, e.g., a first TMP heterodimer and a second TMP heterodimer, where the first TMP heterodimer and the second TMP heterodimer are the same (i.e., the first polypeptide of the first TMP heterodimer and the second TMP heterodimer have the same amino acid sequence; and the second polypeptide of the first TMP heterodimer and the second TMP heterodimer have the same amino acid sequence). The first TMP heterodimer and the second TMP heterodimer can be covalently linked to one another via one or more disulfide bonds between, e.g., an Ig Fc polypeptide present in the first TMP heterodimer and an Ig Fc polypeptide present in the second TMP heterodimer.

The present disclosure provides a protein that is a heterodimer comprising two TMP heterodimers, e.g., a first TMP heterodimer and a second TMP heterodimer, where the first TMP heterodimer and the second TMP heterodimer are not the same (i.e., the first polypeptide of the first TMP heterodimer and the second TMP heterodimer have different amino acid sequences; and/or the second polypeptide of the first TMP heterodimer and the second TMP heterodimer have different amino acid sequences). In some cases, the first and the second polypeptides of the first and second TMP heterodimers are identical except that the MODs are different. For example, in some case, the first TMP heterodimer comprises one or more MODs, where the one or more MODs comprise IL-2 polypeptides; and the second TMP heterodimer comprises, one or more MODs, where the one or more MODs comprise one or more PD-L1 polypeptides. As another example, in some case, the first TMP heterodimer comprises one or more MODs, where the one or more MODs comprise IL-2 polypeptides; and the second TMP heterodimer comprises, one or more MODs, where the one or more MODs comprise one or more 4-1BBL polypeptides.

The present disclosure provides a protein that is a homodimer comprising two single-chain TMPs, e.g., a first single-chain TMP and a second single-chain TMP, where the first single-chain TMP and the second single-chain TMP are the same (have the same amino acid sequence). The first single-chain TMP and the second single-chain TMP can be covalently linked to one another via one or more disulfide bonds between, e.g., an Ig Fr polypeptide present in the first single-chain TMP and an Ig Fc polypeptide present in the second single-chain TMP.

The present disclosure provides a protein that is a heterodimer comprising two single-chain TMPs, e.g., a first single-chain TMP and a second single-chain TMP, where the first single-chain TMP and the second single-chain TMP are not the same (have different amino acid sequences). In such cases, the two single-chain TMPs can comprise polypeptides having interspecific binding sequences as discussed above. e.g., Ig Fc polypeptides comprising KiH sequences. In some cases, the first and the second polypeptides of the first and second single-chain TMPs are identical except that the MODs are different. For example, in some case, the first single-chain TMP comprises one or more MODs, where the one or more MODs comprise IL-2 polypeptides; and the second single-chain TMP comprises, one or more MODs, where the one or more MODs comprise one or more PD-L1 polypeptides. As another example, in some case, the first single-chain TMP comprises one or more MODs, where the one or more MODs comprise IL-2 polypeptides; and the second single-chain TMP comprises, one or more MODs, where the one or more MODs comprise one or more 4-1BBL polypeptides. The first single-chain TMP and the second single-chain TMP can be covalently linked to one another via one or more disulfide bonds between, e.g., an Ig Fc polypeptide present in the first single-chain TMP and an Ig Fc polypeptide present in the second single-chain TMP.

### Immunomodulatory Polypeptides (“MODs”)

MODs that are suitable for inclusion in a TMP of the present disclosure include, but are not limited to IL-2, CD7, 137-1 (CD80), B7-2 (CD86), PD-L1. PD-L2, 4-1BBL. OX40L, Fas ligand (FasL), inducible costimulatory ligand (ICOS-L), intercellular adhesion molecule (ICAM), CD30L. CD40, CD70. CD83, HLA-G. MICA, MICB. HVEM, lymphotoxin beta receptor, 3/TR6. ILT3, ILT4, and HVEM. In some cases, the MOD is selected from an IL-2 polypeptide, a 4-1 BBL polypeptide, an ICOS-L polypeptide, an OX-40L polypeptide, a CD80 polypeptide, a CD86 polypeptide, a PD-L1 polypeptide, a FasL polypeptide, a and a PD-L2 polypeptide. In some cases, the MOD is selected from an IL-2 polypeptide, a PD-L1 polypeptide, a 4-1BBL polypeptide and a FasL polypeptide.

As also noted above, the MOD can comprise a wild-type amino acid sequence, or can comprise one or more amino acid substitutions relative to a wild-type amino acid sequence. The MOD can comprise only the extracellular portion of a full-length MOD. Thus, for example, the MOD can in some cases exclude one or more of a signal peptide, a transmembrane domain, and an intracellular domain normally found in a naturally-occurring MOD.

In some cases, a MOD suitable for inclusion in a TMP comprises all or a portion of (e.g., an extracellular portion of) the amino acid sequence of a naturally-occurring MOD. In other instances, a MOD suitable for inclusion in a TMP is a variant MOD that comprises at least one amino acid substitution compared to the amino acid sequence of a naturally-occurring MOD. In some instances, a variant MOD exhibits a binding affinity for a co-immunomodulatory polypeptide (“co-MOD”) that is lower than the affinity of a corresponding naturally-occurring MOD (e.g., a MOD not comprising the amino acid substitution(s) present in the variant) for the co-MOD.

Suitable MODs that exhibit reduced affinity for a co-MOD can have from 1 amino acid (aa) to 20 aa differences from a wild-type MOD. For example, in some cases, a variant MOD present in a TMP differs in amino acid sequence by 1 aa, 2 aa, 3 aa, 4 aa, 5 aa, 6 aa, 7 aa, 8 aa, 9 aa, or 10 aa, from a corresponding wild-type MOD. As another example, in some cases, a variant MOD present in a TMP differs in amino acid sequence by 11 aa, 12 aa, 13 aa, 14 aa, 15 aa, 16 aa, 17 aa, 18 aa, 19 aa, or 20 aa, from a corresponding wild-type MOD. As an example, in some cases, a variant MOD present in a TMP includes 1, 2, 3, 4, 5, 6, 7, 8, 9, or 10 amino acid substitutions, compared to a corresponding reference (e.g., wild-type) MOD.

A variant MOD suitable for inclusion in a TMP exhibits reduced affinity for a cognate co-MOD, compared to the affinity of a corresponding wild-type MOD for the cognate co-MOD.

Exemplary pairs of MOD and cognate co-MOD include, but are not limited to those set out in Table 4, below:

TABLE 4

| Immunomodulatory | Cognate Co-Immunomodulatory |
|---|---|
| Polypeptide (MOD) | Polypeptide (co-MOD) |
| 4-1BBL | 4-1BB |
| PD-L1 | PD-1 |
| IL-2 | IL-2 receptor |
| CD80 | CD28 |
| CD86 | CD28 |
| OX40L (CD252) | OX40 (CD134) |
| Fas ligand | Fas |
| ICOS-L | ICOS |
| ICAM | LFA-1 |
| CD30L | CD30 |
| CD40 | CD40L |
| CD83 | CD83L |
| HVEM (CD270) | CD160 |
| JAG1 (CD339) | Notch |
| JAG1 | CD46 |
| CD80 | CTLA4 |
| CD86 | CTLA4 |
| CD70 | CD27 |
| TGFβ | TGFβ receptor |

Binding affinity between a MOD and its cognate co-MOD can be determined by bio-layer interferometry (BLI) using purified MOD and purified cognate co-MOD, following the procedure set forth in published PCI Application WO 2020/132138 A1.

MODs and variants, including reduced affinity variants, such as PD-L1. CD80, CD86, 4-1BBL, and IL-2 are described in the published literature, e.g., published PCT application WO2020132138A1 and WO2019/051091, the disclosures of which as they pertain to MODs and specific variant MODs of PD-L1, CD80, CD86, 4-1BBL, IL-2 are expressly incorporated herein by reference, including specifically paragraphs [00260]-[00455] of WO2020132138A1 and paragraphs [00157]-[00352] of WO2019/051091.

Of specific interest are MODs that are variants of the cytokine IL-2. Wild-type IL-2 binds to IL-2 receptor (IL-2R) on the surface of a T cell. Wild-type IL-2 has a strong affinity for IL-2R and will bind to activate most or substantially all CD8+ T cells. For this reason, synthetic forms of wild type IL-2 such as the drug Aldesleukin (trade name Proleukin®) are known to have severe side-effects when administered to humans for the treatment of cancer because the IL-2 indiscriminately activates both target and non-target T cells.

An IL-2 receptor is in some cases a heterotrimeric polypeptide comprising an alpha chain (IL-2Rα; also referred to as CD25), a beta chain (IL-2Rβ; also referred to as CD122; and a gamma chain (IL-2Rγ; also referred to as CD132). Amino acid sequences of human IL-2, human IL-2Rα, IL2Rβ, and IL-2Rγ are known. See. e.g., published PCT applications WO2020132138A1 and WO2019/051091, discussed above. For example, a wild-type IL-2 polypeptide can have the amino acid sequence depicted in FIG. 22A. Amino acid sequences of human IL-2Rα, human IL-2Rβ, and human IL-2Rγ are depicted in FIGS. 22B, 22C, and 22D, respectively.

In some cases, an IL-2 variant MOD of this disclosure exhibits decreased binding to IL-2Rα, thereby minimizing or substantially reducing the activation of Tregs by the IL-2 variant. Alternatively, or additionally, in some cases, an IL-2 variant MOD of this disclosure exhibits decreased binding to IL-2Rβ and/or IL-2Rγ such that the IL-2 variant MOD exhibits an overall reduced affinity for IL-2R. In some cases, an IL-2 variant MOD of this disclosure exhibits both properties, i.e., it exhibits decreased or substantially no binding to IL-2Rα, and also exhibits decreased binding to IL-2Rβ and/or IL-2Rγ such that the IL-2 variant polypeptide exhibits an overall reduced affinity for TL-2R. For example, IL-2 variants having substitutions at H16 and F42 have shown decreased binding to IL-2Rα and IL-2Rα See, Quayle et al., Clin Cancer Res; 26(8) Apr. 15, 2020, which discloses that the binding affinity of an IL-2 polypeptide with H16A and F42A substitutions for human IL-2Rα and IL-2Rβ was decreased 110- and 3-fold, respectively, compared with wild-type IL2 binding, predominantly due to a faster off-rate for each of these interactions. TMPs comprising such variants, including variants that exhibit decreased binding to IL-2Rα and IL-2Rβ, have shown the ability to preferentially bind to and activate IL-2 receptors on T cells that contain the target TCR that is specific for the peptide epitope on the TMP, and are thus less likely to deliver IL-2 to non-target T cells, i.e., T cells that do not contain a TCR that specifically hinds the peptide epitope on the TMP. That is, the binding of the IL-2 variant MOD to its costimulatory polypeptide on the T cell is substantially driven by the binding of the MHC-epitope moiety rather than by the binding of the IL-2.

Suitable IL-2 variant MODs thus include a polypeptide that comprises an amino acid sequence having at least 90%, at least 95%, at least 98%, or at least 99% amino acid sequence identity to the wild-type IL-2 amino acid sequence depicted in FIG. 22A; and that have one or more amino acid differences from the wild-type IL-2 amino acid sequence depicted in FIG. 22A. In some cases, such a variant IL-2 polypeptide of this disclosure exhibits reduced binding affinity to IL-2R, compared to the binding affinity of an IL-2 polypeptide comprising the wild-type IL-2 amino acid sequence depicted in FIG. 22A. For example, in some cases, a variant IL-2 polypeptide binds IL-2R with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of an IL-2 polypeptide comprising the wild-type IL-2 amino acid sequence depicted in FIG. 22A for an IL-2R (e.g., an IL-2R comprising polypeptides comprising the amino acid sequences depicted in FIG. 22B-22D), when assayed under the same conditions.

In some cases, a suitable variant IL-2 polypeptide comprises an amino acid sequence having at least 90%, at least 95%, at least 98%, at least 99%, or 100% amino acid sequence identity to the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNR WITFCQSIIS TLT (SEQ ID NO:185), i.e., the variant IL-2 polypeptide has the amino acid sequence of wild-type IL-2 but with H16A and F42A substitutions (shown in bold). Alternatively, the foregoing sequence, but with substitutions other than Ala at H16 and/or F42 may be employed, e.g., H I6T may be employed instead of H16A. In some cases, a variant IL-2 polypeptide present in a TMP comprises the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:185). In some cases, a variant IL-2 polypeptide present in a TMP comprises the amino acid sequence: APTSSSTKKT QLQLETLLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:186). In some cases, a TMP comprises two copies of such a variant IL-2 polypeptide.

In some cases, a MOD present in a TMP is a PD-L1 polypeptide. PD-L1 variants that may be suitable as MODs are disclosed in Published PCT applications WO 2019/051091 and WO 2017/201131. In some cases, a PD-L1 polypeptide of a TMP comprises an amino acid sequence having at least 75%, at least 80%, at least 85%, at least 90%, at least 95%, at least 98%, at least 99%, or 100%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:130). See also FIG. 22E.

In some cases, a MOD present in a TMP is a 4-1BBL polypeptide. In some cases, a 4-1 BBL polypeptide of a TMP comprises an amino acid sequence having at least 75%, at least 80%, at least 85%, at least 90%, at least 95%, at least 98%, at least 99%, or 100%, amino acid sequence identity to the following 4-1BBL amino acid sequence: DPAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPA (SEQ ID NO:131). See also FIG. 22F.

In some cases, a MOD present in a TMP is a FasL polypeptide, e.g., the extracellular domain of a FasL polypeptide. In some cases, a FasL polypeptide of a TMP comprises an amino acid sequence having at least 75%, at least 80%, at least 85%, at least 90%, at least 95%, at least 98%, at least 99%, or 100%, amino acid sequence identity to the following FasL extracellular domain amino acid sequence: QLFHLQKE LAELRESTSQ MHTASSLEKQ IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMM SYCTTGQMWA RSSYLGAVFN LTSADHLYVN VSELSLVNFE ESQTFFGLYK L (SEQ ID NO:187).

### Class II MHC Polypeptides

As noted above, a TMP of the present disclosure includes MHC class II polypeptides. A TMP can include MHC class II polypeptides of various species, including human MHC polypeptides (HLA polypeptides), rodent (e.g., mouse, rat, etc.) MHC polypeptides, and MHC polypeptides of other mammalian species (e.g., lagomorphs, non-human primates, canines, felines, ungulates (e.g., equines, bovines, ovines, caprines, etc.), and the like. Examples of MHC class II polypeptides are presented in FIG. 4-18, FIG. 19A-19N, and FIG. 20A-20J.

MHC proteins are referred to as human leukocyte antigens (HLA) in humans. HLA class II gene loci include HLA-DM (HLA-DMA and HLA-DMB that encode HLA-DM α chain and HLA-DM (i chain, respectively), HLA-DO (HLA-DOA and HLA-DOB that encode HLA-DO α chain and HLA-DO β chain, respectively), HLA-DP (HLA-DPA and HLA-DPB that encode HLA-DP α chain and HLA-DP β chain, respectively), HLA-DQ (HLA-DQA and HLA-DQB that encode HLA-DQ α chain and HLA-DQ β chain, respectively), and HLA-DR (HLA-DRA and HLA-DRB that encode HLA-DR α chain and HLA-DR β chain, respectively).

For the purpose of this disclosure, the term “MHC polypeptide” is meant to include class II MHC polypeptides, including the α- and β-chains or portions thereof. More specifically. MHC class II polypeptides include the α1 and α2 domains of class II MHC α chains, and the β1 and β2 domains of class II MHC β chains, which represent all or most of the extracellular class II protein required for presentation of an epitope peptide. In some cases, both the α and β class II MHC polypeptide sequences in a TMP are of human origin, or variants of such polypeptides.

Unless expressly stated otherwise. MHC class II polypeptides in a TMP are not intended to include membrane anchoring domains (transmembrane regions) of MHC class II α and β chains, or a part of any thereof sufficient to anchor the resulting TMP, or a peptide thereof, in the membrane of a cell (e.g., a eukaryotic cell such as a mammalian cell such as a Chinese Hamster Ovary or “CHO” cell) in which the TMP is expressed. Similarly, unless expressly stated otherwise. MHC class II polypeptides in a TMP described herein do not include the leader and/or intracellular portions (e.g., cytoplasmic tails) that may be present in some naturally-occurring MHC class II proteins.

TMPs of the present disclosure comprise MHC class II polypeptides. Naturally occurring class II MHC polypeptides comprise an α chain and a β chain (e.g., HLA α- and β-chains). MHC Class II polypeptides include MHC Class II DP α and β polypeptides, DM α and β polypeptides. DO α and β polypeptides, DQ α and β polypeptides, and DR α and β polypeptides. As used herein, a “Class II MHC polypeptide” can comprise a class II MHC α chain polypeptide, a class II MHC β chain polypeptide, or only a portion of a class II MHC α and/or β chain polypeptide. For example, a “Class II MHC polypeptide” can be a polypeptide that includes: i) only the α1 domain of a class II MHC α chain; ii) only the α2 domain of a class II MHC α chain; iii) only the α1 domain and the α2 domain of a class II MHC α chain; iv) only the β1 domain of a class II MHC β chain; v) only the β2 domain of a class II MHC β chain; vi) only the β1 domain and the β2 domain of a class II MHC β chain; vii) the α1 domain of a class II MHC α chain, the β1 domain of a class II MHC β chain, and the 12 domain of a class II MHC; and the like. In many instances, an MIC class II α chain polypeptide will include only the α1 domain and the α2 domain of a class II MHC α chain polypeptide; and an MHC class II β chain polypeptide will include only the β1 domain and the β2 domain of a class II MHC β3 chain polypeptide.

The human MHC or HLA locus is highly polymorphic in nature, and the HLA Nomenclature site run by the Anthony Nolan Research Institute, available on the world wide web at hla.alleles.org/nomenclature/index.html indicates that, as of Apr. 8, 2019, there are 7 DRA alleles, 2.479 DRB1 alleles, 1 DRB2 allele, 225 DRB3 alleles, 121 DRB4 alleles, 85 DRB5 alleles, 3 DRB6 alleles, 2 DRB7 alleles, 1 DRB8 allele, 6 DRB9 alleles, 149 DQA1 alleles, 1.561 DQB1 alleles, 106 DPA1, 1.360 DPB1 alleles, 7 DMA alleles, 13 DMB alleles, 12 DOA alleles and 13 DOB alleles. As used herein, the term “Class II MHC polypeptide” includes allelic forms of any known Class II MHC polypeptide.

A TMP may comprise a Class II MHC α chain, without the leader, transmembrane, and intracellular portions (e.g., cytoplasmic tails) that may be present in a naturally-occurring Class II MHC α chain. Thus, a TMP may comprise only the α1 and α2 portions of a Class II MHC α chain; and does not include the leader, transmembrane, and intracellular portions (e.g., cytoplasmic tails) that may be present in a naturally-occurring Class II MHC α chain.

A TMP may comprise a Class II MHC β chain, without the leader, transmembrane, and intracellular portions (e.g., cytoplasmic tails) that may be present in a naturally-occurring Class II MHC β chain. Thus, a TMP may comprise only the β1 and β2 domain portions of a Class II MHC β chain; and does not include the leader, transmembrane, and intracellular portions (e.g., cytoplasmic tails) that may be present in a naturally-occurring Class II MHC β chain.

### a) MHC Class II Alpha Chains

MHC Class II alpha chains comprise an α1 domain and an α2 domain. In some cases, the α1 and α2 domains present in an antigen-presenting cell are from the same MHC Class H α chain polypeptide. In some cases, the α1 and α2 domains present in an antigen-presenting cell are from two different MHC Class II α chain polypeptides. As noted above. References herein to MHC class II a polypeptides may include both the α1 and α2 domains of class II MHC α chains.

MHC Class II alpha chains suitable for inclusion in a TMP may lack a signal peptide. An MHC Class II alpha chain suitable for inclusion in a TMP can have a length of from about 60 amino acids (aas) to about 200 aas; for example, an MHC Class II alpha chain suitable for inclusion in a TMP can have a length of from about from about 60 amino acids to about 80 amino acids, 80 aas to about 100 aas, from about 100 aas to about 140 aas, from about 140 aas to about 170 aas, from about 170 aas to about 200 aas. An MHC Class II α1 domain suitable for inclusion in a TMP can have a length of from about 30 aas to about 95 aas; for example, an MHC Class II α1 domain suitable for inclusion in a TMP can have a length of from about 30 aas to about 50 aas, from about 50 aas to about 70 aas, or from about 70 aas to about 95 aas. In an embodiment, an MHC Class II α1 domain of TMP is from about 70 aas to about 95 aas. An MHC Class II α2 domain suitable for inclusion in a TMP can have a length of from about 30 aas to about 95 aas; for example, an MHC Class II α2 domain suitable for inclusion in a TMP can have a length of from about 30 aas to about 50 aas, from about 50 aas to about 70 aas, or from about 70 aas to about 95 aas. In an embodiment, an MHC Class II α2 domain of a TMP is from about 70 aas to about 95 aas.

### DRA Polypeptides

In some cases, a suitable MHC Class II α chain polypeptide is a DRA polypeptide. A DRA polypeptide can have at least 60%, at least 70%, at least 80%, at least 90%, at least 95%, at least 98%, at least 99%, or 100% aa sequence identity with at least 150, at least 160, or at least 170 contiguous amino acids of the as sequence from aa 26 to as 203 of the DRA as sequence depicted in FIG. 4 including-naturally occurring allelic variants thereof. In some cases, the DRA polypeptide has a length of about 178 aas (e.g., 175, 176, 177, 178, 179, or 180 aas).

A “DRA polypeptide” includes allelic variants, e.g., naturally occurring allelic variants. Thus, in some cases, a suitable DRA polypeptide comprises aas 26-203 of DRA*01:02:01 (see FIG. 4), or an allelic variant thereof. In some cases, the allelic variant is the DRA*01:01:01:01 allelic variant that differs from DRA*01:02 by having a valine in place of the leucine at position 242 (see FIG. 4).

A suitable DRA for inclusion in a TMP can have at least 70%, at least 80%, at least 90%, at least 95%, at least 98%, at least 99%, or 100%, aa sequence identity with at least 160, at least 170, or at least 180 contiguous aas of the sequence from aa 26 to aa 216 of the DRA*01:02 sequence depicted in FIG. 4. A “DRA polypeptide” includes allelic variants, e.g., naturally occurring allelic variants.

Thus, in some cases, a suitable DRA polypeptide comprises the following amino acid sequence: IKEEH VIIQAEFYLN PDQSGEFMFD FDGDEIFHVD MAKKETVWRL EEFGRFASFE AQGALANIAV DKANLEIMTK RSNYTPITNV PPEVTVLTNS PVELREPNVL ICFIDKFTPP VVNVTWLRNG KPVTTGVSET VFLPREDHLF RKFHYLPFLP STEDVYDCRV EHWGLDEPLL KHW (SEQ ID NO:188, amino acids 26-203 of DRA*01:02, see FIG. 4), or an allelic variant thereof. In some cases, the allelic variant is the DRA*01:01:01:01 allelic variant that differs from DRA*01:02:01 by having a valine in place of the leucine at position 242 of the sequence in FIG. 4. In some cases, a DRA polypeptide suitable for inclusion in a TMP comprises an amino acid substitution, relative to a wild-type DRA polypeptide, where the amino acid substitution replaces an amino acid (other than a Cys) with a Cys.

A TMP may comprise a variant DRA polypeptide that comprises a non-naturally occurring Cys residue. For example, a TMP may comprise a variant DRA polypeptide that comprises an amino acid substitution selected from E3C, E4C, F12C, G28C, D29C, I72C, K75C, T80C, P81C, I82C, T93C, N94C, and S95C.

A suitable DRA α1 domain for inclusion in a TMP, including naturally occurring allelic variants thereof, may comprise an aa sequence having at least 70%, at least 80%, at least 90%, at least 95%, at least 98%, at least 99%, or 100% an sequence identity to the following aa sequence: VIIQAEFYLN PDQSGEFMFD FDGDEIFHVD MAKKETVWRL EEFGRFASFE AQGALANIAV DKANLEIMTK RSNYTPITN (SEQ ID NO:189); and can have a length of about 84 aas (e.g., 80, 81, 82, 83, 84, 85, or 86 aac).

A suitable DRA α2 domain for inclusion in a TMP, including naturally occurring allelic variants thereof, may comprise an aa sequence having at least 70%, at least 80%, at least 90%, at least 95%, at least 98%, at least 99%, or 100% aa sequence identity to the following aa sequence: V PPEVTVLTNSPVELREPNVL ICFIDKFTPP VVNVTWLRNG KPVTTGVSET VFLPREDHLF RKFHYLPFLP STEDVYDCRV EHWGLDEPLL KHW (SEQ ID NO:190); and can have a length of about 94 aas (e.g., 90, 91, 92, 93, 94, 95, 96, 97, or 98 aas).

### DMA Polypeptides

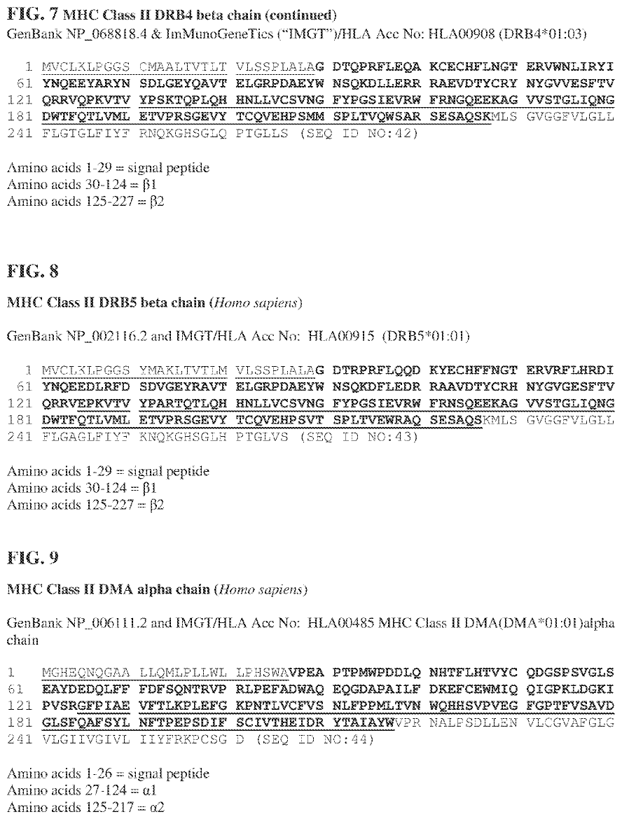

In some cases, a suitable MHC Class II α chain polypeptide is a DMA polypeptide. A DMA polypeptide can have at least 60%, at least 70%, at least 80%, at least 90%, at least 95%, at least 98%, at least 99%, or 100% aa sequence identity with aas 27-217 of the DMA*01:01 aa sequence depicted in FIG. 9, including-naturally occurring allelic variants thereof. In some cases, the DMA polypeptide has a length of about 191 aas (e.g., 188, 189, 190, 191, 192, or 193 aas).

A “DMA polypeptide” includes allelic variants, e.g., naturally occurring allelic variants. Thus, in some cases, a suitable DMA polypeptide comprises aas 27-217 of DMA*01:01:01 (see FIG. 9), or an allelic variant thereof.

A suitable DMA α1 domain, including-naturally occurring allelic variants thereof, may comprise an aa sequence having at least 70%, at least 80%, at least 90%, at least 95%, at least 98%, at least 99%, or 100% aa sequence identity to the following aa sequence: VPEA PTPMWPDDLQ NHTFLHTVYC QDGSPSVGLS EAYDEDQLFF FDFSQNTRVP RLPEFADWAQ EQGDAPAILF DKEFCEWMIQ QIGPKLDGKI PVSR (SEQ ID NO:191); and can have a length of about 98 aas (e.g., 94, 95, 96, 97, 98, 99, 100, or 101 aas).

A suitable DMA α2 domain, including-naturally occurring allelic variants thereof, may comprise an aa sequence having at least 70%, at least 80%, at least 90%, at least 95%, at least 98%, at least 99%, or 100% aa sequence identity to the following an sequence: GFPIAE VFTLKPLEFG KPNTLVCFVS NLFPPMLTVN WQHHSVPVEG FGPTFVSAVD GLSFQAFSYL NFTPEPSDIF SCIVTHEIDR YTAIAYW (SEQ ID NO:192); and can have a length of about 93 aas (e.g., 90, 91, 92, 93, 94, 95, 96, or 97 aas).

### DOA Polypeptides

In some cases, a suitable MHC Class II α chain polypeptide is a DOA polypeptide. A DOA polypeptide can have at least 60%, at least 70%, at least 80%, at least 90%, at least 95%, at least 98%, at least 99%, or 100% aa sequence identity with aas 26-204 of the DOA*01:01 aa sequence depicted in FIG. 11. In some cases, the DOA polypeptide has a length of about 179 aas (e.g., 175, 176, 177, 178, 179, 180, 181, or 182 aas).

A “DOA polypeptide” includes allelic variants, e.g., naturally occurring allelic variants. Thus, in some cases, a suitable DOA polypeptide comprises aas 26-204 of DOA*01:011 (see FIG. 11), or an allelic variant thereof. In some cases, the allelic variant may be the DOA*01:02 by having an arginine in place of the cysteine (R80C) at position 80 or the DOA*01:03 variant having a valine in place of the leucine at position 74 (L74V) relative to DOA*01:01.

A suitable DOA α1 domain, including-naturally occurring allelic variants thereof, may comprise an aa sequence having at least 70%, at least 80%, at least 90%, at least 95%, at least 98%, at least 99%, or 100% aa sequence identity to the following aa sequence: TKADH MGSYGPAFYQ SYGASGQFTH EFDEEQLFSV DLKKSEAVWR LPEFGDFARF DPQGGLAGIA AIKAHLDILV ERSNRSRAIN (SEQ ID NO:193); and can have a length of about 85 aas (e.g., 83, 84, 85, 86, 87, or 88 aas). Suitable α1 domain sequences may incorporate the L74V and/or R80C substitutions found in DOA*01:02 and DOA*01:03 (the aas corresponding to L74 and R80 are shown italicized and bolded).

A suitable DOA α2 domain, including-naturally occurring allelic variants thereof, may comprise an aa sequence having at least 70%, at least 80%, at least 90%, at least 95%, at least 98%, at least 99%, or 100% aa sequence identity to the following an sequence: VPPRVTVLPK SRVELGQPNI LICIVDNIFP PVINITWLRN GQTVTEGVAQ TSFYSQPDHL FRKFHYLPFV PSAEDVYDCQ VEHWGLDAPL LRHW (SEQ ID NO:194); and can have a length of about 94 aas (e.g., 91, 92, 93, 94, 95, 96, or 97 aas).

### DPA1 Polypeptides

In some cases, a suitable MHC Class II α chain polypeptide is a DPA1 polypeptide. A DPA1 polypeptide can have at least 60%, at least 70%, at least 80%, at least 90%, at least 95%, at least 98%, at least 99%, or 100% an sequence identity with aas 29-209 of the DPA1 an sequence depicted in FIG. 13. In some cases, the DPA1 polypeptide has a length of about 181 aas (e.g., 178, 179, 180, 181, 182, 183, or 184 aas).

A “DPA1 polypeptide” includes allelic variants, e.g., naturally occurring allelic variants. Thus, in some cases, a suitable DPA1 polypeptide comprises aas 29-209 of DPA1*01:03 (see FIG. 13), or an allelic variant thereof.

A suitable DPA1 α1 domain, including-naturally occurring allelic variants thereof, may comprise an aa sequence having at least 70%, at least 80%, at least 90%, at least 95%, at least 98%, at least 99%, or 100% an sequence identity to the following aa sequence: AIKADHVSTY AAFVQTHRPT GEFMFEFDED EMFYVDLDKK ETVWHLFEFG QAFSFEAQCIG LANIAILNNN INTILIQRSNH TQATN (SEQ ID NO:195); and can have a length of about 87 aas (e.g., 84, 85, 86, 87, 88, or 89 aas).

A suitable DPA1 α2 domain, including-naturally occurring allelic variants thereof, may comprise an aa sequence having at least 70%, at least 80%, at least 90%, at least 95%, at least 98%, at least 99%, or 100% aa sequence identity to the following aa sequence: DPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO:196); and can have a length of about 97 aas (e.g., 91, 92, 93, 94, 95, 96, or 97 aas).

Another DPA1 polypeptide comprises aas 29-209 of DPA1*02:01 (see FIG. 13), or a variant thereof having at least 70%, at least 80%, at least 90%, at least 95%, at least 98%, at least 99%, or 100% aa sequence identity. A suitable DPA1 α1 domain, including-naturally occurring allelic variants thereof, may comprise an aa sequence having at least 70%, at least 80%, at least 90%, at least 95%, at least 98%, at least 99%, or 100% aa sequence identity to aas 29-115 of DPA1*02:01; and can have a length of about 87 aas (e.g., 84, 85, 86, 87, 88, or 89 aas). A suitable DPA1 α2 domain, including-naturally occurring allelic variants thereof, may comprise an aa sequence having at least 70%, at least 80%, at least 90%, at least 95%, at least 98%, at least 99%, or 100% aa sequence identity to aas 116 to 209 of DPA1*02:01:01:01; and can have a length of about 97 aas (e.g., 91, 92, 93, 94, 95, 96, or 97 aas).

### DQA1 Polypeptides

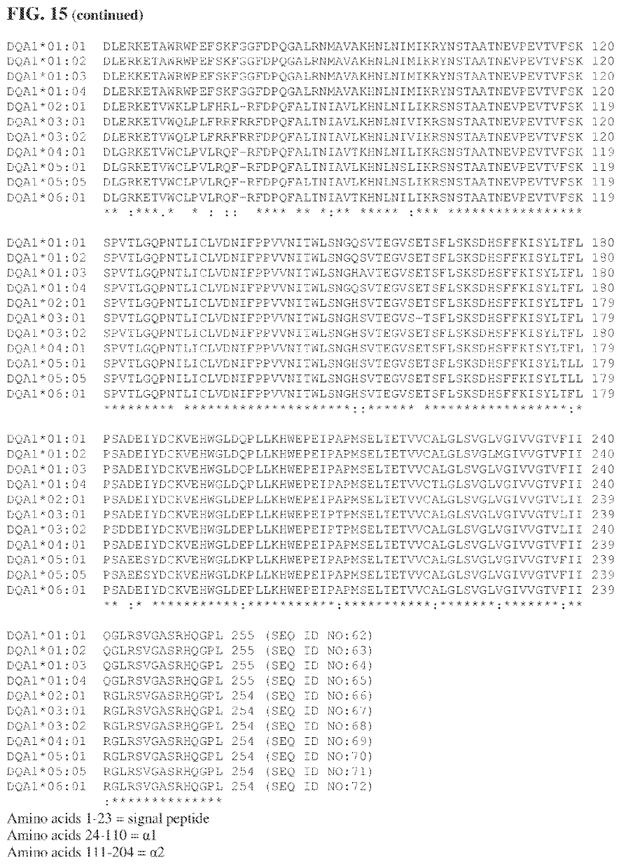

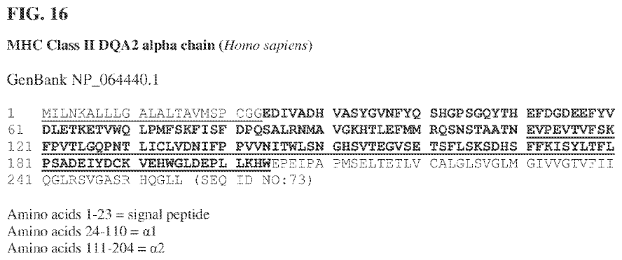

In some cases, a suitable MHC Class II α chain polypeptide is a DQA1 polypeptide. A suitable DQA1 polypeptide, including-naturally occurring allelic variants thereof, may comprise an aa sequence having at least 60%, at least 70%, at least 80%, at least 90%, at least 95%, at least 98%, at least 99%, or 100% aa sequence identity with aas 24-204 of any of the DQA1 aa sequences depicted in FIG. 15. In some cases, the DQA1 polypeptide has a length of about 181 aas (e.g., 177, 178, 179, 180, 181, 182, or 183 aas). In an embodiment, a DQA1 α chain polypeptide can have at least 60%, at least 70%, at least 80%, at least 90%, at least 95%, at least 98%, at least 99%, or 100% aa sequence identity with aas 24-204 of the DQA1*01:01 α chain as sequence in FIG. 15. ImMunoGeneTics (“IMGT”)/HLA Acc No:HLA00601. In an embodiment, a DQA1 α chain polypeptide can have at least 60%, at least 70%, at least 80%, at least 90%, at least 95%, at least 98%, at least 99%, or 100% aa sequence identity with aas 24-204 of the DQA1*01:02 α chain aa sequence in FIG. 15, IMGT/HLA Ace No:HLA00603, GenBank NP_002113. In an embodiment, a DQA1 α chain polypeptide can have at least 60%, at least 70%, at least 80%, at least 90%, at least 95%, at least 98%, at least 99%, or 100% aa sequence identity with aas 24-204 of the DQA1*02:01 α chain an sequence in FIG. 15. IMGT/HLA Ace No:HLA00607. In an embodiment, a DQA1 α chain polypeptide can have at least 60%, at least 70%, at least 80%, at least 90%, at least 95%, at least 98%, at least 99%, or 100% aa sequence identity with aas 24-204 of the DQA1*03:01: α chain aa sequence in FIG. 15. IMGT/HLA Ace No:HLA00609. In an embodiment, a DQA1 α chain polypeptide can have at least 60%, at least 70%, at least 80%, at least 90%, at least 95%, at least 98%, at least 99%, or 100% as sequence identity with aas 24-204 of the DQA1*04:01 α chain aa sequence in FIG. 15, IMGT/HLA Ace No:HLA00612. In an embodiment, a DQA1 α chain polypeptide can have at least 60%, at least 70%, at least 80%, at least 90%, at least 95%, at least 98%, at least 99%, or 100% aa sequence identity with aas 24-204 of the DQA1*05:01 α chain aa sequence in FIG. 15, IMGT/HLA Ace No:HLA00613. In an embodiment, a DQA1 α chain polypeptide can have at least 60%, at least 70%, at least 80%, at least 90%, at least 95%, at least 98%, at least 99%, or 100% an sequence identity with aas 24-204 of the DQA1*06:01 α chain as sequence in FIG. 15, IMGT/HLA Ace No:HLA00620.

A “DQA1 polypeptide” includes allelic variants, e.g., naturally occurring allelic variants. Thus, in some cases, a suitable DQA1 polypeptide comprises the following aa sequence: EDIVADH VASCGVNLYQ FYGPSGQYTH EFDGDEQFYV DLERKETAWR WPEFSKFGGF DPQGALRNMA VAKHNLNIMI KRYNSTAATN EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO:197), or an allelic variant thereof.

A suitable DQA1 α1 domain, including-naturally occurring allelic variants thereof, may comprise an aa sequence having at least 70%, at least 80%, at least 90%, at least 95%, at least 98%, at least 99%, or 100% aa sequence identity to the following aa sequence: EDIVADH VASCGVNLYQ FYGPSGQYTH EFDGDEQFYV DLERKETAWR WPEFSKFGGF DPQGALRNMA VAKHNLNIMI KRYNSTAATN (SEQ ID NO:198); and can have a length of about 87 aas (e.g., 84, 85, 86, 87, 88, or 89 aas).

A suitable DQA1 α2 domain, including-naturally occurring allelic variants thereof, may comprise an aa sequence having at least 70%, at least 80%, at least 90%, at least 95%, at least 98%, at least 99%, or 100% aa sequence identity to the following aa sequence: EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO:199); and can have a length of about 94 aas (e.g., 91, 92, 93, 94, 95, 96, or 97 aas).

### DQA2 Polypeptides

In some cases, a suitable MHC Class II α chain polypeptide is a DQA2 polypeptide. A DQA2 polypeptide can have at least 60%, at least 70%, at least 80%, at least 90%, at least 95%, at least 98%, at least 99%, or 100% an sequence identity with aas 24-204 of the DQA2 aa sequence depicted in FIG. 16. In some cases, the DQA2 polypeptide has a length of about 181 aas (e.g., 177, 178, 179, 180, 181, 182, or 183 aas).

A “DQA2 polypeptide” includes allelic variants. e.g., naturally occurring allelic variants. Thus, in some cases, a suitable DQA2 polypeptide comprises the following an sequence: EDIVADH VASYGVNFYQ SHGPSGQYIH EFDGDEEFYV DLETKEIV WQ LPMFSKFISF DPQSALRNMA VGKHTLEFMM RQSNSTAATN EVPEVTVFSK FPVTLGQPNT LICLVDNIFP PVVNITWLSN GHSVTEGVSE TSFLSKSDHS FFKISYLTFL PSADEIYDCK VEHWGLDEPL LKHW (SEQ ID NO:200), or an allelic variant thereof.

A suitable DQA2 α1 domain, including-naturally occurring allelic variants thereof, may comprise an aa sequence having at least 70%, at least 80%, at least 90%, at least 95%, at least 98%, at least 99%, or 100% an sequence identity to the following an sequence: EDIVADH VASYGVNFYQ SHGPSGQYTH EFDGDEEFYV DLETKEIVWQ LPMFSKFISF DPQSALRNMA VGKHTLEFMM RQSNSTAATN (SEQ ID NO:201); and can have a length of about 87 aas (e.g., 84, 85, 86, 87, 88, or 89 aas).

A suitable DQA2 α2 domain, including-naturally occurring allelic variants thereof, may comprise an aa sequence having at least 70%, at least 80%, at least 90%, at least 95%, at least 98%, at least 99%, or 100% aa sequence identity to the following aa sequence: EVPEVTVFSK FPVTLGQPNT LICLVDNIFP PVVNITWLSN GHSVTEGVSE TSFLSKSDHS FFKISYLTFL PSADEIYDCK VEHWGLDEPL LKHW (SEQ ID NO:202); and can have a length of about 94 aas (e.g., 91, 92, 93, 94, 95, 96, or 97 aas).

### b) MHC Class II Beta Chains

MHC Class II beta chains comprise a β1 domain and a β2 domain. In some cases, the β1 and β2 domains present in a TMP are from the same MHC Class II β chain polypeptide. In some cases, the β1 and β2 domains present in a TMP are from two different MHC Class II β chain polypeptides. As noted above, references herein to MHC class II β polypeptides may include both the β1 and β2 domains of class II MHC β chains.

MHC Class II beta chain sequences suitable for inclusion in a TMP lack a signal peptide. An MHC Class II beta chain suitable for inclusion in a TMP can have a length of from about 60 aas to about 210 aas; for example, an MHC Class II beta chain suitable for inclusion in a TMP can have a length of from about 60 aas to about 90 aas, from about 90 aas to about 120 aas, from about 120 aas to about 150 aas, from about 150 aas to about 180 aas, from about 180 aas to 210 aas. An MHC Class II β1 domain suitable for inclusion in a TMP can have a length of from about 30 aas to about 105 aas; for example, an MHC Class II β1 domain suitable for inclusion in a TMP can have a length of from about 30 aas to about 50 aas, from about 50 aas to about 70 aas, from about 70 aas to about 90 aas, from about 90 aas to about 105 aas. An MHC Class II β2 domain suitable for inclusion in a TMP can have a length of from about 30 aas to about 105 aas; for example, an MHC Class II 02 domain suitable for inclusion in a TMP can have a length of from about 30 aas to about 50 aas, from about 50 aas to about 70 aas, from about 70 aas to about 90 aas, from about 90 aas to about 105 aas.

MHC class II β chain polypeptide suitable for inclusion in a TMP may comprises an aa substitution, relative to a wild-type MHC class II β chain polypeptide, where the aa substitution replaces an aa (other than a Cys) with a Cys. For example, in some cases, the MHC class II β chain polypeptide is a variant DRB1 MHC class II polypeptide that comprises an aa substitution selected from the group consisting of P5C, F7C, Q10C, N19C, G20C, 1133C, G151C, D152C, and W153C. In some cases, the MHC class II β chain polypeptide is a variant DRB1 polypeptide comprising an aa sequence having at least 60%, at least 70%, at least 80%, at least 90%, at least 95%, at least 98%, or at least 99%, aa sequence identity to the following DRB1 aa sequence: GDTRPRFLEQVKHECHFFNGTERVRFLDRYFYHQEEYVRFDSDVGEYRAVTELGRPDAEYWNS QKDLLEQKRAAVDTYCRHNYGVGESFTVQRRVYPEVTVYPAKTQPLQHHNLLVCSVNGFYPA SIEVR WFRNGQEEKTGVVSTGLIQNGDWTFQTLVMLETVPRSGEV YTCQVEHPSLTSPLTVEWR ARSESAQSKM (SEQ ID NO:103), and comprising an aa substitution selected from the group consisting of P5C, F7C, Q10C, N19C. G20C, H33C. G151C, D152C, and W153C. In some cases, the MHC class II β chain polypeptide is a variant DRB3 polypeptide, a variant DRB4 polypeptide, or a variant DRB5 polypeptide comprising an aa substitution corresponding to any of the foregoing aa substitutions in a variant DRB1 polypeptide. For example, as depicted in FIG. 7B: i) an aa corresponding to P5 of DRB1 would be P5 of a mature DRB3 polypeptide (lacking the N-terminal signal peptide MVCLKLPGGSSLAALTVTLMVLSSRLAFA (SEQ ID NO:203)); ii) an aa corresponding to F7 of DRB1 would be F7 of a mature DRB3 polypeptide; iii) an aa corresponding to Q10 of DRB1 would be L10 of a mature DRB3 polypeptide; iv) an aa corresponding to N19 of DRB1 would be N19 of a mature DRB3 polypeptide; v) an aa corresponding to G20 of DRB1 would be G20 of a mature DRB3 polypeptide; vi) an aa corresponding to 1133 of DRB1 would be N33 of a mature DRB3 polypeptide; vii) an aa corresponding to G151 of DRB1 would be G151 of a mature DRB3 polypeptide; viii) an aa corresponding to D152 of DRB1 would be D152 of a mature DRB3 polypeptide; and ix) an aa corresponding to W153 of DRB1 would be W153 of a mature DRB3 polypeptide. As another example, as depicted in FIG. 7C, i) an aa corresponding to P5 of DRB1 would be P15 of a mature DRB4 polypeptide (lacking the N-terminal signal peptide MVCLKLPGGSCMAALTVTL (SEQ ID NO:204)); ii) an aa corresponding to F7 of DRB1 would be F17 of a mature DRB4 polypeptide; iii) an aa corresponding to Q10 of DRB1 would be Q20 of a mature DRB4 polypeptide; iv) an aa corresponding to N19 of DRB1 would be N29 of a mature DRB4 polypeptide; v) an aa corresponding to G20 of DRB1 would be G30 of a mature DRB4 polypeptide; vi) an aa corresponding to H33 of DRB1 would be N43 of a mature DRB4 polypeptide; vii) an aa corresponding to G151 of DRB1 would be G161 of a mature DRB4 polypeptide; viii) an aa corresponding to D152 of DRB1 would be D162 of a mature DRB4 polypeptide; and ix) an aa corresponding to W153 of DRB1 would be W153 of a mature DRB4 polypeptide. As another example, as depicted in FIG. 7D, i) an aa corresponding to P5 of DRB1 would be P15 of a mature DRB5 polypeptide (lacking the N-terminal signal peptide MVCLKLPGGSYMAKLTVTL (SEQ ID NO:205)); ii) an aa corresponding to F7 of DRB1 would be F17 of a mature DRB5 polypeptide; iii) an aa corresponding to Q10 of DRB1 would be Q20 of a mature DRB5 polypeptide; iv) an aa corresponding to N19 of DRB1 would be N29 of a mature DRB5 polypeptide; v) an aa corresponding to G20 of DRB1 would be G30 of a mature DRB5 polypeptide; vi) an aa corresponding to 1133 of DRB1 would be N43 of a mature DRB5 polypeptide; vii) an aa corresponding to G151 of DRB1 would be G161 of a mature DRB5 polypeptide; viii) an aa corresponding to D152 of DRB1 would be D162 of a mature DRB5 polypeptide; and ix) an aa corresponding to W153 of DRB1 would be W163 of a mature DRB5 polypeptide.

### DRB1 Polypeptides

In some cases, a suitable MHC Class II § chain polypeptide is a DRB1 polypeptide. In an embodiment, a DRB1 polypeptide can have at least 60%, at least 70%, at least 80%, at least 90%, at least 95%, at least 98%, at least 99%, or 100%, aa sequence identity with at least 170, at least 180, or at least 190, contiguous aas of the sequence from aa 30 to aa 227 of any DRB1 aa sequence depicted in FIG. 5, including naturally occurring allelic variants. FIG. 5 displays the DRB1 precursor proteins in which aas 1-29 are the signal sequence (underlined), 30-124 form the 01 region (bolded), 125-227 for the β2 region (bolded and underlined), and 228-250 the transmembrane region. In some cases, a DRB1 polypeptide suitable for inclusion in a TMP comprises an aa substitution, relative to a wild-type DRB1 polypeptide, where the aa substitution replaces an aa (other than a Cys) with a Cys.

In an embodiment, a DRB1 β chain polypeptide of a TMP can have at least 60%, at least 70%, at least 80%, at least 90%, at least 95%, at least 98%, at least 99%, or 100% aa sequence identity with at least 170, at least 180, or at least 190, contiguous aas of the sequence from an 30 to aa 227 of the DRB1-1 (DRB1*01:01) beta chain aa sequence Swiss-Prot/UniProt reference (“sp”) P04229.2 in FIG. 5. In an embodiment, a DRB1 β chain polypeptide of a TMP can have at least 60%, at least 70%, at least 80%, at least 90%, at least 95%, at least 98%, at least 99%, or 100% aa sequence identity with at least 170, at least 180, or at least 190, contiguous aas of the sequence from an 30 to aa 227 of the DRB1-3 (DRB1*03:01) beta chain aa sequence sp P01912.2 in FIG. 5. In an embodiment, a DRB1 β chain polypeptide of a TMP can have at least 60%, at least 70%, at least 80%, at least 90%, at least 95%, at least 98%, at least 99%, or 110% aa sequence identity with at least 170, at least 180, or at least 190, contiguous aas of the sequence from an 30 to aa 227 of the DRB1-4 (DRB1*04:01) beta chain an sequence sp P13760.1 in FIG. 5. In an embodiment, a DRB13 chain polypeptide of a TMP can have at least 60%, at least 70%, at least 80%, at least 90%, at least 95%, at least 98%, at least 99%, or 100% aa sequence identity with at least 170, at least 180, or at least 190, contiguous aas of the sequence from aa 30 to aa 227 of the DRB1-7 (DRB1*07:01) beta chain aa sequence sp P13761.1 in FIG. 5. In an embodiment, a DRB10β chain polypeptide of a TMP can have at least 60%, at least 70%, at least 80%, at least 90%, at least 95%, at least 98%, at least 99%, or 100% an sequence identity with at least 170, at least 180, or at least 190, contiguous aas of the sequence from an 30 to an 227 of the DRB1-8 (DRB1*08:01) beta chain aa sequence sp Q30134.2 in FIG. 5. In an embodiment, a DRB1 β chain polypeptide of a TMP can have at least 60%, at least 70%, at least 80%, at least 90%, at least 95%, at least 98%, at least 99%, or 100%, an sequence identity with at least 170, at least 180, or at least 190, contiguous aas of the sequence from aa 30 to aa 227 of the DRB1-9 (DRB1*09:01) beta chain aa sequence sp Q9TQE0.1 in FIG. 5. In an embodiment, a DRB1β chain polypeptide of a TMP can have at least 60%, at least 70%, at least 80%, at least 90%, at least 95%, at least 98%, at least 99%, or 100% an sequence identity with at least 170, at least 180, or at least 190, contiguous aas of the sequence from an 30 to aa 227 of the DRB1-10 (DRB1*10:01) beta chain aa sequence sp Q30167.2 in FIG. 5. In an embodiment, a DRB1β chain polypeptide of a TMP can have at least 60%, at least 70%, at least 80%, at least 90%, at least 95%, at least 98%, at least 99%, or 100% aa sequence identity with at least 170, at least 180, or at least 190, contiguous aas of the sequence from aa 30 to aa 227 of the DRB1-11 (DRB1*11:01) beta chain aa sequence sp P20039.1 in FIG. 5. In an embodiment, a DRB1 β chain polypeptide of a TMP can have at least 60%, at least 70%, at least 80%, at least 90%, at least 95%, at least 98%, at least 99%, or 100% aa sequence identity with at least 170, at least 180, or at least 190, contiguous aas of the sequence from aa 30 to aa 227 of the DRB1-12 (DRB1*12:01) beta chain aa sequence sp Q95IE3.1 in FIG. 5. In an embodiment, a DRB1 β chain polypeptide of a TMP can have at least 60%, at least 70%, at least 80%, at least 90%, at least 95%, at least 98%, at least 99%, or 100% aa sequence identity with at least 170, at least 180, or at least 190, contiguous aas of the sequence from aa 30 to an 227 of the DRB1-13 (DRB1*13:01) beta chain an sequence sp Q5Y7A7.1 in FIG. 5. In an embodiment, a DRB1 β chain polypeptide of a TMP can have at least 60%, at least 70%, at least 80%, at least 90%, at least 95%, at least 98%, at least 99%, or 100% aa sequence identity with at least 170, at least 180, or at least 190, contiguous aas of the sequence from aa 30 to an 227 of the DRB1-14 (DRB*14:01) beta chain aa sequence sp Q9GIY3.1 in FIG. 5. In an embodiment, a DRB1 β chain polypeptide of a TMP can have at least 60%, at least 70%, at least 80%, at least 90%, at least 95%, at least 98%, at least 99%, or 100% aa sequence identity with at least 170, at least 180, or at least 190, contiguous aas of the sequence from aa 30 to aa 227 of the DRB11-15 (DRB1*15:01) beta chain aa sequence sp P01911 in FIG. 5. In an embodiment, a DRB1 β chain polypeptide of a TMP can have at least 60%, at least 70%, at least 80%, at least 90%, at least 95%, at least 98%, at least 99%, or 100% aa sequence identity with at least 170, at least 180, or at least 190, contiguous aas of the sequence from aa 30 to an 227 of the DRB1-16 (DRB1*16:01) beta chain an sequence sp Q29974.1 in FIG. 5. In some cases, the DRB1 β chain polypeptide has a length of about 198 aas (e.g., 195, 196, 197, 198, 199, 200, 201, or 202 aas).

A suitable “DRB1 polypeptide” for inclusion in a TMP may include allelic variants, e.g., naturally occurring allelic variants. Thus, in some cases, a suitable DRB1 polypeptide comprises aas 31-227 of DRB1*04:01 (DRB1-4) provided in FIG. 5 or an allelic variant thereof.

Another suitable DRB1 polypeptide may comprise a sequence having at least 70%, at least 80%, at least 90%, at least 95%, at least 98%, at least 99%, or 100% as sequence identity to at least 170, at least 180, or at least 190 contiguous aas of the following DRB1*04:01 aa sequence: GDTRPRFLEQVKHECHFFNGTERVRFLDRYFYHQEEYVRFDSDVGEYRAVTELGRPDAEYWNS QKDLLEQKRAAVDTYCRHNYGVGESFTVQRRVYPEVTVYPAKTQPLQHHNLLVCSVNGFYPA SIEVRWFRNGQEEKTGVVSTGLIQNGDWTFQTLVMLETVPRSGEVYTCQVEHPSLTSPLTVEWR ARSESAQSKM (SEQ ID NO:103), which may bear one or more cysteine substitutions. In an embodiment the cysteine substitution is a P5C substitution. In an embodiment the cysteine substitution is a G151C substitution. In an embodiment the cysteine substitution is a W153C substitution.

A suitable DRB1β1 domain, including-naturally occurring allelic variants thereof, may comprise an aa sequence having at least 70%, at least 80%, at least 90%, at least 95%, at least 98%, at least 99%, or 100% aa sequence identity to the following aa sequence: DTRPRFLEQVKHECHFFNGTERVRFLDRYFYIIQEEYVRFDSDVGEYRAVTELGRPDAEYWNSQ KDLLEQKRAAVDTYCRHNYGVGESFTVQRRV (SEQ ID NO:206); and can have a length of about 95 aas (e.g., 92, 93, 94, 95, 96, 97, or 98 aas).

A suitable DRB1β1 domain can comprise the following amino acid sequence: GDTRCRFLEQVKHECHFFNGTERVRFLDRYFYHQEEYVRFDSDVGEYRAVTELGRPDAEYWNS QKDLLEQKRAAVDTYCRHNYGVGESFIVQRRV (SEQ ID NO:207), where P5 is substituted with a Cys (shown in bold and italics text).

A suitable DRB1β2 domain, including-naturally occurring allelic variants thereof, may comprise an aa sequence having at least 70%, at least 80%, at least 90%, at least 95%, at least 98%, at least 99%, or 100% an sequence identity to the following an sequence: YPEVTVYPAKTQPLQHHNLLVCSVNGFYPGSIEVRWFRNGQEEKTGVVSTGLIQNGDWTFQTL VMLETVPRSGEVYTCQVEHPSLTSPLTVEWRARSFSAQSK (SEQ TD NO:208); and can have a length of about 103 aas (e.g., 100, 101, 102, 103, 104, 105, or 106 aas).

A suitable DRB1 β2 domain can comprise the following amino acid sequence: YPEVTVYPAKTQPLQHHNLLVCSVNGFYPASIEVRWFRNGQEEKTGVVSTGLIQNGDCTFQTLV MLETVPRSGEVYTCQVEHPSLTSPLTVEWRARSESAQSKM (SEQ ID NO:209), where W153 is substituted with a Cys (shown in bold and italics text).

### DRB3 Polypeptides

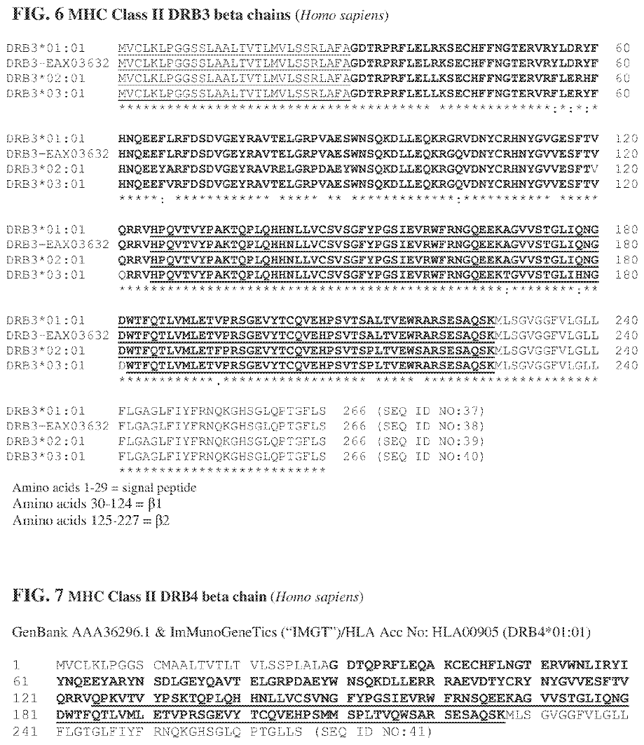

In some cases, a suitable MHC Class II β chain polypeptide is a DRB3 polypeptide. In an embodiment, a DRB3 polypeptide can have at least 60%, at least 70%, at least 80%, at least 90%, at least 95%, at least 98%, at least 99%, or 100% aa sequence identity with aas 30-227 of any DRB3 aa sequence depicted in FIG. 6, which displays the DRB3 precursor proteins in which aas 1-29 are the signal sequence (underlined), 30-124 form the β1 region (shown bolded), 125-227 form the β2 region, and 228-250, the transmembrane region. In an embodiment, a DRB3β3 chain polypeptide can have at least 60%, at least 70%, at least 80%, at least 90%, at least 95%, at least 98%, at least 99%, or 100% an sequence identity with aas 30-227 of the DRB1-3 (DRB3*01:01) beta chain aa sequence GenBank NP_0.072049.1 in FIG. 6. In an embodiment, a DRB3 D chain polypeptide can have at least 60%, at least 70%, at least 80%, at least 90%, at least 95%, at least 98%, at least 99%, or 100% an sequence identity with aas 30-227 of the DRB1-3 beta chain aa sequence in GenBank accession EAX03632.1 in FIG. 6. In an embodiment, a DRB3 β chain polypeptide can have at least 60%, at least 70%, at least 80%, at least 90%, at least 95%, at least 98%, at least 99%, or 100% aa sequence identity with aas 30-227 of the DRB1-3 (DRB3*02:01) beta chain aa sequence GenBank CAA23781.1 in FIG. 6. In an embodiment, a DRB3 β chain polypeptide can have at least 60%, at least 70%, at least 80%, at least 90%, at least 95%, at least 98%, at least 99%, or 100% aa sequence identity with aas 30-227 of the DRB1-3 (DRB3*03:01) beta chain aa sequence GenBank AAN15205.1 in FIG. 6. In some cases, a DRB3 polypeptide suitable for inclusion in a TMP comprises an aa substitution, relative to a wild-type DRB3 polypeptide, where the aa substitution replaces an aa (other than a Cys) with a Cys.

A suitable “DRB3 polypeptide” for incorporation into a TMP may include allelic variants, e.g., naturally occurring allelic variants. Thus, in some cases, a suitable DRB3 polypeptide comprises aas 30 to 227 of DRB3*01:01 provided in FIG. 6, or an allelic variant thereof. Thus, in some cases, a suitable DRB3 polypeptide comprises a sequence having at least 80%, at least 90%, at least 95%, at least 98%, at least 99%, or 100% sequence identity to at least 170, at least 180, or at least 190 contiguous aas of the following sequence: DTRPRFLELR KSECHFFNGT ERVRYLDRYF HNQEEFLRFD SDVGEYRAVT ELGRPVAESW NSQKDLLEQK RGRVDNYCRH NYGVGESIFV QRRVHPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:210), or an allelic variant thereof. In some cases, a DRB3 polypeptide suitable for inclusion in a TMP comprises an aa substitution, relative to a wild-type DRB3 polypeptide, where the aa substitution replaces an aa (other than a Cys) with a Cys. Thus, e.g., in some cases, the MHC class II β chain polypeptide is a variant DRB3 MHC class II polypeptide that comprises a non-naturally occurring Cys at an aa selected from the group consisting of P5C, F7C, L10C, N19C, G20C, N33C, G151C, D152C, and W153C (of a mature DRB3 polypeptide (lacking the N-terminal signal peptide MVCLKLPGGSSLAALTVTLMVLSSRLAFA (SEQ ID NO:203) depicted in FIG. 6).

A suitable DRB3β1 domain, including-naturally occurring allelic variants thereof, may comprise an aa sequence having at least 70%, at least 80%, at least 90%, at least 95%, at least 98%, at least 99%, or 100% aa sequence identity to the following an sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRV (SEQ ID NO:211); and can have a length of about 95 aas (e.g., 93, 94, 95, 96, 97, or 98 aus). A suitable DRB3β1 domain can comprise the following aa sequence: DTIRPRFLELR KSECHFFNGT ERVRYLDRYF HNQEEFLRFD SDVGEYRAVT ELGRPVAESW NSQKDLLEQK RGRVDNYCRH NYGVGES-TV QRRV (SEQ ID NO:211), or a naturally-occurring allelic variant A suitable DRB3β2 domain, including-naturally occurring allelic variants thereof, may comprise an aa sequence having at least 70%, at least 80%, at least 90%, at least 95%, at least 98%, at least 99%, or 100% aa sequence identity to the following aa sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:212); and can have a length of about 103 aas (e.g., 100, 101, 102, 103, 104, or 105 aas). A suitable DRB3β2 domain can comprise the following aa sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:212), or a naturally-occurring allelic variant thereof.

### DRB4 Polypeptides

In some cases, a suitable MHC Class II β chain polypeptide is a DRB4 polypeptide. A DRB4 polypeptide can have at least 60%, at least 70%, at least 80%, at least 90%, at least 95%, at least 98%, at least 99%, or 100% aa sequence identity with aas 30-227 of a DRB4 aa sequence depicted in FIG. 7. In some cases, the DRB4 polypeptide has a length of about 198 aas (e.g., 195, 196, 197, 198, 199, 200, 201, or 202 aas). In some cases, a DRB4 polypeptide suitable for inclusion in a TMP comprises an amino acid substitution, relative to a wild-type DRB4 polypeptide, where the amino acid substitution replaces an amino acid (other than a Cys) with a Cys.

A suitable “DRB4 polypeptide” for inclusion in a TMP may include allelic variants, e.g., naturally occurring allelic variants. Thus, in some cases, a suitable DRB4 polypeptide comprises aas 30 to 227 of DRB4*01:01 (SEQ ID NO:41) or DRB4*01:03 (SEQ ID NO:42) provided in FIG. 7, or an allelic variant thereof. In some cases, a DRB4 polypeptide suitable for inclusion in a TMP comprises an amino acid substitution, relative to a wild-type DRB4 polypeptide, where the amino acid substitution replaces an amino acid (other than a Cys) with a Cys. Thus, e.g., in some cases, the MHC class II β chain polypeptide is a variant DRB4 MHC class II polypeptide that comprises a non-naturally occurring Cys residue; e.g., where the variant DRB4 MHC class II polypeptide comprises an amino acid substitution selected from the group consisting of P15C, F17C, Q20C. N29C, G30C, N43C, G161C. D162C, and W163C of a mature DRB4 polypeptide (lacking the N-terminal signal peptide MVCLKLPGGSCMAALTVTL (SEQ ID NO:204) depicted in FIG. 7).

A suitable DRB4 β1 domain, including-naturally occurring allelic variants thereof, may comprise an as sequence having at least 70%, at least 80%, at least 90%, at least 95%, at least 98%, at least 99%, or 100% aa sequence identity to the following aa sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESF-TV QRRV (SEQ ID NO:213); and can have a length of about 95 aas (e.g., 93, 94, 95, 96, 97, or 98 aas).

A suitable DRB4β2 domain, including-naturally occurring allelic variants thereof, may comprise an aa sequence having at least 70%, at least 80%, at least 90%, at least 95%, at least 98%, at least 99%, or 100% aa sequence identity to the following aa sequence: QPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO:214); and can have a length of about 103 aas (e.g., 100, 101, 102, 103, 104, or 105 aas).

### DRB5 Polypeptides

A TMP may comprise an MHC Class II β chain polypeptide of a DRB5 allele. In some cases, the DRB5 polypeptide has a length of about 198 aas (e.g., 195, 196, 197, 198, 199, 200, 201, or 202 aas). In some cases, a DRB5 polypeptide suitable for inclusion in a TMP comprises an aa substitution, relative to a wild-type DRB5 polypeptide, where the aa substitution replaces an aa (other than a Cys) with a Cys.

DRB5*01:01

A TMP may comprise a DRB5*01:01 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB5*01:01 aa sequence provided in FIG. 8. In some cases, a DRB5 polypeptide suitable for inclusion in a TMP comprises an aa substitution, relative to a wild-type DRB5 polypeptide, where the aa substitution replaces an aa (other than a Cys) with a Cys. Thus. e.g., in some cases, the MHC class II β chain polypeptide is a variant DRB5 MHC class II polypeptide that comprises a non-naturally occurring Cys residue; e.g., where the variant DRB5 MHC class II polypeptide comprises an aa substitution selected from the group consisting of P15C, F17C, Q20C, N29C, G30C, N43C, G161C, D162C, and W163C (of a mature DRB5 polypeptide (lacking the N-terminal signal peptide MVCLKLPGGSYMAKLTVTL (SEQ ID NO:205) depicted in FIG. 8), or a naturally-occurring allelic variant thereof.

A TMP may comprise a DRB5*01:01 β1 domain polypeptide comprising an aa sequence having at least 70%, at least 80%, at least 90%, at least 95%, at least 98%, at least 99%, or 100%, aa sequence identity to aas 30-124 of the DRB5*01:01 aa sequence provided in FIG. 8. A TMP may comprise a DRB5*01:01 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB5*01:01 β2 domain aa sequence depicted in FIG. 8, or a naturally-occurring allelic variant thereof.

### DMB Polypeptides

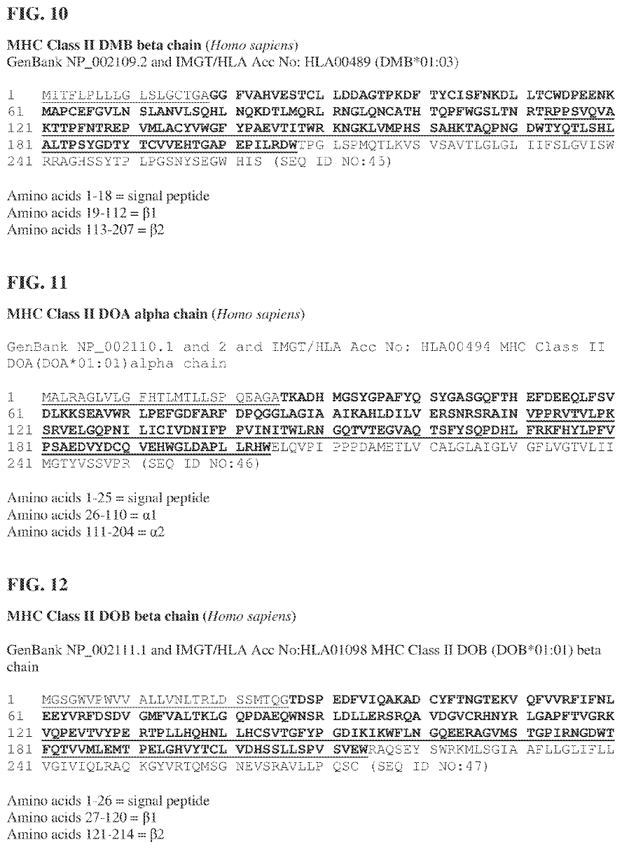

In some cases, a suitable MHC Class II D chain polypeptide is a DMB polypeptide. A DMB polypeptide can have at least 60%, at least 70%, at least 80%, at least 90%, at least 95%, at least 98%, at least 99%, or 100%, an sequence identity with aas 19-207 of the DMB aa sequence depicted in FIG. 10. In some cases, the DMB polypeptide has a length of about 189 aas (e.g., 187, 188, 189, 190, or 191 aas).

A “DMB polypeptide” includes allelic variants, e.g., naturally occurring allelic variants. Thus, in some cases, a suitable DMB polypeptide comprises aas 19 to 207 of DMB*01:03 (SEQ ID NO:45) provided in FIG. 10, or an allelic variant thereof.

A suitably DMB β1 domain, including-naturally occurring allelic variants thereof, may comprise an aa sequence having at least 70%, at least 80%, at least 90%, at least 95%, at least 98%, at least 99%, or 100%, aa sequence identity to the following aa sequence: GG FVAHVESTCL LDDAGTPKDF TYCISFNKDL LTCWDPEENK MAPCEFGVLN SLANVLSQHL NQKDTLMQRL RNGLQNCATH TQPFWGSLTN RT (SEQ ID NO:215); and can have a length of about 94 aas (e.g., 92, 93, 94, 95, 96, or 97 aas).

A suitable DMB β2 domain, including-naturally occurring allelic variants thereof, may comprise an aa sequence having at least 70%, at least 80%, at least 90%, at least 95%, at least 98%, at least 99%, or 100%, an sequence identity to the following an sequence: RPPSVQVA KTTPFNTREP VMLACYVWGF YPAEVTITWR KNGKLVMPHS SAHKTAQPNG DWTYQTLSHL ALTPSYGDTY TCVVEHTGAP EPILRDW (SEQ ID NO:216); and can have a length of about 95 aas (e.g., 93, 94, 95, 96, 97, or 98 aas).

### DOB Polypeptides

In some cases, a suitable MHC Class II β chain polypeptide is a DOB polypeptide. A DOB polypeptide can have at least 60%, at least 70%, at least 80%, at least 90%, at least 95%, at least 98%, at least 99%, or 100%, aa sequence identity with aas 27-214 of the DOB aa sequence depicted in FIG. 12. In some cases, the DOB polypeptide has a length of about 188 aas (e.g., 186, 187, 188, 189, or 190 aas).

A “DOB polypeptide” includes allelic variants, e.g., naturally occurring allelic variants. Thus, in some cases, a suitable DOB polypeptide comprises aas 27-214 of DOB*01:01 (SEQ ID NO:47) provided in FIG. 12, or an allelic variant thereof.

A suitable DOB 01 domain, including-naturally occurring allelic variants thereof, may comprise an aa sequence having at least 70%, at least 80%, at least 90%, at least 95%, at least 98%, at least 99%, or 100%, aa sequence identity to the following aa sequence: TDSP EDFVIQAKAD CYI-TNGTEKV QFVVRFIFNL EEYVRFDSDV GMFVALTKLG QPDAEQWNSR LDLLERSRQA VDGVCRHNYR LGAPFTVGRK (SEQ ID NO:217); and can have a length of about 94 aas (e.g., 92, 93, 94, 95, 96, or 97 aas).

A suitable DOB 02 domain, including-naturally occurring allelic variants thereof, may comprise an aa sequence having at least 70%, at least 80%, at least 90%, at least 95%, at least 98%, at least 99%, or 100%, aa sequence identity to the following aa sequence: VQPEVTVYPE RTPLLHQHNL LHCSVTGFYP GDIKIKWFLN GQEERAGVMS TGPIRNGDWT FQTVVMLEMT PELGHVYTCL VDHSSLLSPV SVEW (SEQ ID NO:218); and can have a length of about 94 aas (e.g., 92, 93, 94, 95, 96, or 97 aas).

### DPB1 Polypeptides

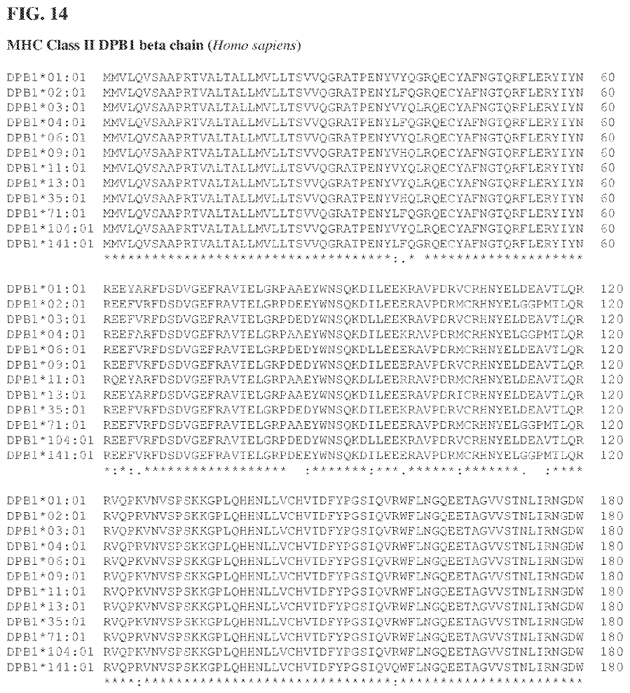

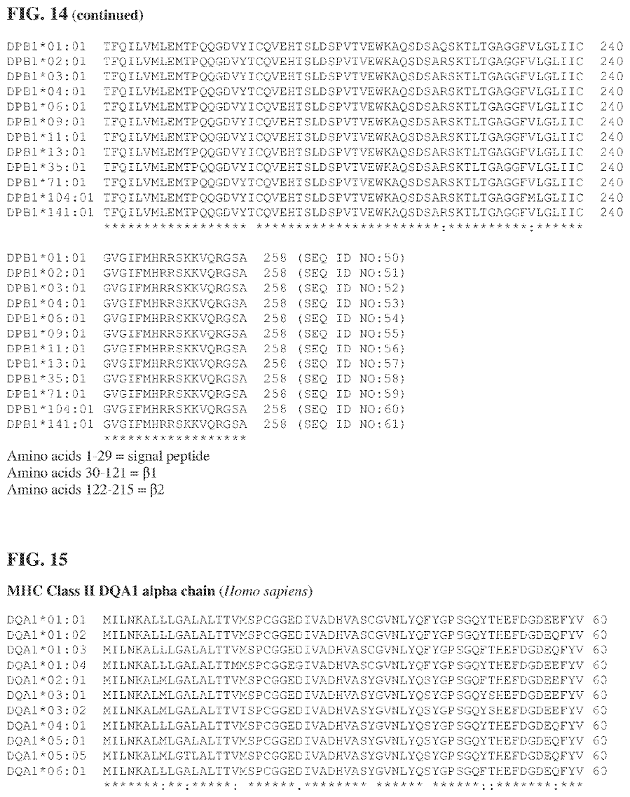

In some cases, a suitable MHC Class II β chain polypeptide is a DPB1 polypeptide. A DPB1 polypeptide can have at least 60%, at least 70%, at least 80%, at least 90%, at least 95%, at least 98%, at least 99%, or 100%, aa sequence identity with aas 30-215 of any of the DPB1 aa sequences depicted in FIG. 14. In some cases, the DPB1 polypeptide has a length of about 186 aas (e.g., 184, 185, 186, 187, or 188 aas). In an embodiment, a DRB3β chain polypeptide can have at least 60%, at least 70%, at least 80%, at least 90%, at least 95%, at least 98%, at least 99%, or 100%, aa sequence identity with aas 30-227 of the DPB1*0:01 β chain aa sequence in FIG. 14. IMGT/HLA Ace No: HLA00514. In an embodiment, a DRB3 β chain polypeptide can have at least 60%, at least 70%, at least 80%, at least 90%, at least 95%, at least 98%, at least 99%, or 100%, aa sequence identity with aas 30-227 of the DPB1*02:01 β chain aa sequence in FIG. 14, IMGT/HLA Ace No: HLA00517. In an embodiment, a DRB3 β chain polypeptide can have at least 60%, at least 70%, at least 80%, at least 90%, at least 95%, at least 98%, at least 99%, or 100%, an sequence identity with aas 30-227 of the DPB1*03:01 chain an sequence in FIG. 14, IMGT/HLA Ace No: HLA00520. In an embodiment, a DRB3 β chain polypeptide can have at least 60%, at least 70%, at least 80%, at least 90%, at least 95%, at least 98%, at least 99%, or 100%, aa sequence identity with aas 30-227 of the DPB1*04:01 β chain aa sequence in FIG. 14, IMGT/HLA Acc No: HLA00521, GenBank NP_002112.3. In an embodiment, a DRB3 β chain polypeptide can have at least 60%, at least 70%, at least 80%, at least 90%, at least 95%, at least 98%, at least 99%, or 100%, aa sequence identity with aas 30-227 of the DPB1*06:01 β3 chain aa sequence in FIG. 14. IMGT/HLA Acc No: HLA00524. In an embodiment, a DRB3β chain polypeptide can have at least 60%, at least 70%, at least 80%, at least 90%, at least 95%, at least 98%, at least 99%, or 100%, aa sequence identity with aas 30-227 of the DPB1*l 1:01 β chain aa sequence in FIG. 14, IMGT/HLA Ace No: HLA00528. In an embodiment, a DRB3β chain polypeptide can have at least 60%, at least 70%, at least 80%, at least 90%, at least 95%, at least 98%, at least 99%, or 100%, aa sequence identity with aas 30-227 of the DPB1*71:01 β chain an sequence in FIG. 14, IMGT/HLA Ace No:HLA00590. In an embodiment, a DRB3β chain polypeptide can have at least 60%, at least 70%, at least 80%, at least 90%, at least 95%, at least 98%, at least 99%, or 100%, aa sequence identity with aas 30-227 of the DPB1*104:01 chain an sequence in FIG. 14. IMGT/HLA Ace No: HLA02046. In an embodiment, a DRB3 β chain polypeptide can have at least 60%, at least 70%, at least 80%, at least 90%, at least 95%, at least 98%, at least 99%, or 100%, an sequence identity with aas 30-227 of the DPB1*141:01 beta chain aa sequence in FIG. 14, IMGT/HLA Acc No: HLA10364.

A “DPB1 polypeptide” includes allelic variants, e.g., naturally occurring allelic variants. Thus, in some cases, a suitable DPB1 polypeptide comprises the following aa sequence: R ATPENYLFQG RQECYAFNGT QRFLERYIYN REEFARFDSD VGEFRAVTEL GRPAAEYWNS QKDILEEKRA VPDRMCRHNY ELGGPMTLQR RVQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO:219), or an allelic variant thereof.

A suitable DPB1 β1 domain, including-naturally occurring allelic variants thereof, may comprise an aa sequence having at least 70%, at least 80%, at least 90%, at least 95%, at least 98%, at least 99%, or 100%, aa sequence identity to the following aa sequence: R ATPENYLFQG RQECYAFNGT QRFLERYIYN REEFARFDSD VGEFRAVTEL GRPAAEYWNS QKDILEEKRA VPDRMCRHNY ELGGPMTLQR R (SEQ ID NO:220); and can have a length of about 92 aas (e.g., 90, 91, 92, 93, or 94 aas).

A suitable DPB1β2 domain, including-naturally occurring allelic variants thereof, may comprise an aa sequence having at least 70%, at least 80%, at least 90%, at least 95%, at least 98%, at least 99%, or 100%, an sequence identity to the following an sequence: VQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO:221); and can have a length of about 94 aas (e.g., 92, 93, 94, 95, 96, or 97 aas).

### DQB1 Polypeptides

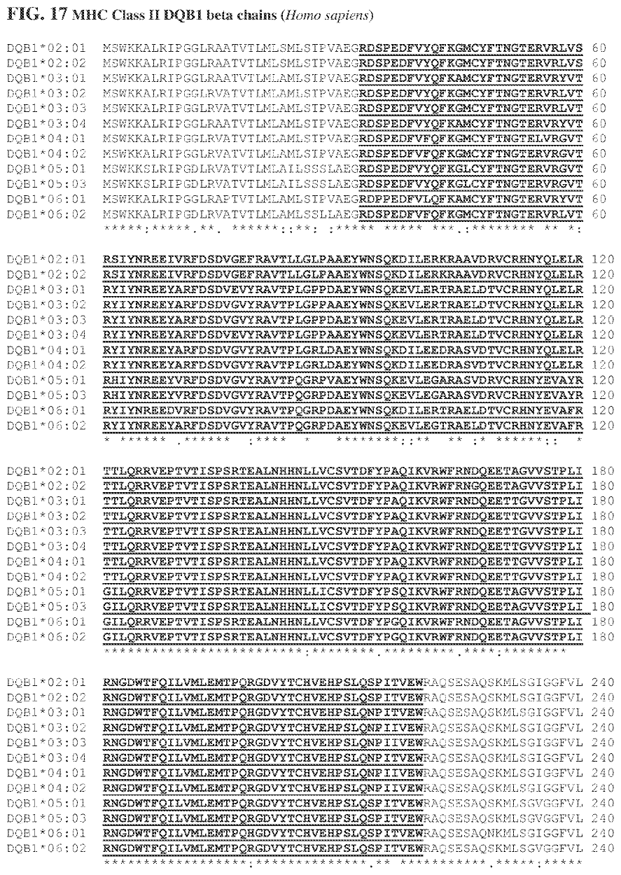

In some cases, a suitable MHC Class II β chain polypeptide is a DQB1 polypeptide. A DQB1 polypeptide can have at least 60%, at least 70%, at least 80%, at least 90%, at least 95%, at least 98%, at least 99%, or 100%, aa sequence identity with aas 33-220 of the DQB1 aa sequence depicted in FIG. 17. In some cases, the DQB1 polypeptide has a length of about 188 aas (e.g., 186, 187, 188, 190, 191, or 192 aas).

A “DQB1 polypeptide” includes allelic variants, e.g., naturally occurring allelic variants. Thus, in some cases, a suitable DQB1 polypeptide comprises aas 33-220 of DQB1*06:02 provided in FIG. 17 (SEQ ID NO:85), or an allelic variant thereof.

A suitable DQB1 β1 domain, including-naturally occurring allelic variants thereof, may comprise an aa sequence having at least 70%, at least 80%, at least 90%, at least 95%, at least 98%, at least 99%, or 100%, aa sequence identity to the following an sequence: RDSPEDFV FQFKGMCYFT NGTERVRLVT RYIYNREEYA RFDSDVGVYR AVTPQGRPDA EYWNSQKEVL EGTRAELDTV CRHNYEVAFR GILQRR (SEQ ID NO:222); and can have a length of about 94 aas (e.g., 92, 93, 94, 95, or 96 aas).

A suitable DQB1β2 domain, including-naturally occurring allelic variants thereof, may comprise an aa sequence having at least 70%, at least 80%, at least 90%, at least 95%, at least 98%, at least 99%, or 100%, aa sequence identity to the following aa sequence: VEPT VTISPSRTEA LNHHNLLVCS VTDFYPGQIK VRWFRNDQEE TAGVVSTPLI RNGDWTFQIL VMLEMTPQRG DVYTCHVEHP SLQSPITVEW (SEQ ID NO:223); and can have a length of about 94 aas (e.g., 92, 93, 94.95, or 96 aas).

### DQB2 Polypeptides

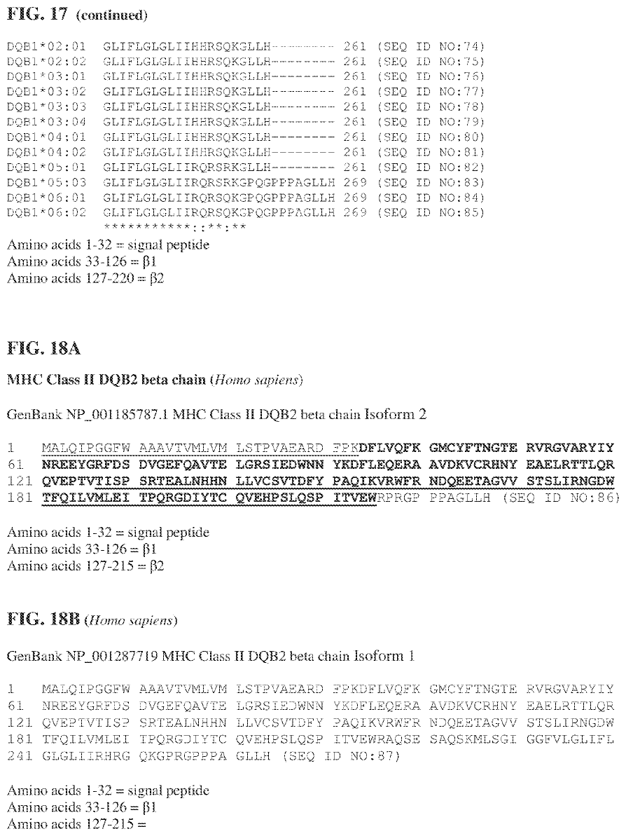

In some cases, a suitable MHC Class II β chain polypeptide is a DQB2 polypeptide. A DQB2 polypeptide can have at least 60%, at least 70%, at least 80%, at least 90%, at least 95%, at least 98%, at least 99%, or 100%, aa sequence identity with aas 33-215 of the DQB2 aa sequence depicted in FIG. 18A or FIG. 18B. In some cases, the DQB2 polypeptide has a length of about 182 aas (e.g., 175, 176, 177, 178, 179, 180, 181, or 182 aas).

A “DQB2 polypeptide” includes allelic variants, e.g., naturally occurring allelic variants. Thus, in some cases, a suitable DQB2 polypeptide comprises the following aa sequence: DFLVQFK GMCYFTNGTE RVRGVARYIY NREEYGRFDS DVGEFQAVTE LGRSIEDWNN YKDFLEQERA AVDKVCRHNY EAELRTTLQR QVEFTVTISP SRTEALNHHN LLVCSVTDFY PAQIKVRWFR NDQEETAGVV STSLIRNGDW TFQILVMLEI TPQRGDIYTC QVEHPSLQSP ITVEW (SEQ ID NO:224), or an allelic variant thereof.

A suitable DQB2 β2 domain, including-naturally occurring allelic variants thereof, may comprise an aa sequence having at least 70%, at least 80%, at least 90%, at least 95%, at least 98%, at least 99%, or 100%, aa sequence identity to the following aa sequence: DFLVQFK GMCYFINGIE RVRGVARYIY NREEYGRFDS DVGEFQAVTE LGRSIEDWNN YKDFLEQERA AVDKVCRHNY EAELRTTLQR QVEPTV (SEQ ID NO:225); and can have a length of about 94 aas (e.g., 92 93, 94, 95, 96, or 97 aas).

A suitable DQB2 β2 domain, including-naturally occurring allelic variants thereof, may comprise an aa sequence having at least 70%, at least 80%, at least 90%, at least 95%, at least 98%, at least 99%, or 100%, aa sequence identity to the following an sequence: TISP SRTEALNHHN LLVCSVIDFY PAQIKVRWFR NDQEETAGVV STSLIRNGDW TFQILVMLEI TPQRGDIYTC QVEHPSLQSP ITVEW (SEQ ID NO:226); and can have a length of about 94 aas (e.g., 92, 93, 94, 95, 96, or 97 aas).

### MHC Class II Disease Risk-Associated Alleles and Haplotypes

Certain alleles and haplotypes of MHC class II have been associated with disease, e.g., increased risk of developing a particular disease. See, e.g., Erlich et al. (2008) Diabetes 57:1084; Gough and Simmonds (2007) Curr. Genomics 8:453; Mitchell et al. (2007) Robbins Basic Pathology Philadelphia; Saunders, 8^{th} ed.; Margaritte-Jeannin et al. (2004) Tissue Antigens 63:562; and Kurko et al. (2013) Clin. Rev. Allergy Immunol, 45:170.

### MHC Class H Polypeptides in Type 1 Diabetes Mellitus (T1D)

Alleles/isoforms showing increased association with T1D represent suitable sources of MHC II α1. α2, (1, and β2 polypeptide sequences for incorporation into TMPs directed to the treatment of T1D. T1D is associated with alleles belonging to the HLA-DR3 and HLA-DR4 haplotypes/serotypes, with the strongest risk associated with the HLA-DQ8, (e.g., HLA-DQB1*03:02) and alleles of the HLA-DQ2 serotype. Some high and moderate risk haplotypes and their association with various DR serotypes are shown in Table 5, below, which is adapted from Kantárová and Buc, Physiol. Res. 56: 255-266 (2007).

TABLE 5

| | | | | |
|---|---|---|---|---|
| | | DQ | | |
| Haplotype | DRB allele | serotype | DQA allele | DQB allele |

High risk T1D haplotypes

| | | | | |
|---|---|---|---|---|
| DR3 | DRB1*0301 | DQ 2.5 | DQA1*0501 | DQB1*0201 |
| DR4 | DRB1*0401 | DQ 8.1 | DQA1*0301 | DQB1*0302 |
| DR4 | DRB1*0402 | DQ 8.1 | DQA1*0301 | DQB1*0302 |
| DR4 | DRB1*0405 | DQ 8.1 | DQA1*0301 | DQB1*0302 |

Moderate risk T1D haplotypes

| | | | | |
|---|---|---|---|---|
| DR1 | DRB1*01 | DQ 5 | DQA1*0101 | DQB1*0501 |
| DR8 | DRB1*0801 | | DQA1*0401 | DQB1*0402 |
| DR9 | DRB1*0901 | | DQA1*0301 | DQB1*0303 |

Recognized associations

DPB allele
DPB1*02:02
DPB1*03:01

The stereotypically defined DR3 and DR4 protein isoforms/haplotypes of the DRB1 gene are associated with increased risk that an individual expressing such alleles will develop T1D. The DR3 serotype includes the alleles encoding the DRB1*03:01, *03:02, *03:03, and *03:04 proteins, with the HLA-DRB1*0301 allele often found associated with a predisposition to T1D. The DR4 serotype includes the alleles encoding the DRB1*04:01, *04:02, *04:03, *04:04, *04:05, *04:06, *04:07, *04:08, *04:09, *04:10, *04:11, *04:12, and *04:13 proteins. Certain HLA-DR4 alleles (e.g., HLA-DRB1*0401, HLA-DRB1*0402, and HLA-DRB1*0405) predispose individuals to T1D, whereas HLA-DRB1*04:03 allele/isoform may afford protection. DRB1*16:01 also show an increased frequency in diabetic children relative to healthy controls (Deja, et al., Mediators of Inflammation 2006:1-7 (2006)).

While T1D is associated with DR3 and DR4 alleles as discussed above, among the strongest associated risk factors for T1D, the presence of alleles belonging to the HLA-DQ8 serotype (e.g., the HLA-DQB1*03:02 isoform) and the HLA DQ2 serotype (e.g., HLA-DQB1*0201). HLA-DQ8 T1D susceptible serotypes include the HLA-DQ8.1 serotype (HLA-DQA1*03:01/DQB1*03:02), The HLA-DQ2 serotype (HLA-DQB1*02) associated with T1D include DQB1*02:01). Jones, et al., Nat. Rev. Immunol. 2006, 6: 271-282. By contrast, individuals that carry the HLADQB1*0602 allele appear to be protected against type 1 diabetes. Id. DQ2 is most common in Western Europe, North Africa and East Africa, with the highest frequencies observed in parts of Spain and Ireland.

The DQB1 locus alone has also been reported to be associated with T1D when position β57 is a neutral residue such as Ala or Ser. Both the DQ2 and DQ8 serotypes, which are associated with T1D, lack an Asp at the 57β position, and instead have an Ala in its place (see e.g., Ala 89 in FIG. 19B HLA-DQB1*02:01 and 19C. HLA-DQB1**03:2 respectively) conferred T1D susceptibility. In contrast, DQB1*06:02, which has an Asp) at position 057 of DQB1 (position 89 in FIG. 11A-11B) was found to be associated with resistance to T1D. Jones et al, Nat. Rev. Immunol. 2006, 6: 271-282. Position 057 of the molecule forms a critical residue in peptide binding pocket nine (P9) of the DQB1, which is involved in antigen presentation and T cell receptor (TCR) interaction.

Individuals with the HLA haplotype DQA1*03:01-DRB1*03:02, especially when combined with DQA1*05:01-DRB1*02:01, are highly susceptible (10-20-fold increase) to T1D, see Notkins. A. L. J. Biol. Chem., 2002, 277(46): 43545-48. Among the stereotypically defined groups showing susceptibility to T1D are HLA-DR4.1 (HLA-DRA1*01:01/DRB1*04:01). HLA-DR4.5 (HLA-DRA1*01:01/DRB1*04:05). HLA-DQ2.5 (HLA-DQA1*05:01/DQB1*02:01), and HLA-DQ8.1 (HLA-DQA1*03:01/DQB1*03:02). (sec e.g., Jones et al., Nat. Rev. Immunol. 2006, 6: 271-282). The DRβ1*04:05-DQβ1*04:01/DRβ1*08:02-DQβ1*03:02 genotype has shown to be associated with acute-onset and slow progressive T1D. Fulminant diabetes has been associated with DRβ1*04:05-DQβ1*04:01/DRβ1*04:05-DQβ1*04:01 genotype, in a Japanese population study Kawabata, et al., Diabetologia 2009, 52:2513-21.

Although the HLA-DR associations with T1D are not as strong as those of HLA-DQ, insulin-reactive T cells derived from lymph nodes draining the pancreas of patients with T1D appear to be HLA-DR4.1 restricted rather than HLA-DQ8 or HLA-DQ2 restricted (Kent wt al. Nature 2005 435: 224-228).

The above-mentioned alleles associated with an increased risk of T1D represent suitable candidates from which the α1. α2. β1, and/or β2 polypeptide sequences present in a TMP of the present disclosure may be taken. In an embodiment, the TMP is DQ2.5-like with the α1 and α2 polypeptides from DQA1*0501, and the β1 and β2 polypeptides taken from DQB1*0201. In an embodiment, the TMP is DQ8.1-like with the α1 and α2 polypeptides from DQA1*0301, and the β1 and D2 polypeptides taken from DQB1*0302.

### Scaffold Polypeptides

A TMP of the present disclosure, whether a heterodimeric TMP or a single-chain TMP, can comprise an immunoglobulin or non-immunoglobulin scaffold. A TMP polypeptide of the present disclosure, whether a heterodimeric TMP or a single-chain TMP, can comprise an Fc polypeptide, or can comprise another suitable scaffold polypeptide.

Suitable scaffold polypeptides include antibody-based scaffold polypeptides and non-antibody-based scaffolds. Non-antibody-based scaffolds include, e.g., albumin, an XTEN (extended recombinant) polypeptide, transferrin, an Fe receptor polypeptide, an elastin-like polypeptide (see. e.g., Hassounch et al. (2012) Methods Enzymol. 502:215; e.g., a polypeptide comprising a pentapeptide repeat unit of (Val-Pro-Gly-X-Gly; SEQ ID NO:227), where X is any amino acid other than proline), an albumin-binding polypeptide, a silk-like polypeptide (see, e.g., Valluzzi et al. (2002) Philos Trans R Soc Land B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human scrum albumin.

Suitable scaffold polypeptides will in some cases be half-life extending polypeptides. Thus, in some cases, a suitable scaffold polypeptide increases the in vivo half-life (e.g., the serum half-life) of the polypeptide (e.g., the TMP), compared to a control polypeptide lacking the scaffold polypeptide. For example, in some cases, a scaffold polypeptide increases the in vivo half-life (e.g., the serum half-life) of the polypeptide, compared to a control polypeptide lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, an Fc polypeptide increases the in vivo half-life (e.g., the serum half-life) of the polypeptide, compared to a control polypeptide lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

Fc Polypeptides

In some cases, a TMP comprises an Ig Fc polypeptide. An Ig Fc polypeptide is also referred to herein as an “Fc polypeptide.” The Ig Fc polypeptide of a TMP can be a human IgG1 Fe, a human IgG2 Fe, a human IgG3 Fc, a human IgG4 Fc, etc., or a variant of a wild-type Ig Fc polypeptide. Variants include naturally-occurring variants, non-naturally-occurring variants, and combinations thereof. Exemplary Ig Fc polypeptides are discussed below, any of which may further include interspecific binding sequences, if desired.

In some cases, the Fe polypeptide present in a TMP comprises an amino acid sequence having at least about 70%, at least about 75%, at least about 80%, at least about 85%, at least about 90%, at least about 95%, at least about 98%, at least about 99%, or 100%, amino acid sequence identity to the Fe amino acid sequence depicted in any one of FIG. 21A-21M.

In some cases, the Fc polypeptide present in a TMP is an IgG1 Fc polypeptide, or a variant of an IgG1 Fc polypeptide. For example, in some cases, the Fc polypeptide present in a TMP comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fe polypeptide depicted in FIG. 21A. As another example, in some cases, the Fc polypeptide present in a TMP comprises an amino acid sequence having at least about 70%, at least about 75%, at least about 80%, at least about 85%, at least about 90%, at least about 95%, at least about 98%, at least about 99%, or 100%, amino acid sequence identity to the Fc polypeptide depicted in FIG. 21B; where the Ig Fc polypeptide comprises an Ala at position 14 and an Ala at position 15. In any of the above embodiments, the Ig Fc polypeptide can have an N77 substitution; i.e., the Ig Fc polypeptide can have an amino acid other than Asn at position 77, where in some cases, the Ig Fc polypeptide has an Ala at position 77. In some cases, an Fc polypeptide present in a TMP comprises the amino acid sequence depicted in FIG. 21A. In some cases, an Fc polypeptide present in a TMP comprises the amino acid sequence depicted in FIG. 21B.

In some cases, the Fc polypeptide present in a TMP is an IgG1 Fc polypeptide, or a variant of an IgG1 Fc polypeptide, where variants include naturally-occurring variants, non-naturally-occurring variants, and combinations thereof. For example, in some cases, the Fe polypeptide present in a TMP comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fe polypeptide depicted in FIG. 21C; where the Ig Fc polypeptide comprises a Glu at position 136 and a Met at position 138. As another example, in some cases, the Fe polypeptide present in a TMP comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fe polypeptide depicted in FIG. 21D; where the Ig Fc polypeptide has Ala at positions 14 and 15; and where the Fe polypeptide comprises a Glu at position 136 and a Met at position 138. In any of the above embodiments, the Ig Fc polypeptide can have an N77 substitution; i.e., the Ig Fc polypeptide can have an amino acid other than Asn at position 77, where in some cases, the Ig Fc polypeptide has an Ala at position 77. In some cases, an Fc polypeptide present in a TMP comprises the amino acid sequence depicted in FIG. 21C. In some cases, an Fc polypeptide present in a TMP comprises the amino acid sequence depicted in FIG. 21D.

In some cases, the Fe polypeptide present in a TMP comprises the amino acid sequence depicted in FIG. 21E (human IgG1 Fc comprising an L234F substitution, an L235E substitution, and a P331S substitution; where L234 corresponds to amino acid 14 of the amino acid sequence depicted in FIG. 21E; L235 corresponds to amino acid 15 of the amino acid sequence depicted in FIG. 21E; and P331 corresponds to amino acid 111 of the amino acid sequence depicted in FIG. 21E). In some cases, the Fc polypeptide present in a TMP comprises the amino acid sequence depicted in FIG. 21F, comprising an N279A substitution (N77A of the amino acid sequence depicted in FIG. 21F).

In some cases, the Fe polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG2 Fe polypeptide depicted in FIG. 21G; e.g., the Fe polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 99-325 of the human IgG2 Fe polypeptide depicted in FIG. 21G (e.g., where the Ig Fc polypeptide has a length of about 227 amino acids). In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG3 Fe polypeptide depicted in FIG. 21H; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 19-246 of the human IgG3 Fc polypeptide depicted in FIG. 21H (e.g., where the Ig Fc polypeptide has a length of about 228 amino acids). In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgM Fc polypeptide depicted in FIG. 21J; e.g., the Fe polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-276 to the human IgM Fe polypeptide depicted in FIG. 21J. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgA Fc polypeptide depicted in FIG. 21K; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-234 to the human IgA Fe polypeptide depicted in FIG. 21K.

In some cases, the Fe polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG4 Fc polypeptide depicted in FIG. 21M. In some cases, the Fe polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 100 to 327 of the human IgG4 Fe polypeptide depicted in FIG. 21M (e.g., where the Ig Fc polypeptide has a length of about 228 amino acids).

In some cases, the IgG4 Fc polypeptide comprises the following amino acid sequence:

(SEQ ID NO: 228)
PPCPSCPAPEFLGGPSVFLFPPKPKDTLMISRTPEVTCVVVDVSQEDPE
VQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQE
GNVFSCSVMHEALHNHYTQKSLSLSPG.

In some cases, the Ig Fc employed in a TMP will comprise one or more substitutions of amino acids in the wild-type sequence, such that that Ig Fc that substantially does not induce cell lysis. For example, in some cases the Fc polypeptide present in a TMP comprises the amino acid sequence depicted in FIG. 21A (human IgG1 Fe), except for a substitution of L234 (L14 of the amino acid sequence depicted in FIG. 21A) with an amino acid other than leucine, or a substitution of L235 (L15 of the amino acid sequence depicted in FIG. 21A) with an amino acid other than leucine. Examples include an L234A (L14A) substitution; and an L235A (L15A) substitution.

### TGF-β Polypeptides

As discussed above, a TMP of the present disclosure comprises at least one TGF-β polypeptide reversibly masked by a polypeptide (a “masking polypeptide”) that binds to the TGF-β polypeptide, which together form a masked TGF-β MOD. The masking polypeptide can be, for instance, a TGF-β receptor polypeptide or an antibody that functions to reversibly mask the TGF-β polypeptide present in the TMP, where the TGF-β polypeptide is otherwise capable of acting as an agonist of a cellular TGF receptor. The masked TGF-β MODs provide active TGF-β polypeptides (e.g., TGF-β signaling pathway agonists). The TGF-β polypeptides and masking polypeptides (e.g., a TGF-β receptor fragment) interact with each other to reversibly mask the TGF-β polypeptide, thereby permitting the TGF-β polypeptide to interact with its cellular receptor. In addition, the masking sequence competes with cellular receptors that can scavenge TGF-β, such as the non-signaling TβRIII, thereby permitting the TGF-β MOD (and thus the TMP) to effectively deliver active TGF-β agonist to target cells. While the TMP constructs discussed herein permit epitope-specific presentation of a reversibly masked TGF-β to a target T cell, they also provide sites for the presentation of one or more additional MODs. The ability of the TMP construct to include one or more additional MODs thus permits the combined presentation of TGF-β and the additional MOD(s) to direct a target T cell's response in a substantially epitope-specific/selective manner in order to provide modulation of the target T cell. The TMP thereby permits delivery of one or more masked TGF-β MODs in an epitope-selective (e.g., dependent/specific) manner that permits (i) formation of an active immune synapse with a target T cell, such as a CD4+ cell selective for the epitope, and (ii) modulation (e.g., control/regulation) of the target T cell's response to the epitope. Once engaged with the TCR of a T cell, the effect of a masked TGF-β MOD-containing TMP on the T cell will depend on whether any additional MODs are present as part of the TMP and, if so, which additional MOD(s) is/are present.

Further, although the TMPs of this disclosure may comprise both one or more masked TGF-β MODs and one or more additional MODs such as a wt or variant IL-2, PD-L1 and/or a 4-1BBL MOD (as discussed above), if desired, the TMPs of this disclosure may comprise only one or more masked TGF-β MODs. That is, the one or more additional MODs such as the wt or variant IL-2, PD-L1 and/or a 4-1BBL MOD need not be included in a TMP of this disclosure. The masked TGF-β MOD-containing TMPs of the present disclosure can function as a means of producing TGF-β-driven T cell responses. For example TGF-β by itself can inhibit the development of effector cell functions of T cells, activate macrophages, and/or promote tissue the repair after local immune and inflammatory actions subside.

Although masked TGF-β MODs comprise a TGF-β polypeptide that is masked, the TGF-β polypeptide can still act as TβR agonist because the TGF-β polypeptide-mask complex is reversible and “breathes” between an open state where the TGF-beta polypeptide is available to cellular receptor, and a closed state where the mask engages the TGF-β polypeptide. Accordingly, the masking polypeptide functions to bind TGF-β polypeptide and prevent it from entering into tight complexes with, for example, ubiquitous non-signaling TβR3 molecules that can scavenge otherwise free TGF-β. Moreover, because the active forms of TGF-β are dimers that have higher affinity for TBR3, substitutions that limit dimerization (e.g., a C77Substitution of the cysteine at position 77 with a serine) can be incorporated into TGF-β sequences in order to avoid scavenging by that receptor.

One effect of the masking sequence is to reduce the effective affinity of TGF-β1. TGF-β2, and TGF-β3 polypeptides for TβRs. At the same time, the affinity of the masking polypeptide for the TGF-β polypeptide can be altered so that it dissociates more readily from the TGF-β polypeptide, making the TGF-5 polypeptide more available to cellular TDR proteins. That is, where the affinity of a masking polypeptide for a TGF-β polypeptide is reduced, the masked TGF-β MOD will spend more time in the open state. Although in the open state with the TGF-β polypeptide available for binding to cellular receptors, because the TβRII protein is generally the first peptide of the heteromeric TβR1/TβR2 signaling complex to interact with TGF-0, control of the affinity of the TGF-β polypeptide for TβRII effectively controls entry of TGF-β3 into active signaling complexes. The incorporation of substitution at, for example, one or more, two or more, or all three of Lys 25, Ile 92, and/or Lys 94 of TGF-β2 (or the corresponding positions of TGF-1, TGF-03) reduces affinity for TβRII polypeptides. The reduced affinity permits interactions between the target cell's TCR and the TMPs MHC polypeptides and epitope to effectively control binding and allows for target cell-specific interactions.

When a TβRII polypeptide is used as the masking polypeptide, the possibility of direct interactions with cellular TβR1 receptors and off-target signaling can be addressed by appropriate modifications of the masking sequence. Where it is desirable to block/limit signaling by the masked TGF-β polypeptide through TβR1 and/or modify (e.g., reduce) the affinity of a masking TβRII polypeptide for TGF-β, it is possible to incorporate N-terminal deletions and/or aa substitutions in the masking TβRII polypeptide. Modifications that can be made include deletions of N-terminal amino acids (e.g., N-terminal Δ14 or Δ25 deletions), and/or substitutions at one or more of L27, F30. D32. S49, 150. T51, S52, 153, E55. V77, D118, and/or E119. Some specific TβRII modifications resulting in a reduction in TORI association with TβRII and reduced affinity for TGF-β include any one or more of L27A, F30A, D32A, D32N, S49A, 150A, T51A, S52A, S52L, 153A, E55A, V77A, D118A, D118R, E119A, and/or E119Q.

The TGF-β polypeptide present in a TMP is in some cases a variant TGF-β polypeptide, including a variant TGF-β polypeptide that has a lower affinity for at least one class of TGF-β receptors, or is selective for at least one class of TGF-β receptors, compared to a wild-type TGF-β polypeptide.

While a TGF-β1 polypeptide, a TGF-β2 polypeptide, or a TGF-β3 polypeptide can be incorporated into a TMP as part of a masked TGF-β polypeptide, a variety of factors may influence the choice of the specific TGF-β polypeptide, and the specific sequence and aa substitutions that will be employed. Fore example, TGF-β1 and TGF-β3 polypeptides are subject to “clipping” of their amino acid sequences when expressed in a certain mammalian cell lines (e.g., CHO cells). In addition, dimerized TGF-β (e.g., TGF-β2) has a higher affinity for the TβR3 (beta glycan receptor) than for the TβR2 receptor, which could lead to off target binding and loss of biologically active masked protein to the large in vivo pool of non-signaling TβR3 molecules. To minimize high-affinity off target binding to TβR3, it may be desirable to substitute the residues leading to dimeric TGF-β molecules, which are joined by a disulfide bond. Accordingly, cysteine 77 (C77) may be substituted by an amino acid other than cysteine (e.g., a serine forming a C77S substitution).

Amino acid sequences of TGF-β polypeptides are known in the art. In some cases, the TGF-β polypeptide present in a masked TGF-β polypeptide is a TGF-β1 polypeptide. In some cases, the TGF-β polypeptide present in a masked TGF-β polypeptide is a TGF-β2 polypeptide. In some cases, the TGF-β polypeptide present in a masked TGF-β polypeptide is a TGF-β3 polypeptide.

A suitable TGF-β polypeptide can have a length from about 70 aas to about 125 aas; for example, a suitable TGF-β polypeptide can have a length from about 70 aas to about 80 aas from about 80 aas to about 90 aas; from about 90 aas to about 100 aas; from about 100 aas to about 105 aas, from about 105 aas to about 110 aas, from about 110 aas to about 112 aas, from about 113 aas to about 120 aas, or from about 120 aas to about 125 aas. A suitable TGF-β polypeptide can comprise an amino acid sequence having at least 60%, at least 70%, at least 80%, at least 80%, at least 85%, at least 90%, at least 95%, at least 98%, at least 99%, or 100%, aa sequence identity to at least 80, at least 90, at least 100, or at least 110 contiguous aas of the mature form of a human TGF-β polypeptide, a human TGF-β2 polypeptide, or a human TGF-β33 polypeptide.

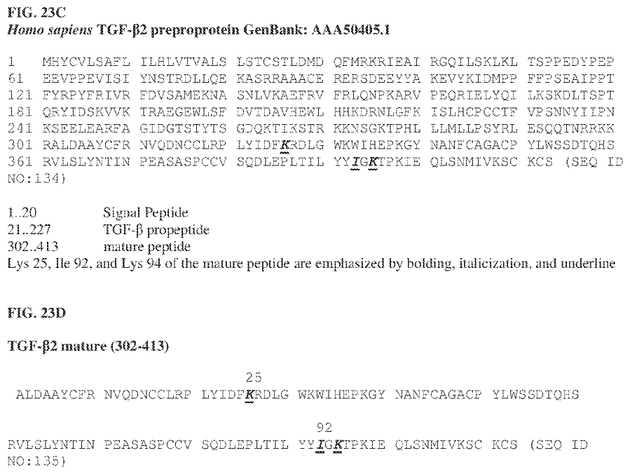

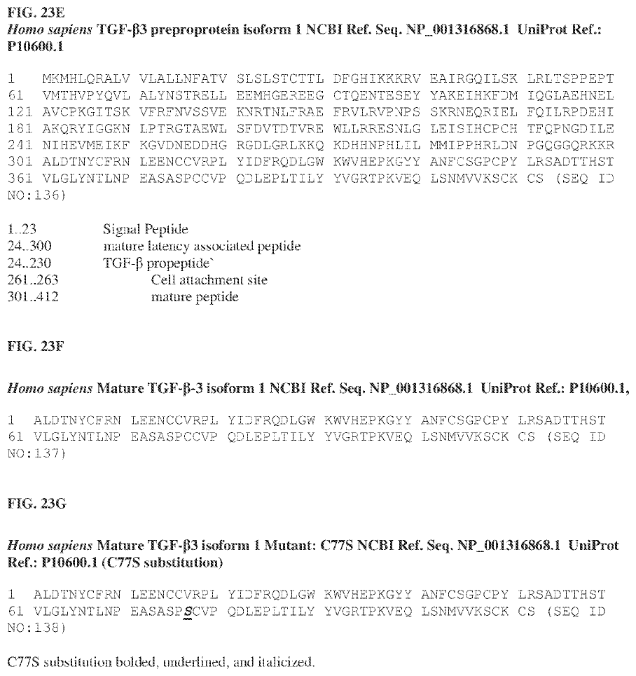

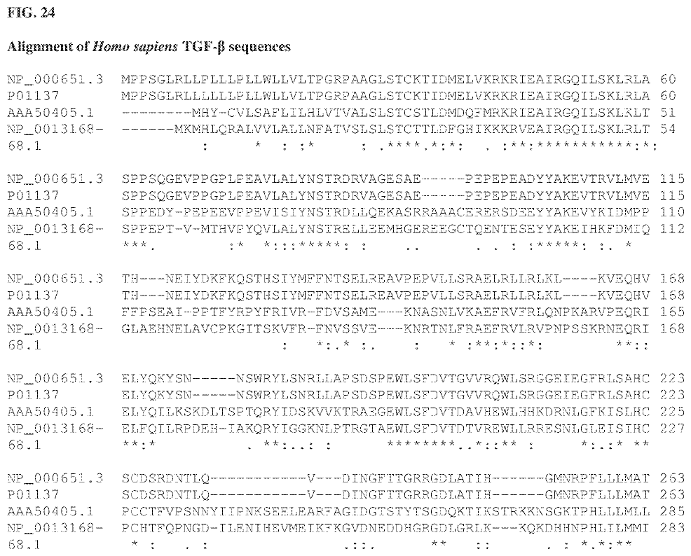

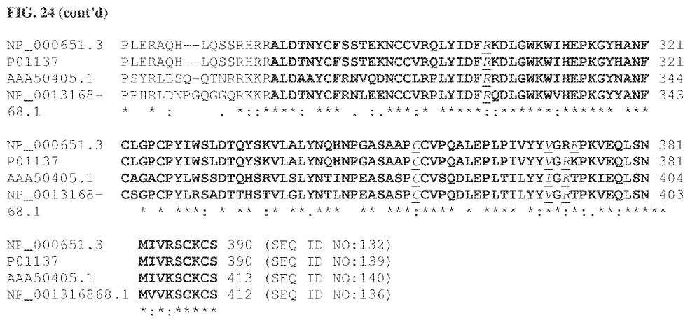

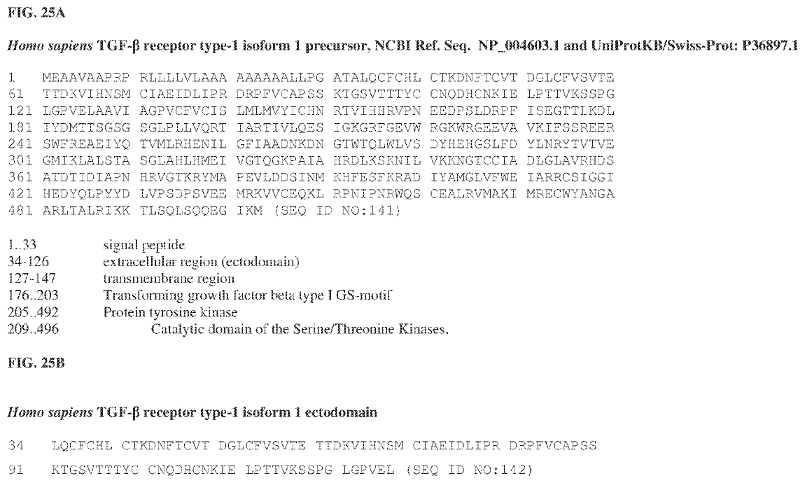

FIG. 23A-23G provide amino acid sequences of TGF-β polypeptides, including: TGF-β1 preproprotein (FIG. 23A), TGF-β2 preprotein (FIG. 23C), and TGF-β3 preprotein (FIG. 23E); TGF-β1 mature form (FIG. 23B), TGFβ2 mature form (FIG. 23D), and TGFβ3 mature form (FIG. 23F). The mature form of TGF-β3 with a C77S substitution is provided in FIG. 23G. An alignment of amino acid sequences of Homo sapiens TGF-β1, TGF-β2, and TGF-β3 polypeptides is provided in FIG. 24.

(i) TGF-β1 Polypeptides

A suitable TGF-01 polypeptide can comprise an amino acid sequence having at least 60%, at least 70%, at least 80%, at least 90%, at least 95%, at least 98%, at least 99%, or 100%, aa sequence identity to at least 70, at least 80, at least 90, at least 100, at least 110, or 112 aas of the following TGF-β1 amino acid sequence: AL DTNYCFSSTE KNCCVRQLYI DFRKDLGWKW IHEPKGYHAN FCLGPCPYIW SLDTQYSKVL ALYNQHNPGA SAAPCCVPQA LEPLPIVYYVGRKPKVEQLS NMIVRSCKCS (SEQ ID NO:133; 112 aas in length); where the TGF-β1 polypeptide has a length of about 112 aas. A TGF-β1 preproprotein is provided in FIG. 23A. Amino acids R25, C77, V92 and R94 are bolded and italicized in FIG. 23A.

In some cases, a suitable TGF-β1 polypeptide comprises a C77S substitution. Thus, in some cases, a suitable TGF-β1 polypeptide comprises an amino acid sequence having at least 60%, at least 70%, at least 80%, at least 90%, at least 95%, at least 98%, at least 99%, or 100%, aa sequence identity to at least 70, at least 80, at least 90, at least 100, at least 110, or 112 aas of the following TGF-01 amino acid sequence: AL DTNYCFSSTE KNCCVRQLYI DFRKDLGWKW IHEPKGYHAN FCLGPCPYIW SLDTQYSKVL ALYNQHNPGA SAAPSCVPQA LEPLPIVYYV GRKPKVEQLS NMIVRSCKCS (SEQ ID NO:229), where amino acid 77 is Ser. Positions 25, 77, 92 and 94 are bolded and italicized.

(ii) TGF-β2 Polypeptides

A suitable TGF-β2 polypeptide can comprise an amino acid sequence having at least 60%, at least 70%, at least 80%, at least 90%, at least 95%, at least 98%, at least 99%, or 100%, aa sequence identity to at least 70, at least 80, at least 90, at least 100, at least 110, or 112 aas of the following TGF-β2 amino acid sequence: ALDAAYCFR NVQDNCCLRP LYIDFKRDLG WKWIHEPKGY NANFCAGACP YLWSSDTQHS RVLSLYNTIN PEASASPCCV SQDLEPLTIL YYIGETPKIE QLSNMIVKSC KCS (SEQ ID NO:135), where the TGF-β2 polypeptide has a length of about 112 aas. A TGF-β2 preproprotein is provided in FIG. 23C. Residues Lys 25, Cys 77, Ile 92, and Lys 94 are bolded and italicized in FIG. 23C.

In some cases, a suitable TGF-β2 polypeptide comprises a C77S substitution. Thus, in some cases, a suitable TGF-β2 polypeptide comprises an amino acid sequence having at least 60%, at least 70%, at least 80%, at least 90%, at least 95%, at least 98%, at least 99%, or 100%, aa sequence identity to at least 70, at least 80, at least 90, at least 100, at least 110, or 112 aas of the following TGF-β2 amino acid sequence: ALDAAYCFR NVQDNCCLRP LYIDFKRDLG WKWIHEPKGY NANFCAGACP YLWSSDTQIIS RVLSLYNTIN PEASASPSCV SQDLEPLTIL YYIGKTPKIE QLSNMIVKSC KCS (SEQ ID NO:230), where amino acid 77 is substituted by a Ser. Ser77 is bolded and italicized.

(iii) TGF-β3 Polypeptides

A suitable TGF-β3 polypeptide can comprise an amino acid sequence having at least 60%, at least 70%, at least 80%, at least 90%, at least 95%, at least 98%, at least 99%, or 100%, aa sequence identity to at least 70, at least 80, at least 90, at least 100, at least 110, or 112 aas of the following TGF-β3 amino acid sequence: ALDTNYCFRN LEENCCVRPL YIDFRQDLGW KWVHEPKGYY ANFCSGPCPY LRSADTTHST VLGLYNTLNP EASASPCCVP QDLEPLTILY YVGRTPKVEQ LSNMVVKSCK CS (SEQ ID NO:137), where the TGF-β3 polypeptide has a length of about 112 aas. A TGF-β3 isoform 1 preproprotein is provided in FIG. 23E. Positions 25, 92 and 94 are bolded and italicized in FIG. 23E.

In some cases, a suitable TGF-β3 polypeptide comprises a C77S substitution. In some cases, a suitable TGF-β3 polypeptide comprises an amino acid sequence having at least 60%, at least 70%, at least 80%, at least 90%, at least 95%, at least 98%, at least 99%, or 100%, aa sequence identity to at least 70, at least 80, at least 90, at least 100, at least 110, or 112 aas of the following TGF-β3 amino acid sequence: ALDTNYCFRN LEENCCVRPL YIDFRQDLGW KWVHEPKGYY ANFCSGPCPY LRSADTTHST VLGLYNTLNP EASASPSCVP QDLEPLTILY YVGRTPKVEQ LSNMVVKSCK CS (SEQ ID NO:138), where amino acid 77 is Ser. Positions 25, 92 and 94 are bolded and italicized.

(iv) Additional TGF-β Polypeptide Sequence Variations

In addition to sequence variations that alter TGF-β dimerization (e.g., cysteine 77 substitutions such as C77S), TGF-β1. TGF-β2, and TGF-β3 polypeptides having sequence variations that affect affinity and other properties may be incorporated into a TGF-β polypeptide. When a variant TGF-β with reduced affinity for the masking polypeptide (e.g., a TβR polypeptide such as a TβRII polypeptide) is present in the TGF-β polypeptide, those components dissociate more readily, making the TGF-β polypeptide more available to cellular TβR proteins. Because the TβRII protein is generally the first peptide of the heteromeric TβR signaling complex to interact with TGF-β, interactions with TβRII effectively controls entry of TGF-β into active signaling complexes. Accordingly, variants controlling the affinity of TGF-β for TβRII may effectively control entry of masked TGF-β MODs into active signaling complexes.

The present disclosure provides a TMP comprising a variant masking TβR (e.g., TβRII) polypeptide sequence and/or a variant TGF-β polypeptide having altered (e.g., reduced) affinity for each other (relative to an otherwise identical TGF-β polypeptide without the sequence variation(s)). Affinity between a TGF-β polypeptide and a TβR (e.g., TβRII) polypeptide may be determined using (BLI) as described above for MODs and their co-MODs.

(a) Additional TGF-β2 Sequence Variants

The present disclosure provides a TMP comprising either a wild-type (wt) or a variant TGF-β2 polypeptide; where the variant polypeptide has a reduced affinity for the masking TβR present in the TMP (relative to an otherwise identical wt TGF-β polypeptide without the sequence variations).

In some cases, a TMP comprises a variant TGF-β2 polypeptide having greater than 85% (e.g., greater than 90%, 95%, 98% or 99%) sequence identity to at least 100 contiguous aa of the amino acid sequence depicted in FIG. 23D and comprising a substitution reducing the affinity of the variant TGF-β2 polypeptide for the TORII receptor sequence.

In some cases, a TMP comprises a variant TGF-β (e.g., TGF-β2) polypeptide comprising a substitution of one or more, two or more, or all three of Lys 25, Ile 92, and/or Lys 94 (see FIG. 23D for the location of the residues, and FIG. 24 for the corresponding residues in TGF-01 and TGF-β3). Those aa residues have been shown to affect the affinity of TGF-β2 for TORII polypeptides (see Crescenzo et al., J. Mol. Biol. 355: 47-62 (2006)). In one instance, a TMP comprises a TGF-β2 polypeptide having an an other than Lys or Arg at position 25 (FIG. 23D). A TMP may comprise a TGF-β2 polypeptide having an aa other than Ile or Val at position 92 of the amino acid sequence depicted in FIG. 23D (or an aa other than Ile, Val, or Leu at position 92). A TMP may comprise a TGF-β2 polypeptide having an aa other than Lys or Arg at position 94 of the amino acid sequence depicted in FIG. 23D. A TMP may comprise a TGF-β2 polypeptide comprising a substitution at one or more, two or more or all three of Lys 25. Ile 92, and/or Lys 94 of the amino acid sequence depicted in FIG. 23D.

(b) Additional TGF-β1 and TGF-β3 Sequence Variants

In some cases, a TMP comprises a variant TGF-β1 or TGF-β3 polypeptide comprising a substitution at one or more, two or more or all three aa positions corresponding to Lys 25, Ile 92, and/or Lys 94 in TGF-02. In TGF-β1 or TGF-β3, the as that corresponds to: Lys 25 is an Arg 25. Ile 92 is Val 92, and Lys 94 is Arg 94, each of which is a conservative substitution. See e.g., FIG. 23B for TGF-β1 (mature form) and FIG. 23F for TGF-β3 (mature form).

As an example, in some cases, a TMP comprises a TGF-1 or β3 polypeptide having an as other than Arg or Lys at position 25. As another example, in some cases, a TMP comprises a TGF-β1 or β3 polypeptide having an aa other than Val or lie at position 92 (or an aa other than lie. Val, or Leu at position 92). As another example, in some cases, a TMP comprises a TGF-β2 polypeptide having an as other than Arg or Lys. As another example, in some cases, a TMP comprises a TGF-β1 or β3 polypeptide comprising a substitution at one or more, two or more or all three of Arg 25, Val 92, and/or Arg 94. As another example, in some cases, a TMP comprises a TGF-β1 or β3 polypeptide comprising a substitution at one or more, two or more or all three of Arg 25, Val 92, and/or Arg 94.

### Masking Polypeptides

As noted above, a TMP of the present disclosure comprises a TGF-β polypeptide and a masking polypeptide. Polypeptides or polypeptide complexes that bind to and mask the TGF-β polypeptide (the “masking polypeptide”) can take a variety of forms, including. e.g., TGF-β RI (“TβRI”), TGF-βII (“TβRII”), TGF-βRIII (“TβRIII”), and anti-TGF-β antibodies.

### TGF-β Receptor Polypeptides

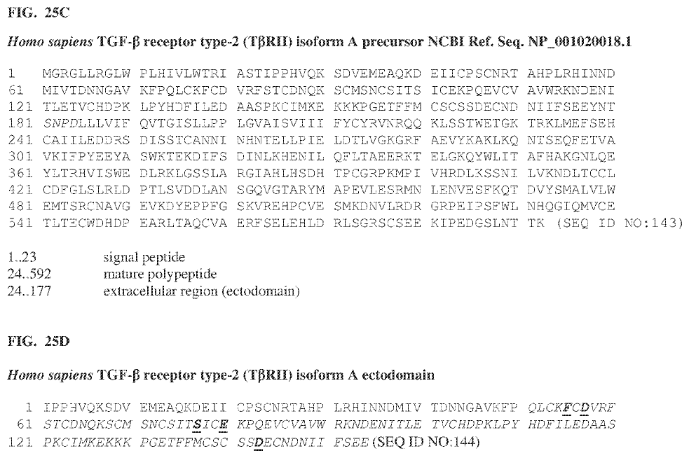

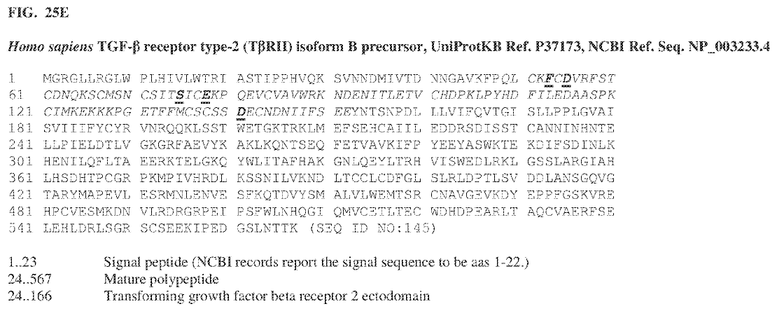

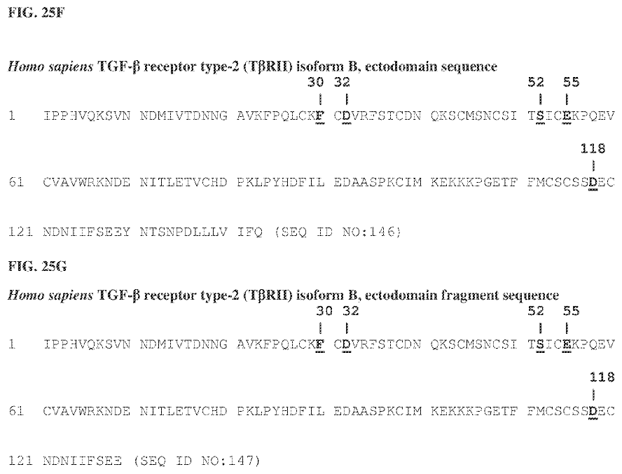

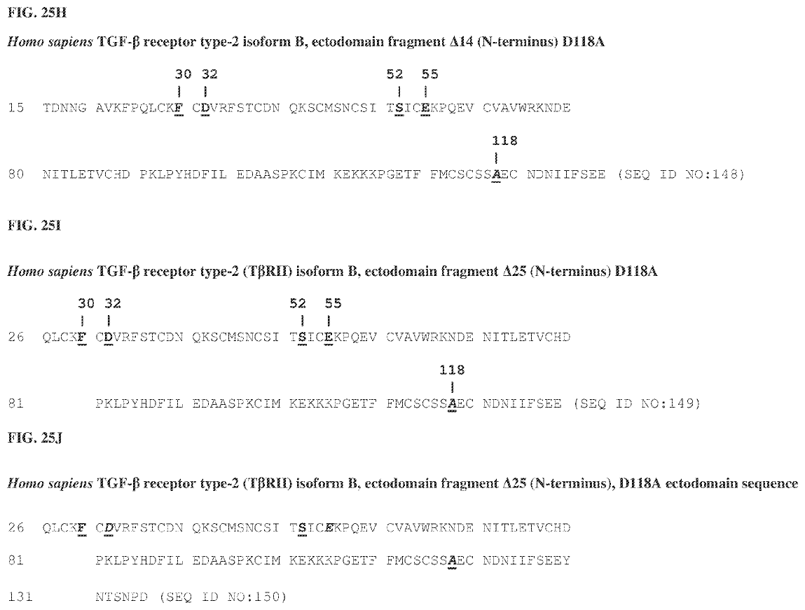

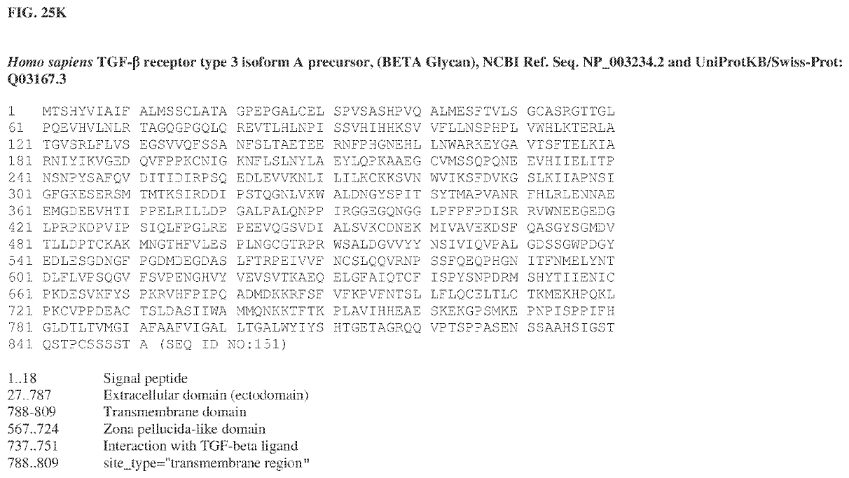

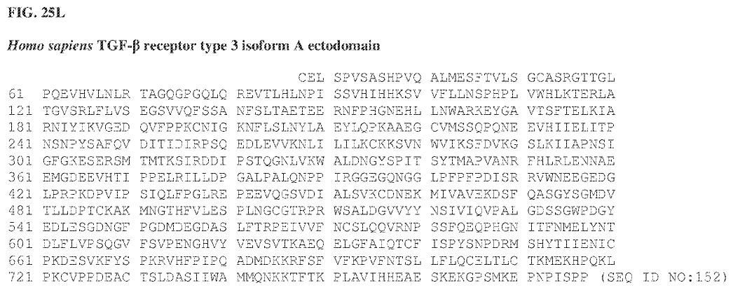

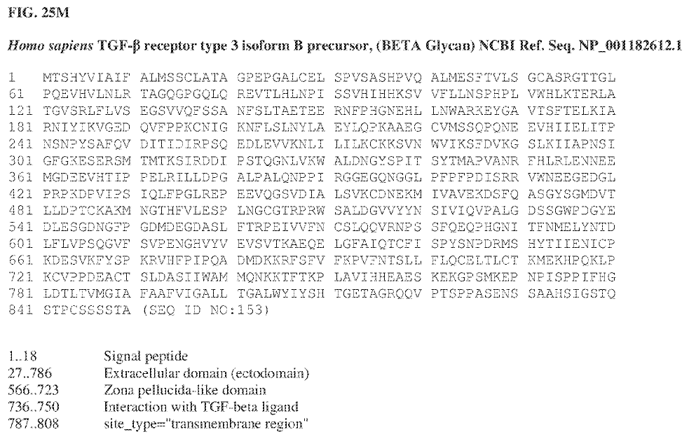

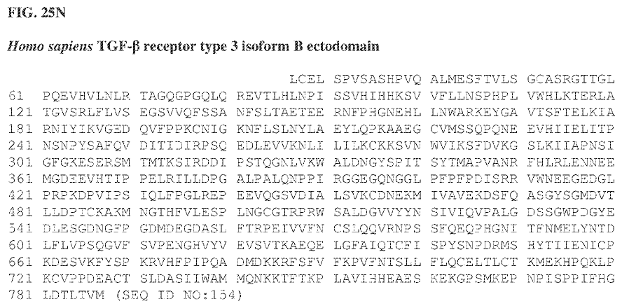

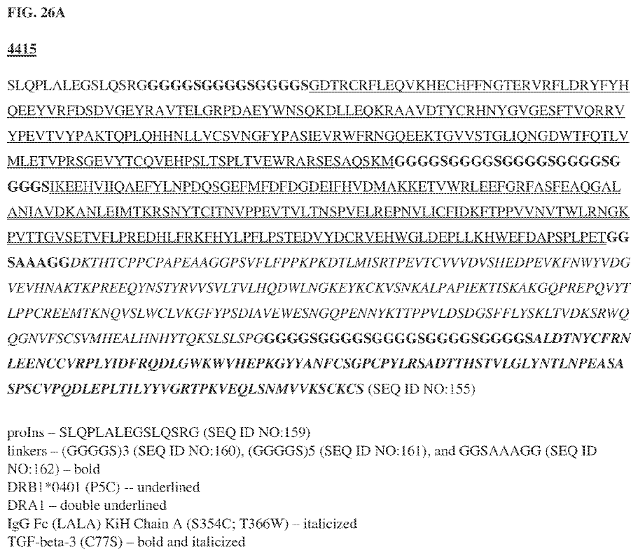

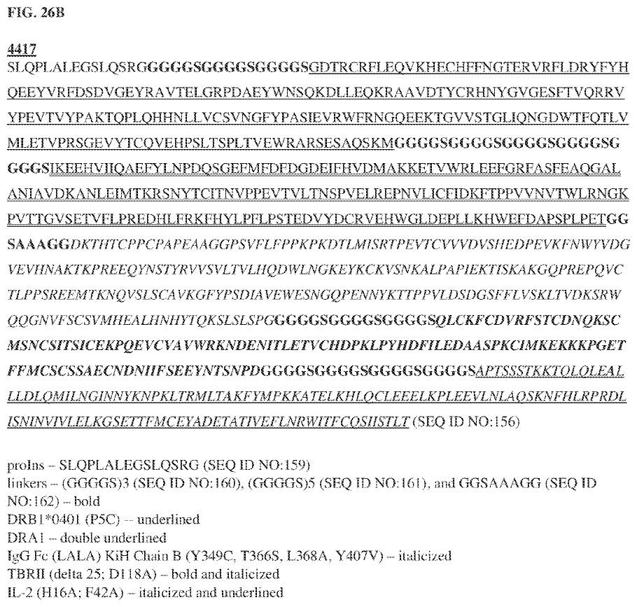

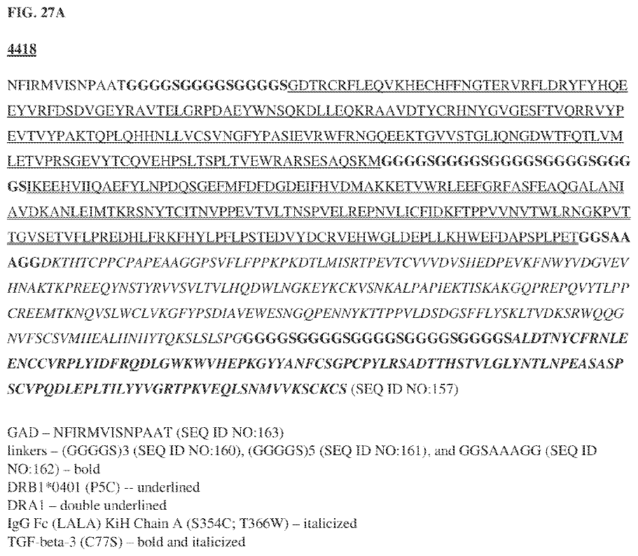

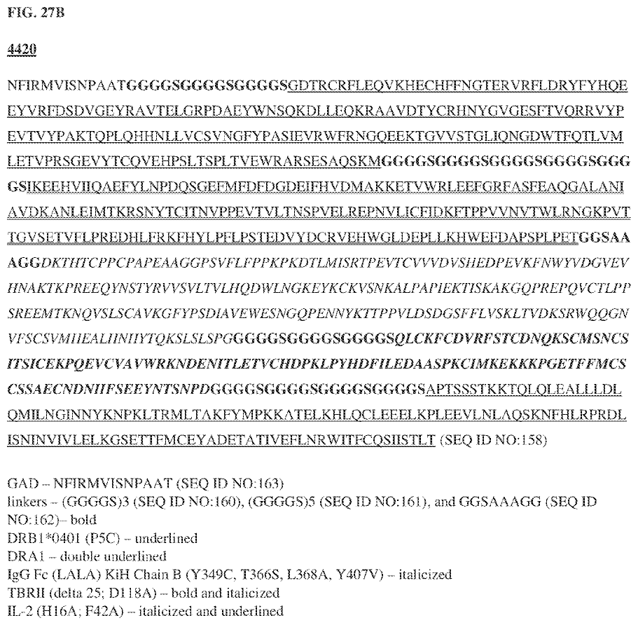

The masking of TGF-β in masked TGF-β polypeptides may be accomplished by utilizing a TGF-β receptor fragment (e.g., the ectodomain sequences of TβRI, TβRII or TβRIII) that comprises polypeptide sequences sufficient to bind a TGF-β polypeptide (e.g., TGF-β1. TGF-β2 or TGF-β3). In an embodiment, the masking sequence comprises all or part of the TβRI, TβRII, or TβRIII ectodomain. Examples of amino acid sequences of TGF-β receptor polypeptides are provided in FIG. 25A-25N. An amino acid sequence of a TβRI ectodomain polypeptide is provided in FIG. 25B. Amino acid sequences of TβRII ectodomain polypeptides are provided in FIG. 25D and FIG. 25F-J.

### (1) TGF-β Receptor I (TβRI)

A masking polypeptide may be derived from a TORI (e.g., isoform 1; SEQ ID NO:141) and may comprise all or part of the TORI ectodomain (aas 34-126). A suitable TβRI polypeptide for masking TGF-β may comprise an amino acid sequence having at least 60%, at least 70%, at least 80%, at least 90%, at least 95%, at least 98%, at least 99%, or 100%, aa sequence identity to at least 70, at least 80, at least 90, at least 100, or 103 aas of the following TβRI ectodomain aa sequence: LQCFCHL CTKDNFTCVT DGLCFVSVTE TTDKVIHNSM CIAEIDLIPR DRPFVCAPSS KTGSVTTTYC CNQDHCNKIE LPTTVKSSPG LGPVEL (SEQ ID NO:142).

### (2) TGF-β Receptor II (TβRII)

A masking polypeptide may be derived from a TβRII (e.g., isoform A), and may comprises all or part of the TβRII ectodomain sequence (aas 24 to 177). A suitable TβRII isoform A polypeptide for masking TGF-β may comprise an amino acid sequence having at least 60%, at least 70%, at least 80%, at least 90%, at least 95%, at least 98%, at least 99%, or 100%, an sequence identity to at least 70, at least 80, at least 90, at least 100, at least 110, at least 120, at least 130, at least 140, at least 150 or at least 154 aas of the following Tβ3RII isoform A octodomain aa sequence: IPPHVQK SDVEMEAQKD EIICPSCNRT AHPLRHINND MIVTDNNGAV KFPQLCKFCD VRFSTCDNQK SCMSNCSITS ICEKPQEVCV AVWRKNDENI TLETVCHDPK LPYHDFILED AASPKCIMKE KKKPGETFFM CSCSSDECND NUFSEE (SEQ ID NO:144). The location of the aspartic acid residue corresponding to D118 in the B isoform is bolded and italicized.

A masking polypeptide may be derived from TORII isoform B SEQ ID NO:145) and may comprises all or part of the TβRII ectodomain sequence (aas 24 to 166). A suitable TβRII isoform B polypeptide for masking TGF-β may comprise an amino acid sequence having at least 60%, at least 70%, at least 80%, at least 90%, at least 95%, at least 98%, at least 99%, or 100%, an sequence identity to at least 70, at least 80, at least 90, at least 100, at least 110, at least 120, at least 130, at least 140, or 143 aas of the TβRII isoform B ectodomain aa sequence: IPPHVQKSVN NDMIVTDNNG AVKFPQLCKF CDVRFSTCDN QKSCMSNCSI TSICEKPQEV CVAVWRKNDE NITLETVCHD PKLPYHDFIL EDAASPKCIM KEKKKPGETF FMCSCSSDEC NDNIIFSEEY NTSNPDLLLV IFQ (SEQ ID NO:146). As discussed below, any one or more of F30, D32, 552, E55, or D118 (italicized and bolded) may be substituted by an amino acid other than the naturally occurring aa at those positions (e.g., alanine). A masking polypeptide can include a D118A or D118R substitution. A masking polypeptide can include a D118A or D118R substitution and one or more of a F30A, D32N, S52L and/or E55A substitution.

Although TβRII's ectodomain may be utilized as a masking polypeptide, that region of the protein has charged and hydrophobic patches that can lead to an unfavorable pI and can be toxic to cells expressing the polypeptide. In addition, combining a TβRII ectodomain with an active TGF-β polypeptide can result in a complex that could combine with cell surface TβRI and cause activation of that signaling receptor (e.g., signaling through the Smad pathway). Modifying TβRII ectodomain sequences used to mask TGF-β by removing or altering sequences involved in TORI association can avoid the unintentional stimulation of cells by the masked TGF-β except through their own cell surface heterodimeric TβRI/TβRII complex. Modifications of TβRII may also alter (e.g., reduce) the affinity of the TβRII for TGF-β (e.g., TGF-03), thereby permitting control of TGF-β unmasking and its availability as a signaling molecule. Polypeptides comprising TβR (e.g., TβRII) peptides with the highest affinity for TGF-β (e.g., TGF-β 3) most tightly mask the TGF-β polypeptide and require higher doses to achieve the same effect. In contrast, aa substitutions in TβRII that lower the affinity unmask the TGF-β polypeptide and are biologically effective at lower doses.

Accordingly, where it is desirable to block/limit signaling by a TGF-β polypeptide through TβRI and/or modify (e.g., reduce) the affinity of a masking TβRII polypeptide for TGF-β, a number of alterations to TβRII may be incorporated into the TβRII polypeptide sequence. Modifications that can be made include the above-mentioned deletions of N-terminal amino acids, such as 14 or 25 N-terminal amino acids (from 1 to 14 aas or from 1 to 25 aas; A14, A25 modifications), and/or substitutions at one or more of L27. F30, D32, 549, 150, T51, 552, 153, E55, V77, D118, and/or E119. Some specific TβRII modifications resulting in a reduction in TβRI association with TβRII and reduced affinity for TGF-β include any one or more of L27A, F30A, D32A, D32N, S49A, 150A. T51A, S52A. S52L, 153A, E55A, V77A. D118A, D118R, E119A, and/or E119Q based on the amino acid numbering of the amino acid sequence depicted in FIG. 25F. See e.g., J. Groppe et al. Mol Cell 29, 157-168, (2008) and De Crescenzo et al. JMB 355, 47-62 (2006) for the effects of those substitutions on TGF-β3-TORII and TβRI-TβRII complexes. Modifications of TβRII the including an N-terminal Δ25 deletion and/or substitutions at F24 (e.g., an F24A substitution) substantially or completely block signal through the canonical SMAD signaling pathway). In one aspect, the aspartic acid at position 118 (D118) of the mature TβRII B isoform (e.g., the amino acid sequence depicted in FIG. 25F) is replaced by an amino acid other than Asp or Glu, such as Ala giving rise to a “D118A” substitution or by an Arg giving rise to a D118R substitution. The Asp residues corresponding D118 are indicated with bold and underlining in FIG. 25F. N-terminal deletions of from 1 to 25 aa in length (e.g., a A25 deletions) and/or substitutions at F24 (e.g., an F24A substitution) may be combined with D118 substitutions (e.g., D118A or D118R). N-terminal deletions of from 1 to 25 aa in length (e.g., a A25 deletions) and/or substitutions at F24 (e.g., an F24A substitution) may also be combined with substitutions at any of L27. F30, D32, 549, 150, T51, 552, 153, E55, V77, D118, and/or E119 (e.g., D118A) substitutions, and particularly any of the specific substitutions recited for those locations in the amino acid sequence depicted in FIG. 25F described above to alter the affinity.

Deletions of the N-terminus of the TβRII polypeptides may also result in loss of TβRI interactions and prevent masked TGF-β polypeptides comprising a TβRII polypeptide from acting as a constitutively active complex that engages and activates TβRI signaling. A 14 aa deletion (A14) of the TβRII polypeptide substantively reduces the interaction of the protein with TβRI, and a A25 aa deletion of TβRII appears to completely abrogate the interaction with TβRI. N-terminal deletions also substantially alter the pI of the protein, with the A14 TβRII ectodomain mutant displaying a pI of about 4.5-5.0 (e.g., about 4.74). Accordingly, a masking polypeptide may comprise a TβRU ectodomain polypeptide with N-terminal deletions, such as from 14 to 25 aas (e.g., 14, 15, 16, 17, 18, 19, 20, 21, 22, 23, 24, or 25 aa). Modified ectodomain sequences, including those that limit interactions with TβRI, that may be utilized to mask TGF-β polypeptides are described in the paragraphs that follow.

In an embodiment, a masking polypeptide comprises sequence having at least 60%, at least 70%, at least 80%, at least 90%, at least 95%, at least 98%, at least 99%, or 100%, aa sequence identity to at least 70, at least 80, at least 90, at least 100, at least 110, at least 120, at least 130, at least 140, or 142 aas of the TβRII isoform B ectodomain sequence: IPPHVQKSVN NDMIVTDNNG AVKFPQLCKF CDVRFSTCDN QKSCMSNCSI TSICEKPQEV CVAVWRKNDE NITLETVCHD PKLPYHDFIL EDAASPKCIM KEKKKPGETF FMCSCSSDEC NDNIIFSEE (SEQ ID NO:147). Any one or more of F30, D32, 552, E55, or D118 (italicized and bolded) may be substituted by an amino acid other than the naturally occurring aa at those positions (e.g., alanine). In an embodiment, the masking polypeptide comprises the TβRII isoform B ectodomain bearing a D118A substitution. In an embodiment, the sequence masking TGF-β comprises the TβRII isoform B ectodomain sequence bearing a D118A substitution and one or more of a F30A, D32N, S52L and/or E55A substitution.

Combinations of N-terminal deletions of TβRII, such as from 14 to 25 aas (e.g., 14, 15, 16, 17, 18, 19, 20, 21, 22, 23, 24, or 25 aa), that block inadvertent cell signaling due to the masked TGF-β/TβRII complex interacting with TβRI may be combined with other TβRII octodomain substitutions, including those at any one or more of F30, D32, 552, E55, and/or D118. The combination of deletions and substitutions ensures the masked TGF-β MOD does not cause cell signaling except through the cell's membrane bound TβRI & TβRII receptors.

In an embodiment, the masking polypeptide comprises sequence having at least 60%, at least 70%, at least 80%, at least 90%, at least 95%, at least 98%, at least 99%, or 100%, aa sequence identity to at least 70, at least 80, at least 90, at least 100, at least 110, or 114 aas of the TβRI isoform B ectodomain sequence: TDNNG AVKFPQLCKF CDVRFSTCDN QKSCMSNCSI TSICEKPQEV CVAVWRKNDE NITLETVCHD PKLPYHDFIL EDAASPKCIM KEKKKPGETF FMCSCSSDEC NDNIIFSEE (SEQ ID NO:231), which has aas 1-14 (A4) deleted. Any one or more of F30, D32, S52. E55, or D118 (italicized and bolded) may be substituted by an amino acid other than the naturally occurring aa at those positions (e.g., alanine). In an embodiment, the masking polypeptide comprises the TβRII isoform B ectodomain having a D118A substitution. In an embodiment, the sequence masking TGF-β comprises the amino acid sequence TDNNG AVKFPQLCKF CDVRFSTCDN QKSCMSNCSI TSICEKPQEV CVAVWRKNDE NITLETVCHD PKLPYHDFIL EDAASPKCIM KEKKKPGETF FMCSCSSDEC NDNIIFSEE (SEQ ID NO:231), and bearing a D118A substitution and one or more of a F30A, D32N, S52L and/or E55A substitution.

In an embodiment, the masking polypeptide comprises an amino acid sequence having at least 60%, at least 70%, at least 80%, at least 90%, at least 95%, at least 98%, at least 99%, or 100%, aa sequence identity to at least 70, at least 80, at least 90, at least 100, or 104 aas of the sequence: QLCKF CDVRFSTCDN QKSCMSNCSI TSICEKPQEV CVAVWRKNDE NITLETVCHD PKLPYHDFIL EDAASPKCIM KEKKKPGETF FMCSCSSDEC NDNIIFSEE (SEQ ID NO:232), which has aas 1-25 (A25) deleted. Any one or more of F30, D32. S52, E55, or D118 (italicized and bolded) may be substituted by an amino acid other than the naturally occurring aa at those positions (e.g., alanine). In an embodiment, the masking polypeptide has a D118A substitution. In an embodiment, the masking polypeptide has a D118A substitution and one or more of a F30A, D32N, S52L and/or E55A substitution. In an embodiment, the masking polypeptide has D118A and F30A substitutions. In an embodiment, the masking polypeptide has D118A and D32N substitutions. In an embodiment, the masking polypeptide has D118A and S52L substitutions. In an embodiment, the masking polypeptide has D118A and E55A.

### (3) TGF-β Receptor III (TβRIII)

In an embodiment, the masking polypeptide may be derived from a TβRIII and may comprises all or part of a TβRIII ectodomain (aas 27-787 of the A isoform or 27-786 of the B isoform). In some cases, a suitable masking polypeptide comprises an amino acid sequence having at least 60%, at least 70%, at least 80%, at least 90%, at least 95%, at least 98%, at least 99%, or 100%, aa sequence identity to at least 70, at least 80, at least 90, at least 100, or 120 aas of a TβR III A isoform or B isoform ectodomain sequences.

(c) Antibodies

Although TGF-β receptor polypeptides (e.g., ectodomain sequences) can function to bind and mask TGF-β polypeptides in a TMP, other polypeptides that bind to a TGF-β polypeptide can also be employed as masking polypeptides. Among the suitable polypeptides that can be used to mask TGF-β are antibodies with affinity for TGF-α (e.g., antibodies specific for one or more of TGF-β1. TGF-β2, or TGF-β3) where antibodies include antibody fragments, such as nanobodies with affinity for TGF-β polypeptides, and single chain anti-TGF-β antibodies (e.g., any of which may be humanized). Some antibodies, including scFv antibodies, that bind and neutralize TGF-β have been described. See e.g., U.S. Pat. No. 9,090,685. Throughout the embodiments and/or aspects described in this disclosure, TβR (e.g., TβRII) polypeptides used to mask TGF-β polypeptides may be replaced with a masking antibody (e.g., a scFv or a nanobody) with affinity for the TGF-β polypeptide.

In some cases, an antibody (e.g., a single chain antibody) may be used as a masking polypeptide and may be limited to the isoform of the TGF-β polypeptide(s) to be masked. By way of example, a single chain antibody based on Metelimumab (CAT192) directed against TGF-β1 (e.g., Lord et al., mAbs 10(3): 444-452 (2018)) can be used to mask that TGF-β isoform when present in masked TGF-β MODs. In another embodiment, a single chain antibody specific for TGF-β2 is used to mask that TGF-β isoform. In another embodiment, a single chain antibody specific for TGF-β3 is used to mask that TGF-β isoform. Single chain antibodies can also be specific for a combination of TGF-β isoforms (e.g., ectodomains selected from the group consisting of: TGF-β1 & TGF-β2; TGF-β1 & TGF-β3; and TGF-β2 & TGF-β3. The single chain antibodies may also be pan-specific for TGF-01. TGF-β2, and TGF-β3 ectodomain sequences appearing in masked TGF-β MODs See e.g., WO 2014/164709. Antibodies and single chain antibodies that have the desired specificity and affinity for TGF-β isoforms can be prepared by a variety of methods, including screening hybridomas and/or modification (e.g., combinatorial modification) to the variable region sequence of antibodies that have affinity for a target TGF-β polypeptide sequence.

In an embodiment, a masking polypeptide comprises a single chain antibody to mask a TGF-β (e.g., a TGF-β3). In one such embodiment the single chain amino acid sequence is specific for the TGF-β3 comprising a C77S substitution.

### Linkers

As discussed above, a TMP of the present disclosure can include one or more linker peptides between components of the first and second polypeptides of the TMP, e.g., between a T1D peptide and an MHC polypeptide; between an MHC polypeptide and an Ig Fc polypeptide; between a first MHC polypeptide and a second MHC polypeptide; between an MOD and an MHC polypeptide; etc. As used herein, the phrase “an optional peptide linker between any two of the components of a TMP” refers to a peptide linker between any two adjacent polypeptides within the TMP. For example, as used herein, the phrase “an optional peptide linker between any two of the components of a TMP” refers to a peptide linker between one or more of: i) a peptide and an MHC class II polypeptide; ii) a first MHC class II polypeptide and a second MHC class II polypeptide; iii) an MHC class II polypeptide and an Ig Fc polypeptide; iv) an MHC class II polypeptide and a masking polypeptide; v) an MHC class II polypeptide and a TGF-β polypeptide; vi) an Ig Fc polypeptide and a MOD; vii) an Ig Fc polypeptide and a TGF-β polypeptide; viii) a first MOD and a second MOD; ix) a masking polypeptide and a TGF-β polypeptide; and x) a TGF-β polypeptide and a MOD.

As also noted above, in some cases, a TMP can include a Cys-containing peptide linker between the T1D peptide and an MHC class II polypeptide. e.g., between the T1D peptide and an MHC class II β chain polypeptide. Generally, a Cys-containing peptide linker will be used in either the first or second polypeptide of a TMP in order to intentionally facilitate formation of a disulfide bond between the linker and a desired site on the other polypeptide. Where a Cys-containing linker is inserted in one polypeptide of a TMP, the remaining linkers in the TMP will not include a Cys in order to prevent formation of a disulfide bond at an unwanted site in the TMP, with the exception that a Cys-containing linker could be used in each of the first and second polypeptides when it is desired to link the first and second polypeptides through a disulfide bond formed between the linkers. A TMP thus can include: a) a Cys-containing peptide linker between the T1D peptide and an MHC class II polypeptide, e.g., between the T1D peptide and an MHC class II β chain polypeptide; and b) at least one additional peptide linker, where the at least one additional peptide linker does not include a Cys.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. A suitable linker can be from 25 to 35 amino acids in length. A suitable linker can be 25, 26, 27, 28, 29, 30, 31, 32, 33, 34, or 35 amino acids in length. A suitable linker can be from 35 to 45 amino acids in length. A suitable linker can be 35, 36, 37, 38, 39, 40, 41, 42, 43, 44, or 45 amino acids in length. A suitable linker can be from 45 to 50 amino acids in length. A suitable linker can be 45, 46, 47, 48, 49, or 50 amino acids in length.

Linkers Containing a Cys

A peptide linker comprising a Cys can comprise an amino acid sequence selected from (CGGGS)(GGGGS)ₙ (SEQ ID NO:178), (GCGGS)(GGGGS)ₙ (SEQ ID NO:179), (GGCGS)(GGGGS)ₙ (SEQ ID NO:180), (GGGCS)(GGGGS)ₙ (SEQ ID NO:181), and (GGGGC)(GGGGS)ₙ (SEQ ID NO:182), where n is an integer from 1 to 10 (e.g., where n is 1, 2, 3, 4, 5, 6, 7, 8, 9, or 10). In some cases, a TMP of the present disclosure comprises a heterodimer comprising a first and a second polypeptide, where the first polypeptide comprises a T1D peptide and an MHC class II polypeptide (e.g., an MHC class II β chain polypeptide), and comprises a linker between the T1D peptide and the MHC class II polypeptide (e.g., the MHC class II β chain polypeptide), where the linker comprises an amino acid sequence selected from (CGGGS)(GGGGS)ₙ (SEQ ID NO:178), (GCGGS)(GGGGS)ₙ (SEQ ID NO:179), (GGCGS)(GGGGS)ₙ (SEQ ID NO:180). (GGGCS)(GGGGS)ₙ (SEQ ID NO:181), and (GGGGC)(GGGGS)ₙ (SEQ ID NO:182), where n is an integer from 1 to 10 (e.g., where n is 1, 2, 3, 4, 5, 6, 7, 8, 9, or 10).

Linkers Not Containing a Cys

Exemplary linkers include glycine polymers (G)ₙ, glycine-serine polymers (including, for example, (GS)ₙ, (GSGGS)ₙ (SEQ ID NO:233) and (GGGS)ₙ(SEQ ID NO:234), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:235), GGSGG (SEQ ID NO:236), GSGSG (SEQ ID NO:237), GSGGG (SEQ ID NO:238). GGGSG (SEQ ID NO:239). GSSSG (SEQ ID NO:240), and the like. Exemplary linkers can include, e.g., Gly(Ser₄)ₙ, (SEQ ID NO:241) where n is 1, 2, 3, 4, 5, 6, 7, 8, 9, or 10. In some cases, a linker comprises the amino acid sequence (GSSSS)ₙ (SEQ ID NO:242), where n is 4. In some cases, a linker comprises the amino acid sequence (GSSSS)ₙ (SEQ ID NO:243), where n is 5. Exemplary linkers can include, e.g., (GlyGlyGlyGlySer)ₙ (SEQ ID NO:244) (also referred to as “G4S” linkers), where n is 1, 2, 3, 4, 5, 6, 7, 8, 9, or 10. In some cases, a linker comprises the amino acid sequence (GGGGS)ₙ (SEQ ID NO:245), where n is 1. In some cases, a linker comprises the amino acid sequence (GGGGS)ₙ (SEQ ID NO:246), where n is 2. In some cases, a linker comprises the amino acid sequence (GGGGS)ₙ (SEQ ID NO:160), where n is 3. In some cases, a linker comprises the amino acid sequence (GGGGS)ₙ (SEQ ID NO:171), where n is 4. In some cases, a linker comprises the amino acid sequence (GGGGS)ₙ (SEQ ID NO:161), where n is 5. In some cases, a linker comprises the amino acid sequence (GGGGS)ₙ (SEQ ID NO:247), where n is 6. In some cases, a linker comprises the amino acid sequence (GGCGGS)ₙ (SEQ ID NO:248), where n is 7. In some cases, a linker comprises the amino acid sequence (GGGGS)ₙ (SEQ ID NO:249), where n is 8. In some cases, a linker comprises the amino acid sequence (GGGGS)ₙ (SEQ ID NO:250), where n is 9. In some cases, a linker comprises the amino acid sequence (GGGGS)ₙ (SEQ ID NO:259), where n is 10. In some cases, a linker comprises the amino acid sequence AAAGG (SEQ ID NO:184). In some cases, a linker comprises the amino acid sequence GGSAAAGG (SEQ ID NO:162). The AAAGG (SEQ ID NO:184) and GGSAAAGG (SEQ ID NO:162) linkers have been found to be useful, e.g., for linking an MHC class II alpha chain polypeptide (e.g., a DRA class II polypeptide) to an Ig Fc polypeptide.

### Peptides Presenting T1D-Associated Epitopes (“T1D Peptides”)

As used herein, a “T1D peptide” is a peptide that, when present in a TMP of this disclosure, presents a T1D-associated epitope capable of being bound by a TCR on the surface of a T cell. A T1D peptide can have a length of from about 4 amino acids to about 25 amino acids, e.g., the T1D peptide can have a length of from 4 amino acids (aa) to 10 aa, from 8 aa to 12 aa, from 10 au to 15 aa, from 12 aa to 20 aa, from 15 aa to 20 aa, from 15 aa to 25 aa, or from 20 aa to 25 aa. For example, a T1D peptide present in a TMP can have a length of 4 amino acids (aa), 5 aa, 6 aa, 7, aa, 8 as, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa. In some cases, a T1D peptide present in a TMP has a length of from 9 amino acids to 20 amino acids, e.g., 8 aa, 9 aa, 10 aa, 11 aa, 12 aa, 13 aa, 14 aa, 15 aa, 16 aa, 17 aa, 18 aa, 19 aa, or 20 aa.

Antigens associated with type 1 diabetes (T1D) include, e.g., preproinsulin, proinsulin, insulin, insulin B chain, insulin A chain, 65 kDa isoform of glutamic acid decarboxylase (GAD65), 67 kDa isoform of glutamic acid decarboxylase (GAD67), tyrosine phosphatase (IA-2), heat-shock protein HSP65, islet-specific glucose6-phosphatase catalytic subunit related protein (IGRP), islet antigen 2 (IA2), and zinc transporter (ZnT8). See, e.g., Mallone et al. (2011) Clin. Dev. Immunol. 2011:513210; and U.S. Patent Publication No. 2017/0045529. An antigen “associated with” a particular autoimmune disorder is an antigen that is a target of autoantibodies and/or autoreactive T cells present in individuals with that autoimmune disorder, where such autoantibodies and/or autoreactive T cells mediate a pathological state associated with the autoimmune disorder. A suitable T1D peptide for inclusion in a TMP of the present disclosure can be an epitope-presenting T1D peptide of from 4 amino acids to about 25 amino acids in length of any one of the aforementioned T1D-associated antigens.

As one non-limiting example, a T1D peptide is proinsulin 73-90 (GAGSLQPLALEGSLQKR; SEQ ID NO:251) As another non-limiting example, a T1D peptide is the following insulin (InsA (1-15) peptide; GIVDQCCTSICSLYQ (SEQ ID NO:252). As another non-limiting example, a T1D peptide is the following insulin (InsA(1-15; D4E) peptide: GIVEQCCTSICSLYQ (SEQ ID NO:253). As another non-limiting example, a T1D peptide is the following GAD65 (555-567) peptide; NFFRMVISNPAAT (SEQ ID NO:177). As another non-limiting example, a T1D peptide is the following GAD65 (555-567; F557I) peptide; NFIRMVISNPAAT (SEQ ID NO:163). As another non-limiting example, a T1D peptide is the following islet antigen 2 (IA2) peptide; SFYLKNVQTQETRTLTQFIIF (SEQ ID NO:254). As another non-limiting example, a T1D peptide is the following proinsulin peptide; SLQPLALEGSLQSRG (SEQ ID NO:159). As another non-limiting example, a T1D peptide is the following proinsulin peptide GSLQPLALEGSLQSRGIV (SEQ ID NO:255; proIns 75-92(K88S)).

In some cases, a suitable T1D peptide comprises from 4 to 25 contiguous amino acids of an amino acid sequence having at least 90%, at least 95%, at least 98%, at least 99%, or 100%, amino acid sequence identity to amino acids 25-110 of the following human preproinsulin amino acid sequence (wherein amino acids 1-24 (underlined) is a signal peptide): MALWMRLLPL LALLALWGPD PAAA FVNQHL CGSHLVEALY LVCGERGFFY TPKTRREAED LQVGQVELGG GPGAGSLQPL ALEGSLQKRG IVEQCCTSIC SLYQLENYCN (SEQ ID NO:256); where the T1D peptide has a length of 4 amino acids (aa), 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa. In some cases, the T1D peptide has the amino acid sequence: GAGSLQPLALEGSLQKRG (SEQ ID NO:176) (proIns 73-90). In some cases, the T1D peptide has the amino acid sequence: SLQPLALEGSLQKRG (SEQ ID NO:175) (proIns 76-90). In some cases, the T1D peptide has the amino acid sequence: SLQPLALECTSLQSRG (SEQ ID NO:159) (prolns 76-90: K88S). In some cases, the T1D peptide has the amino acid sequence: QPLALEGSLQKRG (SEQ ID NO:257). In some cases, the T1D peptide has the amino acid sequence: QPLALEGSLQSRG (SEQ ID NO:258).

### Nucleic Acids

The present disclosure provides a nucleic acid comprising a nucleotide sequence encoding a TMP of the present disclosure. In some cases, where a TMP comprises two different polypeptide chains, the present disclosure provides a single nucleic acid comprising nucleotide sequences encoding both the first polypeptide and the second polypeptide of the TMP. In other cases, where a TMP comprises two different polypeptide chains, the present disclosure provides: a) a first nucleic acid comprising a nucleotide sequence encoding the first polypeptide of a TMP; and b) a second nucleic acid comprising a nucleotide sequence encoding the second polypeptide of a TMP. In other cases, where a TMP is a single-chain TMP, the present disclosure provides a nucleic acid comprising a nucleotide sequence encoding the single-chain TMP. In some cases, the nucleic acid is a recombinant expression vector; thus, the present disclosure provides a recombinant expression vector comprising a nucleotide sequence encoding a TMP.

### Separate Nucleic Acids Encoding Individual Polypeptide Chains of a TMP

The present disclosure provides nucleic acids comprising nucleotide sequences encoding a TMP of the present disclosure. As noted above, in some cases, where a TMP comprises two different polypeptide chains, the individual polypeptide chains of the TMP are encoded in separate nucleic acids. In some cases, nucleotide sequences encoding the separate polypeptide chains of a TMP are operably linked to transcriptional control elements, e.g., promoters, such as promoters that are functional in a eukaryotic cell, where the promoter can be a constitutive promoter or an inducible promoter.

For example, the present disclosure provides a first nucleic acid and a second nucleic acid, where the first nucleic acid comprises a nucleotide sequence encoding the first polypeptide of a TMP, and where the second nucleic acid comprises a nucleotide sequence encoding the second polypeptide of the TMP. In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

### Single Nucleic Acid Encoding Two or More Polypeptides Present in a TMP

The present disclosure provides a nucleic acid comprising nucleotide sequences encoding the first polypeptide and the second polypeptide of a TMP. In some cases, the nucleotide sequences encoding the first polypeptide and the second polypeptide of a TMP includes a proteolytically cleavable linker interposed between the nucleotide sequence encoding the first polypeptide and the nucleotide sequence encoding the second polypeptide. In some cases, the nucleotide sequences encoding the first polypeptide and the second polypeptide of a TMP includes an internal ribosome entry site (IRES) interposed between the nucleotide sequence encoding the first polypeptide and the nucleotide sequence encoding the second polypeptide. In some cases, the nucleotide sequences encoding the first polypeptide and the second polypeptide of a TMP includes a ribosome skipping signal (or cis-acting hydrolase element, CHYSEL) interposed between the nucleotide sequence encoding the first polypeptide and the nucleotide sequence encoding the second polypeptide. Examples of nucleic acids are described below, where a proteolytically cleavable linker is provided between nucleotide sequences encoding the first polypeptide and the second polypeptide of a TMP; in any of these embodiments, an IRES or a ribosome skipping signal can be used in place of the nucleotide sequence encoding the proteolytically cleavable linker.

In some cases, a first nucleic acid (e.g., a recombinant expression vector, an mRNA, a viral RNA, etc.) comprises a nucleotide sequence encoding a first polypeptide chain of a TMP; and a second nucleic acid (e.g., a recombinant expression vector, an mRNA, a viral RNA, etc.) comprises a nucleotide sequence encoding a second polypeptide chain of a TMP. In some cases, the nucleotide sequence encoding the first polypeptide, and the second nucleotide sequence encoding the second polypeptide, are each operably linked to transcriptional control elements, e.g., promoters, such as promoters that are functional in a eukaryotic cell, where the promoter can be a constitutive promoter or an inducible promoter.

### Nucleic Acid Encoding a Single-Chain TMP

The present disclosure provides a nucleic acid comprising a nucleotide sequence encoding a single-chain TMP described herein. In some cases, the nucleotide sequence encoding the single-chain TMP is operably linked to one or more transcriptional control elements. In some cases, the transcriptional control element is a promoter that is functional in a eukaryotic cell.

### Recombinant Expression Vectors

The present disclosure provides recombinant expression vectors comprising nucleic acids of the present disclosure. In some cases, the recombinant expression vector is a non-viral vector. In some cases, the recombinant expression vector is a viral construct. e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson. PNAS 92:7700 7704, 1995; Sakamoto et al., H Gene Ther 5:1088 1097, 1999; WO 94/12649. WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239. Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus. Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available.

Depending on the host/vector system utilized, any of a number of suitable transcription and translation control elements, including constitutive and inducible promoters, transcription enhancer elements, transcription terminators. etc. may be used in the expression vector (see e.g., Bitter et al. (1987) Methods in Enzymology, 153:516-544).

In some cases, a nucleotide sequence encoding a TMP is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some cases, a nucleotide sequence encoding TMP is operably linked to multiple control elements that allow expression of the nucleotide sequence in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### Genetically Modified Host Cells

The present disclosure provides a genetically modified host cell, where the host cell is genetically modified with a nucleic acid(s) of the present disclosure. The present disclosure provides a genetically modified host cell, where the host cell is genetically modified with a recombinant expression vector of the present disclosure.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells. COS-7 cells (ATCC No. CRL1651). RAT1 cells, mouse L cells (ATCC No. CCL1.3), human embryonic kidney (HEK) cells (ATCC No. CRL1573). HLHepG2 cells, and the like.

Genetically modified host cells can be used to produce a TMP. An expression vector(s) comprising nucleotide sequence(s) encoding the polypeptide(s) is/are introduced into a host cell, generating a genetically modified host cell, which genetically modified host cell produces the polypeptide(s). For example, the genetically modified host cell is cultured in a suitable culture medium under conditions such that the TMP is synthesized by the genetically modified host cell. The TMP can then be obtained from the culture medium and/or a lysate of the genetically modified host cell. The TMP can be purified from the cell culture medium and/or the cell lysate.

### Methods of Producing a TMP

The present disclosure provides methods of producing a TMP of the present disclosure. The methods generally involve culturing, in a culture medium, a host cell (e.g., a genetically modified host cell of the present disclosure) that is genetically modified with a recombinant expression vector(s) comprising a nucleotide sequence(s) encoding the TMP; and isolating the TMP from the genetically modified host cell and/or the culture medium. As noted above, in some cases, the individual polypeptide chains of a TMP are encoded in separate recombinant expression vectors. In some cases, all polypeptide chains of a TMP are encoded in a single recombinant expression vector. Where a TMP is a single-chain TMP, a single recombinant expression vector encodes the single-chain TMP.

Isolation of the TMP from the expression host cell (e.g., from a lysate of the genetically modified host cell) and/or the culture medium in which the host cell is cultured, can be carried out using standard methods of protein purification.

For example, a lysate may be prepared of the expression host and the lysate purified using high performance liquid chromatography (HPLC), exclusion chromatography (e.g., size exclusion chromatography), gel electrophoresis, affinity chromatography, or other purification technique. Alternatively, where the TMP is secreted from the expression host cell into the culture medium, the TMP can be purified from the culture medium using HPLC, exclusion chromatography, gel electrophoresis, affinity chromatography, or other purification technique. In some cases, the TMP is purified, e.g., a composition is generated that comprises at least 80% by weight, at least about 85% by weight, at least about 95% by weight, or at least about 99.5% by weight, of the TMP in relation to contaminants related to the method of preparation of the product and its purification. The percentages can be based upon total protein.

In some cases, e.g., where the TIP comprises an affinity tag, the TMP can be purified using an immobilized binding partner of the affinity tag. For example, where a TMP comprises an Ig Fc polypeptide, the TMP can be isolated from genetically modified mammalian host cell and/or from culture medium in which the mammalian cells are cultured, where isolation of the TMP or APP can be carried out by affinity chromatography, e.g., on a Protein A column, a Protein G column, or the like. An example of a suitable mammalian cell is a CHO cell; e.g., an Expi-CHO-S™ cell (e.g., ThermoFisher Scientific, Catalog #A29127).

In some cases, the first and second polypeptides of a TMP will self-assemble into heterodimers by spontaneously forming disulfide bonds between the above-discussed Cys residues in the first and second polypeptides. As also noted above, when both heterodimers include Ig Fc polypeptides, disulfide bonds will spontaneously form between the respective Ig Fc polypeptides to covalently link the two heterodimers to one another.

### Compositions

The present disclosure provides compositions, including pharmaceutical compositions, comprising a TMP of the present disclosure. The present disclosure provides compositions, including pharmaceutical compositions, comprising a nucleic acid or a recombinant expression vector of the present disclosure.

Compositions Comprising a TMP

A composition of the present disclosure can comprise, in addition to a TMP, one or more pharmaceutically acceptable components/excipients such as: a salt. e.g., NaCl, MgCl₂, KCl, MgSO₄, etc.; a buffering agent. e.g., a Tris buffer. N-(2-Hydroxyethyl)piperazine-N′-(2-ethanesulfonic acid) (HEPES). 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent: a detergent, e.g., a non-ionic detergent such as Tween-20, etc.; a protease inhibitor; glycerol; and the like. Pharmaceutically acceptable excipients have been amply described in a variety of publications, including, for example, “Remington: The Science and Practice of Pharmacy”. 19^{th} Ed. (1995), or latest edition. Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott. Williams, & Wilkins; and Handbook of Pharmaceutical Excipients (2000) A. H. Kibbe et al., eds., 3^{rd} ed. Amer. Pharmaceutical Assoc.

A pharmaceutical composition can comprise: i) a TMP of the present disclosure; and ii) a pharmaceutically acceptable excipient. In some cases, a subject pharmaceutical composition will be suitable for administration to a subject, e.g., will be sterile. For example, in some cases, a subject pharmaceutical composition will be suitable for administration to a human subject, e.g., where the composition is sterile and is free of detectable pyrogens and/or other toxins, or the amount of detectable pyrogens and/or other toxins are below a permissible limit.

For example, compositions may include aqueous solution, powder form, granules, tablets, pills, suppositories, capsules, suspensions, sprays, and the like. The composition may be formulated according to the various routes of administration described below.

Where a TMP is administered as an injectable (e.g. subcutaneously, intraperitoneally, intramuscularly, intralymphatically, and/or intravenously) directly into a tissue, a formulation can be provided as a ready-to-use dosage form, or as non-aqueous form (e.g., a reconstitutable storage-stable powder) or aqueous form, such as liquid composed of pharmaceutically acceptable carriers and excipients. The protein-containing formulations may also be provided so as to enhance serum half-life of the subject protein following administration. For example, the protein may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

In some cases, a composition of the present disclosure comprises: a) a TMP; and b) saline (e.g., 0.9% NaCl). In some cases, the composition is sterile. In some cases, the composition is suitable for administration to a human subject, e.g., where the composition is sterile and is free of detectable pyrogens and/or other toxins. Thus, the present disclosure provides a composition comprising: a) a TMP of the present disclosure; and b) saline (e.g., 0.9% NaCl), where the composition is sterile and is free of detectable pyrogens and/or other toxins, or where the detectable pyrogens and/or other toxins are below a permissible limit.

The concentration of a TMP in a formulation can vary widely (e.g., from less than about 0.1%, usually at or at least about 2% to as much as 20% to 50% or more by weight) and will usually be selected primarily based on fluid volumes, viscosities, and patient-based factors in accordance with the particular mode of administration selected and the patient's needs.

The present disclosure provides a container comprising a composition of the present disclosure, e.g., a liquid composition. The container can be, e.g., a syringe, an ampoule, and the like. In some cases, the container is sterile. In some cases, both the container and the composition are sterile.

### Compositions Comprising a Nucleic Acid or a Recombinant Expression Vector

The present disclosure provides compositions, e.g., pharmaceutical compositions, comprising a nucleic acid or a recombinant expression vector of the present disclosure. A wide variety of pharmaceutically acceptable excipients is known in the art and need not be discussed in detail herein. Pharmaceutically acceptable excipients have been amply described in a variety of publications, including, for example A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott. Williams. & Wilkins; and Handbook of Pharmaceutical Excipients (2000) A. H. Kibbe et al., eds., 3^{rd} ed. Amer. Pharmaceutical Assoc.

A pharmaceutical formulation of the present disclosure can include a nucleic acid or recombinant expression vector in an amount of from about 0.001% to about 90% (w/w). In the description of formulations, below. “subject nucleic acid or recombinant expression vector” will be understood to include a nucleic acid or recombinant expression vector. For example, in some cases, a subject formulation comprises a nucleic acid or recombinant expression vector.

A subject nucleic acid or recombinant expression vector can be admixed, encapsulated, conjugated or otherwise associated with other compounds or mixtures of compounds; such compounds can include, e.g., liposomes or receptor-targeted molecules. A subject nucleic acid or recombinant expression vector can be combined in a formulation with one or more components that assist in uptake, distribution and/or absorption.

### Methods

A TMP of the present disclosure is useful for modulating an activity of a T cell. Thus, the present disclosure provides methods of modulating an activity of a T cell, the methods generally involving contacting a target T cell with a TMP of the present disclosure.

The present disclosure provides a method of selectively modulating the activity of an epitope-specific T cell, the method comprising contacting the T cell with a TMP, where contacting the T cell with a TMP selectively modulates the activity of the epitope-specific T cell. In some cases, the contacting occurs in vivo. In some cases, the contacting occurs in vitro.

The present disclosure thus also provides a method of selectively delivering a TGF-β polypeptide to an epitope-specific target T cell, the method comprising contacting the T cell with a TMP comprising a masked TGF-β MOD, where contacting the epitope-specific T cell with a TMP comprising a masked TGF-β MOD selectively provides TGF-β modulation to the epitope-specific T cell. In some cases, the contacting occurs in vivo. In some cases, the contacting occurs in vitro. As used herein, the phrases “selectively delivers” and “selectively provides” means that the majority of T cells for which the TMP provides TGF-β modulation comprise a TCR that specifically or preferentially binds the epitope of the TMP.

In some cases, a TMP reduces activity of an autoreactive T cell and/or an autoreactive B cell. In some cases, a TMP increases the number and/or activity of a regulator T cell (Treg), resulting in reduced activity of an autoreactive T cell and/or an autoreactive B cell.

In some cases, the T cell being contacted with a TMP is a regulatory T cell (Treg). Tregs are CD4⁺, FOXP3⁺, and CD25⁺. Tregs can suppress autoreactive T cells. In some cases, a method of the present disclosure activates Tregs, thereby reducing autoreactive T cell activity.

The present disclosure provides a method of increasing proliferation of Tregs, the method comprising contacting Tregs with a TMP, where the contacting increases proliferation of Tregs. The present disclosure provides a method of increasing the number of Tregs in an individual, the method comprising administering to the individual a TMP, where the administering results in an increase in the number of Tregs in the individual. For example, the number of Tregs can be increased by at least 5%, at least 10%, at least 15%, at least 20%, at least 25%, at least 30%, at least 40%, at least 50%, at least 60%, at least 70%, at least 80%, at least 90%, at least 2-fold, at least 2.5-fold, at least 5-fold, at least 10-fold, or more than 10-fold.

### Treatment Methods

The present disclosure provides treatment methods, the methods comprising administering to the individual an amount of a TMP of the present disclosure, or one or more nucleic acids or expression vectors encoding the TMP effective to selectively modulate the activity of an epitope-specific (e.g., T1D peptide-specific) T cell in an individual and to treat the individual. In some cases, a treatment method of the present disclosure comprises administering to an individual in need thereof one or more recombinant expression vectors comprising nucleotide sequences encoding a TMP. In some cases, a treatment method comprises administering to an individual in need thereof one or more mRNA molecules comprising nucleotide sequences encoding a TMP. In some cases, a treatment method of the present disclosure comprises administering to an individual in need thereof a TMP e. A TMP is useful for treating type 1 diabetes (T1D).

The present disclosure provides a method of selectively modulating the activity of an epitope-specific T cell in an individual, the method comprising administering to the individual an effective amount of a TMP, or one or more nucleic acids (e.g., expression vectors; mRNA; etc.) comprising nucleotide sequences encoding the TMP, where the TMP selectively modulates the activity of the epitope-specific T cell in the individual. Selectively modulating the activity of an epitope-specific T cell can treat a disease or disorder in the individual. Thus, the present disclosure provides a treatment method comprising administering to an individual in need thereof an effective amount of a TMP. A treatment method of the present disclosure, comprising administering an effective amount of a TMP, is suitable for treating T1D.

The present disclosure provides a method of treating T1D in an individual, the method comprising administering to the individual an effective amount of a TMP, or one or more nucleic acids comprising nucleotide sequences encoding the TMP, where the TMP comprises a T1D peptide (as described above). In some cases, an “effective amount” of a TMP is an amount that, when administered in one or more doses to an individual in need thereof, reduces the number of self-reactive (i.e., reactive with a T1D-associated antigen) CD4⁺ and/or CD8⁺ T cells by at least 10%, at least 15%, at least 20%, at least 25%, at least 30%, at least 40%, at least 50%, at least 60%, at least 70%, at least 80%, at least 90%, or at least 95%, compared to number of self-reactive T cells in the individual before administration of the TMP, or in the absence of administration with the TMP. In some cases, an “effective amount” of a TMP is an amount that, when administered in one or more doses to an individual in need thereof, reduces production of Th2 cytokines in the individual. In some cases, an “effective amount” of a TMP is an amount that, when administered in one or more doses to an individual in need thereof, ameliorates one or more symptoms associated with T1D in the individual. In some instances, the TMP reduces the number of CD4⁺ self-reactive T cells (i.e., the number of CD4⁺ T cells reactive with a T1D-associated antigen), which in turn leads to a reduction in CD8⁺ self-reactive T cells. In some instances, the TMP increases the number of CD4⁺ Tregs, which in turn reduces the number of CD4⁺ self-reactive T cells and/or CD8⁺ T self-reactive T cells.

As noted above, in some cases, in carrying out a subject treatment method, a TMP is administered to an individual in need thereof, as the polypeptide per se. In other instances, in carrying out a subject treatment method, one or more nucleic acids comprising nucleotide sequences encoding a TMP is/are administering to an individual in need thereof. Thus, in other instances, one or more nucleic acids of the present disclosure, e.g., one or more recombinant expression vectors of the present disclosure, is/are administered to an individual in need thereof.

### Dosage

A suitable dosage can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A TMP (as a single heterodimer; as a homodimer comprising two heterodimers linked together; as a heterodimer comprising two heterodimers linked together, as a single-chain TMP; as a homodimer comprising two single-chain TMPs linked together; or as a heterodimer comprising two single-chain TMPs linked together) may be administered in amounts between 1 ng/kg body weight and 20 mg/kg body weight per dose, e.g. between 0.1 mg/kg body weight to 10 mg/kg body weight, e.g. from 0.1 mg/kg body weight to 0.5 mg/kg body weight, from 0.5 mg/kg body weight to 1 mg/kg body weight, from 1.0 mg/kg body weight to 5 mg/kg body weight, from 5 mg/kg body weight to 10 mg/kg body weight, from 10 mg/kg body weight to 15 mg/kg body weight, and from 15 mg/kg body weight to 20 mg/kg body weight; however, doses below 0.1 mg/kg body weight or above 20 mg/kg are envisioned, especially considering the aforementioned factors. Amounts thus include from about 0.1 mg/kg body weight to about 0.5 mg/kg body weight, from about 0.5 mg/kg body weight to about 1 mg/kg body weight, from about 1.0 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight. If the regimen is a continuous infusion, it can also be in the range of 1 μg to 10 mg per kilogram of body weight per minute.

Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the T1D disease state, wherein a TMP is administered in maintenance doses, ranging from those recited above, i.e., 0.1 mg/kg body weight to about 0.5 mg/kg body weight, from about 0.5 mg/kg body weight to about 1 mg/kg body weight, from about 1.0 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight.

Those of skill will readily appreciate that dose levels can vary as a function of the specific TMP, the severity of the symptoms and the susceptibility of the subject to side effects. Preferred dosages for a given compound are readily determinable by those of skill in the art by a variety of means.

In some cases, multiple doses of a TMP, a nucleic acid, or a recombinant expression vector are administered. The frequency of administration of a TMP, a nucleic acid, or a recombinant expression vector can vary depending on any of a variety of factors, e.g., severity of the symptoms, patient response, etc. For example, in some cases, a TMP, a nucleic acid, or a recombinant expression vector is administered less frequently than once per month, e.g., once every two, three, four, six or twelve months, once per month, twice per month, three times per month, every other week (qow), one every three weeks, once every four weeks, once per week (qw), twice per week (biw), three times per week (tiw), four times per week, five times per week, six times per week, every other day (qod), daily (qd), twice a day (qid), or three times a day (tid).

The duration of administration of a TMP, a nucleic acid, or a recombinant expression vector, e.g., the period of time over which a TMP, a nucleic acid, or a recombinant expression vector is administered, can vary, depending on any of a variety of factors. e.g., patient response, etc. For example, 1′MP, a nucleic acid, or a recombinant expression vector can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more, including continued administration for the patient's life.

Where treatment is of a finite duration, following successful treatment, it may be desirable to have the patient undergo maintenance therapy to prevent the recurrence of the T1D disease state, wherein a TMP is administered in maintenance doses, ranging from those recited above, i.e., 0.1 mg/kg body weight to about 0.5 mg/kg body weight, from about 0.5 mg/kg body weight to about 1 mg/kg body weight, from about 1.0 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight.

### Routes of Administration

An active agent (a TMP, a nucleic acid, or a recombinant expression vector) is administered to an individual using any available method and route suitable for drug delivery, including in vivo and in vitro methods, as well as systemic and localized routes of administration. A TMP, a nucleic acid, or a recombinant expression vector can be administered to a host using any available conventional methods and routes suitable for delivery of conventional drugs, including systemic or localized routes. In general, routes of administration contemplated for use in a method of the present disclosure include, but are not necessarily limited to, enteral, parenteral, and inhalational mutes.

Conventional and pharmaceutically acceptable routes of administration include intramuscular, intratracheal, intralymphatic, subcutaneous, intradermal, topical application, intravenous, intraarterial, rectal, nasal, oral, and other enteral and parenteral routes of administration. Of these, intravenous, intramuscular and subcutaneous may be more commonly employed. Routes of administration may be combined, if desired, or adjusted depending upon the TMP and/or the desired effect. A TMP, or a nucleic acid or recombinant expression vector, can be administered in a single dose or in multiple doses.

In some cases, a TMP, a nucleic acid, or a recombinant expression vector is administered intravenously. In some cases, a TMP, a nucleic acid, or a recombinant expression vector is administered intramuscularly. In some cases, a TMP, a nucleic acid of the present disclosure, or a recombinant expression vector is administered intralymphatically. In some cases, a TMP, a nucleic acid of the present disclosure, or a recombinant expression vector is administered subcutaneously.

### Subjects Suitable for Treatment

Subjects suitable for treatment with a method of the present disclosure include individuals who have T1D, including individuals who have been diagnosed as having T1D, and individuals who have been treated for T1 D but who failed to respond to the treatment. Suitable subjects also may include individuals who have been diagnosed as being likely to develop T1D or who have symptoms indicating the imminent onset of T1D.

### Examples of Non-Limiting Aspects of the Disclosure

### Aspects Set A

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A T-cell modulatory polypeptide (TMP) comprising:
a) a peptide that displays a Type 1 Diabetes-associated epitope capable of being bound by a T-cell receptor (a “T1D peptide”);
b) a TGF-β polypeptide;
c) a masking polypeptide, optionally wherein the masking polypeptide comprises a TGF-β receptor polypeptide or an anti-TGF-β polypeptide;
d) an MHC class II α chain polypeptide;
e) an MHC class II β chain polypeptide; and
f) one or more immunomodulatory polypeptides (MOD).
wherein optionally the TMP comprises a scaffold polypeptide, and
wherein optionally the TMP comprises one or mom independently selected linker polypeptides.

Aspect 2. The TMP of aspect 1, wherein at least one of the one or more MODs is selected from the group consisting of an IL-2 polypeptide, PD-L1 polypeptide, a 4-1BBL polypeptide, and combinations thereof.

Aspect 3. The TMP of aspect 1 or 2, wherein at least one of the one or more MODs is a PD-L1 polypeptide, optionally wherein the PD-L1 polypeptide comprises a PD-L1 polypeptide extracellular domain.

Aspect 4. The TMP of aspect 3, wherein the PD-L1 polypeptide comprises an amino acid sequence having at least 95% amino acid sequence identity to the PD-L1 amino acid sequence depicted in FIG. 22D and has a length of from about 215 amino acids to about 220 amino acids.

Aspect 5. The TMP of aspect 1 or aspect 2, wherein at least one of the one or more MODs is a 4-1BBL polypeptide.

Aspect 6. The TMP of aspect 5, wherein the 4-1BBL polypeptide comprises an amino acid sequence having at least 95% amino acid sequence identity to the 4-1BBL amino acid sequence depicted in FIG. 22E, and has a length of from about 160 amino acids to about 170 amino acids.

Aspect 7. The TMP of any one of aspects 1-5, wherein at least one of the one or more MODs is a variant IL-2 polypeptide, optionally wherein the one or more variant IL-2 polypeptides exhibit a binding affinity for an IL-2 receptor, and wherein the binding affinity of such one or more variant IL-2 polypeptides is less than the binding affinity of a wild-type human IL-2 polypeptide for the same IL-2 receptor, when assayed under the same conditions in a bio-layer interferometry (BLI) assay.

Aspect 8. The TMP of aspect 7, wherein the at least one variant IL-2 polypeptide exhibits reduced binding to the α chain of IL-2 receptor and/or the β chain of IL-2 receptor, and optionally wherein the at least one variant IL-2 polypeptide exhibits reduced binding to both the α chain of IL-2 receptor and the β chain of IL-2 receptor.

Aspect 9. The TMP of aspect 8, comprising two variant IL-2 polypeptides that exhibits reduced binding to the α chain of IL-2 receptor and the β chain of IL-2 receptor.

Aspect 10. The TMP of aspect 8 or 9, wherein the variant IL-2 polypeptide comprises: i) an H16A substitution and an F42A substitution; or ii) an H16T substitution and an F42A substitution.

Aspect 11. The TMP of aspect 1, wherein at least one of the one or more MODs is a 4-1BBL polypeptide.

Aspect 12. The TMP of any one of aspects 1-11, wherein the T1D peptide has a length of from about 4 amino acids to about 25 amino acids, or from about 8 amino acids to about 20 amino acids, and comprises a proinsulin peptide or a glutamic acid decarboxylase (GAD) peptide.

Aspect 13. The TMP of aspect 12, wherein the T1D peptide is:
a) a proinsulin peptide selected from SLQPLALEGSLQKRG (SEQ ID NO:175; proIns 76-90). SLQPLALEGSLQSRG (SEQ ID NO:159; proIns 76-90; K88S), and GAGSLQPLALEGSLQKRG (SEQ ID NO:176; proIns 73-90); or
b) a GAD peptide selected from NFFRMVISNPAAT (SEQ ID NO:177; GAD65 555-567) and NFIRMVTSNPAAT (SEQ ID NO:163; GAD65 555-567; F557T).

Aspect 14. The TMP of aspect 13, wherein the T1D peptide comprises the amino acid sequence SLQPLALFGSQSRG (SEQ ID NO:159).

Aspect 15. The TMP of any one of aspects 1-14, wherein the masking polypeptide comprises a TGF-β receptor (“TβR”) polypeptide.

Aspect 16. The TMP of aspect 15, wherein the masking polypeptide comprises at least a portion of a type I TβR (TβRI) polypeptide, a type I1 TβR (TβRID polypeptide, or a type III TβR (TβRIII) polypeptide.

Aspect 17. The TMP of aspect 16, wherein the masking polypeptide comprises at least a portion of an ectodomain of a TORI polypeptide, a TβRII polypeptide, or a TβRIII polypeptide.

Aspect 18. The TMP of aspect 17, optionally wherein the masking polypeptide comprises at least a portion of the ectodomain of a TORIT isoform A polypeptide or the ectodomain of a TβRII isoform B polypeptide.

Aspect 19. The TMP of any one of aspects 15-18, wherein the TβR polypeptide comprises one or more sequence variations relative to the corresponding wild type TβR polypeptide, and wherein the TβR polypeptide exhibits reduced affinity for the TGF-β polypeptide relative to the corresponding wild type TβR polypeptide.

Aspect 20. The TMP of aspect 19, wherein the one or more sequence variations are selected from the group consisting of deletions, insertions, substitutions, and combinations thereof.

Aspect 21. The TMP of any one of aspects 15-20, wherein the TGF-β polypeptide comprises at least a portion of a TGF-β 1 polypeptide, a TGF-β2 polypeptide or a TGF-β3 polypeptide.

Aspect 22. The TMP of aspect 21, wherein the TGF-β polypeptide comprises at least a portion of a TGF-β3 polypeptide.

Aspect 23. The TMP of aspect 18 or aspect 19, wherein the TβR polypeptide comprises an amino acid sequence having at least 85% amino acid sequence identity to the amino acid sequence depicted in any one of FIGS. 25B, 25D, and 25F-25J.

Aspect 24. The TMP of aspect 22, wherein the TGF-β3 polypeptide comprises an amino acid sequence having at least 85% amino acid sequence identity to the amino acid sequence depicted in FIG. 23F.

Aspect 25. The TMP of any one of aspects 1-24, wherein the scaffold polypeptide comprises an immunoglobulin (Ig) Fc polypeptide.

Aspect 26. The TMP of aspect 25, wherein the Ig Fc polypeptide is a variant Ig Fc polypeptide comprising one or more sequence variations relative to the wild type polypeptide, wherein the ability of the Ig Fc polypeptide to induce cell lysis though complement-dependent cytotoxicity (CDC) and/or antibody-dependent cellular cytotoxicity (ADCC) is reduced or substantially eliminated.

Aspect 27. The TMP of aspect 26, wherein the Ig Fc polypeptide is a variant human IgG1 Fe polypeptide comprising comprises an L234A and/or L235A substitutions (L14 and L15 in the amino acid sequence depicted in FIG. 21A.

Aspect 28. The TMP of any one of aspects 25-27, wherein the Ig Fc polypeptide is a variant Ig Fc polypeptide comprising at least about 90%, at least about 95%, at least about 98%, at least about 99%, or 100% aa sequence identity to least 125 contiguous aas (at least 150, at least 175, at least 200, or at least 220 contiguous aas) of wt. IgG1 Fc comprising the amino acid sequence depicted in FIG. 21A.

Aspect 29. The TMP of aspect 25, wherein the Ig Fc polypeptide is an Ig Fc polypeptide comprising at least about 90%, at least about 95%, at least about 98%, at least about 99%, or 100% as sequence identity to the amino acid sequence depicted in any one of FIG. 21A-21M.

Aspect 30. The TMP of any one of aspects 1-29, wherein the MHC class II α polypeptide comprises an amino acid sequence having at least 95% amino acid sequence identity to a DRA1*01:01 polypeptide; and the MHC class II β polypeptide comprises an amino acid sequence having at least 95% amino acid sequence identity to a DRB1*04:01 polypeptide.

Aspect 31. The TMP of any one of aspects 1-30, wherein the TMP comprises at least one heterodimer, wherein each heterodimer comprises:
a) a first polypeptide comprising:
   ii the T1D peptide;
   ii) an MHC class II β polypeptide;
   iii) an MHC class II α polypeptide; and
   iv) a first scaffold polypeptide comprising a fast interspecific binding sequence, and
b) a second polypeptide comprising:
   i) the T1D peptide;
   ii) an MHC class II β polypeptide;
   iii) an MHC class II α polypeptide; and
   iv) a second scaffold polypeptide comprising a counterpart interspecific binding sequence to the interspecific binding sequence of the first polypeptide, wherein the interspecific binding sequence and the counterpart interspecific binding sequence interact with each other in the heterodimer, and wherein, the first and/or the second polypeptide comprises the one or more MODs, the first or the second polypeptide comprises the TGF-β polypeptide, and the first or the second polypeptide comprises the masking polypeptide, and the components of the first polypeptide and/or the components of the second polypeptide optionally may be joined by one or more independently selected linkers.

Aspect 32. The TMP of aspect 31, wherein:
a) the TGF-β polypeptide is on the first polypeptide and the masking polypeptide is on the second polypeptide; or
b) the masking polypeptide is on the first polypeptide and the TGF-β polypeptide is on the second polypeptide.

Aspect 33. The TMP of aspect 32, wherein:
the one or more MODs and the TGF-β polypeptide are both on the same polypeptide, or
the one or more MODs and the masking polypeptide are both on the same polypeptide.

Aspect 34. The TMP of aspect 31, wherein the TGF-β polypeptide and the masking polypeptide are both on the same polypeptide.

Aspect 35. The TMP of aspect 34, wherein the one or more MODs are on the same polypeptide as the TGF-β polypeptide and masking polypeptide.

Aspect 36. The TMP of aspect 34, wherein:
a) the one or more MODs are on the first polypeptide and the TGF-β polypeptide and the masking polypeptide are on the second polypeptide; or
b) the TGF-β polypeptide and the masking polypeptide are on the first polypeptide and the one or more MODs are on the second polypeptide.

Aspect 37. The TMP of any one of aspects 31-36, wherein the interspecific binding sequence and the counterpart interspecific binding sequence comprise knob-in-hole sequences.

Aspect 38. The TMP of any one of aspects 1-30, wherein the TMP comprises at least one heterodimer, wherein each heterodimer comprises:
a) a first polypeptide comprising:
   i) the T1D peptide; and
   ii) an MHC class II α chain polypeptide or an MHC class II β chain polypeptide; and
   iii) optionally a linker that links the T1D peptide to the first MHC class II polypeptide; and
b) a second polypeptide comprising i) an MHC class II α chain polypeptide if the first polypeptide comprises an MHC class II β chain polypeptide, or ii) an MHC class II β chain polypeptide if the first polypeptide comprises an MHC class II α chain polypeptide wherein the first and/or the second polypeptide comprises the one or more MODs, the first or the second polypeptide comprises the TGF-β polypeptide, and the first or the second polypeptide comprises the masking polypeptide, and optionally wherein the first polypeptide and the second polypeptide of the heterodimer are covalently linked to one another via at least one disulfide bond.

Aspect 39. The TMP of aspect 38, wherein the first polypeptide comprises an MHC class II β chain polypeptide.

Aspect 40. The TMP of aspect 38, wherein
a1) the first polypeptide comprises:
   i) the T1D peptide;
   ii) an MHC class II polypeptide; and
b) the second polypeptide comprises:
   i) an MHC class II α polypeptide;
   ii) the TGF-β polypeptide;
   iii) the masking polypeptide;
   iv) the one or more MODs; and
   v) an Ig Fc polypeptide, or
a2) the first polypeptide comprises:
   i) the T1D peptide;
   ii) an MHC class II β polypeptide; and
   iii) the TGF-β polypeptide or the masking polypeptide, and
b2) the second polypeptide comprises:
   i) an MHC class II α polypeptide;
   ii) the masking polypeptide if the first polypeptide comprises the TGF-β polypeptide, or the TGF-β polypeptide if the first polypeptide comprises the masking polypeptide;
   iii) the one or more MODs; and
   iv) an Ig Fc polypeptide, or
a3) the first polypeptide comprises:
   i) the T1D peptide;
   ii) an MHC class II β polypeptide;
   iii) the TGF-β polypeptide or the masking polypeptide; and
   iv) an Ig Fc polypeptide, and
b3) the second polypeptide comprises:
   i) an MHC class II α polypeptide;
   ii) the masking polypeptide if the first polypeptide comprises the TGF-β polypeptide, or the TGF-β polypeptide if the first polypeptide comprises the masking polypeptide;
   iii) the one or more MODs, or
a4) the first polypeptide comprises:
   i) the T1D peptide;
   ii) an MHC class II β polypeptide;
   iii) the TGF-β polypeptide or the masking polypeptide; and
   iv) the one or more MODs, and
b4) the second polypeptide comprises:
   i) an MHC class II α polypeptide;
   ii) the masking polypeptide if the first polypeptide comprises the TGF-β polypeptide, or the TGF-β polypeptide if the first polypeptide comprises the masking polypeptide;
   iii) an Ig Fc polypeptide, or
a5) the first polypeptide comprises:
   i) the T1D peptide;
   ii) an MHC class II β polypeptide;
   iii) the TGF-β polypeptide or the masking polypeptide; and
   iv) the one or more MODs; and
   v) an Ig Fc polypeptide
b5) the second polypeptide comprises:
   i) an MHC class II α polypeptide; and
   ii) the masking polypeptide if the first polypeptide comprises the TGF-β polypeptide, or the TGF-β polypeptide if the first polypeptide comprises the masking polypeptide, or
a6) the first polypeptide comprises:
   i) the T1D peptide;
   ii) an MHC class II β polypeptide;
   iii) the TGF-β polypeptide;
   iv) the masking polypeptide;
   v) the one or more MODs; and
   vi) an Ig Fc polypeptide
b6) the second polypeptide comprises:
   i) an MHC class II α polypeptide,
wherein, in any of the above TMPs, the components of the first polypeptide optionally may be joined by one or more independently selected linkers, and where the second polypeptide comprises more than one component, the components of the second polypeptide optionally may be joined by one or more independently selected linkers.

Aspect 41. The TMP of aspect 39 or 40, wherein the TMP comprises a Cys-containing linker between the T1D peptide and the MHC Class H β chain polypeptide, and a disulfide bond is formed between the Cys in the linker and a Cys in the MHC class II α chain polypeptide.

Aspect 42. The TMP of aspect 41, wherein the linker comprises an amino acid sequence selected from (CGGGS)(GGGGS)ₙ (SEQ ID NO:178), (GCGGS)(GGGGS)ₙ (SEQ ID NO:179), (GGCGS)(GCGGS)ₙ (SEQ ID NO:180), (GGGCS)(GGGGS)ₙ (SEQ ID NO:181), and (GGGGC)(GGGGS)ₙ (SEQ ID NO:182), where n is an integer from 1 to 10.

Aspect 43. The TMP of any one of aspects 38-42, wherein the MHC class II α chain polypeptide is a variant MHC class II α chain polypeptide that comprises a non-naturally occurring Cys residue.

Aspect 44. The TMP of aspect 43, wherein the variant MHC class II α chain polypeptide is a variant of a DRA MHC class II polypeptide.

Aspect 45. The TMP of aspect 43 or 44, wherein the non-naturally occurring Cys residue is located at an amino acid residue from amino acids 55 to 110 of the MHC class II α chain polypeptide.

Aspect 46. The TMP of aspect 43 or 44, wherein the MHC class II α chain polypeptide is a variant DRA MHC class II polypeptide comprising a Cys at position 72 or 75 based on the amino acid numbering depicted in FIG. 19A.

Aspect 47. The TMP of aspect 46, wherein the variant DRA MHC class II polypeptide comprises a K75C substitution.

Aspect 48. The TMP of aspect 46, wherein the variant DRA MHC class II polypeptide comprises an 172C substitution.

Aspect 49. The TMP of any one of aspects 38-40, wherein a disulfide bond is formed between a Cys in the MHC class II α chain polypeptide and a Cys in the MHC class II β chain polypeptide.

Aspect 50. The TMP of aspect 49, wherein the MHC class II α chain polypeptide is a variant MHC class II polypeptide that comprises a non-naturally occurring Cys residue, and the MHC class II β chain polypeptide is a variant MHC class II polypeptide that comprises a non-naturally occurring Cys residue.

Aspect 51. The TMP of aspect 49 or 50, wherein the MHC class II β chain polypeptide is a variant DRB MHC class T1 polypeptide that comprises an amino acid substitution selected from the group consisting of P5C, F7C, Q10C, N19C, G20C, H33C. G151C, D152C, and W153C.

Aspect 52. The TMP of any one of aspects 49-51, wherein the MHC class II α chain polypeptide is a variant DRA MHC class II polypeptide that comprises an amino acid substitution selected from E3C, E4C, F12C, G28C, D29C, 172C, K75C, T80C, P81C, 182C, T93C, N94C, and S95C.

Aspect 53. The TMP of aspect 52, wherein:
a) the amino acid substitution in the DRA MHC class II polypeptide is P81C; and the amino acid substitution in the DRB MHC class II polypeptide is F7C;
b) the amino acid substitution in the DRA MHC class II polypeptide is P81C; and the amino acid substitution in the DRB MHC class II polypeptide is P5C;
c) the amino acid substitution in the DRA MHC class II polypeptide is P81C; and the amino acid substitution in the DRB MHC class II polypeptide is H33C;
d) the amino acid substitution in the DRA MHC class II polypeptide is E4C; and the amino acid substitution in the DRB MHC class II polypeptide is N19C;
e) the amino acid substitution in the DRA MHC class II polypeptide is E4C; and the amino acid substitution in the DRB MHC class II polypeptide is G20C;
f) the amino acid substitution in the DRA MHC class II polypeptide is T93C; and the amino acid substitution in the DRB MHC class II polypeptide is Q156C;
g) the amino acid substitution in the DRA MHC class II polypeptide is T93C; and the amino acid substitution in the DRB MHC class II polypeptide is W153C;
h) the amino acid substitution in the DRA MHC class II polypeptide is F12C; and the amino acid substitution in the DRB MHC class II polypeptide is F7C;
i) the amino acid substitution in the DRA MHC class II polypeptide is F12C; and the amino acid substitution in the DRB MHC class II polypeptide is Q10C;
j) the amino acid substitution in the DRA MHC class II polypeptide is T80C; and the amino acid substitution in the DRB MHC class II polypeptide is F7C;
k) the amino acid substitution in the DRA MHC class II polypeptide is T80C; and the amino acid substitution in the DRB MHC class II polypeptide is P5C;
1) the amino acid substitution in the DRA MHC class II polypeptide is T80C; and the amino acid substitution in the DRB MHC class II polypeptide is 182C;
m) the amino acid substitution in the DRA MHC class II polypeptide is 182C; and the amino acid substitution in the DRB MHC class II polypeptide is F7C;
n) the amino acid substitution in the DRA MHC class II polypeptide is I82C; and the amino acid substitution in the DRB MHC class II polypeptide is P5C;
o) the amino acid substitution in the DRA MHC class II polypeptide is 182C; and the amino acid substitution in the DRB MHC class II polypeptide is H33C;
p) the amino acid substitution in the DRA MHC class II polypeptide is G28C; and the amino acid substitution in the DRB MHC class II polypeptide is G151C;
q) the amino acid substitution in the DRA MHC class II polypeptide is G28C; and the amino acid substitution in the DRB MHC class II polypeptide is D152C;
r) the amino acid substitution in the DRA MHC class II polypeptide is G28C; and the amino acid substitution in the DRB MHC class II polypeptide is W153C;
s) the amino acid substitution in the DRA MHC class II polypeptide is D29C; and the amino acid substitution in the DRB MHC class II polypeptide is G151C;
t) the amino acid substitution in the DRA MHC class II polypeptide is D29C; and the amino acid substitution in the DRB MHC class II polypeptide is D152C;
u) the amino acid substitution in the DRA MHC class II polypeptide is D29C; and the amino acid substitution in the DRB MHC class II polypeptide is W153C;
v) the amino acid substitution in the DRA MHC class II polypeptide is N94C; and the amino acid substitution in the DRB MHC class II polypeptide is Q156C;
w) the amino acid substitution in the DRA MHC class II polypeptide is N94C; and the amino acid substitution in the DRB MHC class II polypeptide is N120C;
y) the amino acid substitution in the DRA MHC class II polypeptide is S95C; and the amino acid substitution in the DRB MHC class II polypeptide is Q156C;
7) the amino acid substitution in the DRA MHC class II polypeptide is S95C; and the amino acid substitution in the DRB MHC class II polypeptide is N120C;
aa) the amino acid substitution in the DRA MHC class II polypeptide is E3C; and the amino acid substitution in the DRB MHC class II polypeptide is N19C; or
bb) the amino acid substitution in the DRA MHC class II polypeptide is E3C; and the amino acid substitution in the DRB MHC class II polypeptide is G20C.

Aspect 54. A TMP comprising a homodimer of two heterodimeric TMPs according to any one of aspects 38-53.

Aspect 55. The TMP of any one of aspects 1-30, wherein the TMP comprises a single polypeptide chain.

Aspect 56. The TMP of aspect 55, wherein the single polypeptide chain comprises one or more intrachain disulfide bonds, and wherein the components optionally may be joined by one or more independently selected linkers.

Aspect 57. The TMP of aspect 55 or 56, wherein the single polypeptide chain comprises, from N-terminus to C-terminus:
a)
   i) the T1D peptide;
   ii) an MHC class II D polypeptide;
   iii) an MHC class II α polypeptide;
   iv) the TGF-β polypeptide or the masking polypeptide;
   v) the masking polypeptide or the TGF-β polypeptide;
   vi) an Ig Fc polypeptide; and
   vii) the one or more MODs; or
b)
   i) the T1D peptide;
   ii) an MHC class II β polypeptide;
   iii) an MHC class II α polypeptide;
   iv) the TGF-β polypeptide or the masking polypeptide;
   v) the masking polypeptide or the TGF-β polypeptide;
   vi) the one or more MODs; and
   vii) an Ig Fc polypeptide; or
c)
   i) the T1D peptide;
   ii) an MHC class II β polypeptide;
   iii) an MHC class II α polypeptide;
   iv) the one or more MODs;
   v) the TGF-β polypeptide or the masking polypeptide;
   vi) the masking polypeptide or the TGF-β polypeptide; and
   vii) an Ig Fc polypeptide; or
d)
   i) the T1D peptide;
   ii) an MHC class II β polypeptide;
   iii) an MHC class II α polypeptide;
   iv) the one or more MODs;
   v) an Ig Fc polypeptide;
   vi) the TGF-β polypeptide or the masking polypeptide; and
   vii) the masking polypeptide or the TGF-β polypeptide; or
e)
   i) the T1D peptide;
   ii) an MHC class II β polypeptide;
   iii) an MHC class II α polypeptide;
   iv) an Ig Fc polypeptide;
   v) the one or more MODs;
   vi) the TGF-β polypeptide or the masking polypeptide; and
   vii) the masking polypeptide or the TGF-β polypeptide; or
e)
   i) the T1D peptide;
   ii) an MHC class II β polypeptide;
   iii) an MHC class II α polypeptide;
   iv) the one or more MODs;
   v) the TGF-β polypeptide or the masking polypeptide;
   vi) the masking polypeptide or the TGF-β polypeptide; and
   vii) an Ig Fc polypeptide; or
g)
i) the T1D peptide;
   ii) an MHC class II β polypeptide;
   iii) an MHC class II α polypeptide;
   iv) an Ig Fc polypeptide;
   v) the TGF-β polypeptide or the masking polypeptide;
   vi) the masking polypeptide or the TGF-β polypeptide; and
   vii) the one or more MODs.

Aspect 58. A TMP comprising a homodimer of two single-chain TMPs according to any one of aspects 55-57.

Aspect 59. A nucleic acid comprising a nucleotide sequence encoding the first and/or the second polypeptide of any one of aspects 31-54.

Aspect 60. A nucleic acid comprising a nucleotide sequence encoding the single-chain TMP according to any one of aspects 55-57.

Aspect 61. An expression vector comprising the nucleic acid of aspect 59.

Aspect 62. An expression vector comprising the nucleic acid of aspect 60.

Aspect 63. A genetically modified host cell, wherein the host cell is genetically modified with a nucleic acid of aspect 59, or an expression vector of aspect 61.

Aspect 64. A genetically modified host cell, wherein the host cell is genetically modified with a nucleic acid of aspect 60, or an expression vector of aspect 62.

Aspect 65. A method of making a TMP, the method comprising culturing the genetically modified host cell of aspect 63 in vitro in a culture medium under conditions such that the host cell synthesizes the TMP.

Aspect 66. A method of making a TMP, the method comprising culturing the genetically modified host cell of aspect 64 in vitro in a culture medium under conditions such that the host cell synthesizes the TMP.

Aspect 67. A pharmaceutical composition comprising the TMP of any one of aspects 1-58.

Aspect 68. A method of increasing the number of regulatory T cells (Tregs) in an individual, the method comprising administering to the individual an effective amount of the TMP of any one of aspects 1-58, or the pharmaceutical composition of aspect 67.

Aspect 69. A method of treating type 1 diabetes in an individual, the method comprising administering to the individual an effective amount of the TMP of the TMP of any one of aspects 1-58, or the pharmaceutical composition of aspect 67.

Aspect 70. The TMP of any one of aspects 31-37, wherein the interspecific binding sequence and the counterpart interspecific binding sequence are a pair of interspecific binding sequence and counterpart interspecific binding sequence selected from the group consisting of KiH, KiHs-s, HA-TF, ZW-1, 7.8.60, DD-KK, EW-RVT, EW-RVTs-s, and A107 sequence pairs as depicted in Table 1.

Aspect 71. The TMP of aspect 70, wherein one of the first polypeptide and the second polypeptide comprises an interspecific IgG1 Fc polypeptide (e.g., a polypeptide having at least 90%, at least 95%, at least 98%, or at least 99%, amino acid sequence identity to the amino acid sequence depicted in FIG. 21A) comprising a T366Y substitution, and the other of the first polypeptide and the second polypeptide comprises an interspecific IgG1 Fc polypeptide (e.g., a polypeptide having at least 90%, at least 95%, at least 98%, or at least 99%, amino acid sequence identity to the amino acid sequence depicted in FIG. 21A) comprising a Y407T substitution, or corresponding substitutions in other interspecific Ig Fc polypeptides (e.g., IgA, IgD, IgE, IgG2, IgG3, or IgG4 Fc polypeptide comprising interspecific binding sequences).

Aspect 72. The TMP of aspect 70, wherein one of the first polypeptide and the second polypeptides comprises an interspecific IgG1 Fc polypeptide (e.g., a polypeptide having at least 90%, at least 95%, at least 98%, or at least 99%, amino acid sequence identity to the amino acid sequence depicted in FIG. 21A) comprising a T366W substitution, and the other of the first polypeptide and the second polypeptide comprises an interspecific IgG1 Fc polypeptide (e.g., a polypeptide having at least 90%, at least 95%, at least 98%, or at least 99%, amino acid sequence identity to the amino acid sequence depicted in FIG. 21A) comprising T366S, L368A and Y407V substitutions, or corresponding substitutions in other interspecific Ig Fc polypeptides (e.g., IgA, IgD, IgE, IgG2. IgG3, or IgG4 Fe polypeptide comprising interspecific binding sequences).

Aspect 73. The TMP of aspect 70, wherein one of the first polypeptide and the second polypeptides comprises an interspecific IgG1 Fc polypeptide (e.g., a polypeptide having at least 90%, at least 95%, at least 98%, or at least 99%, amino acid sequence identity to the amino acid sequence depicted in FIG. 21A) comprising Y349C, T366S, L368A, and Y407V substitutions, and the other of the first polypeptide and the second polypeptide comprises an interspecific IgG Fc polypeptide (e.g., a polypeptide having at least 90%, at least 95%, at least 98%, or at least 99%, amino acid sequence identity to the amino acid sequence depicted in FIG. 21A) comprising S354C and T366W substitutions or corresponding substitutions in other interspecific Ig Fc polypeptides (e.g., IgA. IgD. IgE, IgG2, IgG3, or IgG4 Fe polypeptide comprising interspecific binding sequences).

Aspect 74. The TMP of aspect 70, wherein one of the fit polypeptide and the second polypeptides comprises an interspecific IgG1 Fc polypeptide (e.g., a polypeptide having at least 90%, at least 95%, at least 98%, or at least 99%, amino acid sequence identity to the amino acid sequence depicted in FIG. 21A) comprising a T146W substitution, and the other of the first polypeptide and the second polypeptide comprises an interspecific IgG1 Fc polypeptide (e.g., a polypeptide having at least 90%, at least 95%, at least 98%, or at least 99%, amino acid sequence identity to the amino acid sequence depicted in FIG. 21A) comprising T146S, L148A, and Y187V substitutions, or corresponding substitutions in other interspecific Ig Fc polypeptides (e.g., IgA, IgD, IgE, IgG2. IgG3, or IgG4 Fc polypeptide comprising interspecific binding sequences).

Aspect 75. The TMP of aspect 70, wherein one of the first polypeptide and the second polypeptides comprises an interspecific IgG1 Fc polypeptide (e.g., a polypeptide having at least 90%, at least 95%, at least 98%, or at least 99%, amino acid sequence identity to the amino acid sequence depicted in FIG. 21A) comprising T146W and S134C substitutions, and the other of the first polypeptide and the second polypeptide comprises an interspecific IgG1 Fc polypeptide (e.g., a polypeptide having at least 90%, at least 95%, at least 98%, or at least 99%, amino acid sequence identity to the amino acid sequence depicted in FIG. 21A) comprising T146S, L148A, Y187V and Y129C substitutions, or corresponding substitutions in other interspecific Ig Fc polypeptides (e.g., IgA, IgD. IgE, IgG2, IgG3, or IgG4 Fc polypeptide comprising interspecific binding sequences).

Aspect 76. The TMP of aspect 70, wherein:
i) the first polypeptide comprises an Ig Fc polypeptide having S144H and F185A substitutions, and the second polypeptide comprises an Ig Fc polypeptide having Y129T and T174F substitutions; or
ii) the first polypeptide comprises an Ig Fc polypeptide having T130V, L131Y, F185A, and Y187V substitutions, and the second polypeptide comprises an Ig Fc polypeptide having 130V, T146L, K172L, and T174W substitutions; or
iii) the first polypeptide comprises an Ig Fc polypeptide having K1401D, D179M, and Y187A substitutions, and the second polypeptide comprises an Ig Fc polypeptide having E125R, Q127R, T146V, and K189V substitutions; or
iv) the first polypeptide comprises an Ig Fc polypeptide having K189D, and K172D substitutions, and the second polypeptide comprises an Ig Fc polypeptide having D179K and E136K substitutions; or
v) the first polypeptide comprises an Ig Fc polypeptide having K140E and K189W substitutions, and the second polypeptide comprises an Ig Fc polypeptide having Q127R, D179V, and F185T substitutions; or
vi) the first polypeptide comprises an Ig Fc polypeptide having K140E, K189W, and Y129C substitutions, and the second polypeptide comprises an Ig Fc polypeptide having Q127R, D179V, F185T, and S134C substitutions; or
vii) the first polypeptide comprises an Ig Fc polypeptide having K150E and K189W substitutions, and the second polypeptide comprises an Ig Fc polypeptide having E137N, D179V, and F185T substitutions;
wherein the Ig Fc polypeptide comprise an amino acid sequence having at least 80%, 90%, 95%, 98%, 99%, or 100% sequence identity to at least 170 (e.g., at least 180, at least 190, at least 200, at least 210, at least 220, or all 227) contiguous aas of the IgG1 Fc amino acid sequence depicted in FIG. 21A.

Aspect 77. The TMP of any one of aspects 70-76, with none, one, or both of the Ig Fc polypeptides comprise L14 and L15 substitutions and/or an N77 substitution (based on the amino acid numbering of the Ig Fc polypeptide depicted in FIG. 21A).

Aspect 78. The TMP of any one of aspects 1-58 and 70-76, wherein the masking polypeptide is a TGF-8 receptor (“TOR”) polypeptide that comprises an ectodomain fragment of a type 1 (TβRI), a type II (TβRII) or a type III (TβRIII) TβR.

Aspect 79. The TMP of aspect 78, wherein the masking polypeptide is a TORII ectodomain polypeptide comprising an amino acid sequence having at least 60% (e.g., at least 70%, at least 80%, at least 90%, at least 95%, at least 98%, at least 99%, or 100%) amino acid sequence identity to at least 90 (e.g., at least 100, at least 110, at least 120, at least 130, at least 140, at least 150, or 154) contiguous amino acids of the TβRII isoform A ectodomain amino acid sequence depicted in FIG. 25D.

Aspect 80. The TMP of aspect 78, wherein the masking polypeptide is a TβRIII ectodomain polypeptide comprising an amino acid sequence having at least 60% (e.g., at least 70%, at least 80%, at least 90%, at least 95%, at least 98%, at least 99%, or 100%) amino acid sequence identity to at least 90 (e.g., at least 100, at least 110, at least 120, at least 130, at least 140, at least 150, or 154) contiguous amino acids of the TβRII isoform B ectodomain amino acid sequence depicted in FIG. 25F.

Aspect 81. The TMP of aspect 78, wherein the masking polypeptide is a TORII ectodomain polypeptide comprising an amino acid sequence having at least 60% (e.g., at least 70%, at least 80%, at least 90%, at least 95%, at least 98%, at least 99%, or 100%) amino acid sequence identity to at least 90 (e.g., at least 100, at least 110, at least 120, at least 130, at least 140, or 143) contiguous amino acids of the TβRII isoform B ectodomain amino acid sequence depicted in FIG. 25G.

Aspect 82. The TMP of aspect 78, wherein the masking polypeptide is a TβRII octodomain polypeptide comprising an amino acid sequence having at least 60% (e.g., at least 70%, at least 80%, at least 90%, at least 95%, at least 98%, at least 99%, or 100%) amino acid sequence identity to at least 70, at least 80, at least 90, at least 100, at least 110, or 118) contiguous amino acid sequences of: i) the TβRII isoform B ectodomain polypeptide Δ14(delta 14) amino acid sequence depicted in FIG. 25H; or ii) the TβRII isoform B ectodomain polypeptide Δ25(delta 25) amino acid sequence depicted in FIG. 25I; or iii) the TβRII isoform B ectodomain polypeptide Δ25(delta 25) amino acid sequence depicted in FIG. 25J.

Aspect 83. The TMP of any one of aspects 79-82, wherein the TORI ectodomain polypeptide comprises a substitution of one, two, three, four, or five substitutions selected from the group consisting of F30, D32, S52, E55, and D118 (e.g., with alanine or arginine).

Aspect 84. The TMP of aspect 83, wherein the TβRII ectodomain polypeptide comprises: i) a D118A or a D118R substitution; or ii) a D118A or D118R substitution and one, two, three, or four substitutions selected from the group consisting of F30A. D32N, S52L and E55A.

Aspect 85. The TMP of aspect 83 or 84, wherein the TβRII ectodomain polypeptide comprises an N-terminal deletion up to 14 aas (a A14 aa deletion) of the amino acid sequence depicted in FIG. 25F or FIG. 25G (see e.g., FIG. 25H) and a D118 substitution (e.g., D118A or D118R); or an amino acid sequence having at least 60% (e.g., at least 70%, at least 80%, at least 90%, at least 95%, at least 98%, at least 99%, or 100%) aa sequence identity to at least 70 (e.g., at least 80, at least 90, at least 100, at least 110, or 118) amino acids of any of those TβRII polypeptides.

Aspect 86. The TMP of aspect 85, further comprising one, two, three, or four substitutions selected from the group consisting of F30A, D32N, S52L and E55A.

Aspect 87. The TMP of any one of aspects 83-86, wherein the TORII ectodomain polypeptide comprises an N-terminal deletion up 25 aas (a A25 aa deletion) of the amino acid sequence depicted in FIG. 25F or FIG. 25G and a D118 substitution (e.g., D118A or D118R, see SEQ ID NO:148); or a sequence having at least 60% (e.g., at least 70%, at least 80%, at least 90%, at least 95%, at least 98%, at least 99%, or 100%) aa sequence identity to at least 70 (e.g., at least 80, at least 90, or at least 100) contiguous amino acids of any of those TβRII polypeptides.

Aspect 88. The TMP of aspect 87, further comprising one, two, three, or four substitutions selected from the group consisting of F30A. D32N, S52L and E55A.

Aspect 89. The TMP of aspect 78, wherein the masking polypeptide is a TORI ectodomain polypeptide.

Aspect 90. The TMP of aspect 89, wherein the TβRI ectodomain polypeptide comprises an amino acid sequence having at least 60% (e.g., at least 70%, at least 80%, at least 90%, at least 95%, at least 98%, at least 99%, or 100%) amino acid sequence identity to at least 70 (e.g., at least 80, at least 90, or 93) contiguous amino acids of the amino acid sequence depicted in FIG. 25B.

Aspect 91. The TMP of aspect 78, wherein the masking polypeptide is a TβRIII ectodomain polypeptide.

Aspect 92. The TMP of aspect 91, wherein the TβRI III ectodomain polypeptide comprises an amino acid sequence having at least 60% (e.g., at least 70%, at least 80%, at least 90%, at least 95%, at least 98%, at least 99%, or 100%) amino acid sequence identity to at least 70 (e.g., at least 80, 90, 100, 150, 200, 250,300, 400, 500 or 600) contiguous amino acids of the A isoform amino acid sequence depicted in FIG. 25L or the B isoform amino acid sequence depicted in FIG. 25N.

Aspect 93. The TMP of any one of aspects 1-58 and 70-92, wherein the TGF-β polypeptide comprises an amino acid sequence having at least 60% (e.g., at least 70%, at least 80%, at least 80%, at least 85%, at least 90%, at least 95%, at least 98%, at least 99%, or 100%) aa sequence identity to a polypeptide comprising at least 70 (e.g., at least 80, at least 90, at least 100, or at least 110) contiguous amino acids of the mature form of a human TGF-β1 polypeptide (e.g., FIG. 23B), a human TGF-β2 polypeptide (e.g., FIG. 23D), or a human TGF-β3 polypeptide (e.g., FIG. 23F).

Aspect 94. The TMP of aspect 93, wherein the TGF-β polypeptide comprises an amino acid sequence having at least 60%. (e.g., at least 70%, at least 80%, at least 80%, at least 85%, at least 90%, at least 95%, at least 98%, at least 99%, or 100%) amino acid sequence identity to a polypeptide comprising at least 70 (e.g., at least 80, at least 90, at least 100, at least 110, or 112) contiguous amino acids of the amino acid sequence depicted in FIG. 23B (TGFβ1 mature form).

Aspect 95. The TMP of aspect 93, wherein the TGF-β polypeptide comprises an amino acid sequence having at least 60% (e.g., at least 70%, at least 80%, at least 80%, at least 85%, at least 90%, at least 95%, at least 98%, or at least 99%) amino acid sequence identity to a polypeptide comprising at least 70 (e.g., at least 80, at least 90, at least 100, at least 110, or 112) contiguous amino acids of the amino acid sequence depicted in FIG. 23B and comprises a C77S substitution (i.e., the TGF-0 polypeptide comprises a Ser at position 77.

Aspect 96. The TMP of aspect 93, wherein the TGF-β polypeptide comprises an amino acid sequence having at least 60% (e.g., at least 70%, at least 80%, at least 80%, at least 85%, at least 90%, at least 95%, at least 98%, at least 99%, or 100%) amino acid sequence identity to a polypeptide comprising at least 70 (e.g., at least 80, at least 90, at least 100, at least 110, or 112) contiguous amino acids of the amino acid sequence depicted in FIG. 23D (TGFβ2 mature form).

Aspect 97. The TMP of aspect 93, wherein the TGF-β polypeptide comprises an amino acid sequence having at least 60% (e.g., at least 70%, at least 80%, at least 80%, at least 85%, at least 90%, at least 95%, at least 98%, or at least 99%) amino acid sequence identity to a polypeptide comprising at least 70 (e.g., at least 80, at least 90, at least 100, at least 110, or 112) contiguous amino acids of the amino acid sequence depicted in FIG. 23D and comprises a C77S substitution (i.e., the TGF-β polypeptide comprises a Ser at position 77.

Aspect 98. The TMP of aspect 93, wherein the TGF-β polypeptide comprises an amino acid sequence having at least 60% (e.g., at least 70%, at least 80%, at least 80%, at least 85%, at least 90%, at least 95%, at least 98%, at least 99%, or 100%) amino acid sequence identity to a polypeptide comprising at least 70 (e.g., at least 80, at least 90, at least 100, at least 110, or 112) contiguous amino acids of the amino acid sequence depicted in FIG. 23F (TGFβ3 mature form).

Aspect 99. The TMP of aspect 93, wherein the TGF-β polypeptide comprises an amino acid sequence having at least 60% (e.g., at least 70%, at least 80%, at least 80%, at least 85%, at least 90%, at least 95%, at least 98%, or at least 99%) amino acid sequence identity to a polypeptide comprising at least 70 (e.g., at least 80, at least 90, at least 100, at least 110, or 112) contiguous amino acids of the amino acid sequence depicted in FIG. 23F and comprises a C77S substitution (i.e., the TGF-βpolypeptide comprises a Ser at position 77.

Aspect 100. The TMP of any one of aspects 93-99, wherein the TGF-β polypeptide comprises a substitution at one or more of positions 25, 92 and/or 94 of the mature TGF-β polypeptide (e.g., an amino acid sequence depicted in FIG. 23B. FIG. 23D, or FIG. 23F).

Aspect 101. The TMP of aspect 100, wherein the TGF-β polypeptide comprises:
(i) an amino acid other than Lys or Arg at position 25
(ii) an amino acid other than lie or Val at position 92; and/or
(iii) an amino acid other than Lys or Arg at position 94 (e.g., based on the amino acid sequence depicted in FIG. 23B. FIG. 23D, or FIG. 23F).

### Aspects Set B

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A T-cell modulatory polypeptide (TMP) comprising: a) a peptide that displays a Type 1 Diabetes-associated epitope capable of being bound by a T-cell receptor (a “T1D peptide”); b) a TGF-β polypeptide; c) a masking polypeptide, optionally wherein the masking polypeptide comprises a TGF-β receptor polypeptide or an anti-TGF-β polypeptide; d) an MHC class II α chain polypeptide; e) an MHC class IT chain polypeptide; and f) one or more immunomodulatory polypeptides (MOD), wherein optionally the TMP comprises a scaffold polypeptide, wherein optionally the TMP comprises one or more independently selected linker polypeptides, and optionally wherein the T1D peptide has a length of from about 8 amino acids to about 20 amino acids, and comprises a proinsulin peptide or a glutamic acid decarboxylase (GAD) peptide.

Aspect 2. The TMP of aspect 1, wherein at least one of the one or more MODs is a variant IL-2 polypeptide, optionally wherein the one or more variant IL-2 polypeptides exhibit a binding affinity for an IL-2 receptor, and wherein the binding affinity of such one or more variant IL-2 polypeptides is less than the binding affinity of a wild-type human IL-2 polypeptide for the same IL-2 receptor, when assayed under the same conditions in a bio-layer interferometry (BLI) assay, optionally wherein the at least one variant IL-2 polypeptide exhibits reduced binding to both the α chain of IL-2 receptor and the β chain of IL-2 receptor.

Aspect 3. The TMP of aspect 1, wherein the T1D peptide is: a) a proinsulin peptide selected from SLQPLALEGSLQKRG (SEQ ID NO:175; proIns 76-90), SLQPLALEGSLQSRG (SEQ ID NO:159; proIns 76-90; K88S), and GAGSLQPLALEGSLQKRG (SEQ ID NO:176; proIns 73-90); or b) a GAD peptide selected from NFFRMVISNPAAT (SEQ ID NO:177; GAD65 555-567) and NFIRMVISNPAAT (SEQ ID NO:163; GAD65 555-567; F557I).

Aspect 4. The TMP of aspect 1, wherein the masking polypeptide comprises a TGF-β receptor (“TOR”) polypeptide, optionally wherein the masking polypeptide comprises at least a portion of an ectodomain of a type I T3R (TORI) polypeptide, a type II TβR (TβRII) polypeptide, or a type III TβR (TβRII) polypeptide.

Aspect 5. The TMP of aspect 4, wherein the TβR polypeptide comprises one or more sequence variations relative to the corresponding wild type TβR polypeptide, and wherein the TβR polypeptide exhibits reduced affinity for the TGF-β polypeptide relative to the corresponding wild type TOR polypeptide.

Aspect 6. The TMP of aspect 1, wherein the TMP comprises a scaffold polypeptide that is an immunoglobulin (Ig) Fc polypeptide, optionally wherein the Ig Fc polypeptide is a variant Ig Fc polypeptide comprising one or more sequence variations relative to the wild type polypeptide, and wherein the ability of the Ig Fc polypeptide to induce cell lysis though complement-dependent cytotoxicity (CDC) and/or antibody-dependent cellular cytotoxicity (ADCC) is reduced or substantially eliminated.

Aspect 7. The TMP of aspect 1, wherein the MHC class II α polypeptide comprises an amino acid sequence having at least 95% amino acid sequence identity to a DRA1*01:01 polypeptide; and the MHC class III polypeptide comprises an amino acid sequence having at least 95% amino acid sequence identity to a DRB1*04:01 polypeptide.

Aspect 8. The TMP of aspect 1, wherein the TMP comprises at least one heterodimer, wherein each heterodimer comprises: a) a first polypeptide comprising: i) the T1 D peptide; ii) an MHC class II β polypeptide; iii) an MHC class II α polypeptide; and iv) a first scaffold polypeptide comprising a first interspecific binding sequence, and b) a second polypeptide comprising: i) the T1D peptide; ii) an MHC class II β polypeptide; iii) an MHC class II α polypeptide; and iv) a second scaffold polypeptide comprising a counterpart interspecific binding sequence to the interspecific binding sequence of the first polypeptide, wherein the interspecific binding sequence and the counterpart interspecific binding sequence interact with each other in the heterodimer, and wherein, the first and/or the second polypeptide comprises the one or more MODs, the first or the second polypeptide comprises the TGF-β polypeptide, and the first or the second polypeptide comprises the masking polypeptide, and the components of the first polypeptide and/or the components of the second polypeptide optionally may be joined by one or more independently selected linkers.

Aspect 9. The TMP of aspect 1, wherein the TMP comprises at least one heterodimer, wherein each heterodimer comprises: a) a first polypeptide comprising: i) the T1D peptide; and ii) an MHC class II α chain polypeptide or an MHC class II β chain polypeptide; and iii) optionally a linker that links the T1D peptide to the first MHC class IT polypeptide; and b) a second polypeptide comprising i) an MHC class II α chain polypeptide if the first polypeptide comprises an MHC class II β chain polypeptide, or ii) an MHC class II β chain polypeptide if the first polypeptide comprises an MHC class II α chain polypeptide wherein the first and/or the second polypeptide comprises the one or more MODs, the first or the second polypeptide comprises the TGF-β polypeptide, and the first or the second polypeptide comprises the masking polypeptide, and optionally wherein the first polypeptide and the second polypeptide of the heterodimer are covalently linked to one another via at least one disulfide bond.

Aspect 10. The TMP of aspect 8 or 9, wherein the TMP comprises a Cys-containing linker between the T1D peptide and the MHC Class II chain polypeptide, and a disulfide bond is formed between the Cys in the linker and a Cys in the MHC class II α chain polypeptide, optionally wherein the linker comprises an amino acid sequence selected from (CGGGS)(GCGGGS)ₙ (SEQ ID NO:178), (GCGGS)(GGGGS)ₙ (SEQ ID NO:179). (GGCGS)(GGGGS)ₙ (SEQ ID NO:180), (GGGCS)(GGGGS)ₙ (SEQ ID NO:181), and (GGGGC)(GGGGS)ₙ (SEQ ID NO:182), where n is an integer from 1 to 10.

Aspect 11. The TMP of aspect 1, wherein the MHC class II β chain polypeptide is a variant DRB MHC class II polypeptide that comprises an amino acid substitution selected from the group consisting of P5C, F7C, Q10C, N19C, G20C, H33C. G151C, D152C, and W153C, and/or wherein the MHC class 1 α chain polypeptide is a variant DRA MHC class II polypeptide that comprises an amino acid substitution selected from E3C, E4C, F12C, G28C. D29C, 172C, K75C, T80C. P81C, 182C, T93C, N94C, and S95C.

Aspect 12. The TMP of aspect 11, wherein: a) the amino acid substitution in the DRA MHC class II polypeptide is P81C; and the amino acid substitution in the DRB MHC class II polypeptide is F7C; b) the amino acid substitution in the DRA MHC class II polypeptide is P81C; and the amino acid substitution in the DRB MHC class II polypeptide is P5C; c) the amino acid substitution in the DRA MHC class II polypeptide is P81C; and the amino acid substitution in the DRB MHC class II polypeptide is H33C; d) the amino acid substitution in the DRA MHC class II polypeptide is E4C; and the amino acid substitution in the DRB MHC class II polypeptide is N19C; e) the amino acid substitution in the DRA MHC class IT polypeptide is E4C; and the amino acid substitution in the DRB MHC class II polypeptide is G20C; f) the amino acid substitution in the DRA MHC class II polypeptide is T93C; and the amino acid substitution in the DRB MHC class II polypeptide is Q156C; g) the amino acid substitution in the DRA MHC class II polypeptide is T93C; and the amino acid substitution in the DRB MHC class II polypeptide is W153C; h) the amino acid substitution in the DRA MHC class II polypeptide is F12C; and the amino acid substitution in the DRB MHC class II polypeptide is F7C; i) the amino acid substitution in the DRA MHC class II polypeptide is F12C; and the amino acid substitution in the DRB MHC class II polypeptide is Q10C; j) the amino acid substitution in the DRA MHC class II polypeptide is T80C; and the amino acid substitution in the DRB MHC class II polypeptide is F7C; k) the amino acid substitution in the DRA MHC class II polypeptide is T80C; and the amino acid substitution in the DRB MHC class II polypeptide is P5C; 1) the amino acid substitution in the DRA MHC class II polypeptide is T80C; and the amino acid substitution in the DRB MHC class II polypeptide is 182C; m) the amino acid substitution in the DRA MHC class II polypeptide is 182C; and the amino acid substitution in the DRB MHC class II polypeptide is F7C; n) the amino acid substitution in the DRA MHC class II polypeptide is 182C; and the amino acid substitution in the DRB MHC class II polypeptide is P5C; o) the amino acid substitution in the DRA MHC class II polypeptide is 182C; and the amino acid substitution in the DRB MHC class II polypeptide is H33C; p) the amino acid substitution in the DRA MHC class II polypeptide is G28C; and the amino acid substitution in the DRB MHC class II polypeptide is G151C; q) the amino acid substitution in the DRA MHC class II polypeptide is G28C; and the amino acid substitution in the DRB MHC class II polypeptide is D152C; r) the amino acid substitution in the DRA MHC class II polypeptide is G28C; and the amino acid substitution in the DRB MHC class II polypeptide is W153C; s) the amino acid substitution in the DRA MHC class II polypeptide is D29C; and the amino acid substitution in the DRB MHC class II polypeptide is G151C; t) the amino acid substitution in the DRA MHC class II polypeptide is D29C; and the amino acid substitution in the DRB MHC class II polypeptide is D152C; u) the amino acid substitution in the DRA MHC class II polypeptide is D29C; and the amino acid substitution in the DRB MHC class II polypeptide is W153C; v) the amino acid substitution in the DRA MHC class II polypeptide is N94C; and the amino acid substitution in the DRB MHC class II polypeptide is Q156C; w) the amino acid substitution in the DRA MHC class II polypeptide is N94C; and the amino acid substitution in the DRB MHC class II polypeptide is N120C; y) the amino acid substitution in the DRA MHC class IT polypeptide is S95C; and the amino acid substitution in the DRB MHC class II polypeptide is Q156C; z) the amino acid substitution in the DRA MHC class II polypeptide is S95C; and the amino acid substitution in the DRB MHC class II polypeptide is N120C; aa) the amino acid substitution in the DRA MHC class II polypeptide is E3C; and the amino acid substitution in the DRB MHC class II polypeptide is N19C; or bb) the amino acid substitution in the DRA MHC class II polypeptide is E3C; and the amino acid substitution in the DRB MHC class II polypeptide is G20C.

Aspect 13. A TMP comprising a homodimer of two TMPs according to aspect 8 or 9.

Aspect 14. One or more nucleic acids comprising one or more nucleotide sequences encoding the first and/or the second polypeptide of a TMP according to aspect 8 or 9.

Aspect 15. An expression vector comprising the one or more nucleic acids of aspect 14.

Aspect 16. A genetically modified host cell, wherein the host cell is genetically modified with the one or more nucleic acids of aspect 14, or an expression vector of aspect 15.

Aspect 17. A method of making a TMP, the method comprising culturing the genetically modified host cell of aspect 16 in vitro in a culture medium under conditions such that the host cell synthesizes the TMP.

Aspect 18. A pharmaceutical composition comprising the TMP of any one of aspects 1-9.

Aspect 19. A method of increasing the number of regulatory T cells (Tregs) in an individual, the method comprising administering to the individual an effective amount of the pharmaceutical composition of aspect 18.

Aspect 20. A method of treating type 1 diabetes in an individual, the method comprising administering to the individual an effective amount of the pharmaceutical composition of aspect 18.

### EXAMPLES

The following examples are put forth so as to provide those of ordinary skill in the art with a disclosure and description of how to make and use aspects of the present disclosure, and are not intended to limit the scope of the disclosure nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used. e.g., bp, base pair(s); kb, kilobase(s); pl, picoliter(s); s or sec, second(s); min, minute(s); h or hr. hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

The following TMPs were synthesized:
(1) 3858-3859 (FIG. 31A-31B);
(2) 3858-3869 (FIG. 32A-32B;
(3) 3870-3871 (FIG. 33A-33B);
(4) 4415-4416 (FIG. 29A-29B);
(5) 4415-4417 (FIG. 26A-26B);
(6) 4418-4419 (FIG. 30A-30B;
(7) 4418-4420 (FIG. 27A-27B).

Constructs (1)-(3)

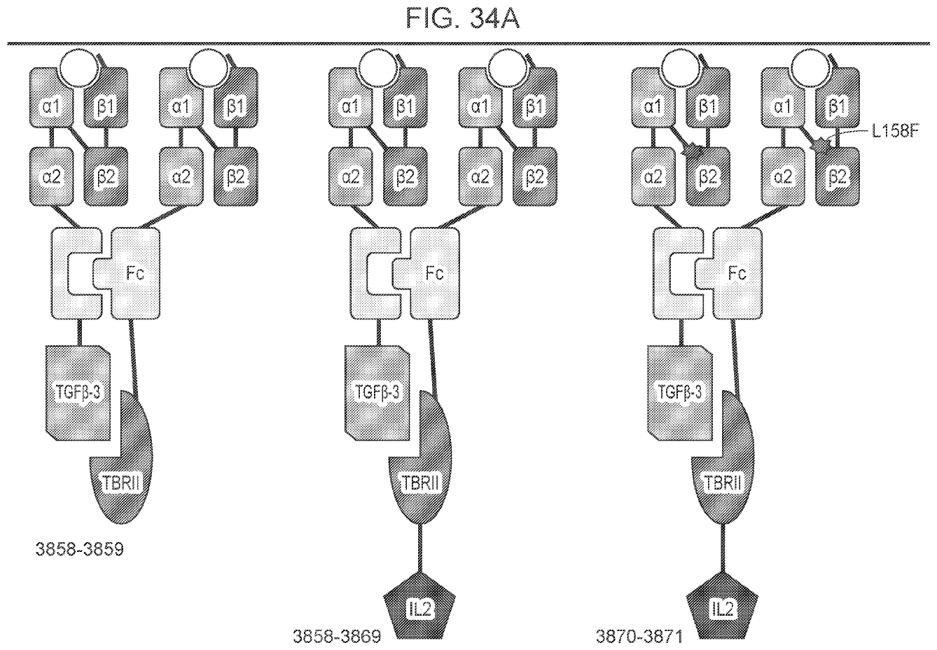

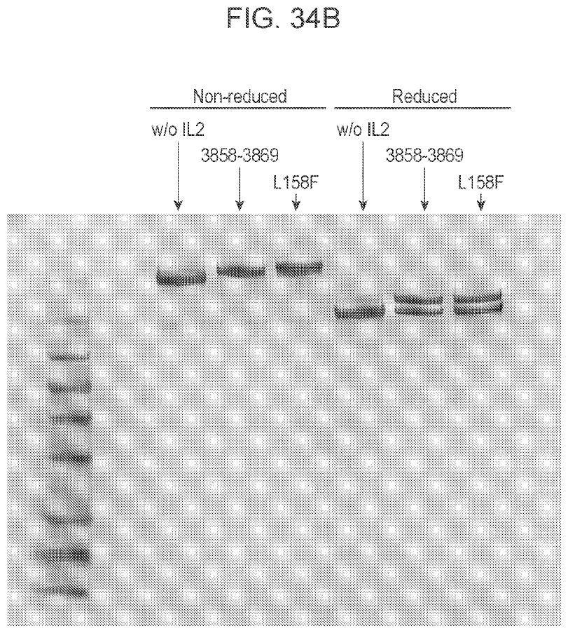

Constructs (1)-(3) are depicted schematically in FIG. 34A. The construct properties are shown in Table 6, below.

TABLE 6

| | 3858-3859 | 3858-3869 | 3870-3871 |
|---|---|---|---|
| Peptide | proIns (76-90; K88S) | proIns (76-90; K88S) | proIns (76-90; K88S) |
| MHC | DR4 single chain | DR4 single chain | DR4 single chain |
| | | | (L158F) |
| TGF-β/TGF-βR; | TGFβ3:TβRII | TGFβ3:TβRII | TGFβ3:TβRII |
| MOD | (no MOD) | IL-2 | IL-2 |
| Harvest Titer (mg/L) | 11.9 | 16.2 | 12.6 |
| Yield (mg/L) | 4.5 | 6.8 | 10.2 |
| aSEC (% monomer) | >95 | >95 | >95 |

The constructs were produced in a mammalian cell line and harvested. The constructs were analyzed on sodium dodecyl sulfate-polyacrylamide gel electrophoresis (SDS-PAGE), under both reducing and non-reducing conditions. The data in Table 6 indicate that TMPs (1)-(3) could be produced in a mammalian cell line. The data shown in FIG. 34B indicate that the constructs, including those containing IL-2, were intact.

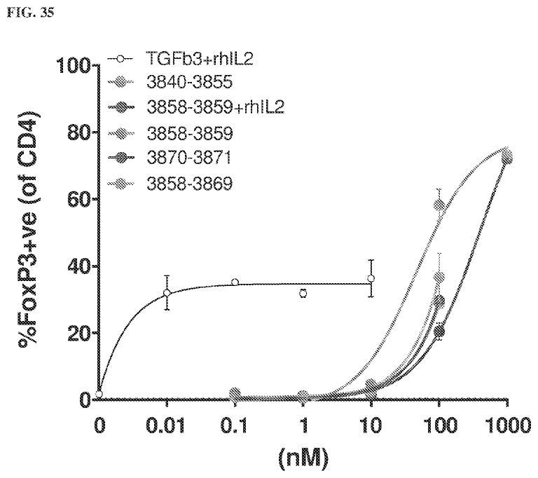

The effect of construct (1)-(3) on induction of FoxP3-positive cell proliferation was tested. As shown in FIG. 35, constructs (2) and (3) induced proliferation of FoxP3-positive cell.

Constructs (4)-(7)

The structure of constructs (5) and (7), which include IL-2 as the MOD), is depicted schematically in FIG. 28. Constructs (4) and (6) have a similar structure, but do not include IL-2.

Constructs (4)-(7) were also produced in a mammalian cell line. Properties of constructs (5) and (7) are provided in Table 7, below.

TABLE 7

| | | | | | | | % | MW |
|---|---|---|---|---|---|---|---|---|
| Lane | construct | epitope | TGFβ | IL-2 | Fc-DS | RT | monomer | kD |
| 1 | 4415-4416 | proIns | + | − | + | 7.473 | 79.1 | 89/88 |
| 2 | 4415-4417 | prolns | + | + | + | 7.255 | 77.8 | 89/105 |
| 3 | 4418-4419 | GAD | + | − | + | 7.478 | 81.2 | 89/88 |
| 4 | 4418-4420 | GAD | + | + | + | 7.28 | 90 | 89/105 |

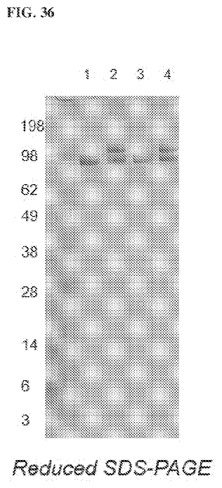

The lane numbers in Table 7 refer to the lanes of the SDS-PAGE results depicted in FIG. 36. “Fc-DS” refers to disulfide-linked Fc. MW refers to the molecular weight of the chains as confirmed by SDS-PAGE. The constructs were analyzed by reducing SDS-PAGE. The results are shown in FIG. 36.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the claims appended hereto.

What is claimed is:

1. A T-cell modulatory polypeptide (TMP) comprising:
a) a peptide that displays a Type 1 Diabetes-associated epitope capable of being bound by a T-cell receptor (a “T1D peptide”);
b) a TGF-β polypeptide;
c) a masking polypeptide, optionally wherein the masking polypeptide comprises a TGF-β receptor polypeptide or an anti-TGF-β polypeptide;
d) an MHC class II α chain polypeptide;
e) an MHC class II β chain polypeptide; and
f) one or more immunomodulatory polypeptides (MOD),
wherein optionally the TMP comprises a scaffold polypeptide,
wherein optionally the TMP comprises one or more independently selected linker polypeptides, and
optionally wherein the T1D peptide has a length of from about 8 amino acids to about 20 amino acids, and comprises a proinsulin peptide or a glutamic acid decarboxylase (GAD) peptide.

2. The TMP of claim 1, wherein at least one of the one or more MODs is a variant IL-2 polypeptide, optionally wherein the one or more variant IL-2 polypeptides exhibit a binding affinity for an IL-2 receptor, and wherein the binding affinity of such one or more variant IL-2 polypeptides is less than the binding affinity of a wild-type human IL-2 polypeptide for the same IL-2 receptor, when assayed under the same conditions in a bio-layer interferometry (BLI) assay, optionally wherein the at least one variant IL-2 polypeptide exhibits reduced binding to both the α chain of IL-2 receptor and the β chain of IL-2 receptor.

3. The TMP of claim 1, wherein the T1D peptide is:
a) a proinsulin peptide selected from SLQPLALEGSLQKRG (SEQ ID NO:175; proIns 76-90), SLQPLALEGSLQSRG (SEQ ID NO:159; proIns 76-90; K885), and GAGSLQPLALEGSLQKRG (SEQ ID NO:176; proIns 73-90); or
b) a GAD peptide selected from NFFRMVISNPAAT (SEQ ID NO:177; GAD65 555-567) and NFIRMVISNPAAT (SEQ ID NO:163; GAD65 555-567; F557I).

4. The TMP of claim 1, wherein the masking polypeptide comprises a TGF-β receptor (“TβR”) polypeptide, optionally wherein the masking polypeptide comprises at least a portion of an ectodomain of a type I TβR (TβRI) polypeptide, a type H TβR (TβRII) polypeptide, or a type III TβR (TβRIII) polypeptide.

5. The TMP of claim 4, wherein the TβR polypeptide comprises one or more sequence variations relative to the corresponding wild type TβR polypeptide, and wherein the TβR polypeptide exhibits reduced affinity for the TGF-β polypeptide relative to the corresponding wild type TβR polypeptide.

6. The TMP of claim 1, wherein the TMP comprises a scaffold polypeptide that is an immunoglobulin (Ig) Fe polypeptide, optionally wherein the Ig Fc polypeptide is a variant Ig Fc polypeptide comprising one or more sequence variations relative to the wild type polypeptide, and wherein the ability of the Ig Fc polypeptide to induce cell lysis though complement-dependent cytotoxicity (CDC) and/or antibody-dependent cellular cytotoxicity (ADCC) is reduced or substantially eliminated.

7. The TMP of claim 1, wherein the MHC class II α polypeptide comprises an amino acid sequence having at least 95% amino acid sequence identity to a DRA1*01:01 polypeptide; and the MHC class II β polypeptide comprises an amino acid sequence having at least 95% amino acid sequence identity to a DRB1*04:01 polypeptide.

8. The TMP of claim 1, wherein the TMP comprises at least one heterodimer, wherein each heterodimer comprises:
a) a first polypeptide comprising:
i) the T1D peptide;
ii) an MHC class II β § polypeptide;
iii) an MHC class II α polypeptide; and
iv) a first scaffold polypeptide comprising a first interspecific binding sequence, and
b) a second polypeptide comprising:
i) the T1D peptide;
ii) an MHC class II polypeptide;
iii) an MHC class II α polypeptide; and
iv) a second scaffold polypeptide comprising a counterpart interspecific binding sequence to the interspecific binding sequence of the first polypeptide, wherein the interspecific binding sequence and the counterpart interspecific binding sequence interact with each other in the heterodimer,
and wherein,
the first and/or the second polypeptide comprises the one or more MODs,
the first or the second polypeptide comprises the TGF-β polypeptide, and
the first or the second polypeptide comprises the masking polypeptide, and
the components of the first polypeptide and/or the components of the second polypeptide optionally may be joined by one or more independently selected linkers.

9. The TMP of claim 1, wherein the TMP comprises at least one heterodimer, wherein each heterodimer comprises:
a) a first polypeptide comprising:
i) the T1D peptide; and
ii) an MHC class II α chain polypeptide or an MHC class II β chain polypeptide; and
iii) optionally a linker that links the T1D peptide to the first MHC class II polypeptide; and
b) a second polypeptide comprising i) an MHC class II α chain polypeptide if the first polypeptide comprises an MHC class II β chain polypeptide, or ii) an MHC class II β chain polypeptide if the first polypeptide comprises an MHC class II α chain polypeptide
wherein
the first and/or the second polypeptide comprises the one or more MODs,
the first or the second polypeptide comprises the TGF-β polypeptide, and
the first or the second polypeptide comprises the masking polypeptide, and
optionally wherein the first polypeptide and the second polypeptide of the heterodimer are covalently linked to one another via at least one disulfide bond.

10. The TMP of claim 8 or 9, wherein
the TMP comprises a Cys-containing linker between the T1D peptide and the MHC Class II β chain polypeptide, and
a disulfide bond is formed between the Cys in the linker and a Cys in the MHC class II α chain polypeptide,
optionally wherein the linker comprises an amino acid sequence selected from (CGGGS)(GGGGS)ₙ (SEQ ID NO:178), (GCGGS)(GGGGS)ₙ (SEQ ID NO:179), (GGCGS)(GGGGS)ₙ (SEQ ID NO:180), (GGGCS)(GGGGS)ₙ (SEQ ID NO:181), and (GGGGC)(GGGGS)ₙ (SEQ ID NO:182), where n is an integer from 1 to 10.

11. The TMP of claim 1, wherein the MHC class II β chain polypeptide is a variant DRB MHC class II polypeptide that comprises an amino acid substitution selected from the group consisting of P5C, F7C, Q10C, N19C, G20C, H33C, G151C, D152C, and W153C, and/or
wherein the MHC class II α chain polypeptide is a variant DRA MHC class II polypeptide that comprises an amino acid substitution selected from E3C, E4C, F12C, G28C, D29C, 172C, K75C, T80C, P81C, 182C, T93C, N94C, and S95C.

12. The TMP of claim 11, wherein:
a) the amino acid substitution in the DRA MHC class II polypeptide is P81C; and the amino acid substitution in the DRB MHC class II polypeptide is F7C;
b) the amino acid substitution in the DRA MHC class II polypeptide is P81C; and the amino acid substitution in the DRB MHC class II polypeptide is P5C;
c) the amino acid substitution in the DRA MHC class II polypeptide is P81C; and the amino acid substitution in the DRB MHC class II polypeptide is H33C;
d) the amino acid substitution in the DRA MHC class II polypeptide is E4C; and the amino acid substitution in the DRB MHC class II polypeptide is N19C;
e) the amino acid substitution in the DRA MHC class II polypeptide is E4C; and the amino acid substitution in the DRB MHC class II polypeptide is G20C;
f) the amino acid substitution in the DRA MHC class II polypeptide is T93C; and the amino acid substitution in the DRB MHC class II polypeptide is Q156C;
g) the amino acid substitution in the DRA MHC class II polypeptide is T93C; and the amino acid substitution in the DRB MHC class II polypeptide is W153C;
h) the amino acid substitution in the DRA MHC class II polypeptide is F12C; and the amino acid substitution in the DRB MHC class II polypeptide is F7C;
i) the amino acid substitution in the DRA MHC class II polypeptide is F12C; and the amino acid substitution in the DRB MHC class II polypeptide is Q10C;
j) the amino acid substitution in the DRA MHC class II polypeptide is T80C; and the amino acid substitution in the DRB MHC class II polypeptide is F7C;
k) the amino acid substitution in the DRA MHC class IT polypeptide is T80C; and the amino acid substitution in the DRB MHC class II polypeptide is P5C;
l) the amino acid substitution in the DRA MHC class II polypeptide is T80C; and the amino acid substitution in the DRB MHC class II polypeptide is 182C;
m) the amino acid substitution in the DRA MHC class II polypeptide is I82C; and the amino acid substitution in the DRB MHC class II polypeptide is F7C;
n) the amino acid substitution in the DRA MHC class II polypeptide is 182C; and the amino acid substitution in the DRB MHC class II polypeptide is P5C;
o) the amino acid substitution in the DRA MHC class II polypeptide is 182C; and the amino acid substitution in the DRB MHC class II polypeptide is H33C;
p) the amino acid substitution in the DRA MHC class II polypeptide is G28C; and the amino acid substitution in the DRB MHC class II polypeptide is G151C;
q) the amino acid substitution in the DRA MHC class II polypeptide is G28C; and the amino acid substitution in the DRB MHC class II polypeptide is D152C;
r) the amino acid substitution in the DRA MHC class II polypeptide is G28C; and the amino acid substitution in the DRB MHC class II polypeptide is W153C;
s) the amino acid substitution in the DRA MHC class II polypeptide is D29C; and the amino acid substitution in the DRB MHC class II polypeptide is G151C;
t) the amino acid substitution in the DRA MHC class II polypeptide is D29C; and the amino acid substitution in the DRB MHC class II polypeptide is D152C;
u) the amino acid substitution in the DRA MHC class II polypeptide is D29C; and the amino acid substitution in the DRB MHC class II polypeptide is W153C;
v) the amino acid substitution in the DRA MHC class II polypeptide is N94C; and the amino acid substitution in the DRB MHC class II polypeptide is Q156C;
w) the amino acid substitution in the DRA MHC class II polypeptide is N94C; and the amino acid substitution in the DRB MHC class II polypeptide is N120C;
y) the amino acid substitution in the DRA MHC class II polypeptide is S95C; and the amino acid substitution in the DRB MHC class II polypeptide is Q156C;
z) the amino acid substitution in the DRA MHC class II polypeptide is S95C; and the amino acid substitution in the DRB MHC class II polypeptide is N120C;
aa) the amino acid substitution in the DRA MHC class II polypeptide is E3C; and the amino acid substitution in the DRB MHC class II polypeptide is N19C; or
bb) the amino acid substitution in the DRA MHC class II polypeptide is E3C; and the amino acid substitution in the DRB MHC class II polypeptide is G20C.

13. A TMP comprising a homodimer of two TMPs according to claim 8 or 9.

14. One or more nucleic acids comprising one or more nucleotide sequences encoding the first and/or the second polypeptide of a TMP according to claim 8 or 9.

15. An expression vector comprising the one or more nucleic acids of claim 14.

16. A genetically modified host cell, wherein the host cell is genetically modified with the one or more nucleic acids of claim 14, or an expression vector of claim 15.

17. A method of making a TMP, the method comprising culturing the genetically modified host cell of claim 16 in vitro in a culture medium under conditions such that the host cell synthesizes the TMP.

18. A pharmaceutical composition comprising the TMP of any one of claims 1-9.

19. A method of increasing the number of regulatory T cells (Tregs) in an individual, the method comprising administering to the individual an effective amount of the pharmaceutical composition of claim 18.

20. A method of treating type 1 diabetes in an individual, the method comprising administering to the individual an effective amount of the pharmaceutical composition of claim 18.


SEQUENCE LISTING (259 sequences)

SEQ ID NO: 1 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 2 - protein, 266 aa, Homo sapiens
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 3 - protein, 266 aa, Homo sapiens [sequence omitted]

SEQ ID NO: 4 - protein, 266 aa, Homo sapiens [sequence omitted]

SEQ ID NO: 5 - protein, 266 aa, Homo sapiens
MVCLRLPGGSCMAVLTVTLMVLSSPLALAGDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKMLSGVGGFVLGLLFLGAGLFIYFRNQKGHSGLQPRGFLS

SEQ ID NO: 6 - protein, 266 aa, Homo sapiens [sequence omitted]

SEQ ID NO: 7 - protein, 266 aa, Homo sapiens [sequence omitted]

SEQ ID NO: 8 - protein, 266 aa, Homo sapiens [sequence omitted]

SEQ ID NO: 9 - protein, 266 aa, Homo sapiens [sequence omitted]

SEQ ID NO: 10 - protein, 266 aa, Homo sapiens
MVCLKFPGGSCMAALTVTLMVLSSPLALAGDTRPRFLEQVKHECHFFNGTERVRFLDRYFYHQEEYVRFDSDVGEYRAVTELGRPDAEYWNSQKDLLEQRRAEVDTYCRHNYGVVESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKMLSGVGGFVLGLLFLGAGLFIYFRNQKGHSGLQPTGFLS

SEQ ID NO: 11 - protein, 266 aa, Homo sapiens [sequence omitted]

SEQ ID NO: 12 - protein, 266 aa, Homo sapiens [sequence omitted]

SEQ ID NO: 13 - protein, 266 aa, Homo sapiens [sequence omitted]

SEQ ID NO: 14 - protein, 266 aa, Homo sapiens [sequence omitted]

SEQ ID NO: 15 - protein, 266 aa, Homo sapiens
MVCLKLPGGSCMAALTVTLMVLSSPLALAGDTQPRFLWQGKYKCHFFNGTERVQFLERLFYNQEEFVRFDSDVGEYRAVTELGRPVAESWNSQKDILEDRRGQVDTVCRHNYGVGESFTVQRRVHPEVTVYPAKTQPLQHHNLLVCSVSGFYPGSIEVRWFRNGQEEKAGVVSTGLIQNGDWTFQTLVMLETVPRSGEVYTCQVEHPSVMSPLTVEWRARSESAQSKMLSGVGGFVLGLLFLGAGLFIYFRNQKGHSGLQPTGFLS

SEQ ID NO: 16 - protein, 266 aa, Homo sapiens [sequence omitted]

SEQ ID NO: 17 - protein, 266 aa, Homo sapiens [sequence omitted]

SEQ ID NO: 18 - protein, 266 aa, Homo sapiens [sequence omitted]

SEQ ID NO: 19 - protein, 266 aa, Homo sapiens [sequence omitted]

SEQ ID NO: 20 - protein, 266 aa, Homo sapiens [sequence omitted]

SEQ ID NO: 21 - protein, 266 aa, Homo sapiens [sequence omitted]

SEQ ID NO: 22 - protein, 266 aa, Homo sapiens
MVCLRLPGGSCMAVLTVTLMVLSSPLALAGDTRPRFLEYSTSECHFFNGTERVRFLDRYFYNQEEYVRFDSDVGEFRAVTELGRPDEEYWNSQKDFLEDERAAVDTYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKMLSGVGGFVLGLLFLGAGLFIYFRNQKGHSGLQPRGFLS

SEQ ID NO: 23 - protein, 266 aa, Homo sapiens [sequence omitted]

SEQ ID NO: 24 - protein, 266 aa, Homo sapiens [sequence omitted]

SEQ ID NO: 25 - protein, 266 aa, Homo sapiens [sequence omitted]

SEQ ID NO: 26 - protein, 266 aa, Homo sapiens [sequence omitted]

SEQ ID NO: 27 - protein, 266 aa, Homo sapiens [sequence omitted]

SEQ ID NO: 28 - protein, 266 aa, Homo sapiens [sequence omitted]

SEQ ID NO: 29 - protein, 266 aa, Homo sapiens [sequence omitted]

SEQ ID NO: 30 - protein, 266 aa, Homo sapiens [sequence omitted]

SEQ ID NO: 31 - protein, 266 aa, Homo sapiens
MVCLKLPGGSCMTALTVTLMVLSSPLALSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 32 - protein, 266 aa, Homo sapiens [sequence omitted]

SEQ ID NO: 33 - protein, 266 aa, Homo sapiens [sequence omitted]

SEQ ID NO: 34 - protein, 266 aa, Homo sapiens [sequence omitted]

SEQ ID NO: 35 - protein, 266 aa, Homo sapiens [sequence omitted]

SEQ ID NO: 36 - protein, 266 aa, Homo sapiens [sequence omitted]

SEQ ID NO: 37 - protein, 266 aa, Homo sapiens
MVCLKLPGGSSLAALTVTLMVLSSRLAFAGDTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSKMLSGVGGFVLGLLFLGAGLFIYFRNQKGHSGLQPTGFLS

SEQ ID NO: 38 - protein, 266 aa, Homo sapiens [sequence omitted]

SEQ ID NO: 39 - protein, 266 aa, Homo sapiens
MVCLKLPGGSSLAALTVTLMVLSSRLAFAGDTRPRFLELLKSECHFFNGTERVRFLERHFHNQEEYARFDSDVGEYRAVRELGRPDAEYWNSQKDLLEQKRGQVDNYCRHNYGVVESFTVQRRVHPQVTVYPAKTQPLQHHNLLVCSVSGFYPGSIEVRWFRNGQEEKAGVVSTGLIQNGDWTFQTLVMLETFPRSGEVYTCQVEHPSVTSPLTVEWSARSESAQSKMLSGVGGFVLGLLFLGAGLFIYFRNQKGHSGLQPTGFLS

SEQ ID NO: 40 - protein, 266 aa, Homo sapiens [sequence omitted]

SEQ ID NO: 41 - protein, 266 aa, Homo sapiens
MVCLKLPGGSCMAALTVTLTVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNSQEEKAGVVSTGLIQNGDWTFQTLVMLETVPRSGEVYTCQVEHPSMMSPLTVQWSARSESAQSKMLSGVGGFVLGLLFLGTGLFIYFRNQKGHSGLQPTGLLS

SEQ ID NO: 42 - protein, 266 aa, Homo sapiens
MVCLKLPGGSCMAALTVTLTVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSKMLSGVGGFVLGLLFLGTGLFIYFRNQKGHSGLQPTGLLS

SEQ ID NO: 43 - protein, 266 aa, Homo sapiens
MVCLKLPGGSYMAKLTVTLMVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRVEPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQSKMLSGVGGFVLGLLFLGAGLFIYFKNQKGHSGLHPTGLVS

SEQ ID NO: 44 - protein, 261 aa, Homo sapiens
MGHEQNQGAALLQMLPLLWLLPHSWAVPEAPTPMWPDDLQNHTFLHTVYCQDGSPSVGLSEAYDEDQLFFFDFSQNTRVPRLPEFADWAQEQGDAPAILFDKEFCEWMIQQIGPKLDGKIPVSRGFPIAEVFTLKPLEFGKPNTLVCFVSNLFPPMLTVNWQHHSVPVEGFGPTFVSAVDGLSFQAFSYLNFTPEPSDIFSCIVTHEIDRYTAIAYWVPRNALPSDLLENVLCGVAFGLGVLGIIVGIVLIIYFRKPCSGD

SEQ ID NO: 45 - protein, 263 aa, Homo sapiens
MITFLPLLLGLSLGCTGAGGFVAHVESTCLLDDAGTPKDFTYCISFNKDLLTCWDPEENKMAPCEFGVLNSLANVLSQHLNQKDTLMQRLRNGLQNCATHTQPFWGSLTNRTRPPSVQVAKTTPFNTREPVMLACYVWGFYPAEVTITWRKNGKLVMPHSSAHKTAQPNGDWTYQTLSHLALTPSYGDTYTCVVEHTGAPEPILRDWTPGLSPMQTLKVSVSAVTLGLGLIIFSLGVISWRRAGHSSYTPLPGSNYSEGWHIS

SEQ ID NO: 46 - protein, 250 aa, Homo sapiens
MALRAGLVLGFHTLMTLLSPQEAGATKADHMGSYGPAFYQSYGASGQFTHEFDEEQLFSVDLKKSEAVWRLPEFGDFARFDPQGGLAGIAAIKAHLDILVERSNRSRAINVPPRVTVLPKSRVELGQPNILICIVDNIFPPVINITWLRNGQTVTEGVAQTSFYSQPDHLFRKFHYLPFVPSAEDVYDCQVEHWGLDAPLLRHWELQVPIPPPDAMETLVCALGLAIGLVGFLVGTVLIIMGTYVSSVPR

SEQ ID NO: 47 - protein, 273 aa, Homo sapiens
MGSGWVPWVVALLVNLTRLDSSMTQGTDSPEDFVIQAKADCYFTNGTEKVQFVVRFIFNLEEYVRFDSDVGMFVALTKLGQPDAEQWNSRLDLLERSRQAVDGVCRHNYRLGAPFTVGRKVQPEVTVYPERTPLLHQHNLLHCSVTGFYPGDIKIKWFLNGQEERAGVMSTGPIRNGDWTFQTVVMLEMTPELGHVYTCLVDHSSLLSPVSVEWRAQSEYSWRKMLSGIAAFLLGLIFLLVGIVIQLRAQKGYVRTQMSGNEVSRAVLLPQSC

SEQ ID NO: 48 - protein, 260 aa, Homo sapiens
MRPEDRMFHIRAVILRALSLAFLLSLRGAGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 49 - protein, 260 aa, Homo sapiens [sequence omitted]

SEQ ID NO: 50 - protein, 258 aa, Homo sapiens [sequence omitted]

SEQ ID NO: 51 - protein, 258 aa, Homo sapiens
MMVLQVSAAPRTVALTALLMVLLTSVVQGRATPENYLFQGRQECYAFNGTQRFLERYIYNREEFVRFDSDVGEFRAVTELGRPDEEYWNSQKDILEEERAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSKTLTGAGGFVLGLIICGVGIFMHRRSKKVQRGSA

SEQ ID NO: 52 - protein, 258 aa, Homo sapiens
MMVLQVSAAPRTVALTALLMVLLTSVVQGRATPENYVYQLRQECYAFNGTQRFLERYIYNREEFVRFDSDVGEFRAVTELGRPDEDYWNSQKDLLEEKRAVPDRVCRHNYELDEAVTLQRRVQPKVNVSPSKKGPLQHHNLLVCHVTDFYPGSIQVRWFLNGQEETAGVVSTNLIRNGDWTFQILVMLEMTPQQGDVYICQVEHTSLDSPVTVEWKAQSDSARSKTLTGAGGFVLGLIICGVGIFMHRRSKKVQRGSA

SEQ ID NO: 53 - protein, 258 aa, Homo sapiens
MMVLQVSAAPRTVALTALLMVLLTSVVQGRATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSKTLTGAGGFVLGLIICGVGIFMHRRSKKVQRGSA

SEQ ID NO: 54 - protein, 258 aa, Homo sapiens [sequence omitted]

SEQ ID NO: 55 - protein, 258 aa, Homo sapiens [sequence omitted]

SEQ ID NO: 56 - protein, 258 aa, Homo sapiens [sequence omitted]

SEQ ID NO: 57 - protein, 258 aa, Homo sapiens [sequence omitted]

SEQ ID NO: 58 - protein, 258 aa, Homo sapiens [sequence omitted]

SEQ ID NO: 59 - protein, 258 aa, Homo sapiens [sequence omitted]

SEQ ID NO: 60 - protein, 258 aa, Homo sapiens [sequence omitted]

SEQ ID NO: 61 - protein, 258 aa, Homo sapiens [sequence omitted]

SEQ ID NO: 62 - protein, 255 aa, Homo sapiens [sequence omitted]

SEQ ID NO: 63 - protein, 255 aa, Homo sapiens
MILNKALLLGALALTTVMSPCGGEDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETVVCALGLSVGLMGIVVGTVFIIQGLRSVGASRHQGPL

SEQ ID NO: 64 - protein, 255 aa, Homo sapiens [sequence omitted]

SEQ ID NO: 65 - protein, 255 aa, Homo sapiens [sequence omitted]

SEQ ID NO: 66 - protein, 254 aa, Homo sapiens
MILNKALMLGALALTTVMSPCGGEDIVADHVASYGVNLYQSYGPSGQFTHEFDGDEEFYVDLERKETVWKLPLFHRLRFDPQFALTNIAVLKHNLNILIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPAPMSELTETVVCALGLSVGLVGIVVGTVLIIRGLRSVGASRHQGPL

SEQ ID NO: 67 - protein, 254 aa, Homo sapiens
MILNKALMLGALALTTVMSPCGGEDIVADHVASYGVNLYQSYGPSGQYSHEFDGDEEFYVDLERKETVWQLPLFRRFRRFDPQFALTNIAVLKHNLNIVIKRSNSTAATNEVPEVTVFSKSPVTLGQPNTLICLVDNIFPPVVNITWLSNGHSVTEGVSTSFLSKSDHSFFKISYLTFLPSADEIYDCKVEHWGLDEPLLKHWEPEIPTPMSELTETVVCALGLSVGLVGIVVGTVLIIRGLRSVGASRHQGPL

SEQ ID NO: 68 - protein, 255 aa, Homo sapiens [sequence omitted]

SEQ ID NO: 69 - protein, 254 aa, Homo sapiens
MILNKALLLGALALTTVMSPCGGEDIVADHVASYGVNLYQSYGPSGQYTHEFDGDEQFYVDLGRKETVWCLPVLRQFRFDPQFALTNIAVTKHNLNILIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPAPMSELTETVVCALGLSVGLVGIVVGTVFIIRGLRSVGASRHQGPL

SEQ ID NO: 70 - protein, 254 aa, Homo sapiens
MILNKALMLGALALTTVMSPCGGEDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 71 - protein, 254 aa, Homo sapiens
MILNKALMLGTLALTTVMSPCGGEDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 72 - protein, 254 aa, Homo sapiens [sequence omitted]

SEQ ID NO: 73 - protein, 255 aa, Homo sapiens
MILNKALLLGALALTAVMSPCGGEDIVADHVASYGVNFYQSHGPSGQYTHEFDGDEEFYVDLETKETVWQLPMFSKFISFDPQSALRNMAVGKHTLEFMMRQSNSTAATNEVPEVTVFSKFPVTLGQPNTLICLVDNIFPPVVNITWLSNGHSVTEGVSETSFLSKSDHSFFKISYLTFLPSADEIYDCKVEHWGLDEPLLKHWEPEIPAPMSELTETLVCALGLSVGLMGIVVGTVFIIQGLRSVGASRHQGLL

SEQ ID NO: 74 - protein, 261 aa, Homo sapiens
MSWKKALRIPGGLRAATVTLMLSMLSTPVAEGRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKMLSGIGGFVLGLIFLGLGLIIHHRSQKGLLH

SEQ ID NO: 75 - protein, 261 aa, Homo sapiens
MSWKKALRIPGGLRAATVTLMLSMLSTPVAEGRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNGQEETAGVVSTPLIRNGDWTFQILVMLEMTPQRGDVYTCHVEHPSLQSPITVEWRAQSESAQSKMLSGIGGFVLGLIFLGLGLIIHHRSQKGLLH

SEQ ID NO: 76 - protein, 261 aa, Homo sapiens
MSWKKALRIPGGLRAATVTLMLAMLSTPVAEGRDSPEDFVYQFKAMCYFTNGTERVRYVTRYIYNREEYARFDSDVEVYRAVTPLGPPDAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQHGDVYTCHVEHPSLQNPITVEWRAQSESAQSKMLSGIGGFVLGLIFLGLGLIIHHRSQKGLLH

SEQ ID NO: 77 - protein, 261 aa, Homo sapiens [sequence omitted]

SEQ ID NO: 78 - protein, 261 aa, Homo sapiens [sequence omitted]

SEQ ID NO: 79 - protein, 261 aa, Homo sapiens [sequence omitted]

SEQ ID NO: 80 - protein, 261 aa, Homo sapiens [sequence omitted]

SEQ ID NO: 81 - protein, 261 aa, Homo sapiens [sequence omitted]

SEQ ID NO: 82 - protein, 261 aa, Homo sapiens [sequence omitted]

SEQ ID NO: 83 - protein, 269 aa, Homo sapiens [sequence omitted]

SEQ ID NO: 84 - protein, 269 aa, Homo sapiens [sequence omitted]

SEQ ID NO: 85 - protein, 269 aa, Homo sapiens
MSWKKALRIPGDLRVATVTLMLAMLSSLLAEGRDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSKMLSGVGGFVLGLIFLGLGLIIRQRSQKGPQGPPPAGLLH

SEQ ID NO: 86 - protein, 227 aa, Homo sapiens
MALQIPGGFWAAAVTVMLVMLSTPVAEARDFPKDFLVQFKGMCYFTNGTERVRGVARYIYNREEYGRFDSDVGEFQAVTELGRSIEDWNNYKDFLEQERAAVDKVCRHNYEAELRTTLQRQVEPTVTISPSRTEALNHHNLLVCSVTDFYPAQIKVRWFRNDQEETAGVVSTSLIRNGDWTFQILVMLEITPQRGDIYTCQVEHPSLQSPITVEWRPRGPPPAGLLH

SEQ ID NO: 87 - protein, 264 aa, Homo sapiens
MALQIPGGFWAAAVTVMLVMLSTPVAEARDFPKDFLVQFKGMCYFTNGTERVRGVARYIYNREEYGRFDSDVGEFQAVTELGRSIEDWNNYKDFLEQERAAVDKVCRHNYEAELRTTLQRQVEPTVTISPSRTEALNHHNLLVCSVTDFYPAQIKVRWFRNDQEETAGVVSTSLIRNGDWTFQILVMLEITPQRGDIYTCQVEHPSLQSPITVEWRAQSESAQSKMLSGIGGFVLGLIFLGLGLIIRHRGQKGPRGPPPAGLLH

SEQ ID NO: 88 - protein, 189 aa, Homo sapiens
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 89 - protein, 189 aa, Homo sapiens
IKCEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 90 - protein, 189 aa, Homo sapiens
IKECHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 91 - protein, 189 aa, Homo sapiens
IKEEHVIIQAECYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 92 - protein, 189 aa, Homo sapiens
IKEEHVIIQAEFYLNPDQSGEFMFDFDCDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 93 - protein, 189 aa, Homo sapiens
IKEEHVIIQAEFYLNPDQSGEFMFDFDGCEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 94 - protein, 189 aa, Homo sapiens
IKEEHVIIQAEFYLNPDQSGEFMFDFDGDEIFHVDMAKKETVWRLEEFGRFASFEAQGALANIAVDKANLECMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 95 - protein, 189 aa, Homo sapiens
IKEEHVIIQAEFYLNPDQSGEFMFDFDGDEIFHVDMAKKETVWRLEEFGRFASFEAQGALANIAVDKANLEIMTCRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 96 - protein, 189 aa, Homo sapiens
IKEEHVIIQAEFYLNPDQSGEFMFDFDGDEIFHVDMAKKETVWRLEEFGRFASFEAQGALANIAVDKANLEIMTKRSNYCPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 97 - protein, 189 aa, Homo sapiens
IKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 98 - protein, 189 aa, Homo sapiens
IKEEHVIIQAEFYLNPDQSGEFMFDFDGDEIFHVDMAKKETVWRLEEFGRFASFEAQGALANIAVDKANLEIMTKRSNYTPCTNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 99 - protein, 189 aa, Homo sapiens
IKEEHVIIQAEFYLNPDQSGEFMFDFDGDEIFHVDMAKKETVWRLEEFGRFASFEAQGALANIAVDKANLEIMTKRSNYTPITNVPPEVTVLCNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 100 - protein, 189 aa, Homo sapiens
IKEEHVIIQAEFYLNPDQSGEFMFDFDGDEIFHVDMAKKETVWRLEEFGRFASFEAQGALANIAVDKANLEIMTKRSNYTPITNVPPEVTVLTCSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 101 - protein, 189 aa, Homo sapiens
IKEEHVIIQAEFYLNPDQSGEFMFDFDGDEIFHVDMAKKETVWRLEEFGRFASFEAQGALANIAVDKANLEIMTKRSNYTPITNVPPEVTVLTNCPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 102 - protein, 220 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 103 - protein, 199 aa, Homo sapiens
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 104 - protein, 199 aa, Homo sapiens
GDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 105 - protein, 199 aa, Homo sapiens
GDTRPRCLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 106 - protein, 199 aa, Homo sapiens
GDTRPRFLECVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 107 - protein, 199 aa, Homo sapiens
GDTRPRFLEQVKHECHFFCGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 108 - protein, 199 aa, Homo sapiens
GDTRPRFLEQVKHECHFFNCTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 109 - protein, 199 aa, Homo sapiens
GDTRPRFLEQVKHECHFFNGTERVRFLDRYFYCQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 110 - protein, 199 aa, Homo sapiens
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNCDWTFQTLVMLETVPRSGEVYTCQVEHPSLTSPLTVEWRARSESAQSKM

SEQ ID NO: 111 - protein, 199 aa, Homo sapiens
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGCWTFQTLVMLETVPRSGEVYTCQVEHPSLTSPLTVEWRARSESAQSKM

SEQ ID NO: 112 - protein, 199 aa, Homo sapiens
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDCTFQTLVMLETVPRSGEVYTCQVEHPSLTSPLTVEWRARSESAQSKM

SEQ ID NO: 113 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 227 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 120 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 122 - protein, 276 aa, Homo sapiens
VTSTLTIKZSDWLGESMFTCRVDHRGLTFQQNASSMCVPDQDTAIRVFAIPPSFASIFLTKSTKLTCLVTDLTTYBSVTISWTREENGAVKTHTNISESHPNATFSAVGEASICEDBDWSGERFTCTVTHTDLPSPLKQTISRPKGVALHRPBVYLLPPARZZLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 123 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 124 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 125 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 126 - protein, 133 aa, Homo sapiens [sequence omitted]

SEQ ID NO: 127 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 128 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 129 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 130 - protein, 219 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 131 - protein, 167 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 132 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 133 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 134 - protein, 413 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSEAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 135 - protein, 112 aa, Homo sapiens
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 136 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 137 - protein, 112 aa, Homo sapiens [sequence omitted]

SEQ ID NO: 138 - protein, 112 aa, Homo sapiens
ALDTNYCFRNLEENCCVRPLYIDFRQDLGWKWVHEPKGYYANFCSGPCPYLRSADTTHSTVLGLYNTLNPEASASPSCVPQDLEPLTILYYVGRTPKVEQLSNMVVKSCKCS

SEQ ID NO: 139 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 140 - protein, 413 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSEAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFRRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 141 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 142 - protein, 93 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 143 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 144 - protein, 154 aa, Homo sapiens
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 145 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 146 - protein, 143 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 147 - protein, 129 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 148 - protein, 114 aa, Homo sapiens
TDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEE

SEQ ID NO: 149 - protein, 104 aa, Homo sapiens
QLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEE

SEQ ID NO: 150 - protein, 111 aa, Homo sapiens
QLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPD

SEQ ID NO: 151 - protein, 851 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 152 - protein, 750 aa, Homo sapiens
CELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPP

SEQ ID NO: 153 - protein, 850 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 154 - protein, 761 aa, Homo sapiens
LCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVM

SEQ ID NO: 155 - protein, 814 aa, synthetic construct
  REGION 1..814: note = synthetic sequence
SLQPLALEGSLQSRGGGGGSGGGGSGGGGSGDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSALDTNYCFRNLEENCCVRPLYIDFRQDLGWKWVHEPKGYYANFCSGPCPYLRSADTTHSTVLGLYNTLNPEASASPSCVPQDLEPLTILYYVGRTPKVEQLSNMVVKSCKCS

SEQ ID NO: 156 - protein, 956 aa, synthetic construct
  REGION 1..956: note = synthetic sequence
SLQPLALEGSLQSRGGGGGSGGGGSGGGGSGDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSQLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPDGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 157 - protein, 812 aa, synthetic construct
  REGION 1..812: note = synthetic sequence
NFIRMVISNPAATGGGGSGGGGSGGGGSGDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSALDTNYCFRNLEENCCVRPLYIDFRQDLGWKWVHEPKGYYANFCSGPCPYLRSADTTHSTVLGLYNTLNPEASASPSCVPQDLEPLTILYYVGRTPKVEQLSNMVVKSCKCS

SEQ ID NO: 158 - protein, 954 aa, synthetic construct
  REGION 1..954: note = synthetic sequence
NFIRMVISNPAATGGGGSGGGGSGGGGSGDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSQLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPDGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 159 - protein, 15 aa, synthetic construct
  REGION 1..15: note = synthetic sequence
SLQPLALEGSLQSRG

SEQ ID NO: 160 - protein, 15 aa, synthetic construct
  REGION 1..15: note = synthetic sequence
GGGGSGGGGSGGGGS

SEQ ID NO: 161 - protein, 25 aa, synthetic construct
  REGION 1..25: note = synthetic sequence
GGGGSGGGGSGGGGSGGGGSGGGGS

SEQ ID NO: 162 - protein, 8 aa, synthetic construct
  REGION 1..8: note = synthetic sequence
GGSAAAGG

SEQ ID NO: 163 - protein, 13 aa, synthetic construct
  REGION 1..13: note = synthetic sequence
NFIRMVISNPAAT

SEQ ID NO: 164 - protein, 803 aa, synthetic construct
  REGION 1..803: note = synthetic sequence
SLQPLALEGSLQSRGGGGGSGGGGSGGGGSGDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSQLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPD

SEQ ID NO: 165 - protein, 801 aa, synthetic construct
  REGION 1..801: note = synthetic sequence
NFIRMVISNPAATGGGGSGGGGSGGGGSGDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSQLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPD

SEQ ID NO: 166 - protein, 809 aa, synthetic construct
  REGION 1..809: note = synthetic sequence
SLQPLALEGSLQSRGGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSALDTNYCFRNLEENCCVRPLYIDFRQDLGWKWVHEPKGYYANFCSGPCPYLRSADTTHSTVLGLYNTLNPEASASPSCVPQDLEPLTILYYVGRTPKVEQLSNMVVKSCKCS

SEQ ID NO: 167 - protein, 798 aa, synthetic construct
  REGION 1..798: note = synthetic sequence
SLQPLALEGSLQSRGGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSQLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPD

SEQ ID NO: 168 - protein, 951 aa, synthetic construct
  REGION 1..951: note = synthetic sequence
SLQPLALEGSLQSRGGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSQLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPDGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 169 - protein, 809 aa, synthetic construct
  REGION 1..809: note = synthetic sequence
SLQPLALEGSLQSRGGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTFVMLETVPRSGEVYTCQVEHPSLTSPLTVEWRARSESAQSKMGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSALDTNYCFRNLEENCCVRPLYIDFRQDLGWKWVHEPKGYYANFCSGPCPYLRSADTTHSTVLGLYNTLNPEASASPSCVPQDLEPLTILYYVGRTPKVEQLSNMVVKSCKCS

SEQ ID NO: 170 - protein, 951 aa, synthetic construct
  REGION 1..951: note = synthetic sequence
SLQPLALEGSLQSRGGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTFVMLETVPRSGEVYTCQVEHPSLTSPLTVEWRARSESAQSKMGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSQLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPDGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 171 - protein, 20 aa, synthetic construct
  REGION 1..20: note = synthetic sequence
GGGGSGGGGSGGGGSGGGGS

SEQ ID NO: 172 - protein, 6 aa, synthetic construct
  REGION 1..6: note = synthetic sequence
LLGGPS

SEQ ID NO: 173 - protein, 226 aa, synthetic construct
  REGION 1..226: note = synthetic sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 174 - protein, 226 aa, synthetic construct
  REGION 1..226: note = synthetic sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 175 - protein, 15 aa, synthetic construct
  REGION 1..15: note = synthetic sequence
SLQPLALEGSLQKRG

SEQ ID NO: 176 - protein, 18 aa, synthetic construct
  REGION 1..18: note = synthetic sequence
GAGSLQPLALEGSLQKRG

SEQ ID NO: 177 - protein, 13 aa, synthetic construct
  REGION 1..13: note = synthetic sequence
NFFRMVISNPAAT

SEQ ID NO: 178 - protein, 55 aa, synthetic construct
  REGION 1..55: note = synthetic sequence
  VARIANT 6..55: note = Each copy of Gly Gly Gly Gly Ser at positions 6 to 55 is either present or absent such that the number of copies ranges from 1 to 10
CGGGSGGGGSGGGGSGGGGSGGGGSGGGGSGGGGSGGGGSGGGGSGGGGSGGGGS

SEQ ID NO: 179 - protein, 55 aa, synthetic construct
  REGION 1..55: note = synthetic sequence
  VARIANT 6..55: note = Each copy of Gly Gly Gly Gly Ser at positions 6 to 55 is either present or absent such that the number of copies ranges from 1 to 10
GCGGSGGGGSGGGGSGGGGSGGGGSGGGGSGGGGSGGGGSGGGGSGGGGSGGGGS

SEQ ID NO: 180 - protein, 55 aa, synthetic construct
  REGION 1..55: note = synthetic sequence
  VARIANT 6..55: note = Each copy of Gly Gly Gly Gly Ser at positions 6 to 55 is either present or absent such that the number of copies ranges from 1 to 10
GGCGSGGGGSGGGGSGGGGSGGGGSGGGGSGGGGSGGGGSGGGGSGGGGSGGGGS

SEQ ID NO: 181 - protein, 55 aa, synthetic construct
  REGION 1..55: note = synthetic sequence
  VARIANT 6..55: note = Each copy of Gly Gly Gly Gly Ser at positions 6 to 55 is either present or absent such that the number of copies ranges from 1 to 10
GGGCSGGGGSGGGGSGGGGSGGGGSGGGGSGGGGSGGGGSGGGGSGGGGSGGGGS

SEQ ID NO: 182 - protein, 55 aa, synthetic construct
  REGION 1..55: note = synthetic sequence
  VARIANT 6..55: note = Each copy of Gly Gly Gly Gly Ser at positions 6 to 55 is either present or absent such that the number of copies ranges from 1 to 10
GGGGCGGGGSGGGGSGGGGSGGGGSGGGGSGGGGSGGGGSGGGGSGGGGSGGGGS

SEQ ID NO: 183 - protein, 40 aa, synthetic construct
  REGION 1..40: note = synthetic sequence
  VARIANT 1..40: note = Each copy of Gly Gly Gly Gly Ser is either present or absent such that the number of copies ranges from 1 to 8
GGGGSGGGGSGGGGSGGGGSGGGGSGGGGSGGGGSGGGGS

SEQ ID NO: 184 - protein, 5 aa, synthetic construct
  REGION 1..5: note = synthetic sequence
AAAGG

SEQ ID NO: 185 - protein, 133 aa, synthetic construct
  REGION 1..133: note = synthetic sequence
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 186 - protein, 133 aa, synthetic construct
  REGION 1..133: note = synthetic sequence
APTSSSTKKTQLQLETLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 187 - protein, 179 aa, Homo sapiens
QLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 188 - protein, 178 aa, Homo sapiens
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 189 - protein, 79 aa, Homo sapiens
VIIQAEFYLNPDQSGEFMFDFDGDEIFHVDMAKKETVWRLEEFGRFASFEAQGALANIAVDKANLEIMTKRSNYTPITN

SEQ ID NO: 190 - protein, 94 aa, Homo sapiens
VPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 191 - protein, 98 aa, Homo sapiens
VPEAPTPMWPDDLQNHTFLHTVYCQDGSPSVGLSEAYDEDQLFFFDFSQNTRVPRLPEFADWAQEQGDAPAILFDKEFCEWMIQQIGPKLDGKIPVSR

SEQ ID NO: 192 - protein, 93 aa, Homo sapiens
GFPIAEVFTLKPLEFGKPNTLVCFVSNLFPPMLTVNWQHHSVPVEGFGPTFVSAVDGLSFQAFSYLNFTPEPSDIFSCIVTHEIDRYTAIAYW

SEQ ID NO: 193 - protein, 85 aa, Homo sapiens
TKADHMGSYGPAFYQSYGASGQFTHEFDEEQLFSVDLKKSEAVWRLPEFGDFARFDPQGGLAGIAAIKAHLDILVERSNRSRAIN

SEQ ID NO: 194 - protein, 94 aa, Homo sapiens
VPPRVTVLPKSRVELGQPNILICIVDNIFPPVINITWLRNGQTVTEGVAQTSFYSQPDHLFRKFHYLPFVPSAEDVYDCQVEHWGLDAPLLRHW

SEQ ID NO: 195 - protein, 85 aa, Homo sapiens
AIKADHVSTYAAFVQTHRPTGEFMFEFDEDEMFYVDLDKKETVWHLEEFGQAFSFEAQGGLANIAILNNNLNTLIQRSNHTQATN

SEQ ID NO: 196 - protein, 94 aa, Homo sapiens
DPPEVTVFPKEPVELGQPNTLICHIDKFFPPVLNVTWLCNGELVTEGVAESLFLPRTDYSFHKFHYLTFVPSAEDFYDCRVEHWGLDQPLLKHW

SEQ ID NO: 197 - protein, 181 aa, Homo sapiens
EDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHW

SEQ ID NO: 198 - protein, 87 aa, Homo sapiens
EDIVADHVASCGVNLYQFYGPSGQYTHEFDGDEQFYVDLERKETAWRWPEFSKFGGFDPQGALRNMAVAKHNLNIMIKRYNSTAATN

SEQ ID NO: 199 - protein, 94 aa, Homo sapiens
EVPEVTVFSKSPVTLGQPNTLICLVDNIFPPVVNITWLSNGQSVTEGVSETSFLSKSDHSFFKISYLTFLPSADEIYDCKVEHWGLDQPLLKHW

SEQ ID NO: 200 - protein, 181 aa, Homo sapiens
EDIVADHVASYGVNFYQSHGPSGQYTHEFDGDEEFYVDLETKETVWQLPMFSKFISFDPQSALRNMAVGKHTLEFMMRQSNSTAATNEVPEVTVFSKFPVTLGQPNTLICLVDNIFPPVVNITWLSNGHSVTEGVSETSFLSKSDHSFFKISYLTFLPSADEIYDCKVEHWGLDEPLLKHW

SEQ ID NO: 201 - protein, 87 aa, Homo sapiens
EDIVADHVASYGVNFYQSHGPSGQYTHEFDGDEEFYVDLETKETVWQLPMFSKFISFDPQSALRNMAVGKHTLEFMMRQSNSTAATN

SEQ ID NO: 202 - protein, 94 aa, Homo sapiens
EVPEVTVFSKFPVTLGQPNTLICLVDNIFPPVVNITWLSNGHSVTEGVSETSFLSKSDHSFFKISYLTFLPSADEIYDCKVEHWGLDEPLLKHW

SEQ ID NO: 203 - protein, 29 aa, Homo sapiens
MVCLKLPGGSSLAALTVTLMVLSSRLAFA

SEQ ID NO: 204 - protein, 19 aa, Homo sapiens
MVCLKLPGGSCMAALTVTL

SEQ ID NO: 205 - protein, 19 aa, Homo sapiens
MVCLKLPGGSYMAKLTVTL

SEQ ID NO: 206 - protein, 94 aa, Homo sapiens
DTRPRFLEQVKHECHFFNGTERVRFLDRYFYHQEEYVRFDSDVGEYRAVTELGRPDAEYWNSQKDLLEQKRAAVDTYCRHNYGVGESFTVQRRV

SEQ ID NO: 207 - protein, 95 aa, Homo sapiens
GDTRCRFLEQVKHECHFFNGTERVRFLDRYFYHQEEYVRFDSDVGEYRAVTELGRPDAEYWNSQKDLLEQKRAAVDTYCRHNYGVGESFTVQRRV

SEQ ID NO: 208 - protein, 103 aa, Homo sapiens
YPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 209 - protein, 104 aa, Homo sapiens
YPEVTVYPAKTQPLQHHNLLVCSVNGFYPASIEVRWFRNGQEEKTGVVSTGLIQNGDCTFQTLVMLETVPRSGEVYTCQVEHPSLTSPLTVEWRARSESAQSKM

SEQ ID NO: 210 - protein, 197 aa, Homo sapiens
DTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSK

SEQ ID NO: 211 - protein, 94 aa, Homo sapiens
DTRPRFLELRKSECHFFNGTERVRYLDRYFHNQEEFLRFDSDVGEYRAVTELGRPVAESWNSQKDLLEQKRGRVDNYCRHNYGVGESFTVQRRV

SEQ ID NO: 212 - protein, 103 aa, Homo sapiens
HPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSK

SEQ ID NO: 213 - protein, 105 aa, Homo sapiens
TVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRV

SEQ ID NO: 214 - protein, 103 aa, Homo sapiens
QPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSK

SEQ ID NO: 215 - protein, 94 aa, Homo sapiens
GGFVAHVESTCLLDDAGTPKDFTYCISFNKDLLTCWDPEENKMAPCEFGVLNSLANVLSQHLNQKDTLMQRLRNGLQNCATHTQPFWGSLTNRT

SEQ ID NO: 216 - protein, 95 aa, Homo sapiens
RPPSVQVAKTTPFNTREPVMLACYVWGFYPAEVTITWRKNGKLVMPHSSAHKTAQPNGDWTYQTLSHLALTPSYGDTYTCVVEHTGAPEPILRDW

SEQ ID NO: 217 - protein, 94 aa, Homo sapiens
TDSPEDFVIQAKADCYFTNGTEKVQFVVRFIFNLEEYVRFDSDVGMFVALTKLGQPDAEQWNSRLDLLERSRQAVDGVCRHNYRLGAPFTVGRK

SEQ ID NO: 218 - protein, 94 aa, Homo sapiens
VQPEVTVYPERTPLLHQHNLLHCSVTGFYPGDIKIKWFLNGQEERAGVMSTGPIRNGDWTFQTVVMLEMTPELGHVYTCLVDHSSLLSPVSVEW

SEQ ID NO: 219 - protein, 186 aa, Homo sapiens
RATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEW

SEQ ID NO: 220 - protein, 92 aa, Homo sapiens
RATPENYLFQGRQECYAFNGTQRFLERYIYNREEFARFDSDVGEFRAVTELGRPAAEYWNSQKDILEEKRAVPDRMCRHNYELGGPMTLQRR

SEQ ID NO: 221 - protein, 94 aa, Homo sapiens
VQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEW

SEQ ID NO: 222 - protein, 94 aa, Homo sapiens
RDSPEDFVFQFKGMCYFTNGTERVRLVTRYIYNREEYARFDSDVGVYRAVTPQGRPDAEYWNSQKEVLEGTRAELDTVCRHNYEVAFRGILQRR

SEQ ID NO: 223 - protein, 94 aa, Homo sapiens
VEPTVTISPSRTEALNHHNLLVCSVTDFYPGQIKVRWFRNDQEETAGVVSTPLIRNGDWTFQILVMLEMTPQRGDVYTCHVEHPSLQSPITVEW

SEQ ID NO: 224 - protein, 182 aa, Homo sapiens
DFLVQFKGMCYFTNGTERVRGVARYIYNREEYGRFDSDVGEFQAVTELGRSIEDWNNYKDFLEQERAAVDKVCRHNYEAELRTTLQRQVEPTVTISPSRTEALNHHNLLVCSVTDFYPAQIKVRWFRNDQEETAGVVSTSLIRNGDWTFQILVMLEITPQRGDIYTCQVEHPSLQSPITVEW

SEQ ID NO: 225 - protein, 93 aa, Homo sapiens
DFLVQFKGMCYFTNGTERVRGVARYIYNREEYGRFDSDVGEFQAVTELGRSIEDWNNYKDFLEQERAAVDKVCRHNYEAELRTTLQRQVEPTV

SEQ ID NO: 226 - protein, 89 aa, Homo sapiens
TISPSRTEALNHHNLLVCSVTDFYPAQIKVRWFRNDQEETAGVVSTSLIRNGDWTFQILVMLEITPQRGDIYTCQVEHPSLQSPITVEW

SEQ ID NO: 227 - protein, 5 aa, synthetic construct
  REGION 1..5: note = synthetic sequence
  VARIANT 4: note = X is any amino acid other than Pro
VPGXG

SEQ ID NO: 228 - protein, 223 aa, Homo sapiens
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 229 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPSCVPQALEPLPIVYYVGRKPKVEQLSNMIVRSCKCS

SEQ ID NO: 230 - protein, 112 aa, Homo sapiens
ALDAAYCFRNVQDNCCLRPLYIDFKRDLGWKWIHEPKGYNANFCAGACPYLWSSDTQHSRVLSLYNTINPEASASPSCVSQDLEPLTILYYIGKTPKIEQLSNMIVKSCKCS

SEQ ID NO: 231 - protein, 114 aa, Homo sapiens
TDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 232 - protein, 104 aa, Homo sapiens
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 233 - protein, 5 aa, synthetic construct
  REGION 1..5: note = synthetic sequence
GSGGS

SEQ ID NO: 234 - protein, 4 aa, synthetic construct
  REGION 1..4: note = synthetic sequence
GGGS

SEQ ID NO: 235 - protein, 4 aa, synthetic construct
  REGION 1..4: note = synthetic sequence
GGSG

SEQ ID NO: 236 - protein, 5 aa, synthetic construct
  REGION 1..5: note = synthetic sequence
GGSGG

SEQ ID NO: 237 - protein, 5 aa, synthetic construct
  REGION 1..5: note = synthetic sequence
GSGSG

SEQ ID NO: 238 - protein, 5 aa, synthetic construct
  REGION 1..5: note = synthetic sequence
GSGGG

SEQ ID NO: 239 - protein, 5 aa, synthetic construct
  REGION 1..5: note = synthetic sequence
GGGSG

SEQ ID NO: 240 - protein, 5 aa, synthetic construct
  REGION 1..5: note = synthetic sequence
GSSSG

SEQ ID NO: 241 - protein, 41 aa, synthetic construct
  REGION 1..41: note = synthetic sequence
  VARIANT 2..41: note = Each copy of Ser Ser Ser Ser at positions 2 to 41 is either present or absent such that the number of copies ranges from 1 to 10
GSSSSSSSSSSSSSSSSSSSSSSSSSSSSSSSSSSSSSSSS

SEQ ID NO: 242 - protein, 20 aa, synthetic construct
  REGION 1..20: note = synthetic sequence
GSSSSGSSSSGSSSSGSSSS

SEQ ID NO: 243 - protein, 25 aa, synthetic construct
  REGION 1..25: note = synthetic sequence
GSSSSGSSSSGSSSSGSSSSGSSSS

SEQ ID NO: 244 - protein, 50 aa, synthetic construct
  REGION 1..50: note = synthetic sequence
GGGGSGGGGSGGGGSGGGGSGGGGSGGGGSGGGGSGGGGSGGGGSGGGGS

SEQ ID NO: 245 - protein, 5 aa, synthetic construct
  REGION 1..5: note = synthetic sequence
GGGGS

SEQ ID NO: 246 - protein, 10 aa, synthetic construct
  REGION 1..10: note = synthetic sequence
GGGGSGGGGS

SEQ ID NO: 247 - protein, 30 aa, synthetic construct
  REGION 1..30: note = synthetic sequence
GGGGSGGGGSGGGGSGGGGSGGGGSGGGGS

SEQ ID NO: 248 - protein, 35 aa, synthetic construct
  REGION 1..35: note = synthetic sequence
GGGGSGGGGSGGGGSGGGGSGGGGSGGGGSGGGGS

SEQ ID NO: 249 - protein, 40 aa, synthetic construct; identical to SEQ ID NO: 183
  REGION 1..40: note = synthetic sequence

SEQ ID NO: 250 - protein, 45 aa, synthetic construct
  REGION 1..45: note = synthetic sequence
GGGGSGGGGSGGGGSGGGGSGGGGSGGGGSGGGGSGGGGSGGGGS

SEQ ID NO: 251 - protein, 17 aa, synthetic construct
  REGION 1..17: note = synthetic sequence
GAGSLQPLALEGSLQKR

SEQ ID NO: 252 - protein, 15 aa, synthetic construct
  REGION 1..15: note = synthetic sequence
GIVDQCCTSICSLYQ

SEQ ID NO: 253 - protein, 15 aa, synthetic construct
  REGION 1..15: note = synthetic sequence
GIVEQCCTSICSLYQ

SEQ ID NO: 254 - protein, 20 aa, synthetic construct
  REGION 1..20: note = synthetic sequence
SFYLKNVQTQETRTLTQFHF

SEQ ID NO: 255 - protein, 18 aa, synthetic construct
  REGION 1..18: note = synthetic sequence
GSLQPLALEGSLQSRGIV

SEQ ID NO: 256 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 257 - protein, 13 aa, synthetic construct
  REGION 1..13: note = synthetic sequence
QPLALEGSLQKRG

SEQ ID NO: 258 - protein, 13 aa, synthetic construct
  REGION 1..13: note = synthetic sequence
QPLALEGSLQSRG

SEQ ID NO: 259 - protein, 50 aa, synthetic construct; identical to SEQ ID NO: 244
  REGION 1..50: note = synthetic sequence